(19) United States
(12) Patent Application Publication
(10) Pub. No.: US 2025/0011868 A1
(43) Pub. Date: Jan. 9, 2025

(54) SMALL RNA PREDICTORS FOR ALZHEIMER'S DISEASE

(71) Applicant: Gatehouse Bio, Inc., Natick, MA (US)

(72) Inventors: David W. SALZMAN, Natick, MA (US); Alan P. Salzman, Natick, MA (US); Neal C. Foster, Natick, MA (US); Nathan S. Ray, Natick, MA (US)

(21) Appl. No.: 18/667,303

(22) Filed: May 17, 2024

Related U.S. Application Data
(63) Continuation of application No. 17/262,045, filed on Jan. 21, 2021, now abandoned.
(60) Provisional application No. 62/703,172, filed on Jul. 25, 2018.

(51) Int. Cl. C12Q 1/6883

(52) U.S. Cl. CPC C12Q 1/6883; C12Q 2600/112; C12Q 2600/158; C12Q 2600/178

(57) ABSTRACT

The present disclosure provides methods and kits for evaluating Alzheimer's disease (AD) activity, including in patients undergoing treatment for AD or a candidate treatment for AD, as well as in animal and cell models. Specifically, the present disclosure provides biomarkers (sRNA predictors) that are binary predictors of disease activity, and are useful for detecting and/or evaluating AD disease stage, grade and progression, prognosis, and response to therapy or candidate therapy. The biomarkers are further useful in the context of drug discovery and clinical trials, to identify candidate pharmaceutical interventions (or other therapies) that are useful for the treatment of disease.

### PRIORITY

This application claims the benefit of, and priority to, U.S. Provisional Application No. 62/703,172, filed Jul. 25, 2018, the contents of which are hereby incorporated by reference in its entirety.

### DESCRIPTION OF THE TEXT FILE SUBMITTED ELECTRONICALLY

The instant application contains a sequence listing, which has been submitted in XML format via EFS-Web. The contents of the XML copy named “SRN-004C1_115987-5004_Sequence_Listing,” which was created on Sep. 23, 2024 and is 569,344 bytes in size, the contents of which are incorporated herein by reference in their entirety.

### BACKGROUND

Alzheimer's disease (AD) is the most common neurodegenerative disease, as it accounts for nearly 70% of all cases of dementia and affects up to 20% of individuals older than 80 years. Various morphological and histological changes in the brain serve as hallmarks of modern day AD neuropathology. Specifically, two neurological phenomena have been observed: amyloid plaques and neurofibrillary tangles. Disease progression can be categorized as Braak stages, with six stages of disease propagation having been distinguished with respect to the location of the tangle-bearing neurons and the severity of changes in the brain: Braak stages I/II: transentorhinal (temporal lobe) stages, clinically silent cases; Braak stages III/IV: limbic stages, incipient Alzheimer's disease; and Braak stages V/VI: neocortical stages, fully developed Alzheimer's disease.

Alzheimer's patients begin presenting early symptoms, such as difficulties with memory like remembering recent events and also forming new memories. Visuospatial and language problems often follow or accompany the onset of early symptoms involving memory. As the disease progresses, individuals slowly lose the ability to perform the activities of daily living, and eventually, attention, verbal ability, problem solving, reasoning, and all forms of memory become seriously impaired. Indeed, progression of AD is often accompanied by changes in personality, such as increased apathy, anger, dependency, aggressiveness, paranoia and occasionally inappropriate sexual behavior. In the latter stages of AD, individuals may be incapable of communication, show signs of complete confusion, and bedridden.

There are two types of Alzheimer's: early-onset and late-onset, and both types have a genetic component. Early-onset AD patients begin to present symptoms between their 30s and mid-60s and is very rare, while late-onset AD, the most common type, see patients presenting signs and symptoms in the patients' mid-60s. Late-onset AD is known to involve a genetic risk factor, a form of apolipoprotein E (APOE), APOE &4, on chromosome 19, that increases a person's risk.

At this time, there is no cure for AD, and available treatments usually offer, at most, a temporary slowing of the symptomatic deterioration. In addition, Alzheimer's can only be absolutely diagnosed after death, by examination of brain tissue and pathology in an autopsy.

Thus, the identification of disease-modifying therapies is the main objective for pharmaceutical intervention and drug discovery. However, these efforts are hampered by the fact that there are no clinically meaningful biomarkers to aid in drug discovery and development. Such biomarkers need to be accessible, prognostic, and/or disease-specific. Discovery and investigation of therapeutic interventions, including pharmaceutical interventions, would benefit from the availability of biomarkers correlative of underlying disease processes.

Diagnostic tests to evaluate Alzheimer's disease activity are needed, for example, to aid treatment and decision making in affected individuals, as well as for use as biomarkers in drug discovery and clinical trials, including for patient enrollment, stratification, and disease monitoring.

### SUMMARY OF THE INVENTION

The present disclosure provides methods and kits for evaluating Alzheimer's disease (AD) activity, including in patients undergoing treatment for AD or a candidate treatment for AD, as well as in animal and cell models. Specifically, the present disclosure provides biomarkers (sRNA predictors) that are binary predictors of disease activity, and are useful for detecting and/or evaluating AD disease stage, grade, progression, prognosis, and response to therapy or candidate therapy. The biomarkers are further useful in the context of drug discovery and clinical trials, to identify candidate pharmaceutical interventions (or other therapies) that are useful for the treatment or management of disease (e.g., treatment or progression monitoring).

In various aspects and embodiments, the invention involves detecting binary small RNA (sRNA) predictors of Alzheimer's disease or Alzheimer's disease activity, in cells or in a biological sample from a subject or patient. The sRNA sequences are identified as being present in samples of an AD experimental cohort, while not being present in any samples of a comparator cohort (“positive sRNA predictors”). The invention thereby detects sRNAs that are binary predictors, exhibiting 100% Specificity for Alzheimer's disease.

In some embodiments, the invention provides a method for evaluating AD activity in a subject or patient. The method comprises providing a biological sample from a subject or patient exhibiting symptoms and signs of AD, and determining the presence, absence, or level of one or more sRNA predictors in the sample. The presence or level of sRNA predictors is correlative with disease activity.

The positive sRNA predictors include one or more sRNA predictors from Table 2A, Table 4A, and Table 7A (SEQ ID NOS: 1-403). For example, the positive sRNA predictors may include one or more sRNA predictors from Table 2A (SEQ ID NOS: 1 to 46), which were identified in sRNA sequence data of brain tissue samples of AD patients, but were absent from non-disease controls, and various other non-Alzheimer's neurodegenerative disease controls (e.g., Parkinson's disease). In some embodiments, the relative or absolute amount of the one or more predictors is correlative with disease stage or severity. In some embodiments, the positive sRNA predictors include one or more sRNA predictors from Table 4A (SEQ ID NOS: 47-254), which were identified in sRNA sequence data of cerebrospinal fluid (CSF) samples of AD patients, but were absent from healthy controls, and various other non-Alzheimer's neurodegenerative disease controls (e.g., Parkinson's disease). In some embodiments, the positive sRNA predictors include one or more sRNA predictors from Table 4A (SEQ ID NOS: 255-403), which were identified in sRNA sequence data of serum samples of AD patients, but were absent from healthy controls, and various other non-Alzheimer's neurodegenerative disease controls (e.g., Parkinson's disease).

In some embodiments, the number of predictors that is present in a sample, or the accumulation of one or more of the predictors, directly correlates with the progression of AD or underlying severity of disease or active symptoms. In some embodiments, the positive sRNA predictors include one or more sRNA predictors from Table 5 (SEQ ID NOS: 58, 189, 78, 172, 193, 97, 122, 215, 248, 164, 120, 93, 126, 253, 112, 144, 213, 244, 123, 222, 150, 240, 52, 220, 221, 169, 165, and 212), which correlate with Braak stages of AD progression (e.g., in CSF samples). In some embodiments, the positive sRNA predictors include one or more from Table 8 (SEQ ID NOS: 257, 270, 272, 273, 279, 286, 288, 314, 319, 325, 332, 341, 374, 391, and 393), which correlate with Braak stages of AD progression (e.g., in serum samples).

In some embodiments, the presence, absence, or level of at least 1, 2, 3, 4, or 5 sRNAs, or at least 10 sRNAs, or at least 40 sRNAs from one or more of Table 2A, Table 4A, and/or Table 7A are determined (SEQ ID NOS: 1-403). In some embodiments, the presence or absence of at least one negative sRNA predictor is also determined, which are identified uniquely in non-AD samples, such as healthy controls. In some embodiments, a panel of sRNAs comprising positive predictors from Table 2A, Table 4A, and/or Table 7A is tested against the sample. In some embodiments, the panel may comprise at least 2, or at least 5, or at least 10, or at least 20, or at least 25 sRNAs from Table 2A, Table 4A, and/or Table 7A. In some embodiments, the panel comprises all sRNAs from Table 2A, Table 4A, and/or Table 7A. For example, a sample may be positive for at least about 2, 3, 4, or 5 sRNA predictors in Table 2A, Table 4A, and/or Table 7A, indicating active disease, with more severe or advanced disease being correlative with about 10, 15 or about 20 sRNA predictors. In some embodiments, the relative or absolute amount of the sRNA predictors in Table 2A, Table 4A, and/or Table 7A are directly correlative with disease grade or severity (e.g., Braak stage).

Generally, the presence of at least 1, 2, 3, 4, or 5 positive predictors is predictive of AD activity. In some embodiments, a panel of 5 to about 100, or about 5 to about 60, sRNA predictors are tested against the sample. . . . While not each experimental sample will be positive for each positive predictor, the panel is large enough to provide 100% Sensitivity against the training cohorts (e.g., the experimental cohort). That is, each sample in the experimental cohort has the presence of one or more positive sRNA predictors. In such embodiments, the presence or absence of the sRNA predictors in the panel provides (by definition) 100% Specificity and 100% Sensitivity against the training set (i.e., the experimental cohort). In still other embodiments, the sRNA predictors are employed in computational classifier algorithms, including non-bootstrapped and/or bootstrapped classification algorithms. Examples including supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naïve Bayes Classifier, Perceptron, Quadratic classifiers, Kernel Estimation, k-Nearest Neighbor, Learning Vector Quantization, and Principal Components Analysis. These classification algorithms may rely on the presence and absence of other sRNAs, other than sRNA predictors. For example, the classifier may rely on the presence of absence of a panel of isoforms (including, but not limited to microRNA isoforms known as ‘isomiRs’), which can optionally include one or more sRNA predictors (i.e., which were identified in sRNA sequence data as unique to a disease condition).

sRNAs can be identified or detected in any biological samples, including solid tissues and/or biological fluids. sRNAs can be identified or detected in animals (e.g., vertebrates and invertebrates), or in some embodiments, cultured cells or the media of cultured cells. For example, the sample may be a biological fluid sample from a human or animal subject (e.g., a mammalian subject), such as blood, serum, plasma, urine, saliva, or cerebrospinal fluid. In some embodiments, the sample is a solid tissue such as brain tissue.

In various embodiments, detection of the sRNAs involves one of various detection platforms, which can employ reverse-transcription, amplification, and/or hybridization of a probe, including quantitative or qualitative PCR, or Real-Time PCR. PCR detection formats can employ stem-loop primers for RT-PCR in some embodiments, and optionally in connection with fluorescently-labeled probes. In some embodiments, sRNAs are detected by a hybridization assay or RNA sequencing (e.g., NextGen sequencing). In some embodiments, RNA sequencing is used in connection with specific primers amplifying the sRNA predictors or other sRNAs in a panel.

The invention involves detection of sRNAs (such as isomiRs) in cells or animals (or samples derived therefrom) that display symptoms and signs of AD. In some embodiments, the invention involves detection of sRNA predictors in cells or animals (or samples derived therefrom) that contain a form of apolipoprotein E (APOE), APOE 84. In various embodiments, the number and/or identity of the sRNA predictors, or the relative amount thereof, is correlative with disease activity for patients, subjects, or cells having a APOE &4 allele. In some embodiments, the sRNA predictor is indicative of AD biological processes in patients or subjects that are otherwise considered Asymptomatic.

In some embodiments, the invention provides a kit comprising a panel of from 2 to about 100 sRNA predictor assays, or from about 5 to about 75 sRNA predictor assays, or from 5 to about 20 sRNA predictor assays. In these embodiments, the kit may comprise sRNA predictor assays (e.g., reagents for such assays) to determine the presence or absence of sRNA predictors from Table 2A, Table 4A, and/or Table 7A. Such assays may comprise reverse transcription (RT) primers, amplification primers and probes (such as fluorescent probes or dual labeled probes) specific for the sRNA predictors over other non-predictive sequences. In some embodiments, the kit is in the form of an array or other substrate containing probes for detection of sRNA predictors by hybridization.

In some aspects, the invention provides kits for evaluating samples for Alzheimer's disease activity. In various embodiments, the kits comprise sRNA-specific probes and/or primers configured for detecting a plurality of sRNAs listed in Table 2A, Table 4A, and/or Table 7A (SEQ ID NOS: 1-403). In some embodiments, the kit comprises sRNA-specific probes and/or primers configured for detecting at least 5, or at least 10, or at least 20, or at least 40 sRNAs listed in Table 2A, Table 4A, and/or Table 7A (SEQ ID NOS: 1-403).

In still other embodiments, the invention involves constructing disease classifiers based on the presence or absence of particular sRNA molecules (e.g., isomiRs or other types of sRNAs). These disease classifiers are powerful tools for discriminating disease conditions that present with similar symptoms, as well as determining disease subtypes, including predicting the course of the disease, predicting response to treatment, and disease monitoring. Generally, sRNA panels (e.g., panels of distinct sRNA variants) will be determined from sequence data in one or more training sets representing one or more disease conditions of interest. sRNA panels and the classifier algorithm can be constructed using, for example, supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naïve Bayes Classifier, Perceptron, Quadratic classifiers, Kernel Estimation, k-Nearest Neighbor, Learning Vector Quantization, and Principal Components Analysis. Once the classifier is trained, independent subjects can be evaluated for the disease conditions by detecting the presence or absence, in a biological sample from the subject, of the sRNA markers in the panel, and applying the classification algorithm. Classifiers can be binary classifiers (i.e., classify among two conditions), or may classify among three, four, five, or more disease conditions. The classifiers rely on the presence and absence of sRNAs in the panel, rather than discriminating normal and abnormal levels of sRNAs.

For example, in some embodiments, the invention provides a method for evaluating a subject for one or more disease conditions. The method comprises providing a biological sample of the subject, and determining the presence or absence of a plurality of sRNAs in the sRNA panel. This profile of “present and absent” sRNAs (binary markers) is used to classify the condition of the subject among two or more disease conditions using the disease classifier. The disease classifier will have been trained based on the presence and absence of the sRNAs in the sRNA panel in a set of training samples. For example, the training samples are annotated as positive or negative for the one or more disease conditions (and may be annotated for disease subtype, grade, or treatment regimen), as well as the presence or absence (and in some embodiment, level) of the sRNAs in the panel.

The presence or absence of the sRNAs in the panel is determined in the training set from sRNA sequence data. That is, individual sRNA sequences are identified in the sRNA sequence data by trimming 3′ sequencing adaptors and without consolidating sRNA sequence variants to a reference sequence or genetic locus. For example, after trimming, the unique sequence reads within each disease condition or comparator condition are compiled (i.e., a read count for each unique sequence is prepared). Thus, the presence or absence of specific sRNA sequences, such as isomiRs, are determined in each disease condition, and these variants are not consolidated to reference sequences. These sequences can be used as “binary” markers, that is, evaluated based on their presence or absence in samples, as opposed to discriminating normal and abnormal levels.

Once identified in the sequence data, and selected for inclusion in the computational classifier, molecular detection reagents for the sRNAs in the panel can be prepared. Such detection platforms include quantitative RT-PCR assays, including those employing stem loop primers and fluorescent probes.

Other aspects and embodiments of the invention will be apparent from the following detailed description.

### BRIEF DESCRIPTION OF THE DRAWINGS

FIGS. 1A, 1B, 1C and 1D depict ROC/AUC curves for the various IBD classes and controls: Control (1A), Crohn's disease (1B), Ulcerative colitis (1C), and Diverticular disease (1D).

FIG. 2 depicts a heat map showing the proportion of accurate multi-class disease predictions against their true reference identies.

### DESCRIPTION OF THE TABLES

Tables 1A to 1B characterize brain tissue sample cohorts, including Alzheimer's disease (AD) cohort (Table 1A), and control cohort including healthy control and various other non-Alzheimer's neurological disorder controls (Table 1B).

Tables 2A shows sRNA positive predictors in brain tissue samples for AD (SEQ ID NOs: 1-46) with read count, specificity, and sensitivity (e.g., frequency). Table 2B shows positive predictors for AD across brain tissue samples, with number of biomarkers per sample and percent coverage.

Tables 3A to 3B characterize cerebrospinal fluid (CSF) sample cohorts, including Alzheimer's disease (AD) cohort (Table 3A), and control cohort including healthy control and various other non-Alzheimer's neurological disorder controls (Table 3B).

Table 4A shows sRNA positive predictors in CSF for AD (SEQ ID NOs: 47-254) with read count, specificity, and sensitivity (e.g., frequency). Table 4B shows positive predictors for AD across CSF samples, with number of biomarkers per sample and percent coverage.

Table 5 shows a panel of 28 identified sRNA biomarkers from CSF that show correlation to Braak Stage that can be used in the monitoring of AD.

Tables 6A to 6B characterize serum sample cohorts, including Alzheimer's disease (AD) cohort (Table 6A), and control cohort including healthy control and various other non-Alzheimer's neurological disorder controls (Table 6B).

Table 7A shows sRNA positive predictors in serum for AD (SEQ ID NOs: 255-403) with read count, specificity, and sensitivity (e.g., frequency). Table 7B shows positive predictors for AD across serum samples, with number of biomarkers per sample and percent coverage.

Table 8 shows a panel of 15 identified sRNA biomarkers from serum that show correlation to Braak Stage that can be used in the monitoring of AD.

Table 9 depicts a panel of sRNA biomarkers from colon epithelium tissue for Controls (“Normal” individuals) of Inflammatory Bowel Disease.

Table 10 shows a panel of sRNA biomarkers from colon epithelium tissue for Crohn's disease.

Table 11 shows a panel of sRNA biomarkers from colon epithelium tissue for Ulcerative colitis.

Table 12 depicts a panel of sRNA biomarkers from colon epithelium tissue for Diverticular disease.

### DETAILED DESCRIPTION OF THE INVENTION

The present disclosure provides methods and kits for evaluating Alzheimer's disease (AD) activity, including in patients undergoing treatment for AD or a candidate treatment for AD, as well as in animal and cell models. Specifically, the present disclosure provides biomarkers (sRNA predictors) that are binary predictors of disease activity, and are useful for detecting and/or evaluating underlying disease processes, disease grade, progression, and response to therapy or candidate therapy. The biomarkers are further useful in the context of drug discovery and clinical trials, to identify candidate therapies that are useful for treatment of AD or AD symptoms, as well as to select or stratify patients, and monitor disease progression or treatment.

In various aspects and embodiments, the invention involves detecting binary small RNA (sRNA) predictors of Alzheimer's disease or Alzheimer's disease activity, in a cell or biological sample. The sRNA sequences are identified as being present in samples of an AD experimental cohort, while not being present in any samples in a comparator cohort. These sRNA markers are termed “positive sRNA predictors”, and by definition provide 100% Specificity. In some embodiments, the method further comprises detecting one or more sRNA sequences that are present in one or more samples of the comparator cohort, and which are not present in any of the samples of the experimental cohort. These predictors are termed “negative sRNA predictors”, and provide additional level of confidence to the predictions. In contrast to detecting dysregulated sRNAs (such as miRNAs that are up- or down-regulated), the invention provides sRNAs that are binary predictors for Alzheimer's disease activity.

small RNA species (“sRNAs”) are non-coding RNAs less than 200 nucleotides in length, and include microRNAs (miRNAs) (including iso-miRs), Piwi-interacting RNAs (piRNAs), small interfering RNAs (siRNAs), vault RNAs (vtRNAs), small nucleolar RNAs (snoRNAs), transfer RNA-derived small RNAs (tsRNAs), ribosomal RNA-derived small RNA fragments (rsRNAs), small rRNA-derived RNAs (srRNA), and small nuclear RNAs (U-RNAs), as well as novel uncharacterized RNA species. Generally, “iso-miR” refers to those sequences that have variations with respect to a reference miRNA sequence (e.g., as used by miRBase). In miRBase, each miRNA is associated with a miRNA precursor and with one or two mature miRNA (−5p and −3p). Deep sequencing has detected a large amount of variability in miRNA biogenesis, meaning that from the same miRNA precursor many different sequences can be generated. There are four main variations of iso-miRs: (1) 5′ trimming, where the 5′ cleavage site is upstream or downstream from the referenced miRNA sequence; (2) 3′ trimming, where the 3′ cleavage site is upstream or downstream from the reference miRNA sequence; (3) 3′ nucleotide addition, where nucleotides are added to the 3′ end of the reference miRNA; and (4) nucleotide substitution, where nucleotides are changed from the miRNA precursor.

U.S. 2018/0258486, filed on Jan. 23, 2018, and PCT/US2018/014856 filed Jan. 23, 2018 (the full contents of which are hereby incorporated by reference), disclose processes for identifying sRNA predictors. The process includes computational trimming of 3′ adapters from RNA sequencing data, and sorting data according to unique sequence reads.

In some embodiments, the invention provides a method for evaluating Alzheimer's disease (AD) activity. The method comprises providing a cell or biological sample from a subject or patient presenting symptoms and signs of AD, or providing RNA extracted therefrom, and determining the presence or absence of one or more sRNA predictors in the cell or sample. The presence of the one or more sRNA predictors is indicative of Alzheimer's disease activity.

The term “Alzheimer's disease activity” refers to active disease processes that result (directly or indirectly) in AD symptoms and overall decline in cognition, behavior, and/or motor skills and coordination. The term Alzheimer's disease activity can further refer to the relative health of affected cells. In some embodiments, the AD activity is indicative of neuron viability.

The positive sRNA predictors include one or more sRNA predictors from Tables 2A, 4A, or 7A (SEQ ID NOS: 1-403). Sequences disclosed herein are shown as the reverse transcribed DNA sequence. For example, the positive sRNA predictors may include one or more sRNA predictors from Table 2A (SEQ ID NOS: 1-46), which are indicative of AD and/or AD stage, as identified in sequence data of brain tissue samples. In some embodiments, the positive sRNA predictors include one or more sRNA predictors from Table 4A (SEQ ID NOS: 47 to 154), which are indicative of AD and/or AD stage, as identified in sequence data of CSF samples. In some embodiments, the positive sRNA predictors include one or more from Table 7A (SEQ ID NOS: 155-403), which are indicative of AD and/or AD stage, as identified in sequence data of serum samples.

Specifically, Tables 2A and 2B show sRNA positive predictors for AD, as identified in brain tissue samples. These sRNA predictors were present in a cohort of AD brain tissue samples (as the Experimental Group), but were not present in any of the Comparator Group samples, which were comprised of non-disease samples, as well as various other non-Alzheimer's neurological disease samples. Table 2A shows positive predictors for AD regardless of Braak stage. The positive predictors each provides 100% Specificity for the presence of AD in the cohort. Tables 2A and 2B shows the average read count across AD brain tissue samples for the positive predictors. In some embodiments, the number of predictors that is present in a sample directly correlates with the Braak stage of AD.

Tables 4A and 4B show sRNA positive predictors for AD, as identified in cerebrospinal fluid (CSF) samples. These sRNA predictors were present in a cohort of AD CSF samples (as the Experimental Group), but were not present in any of the Comparator Group samples, which were comprised of Healthy samples, as well as various other non-Alzheimer's neurological disease samples. Table 4A shows positive predictors for AD regardless of Braak stage. The positive predictors each provides 100% Specificity for the presence of AD in the cohort. Tables 4A and 4B shows the average read count across AD CSF samples for the positive predictors. In some embodiments, the number of predictors that is present in a sample directly correlates with the Braak stage of AD.

Tables 7A and 7B show sRNA positive predictors for AD, as identified in serum samples. These sRNA predictors were present in a cohort of AD serum samples (as the Experimental Group), but were not present in any of the Comparator Group samples, which were comprised of Healthy samples, as well as various other non-Alzheimer's neurological disease samples. Table 7A shows positive predictors for AD regardless of Braak stage. The positive predictors each provides 100% Specificity for the presence of AD in the cohort. Tables 7A and 7B shows the average read count across AD serum samples for the positive predictors. In some embodiments, the number of predictors that is present in a sample directly correlates with the Braak stage of AD.

In various embodiments, the presence, absence, or level of at least five sRNAs are determined, including positive and negative predictors and other potential controls. In some embodiments, the presence or absence of at least 8 sRNAs, or at least 10 sRNAs, or at least about 50 sRNAs are determined. The total number of sRNAs determined, in some embodiments, is less than about 1000 or less than about 500, or less than about 200, or less than about 100, or less than about 50. Therefore, the presence, absence, or level of sRNAs can be determined using any number of specific molecular detection assays.

In some embodiments, the presence, absence, or level of at least 2, or at least 5, or at least 10 sRNAs from Table 2A, Table 4A, and/or Table 7A are determined (SEQ ID NOS: 1-403). In some embodiments, the presence, absence, or level of at least one negative sRNA predictor is also determined. In some embodiments, a panel of sRNAs comprising positive predictors from Table 2A are determined, and the panel may comprise at least 2, at least 5, at least 10, or at least 20 sRNAs from Table 2A. In some embodiments, the panel comprises all sRNAs from Table 2A. In some embodiments, a panel of sRNAs comprising positive predictors from Table 4A are determined, and the panel may comprise at least 2, at least 5, at least 10, or at least 20 sRNAs from Table 4A. In some embodiments, the panel comprises all sRNAs from Table 4A. In some embodiments, a panel of sRNAs comprising positive predictors from Table 7A are determined, and the panel may comprise at least 2, at least 5, at least 10, or at least 20 sRNAs from Table 7A. In some embodiments, the panel comprises all sRNAs from Table 7A.

In some embodiments, the one or more (or all) positive sRNA predictors are each present in at least about 10% of AD samples in the experimental cohort, or at least about 20% of AD samples in the experimental cohort, or at least about 30% of AD samples in the experimental cohort, or at least about 40% of AD samples in the experimental cohort. In some embodiments, the identity and/or number of predictors identified correlates with active disease processes (e.g., Braak stage). For example, a sample may be positive for at least 1, 2, 3, 4, or 5 sRNA predictors in Tables 2A, 4A, and/or 7A, indicating disease from brain tissue, CSF, and/or serum samples, with more severe or advanced disease processes being correlative with about 10, or at least about 15, or at least about 20 sRNA predictors in Table 4A or 7A. In some embodiments, the absolute level (e.g., sequencing read count) or relative level (e.g., using a qualitative assay such as Real Time PCR) is determined for the sRNA predictors in Table 4A or Table 7A, which can be correlative with Braak stage.

In some embodiments, samples that test negative for the presence of the positive sRNA predictors, test positive for at least 1, or at least about 5, or at least about 10, or at least about 20, or at least about 30, or at least about 40, or at least about 50, or at least about 100 negative sRNA predictors. Negative predictors can be specific for healthy individuals or other disease states (such as PD or dementia). Individuals testing positive for AD, will typically not test positive for the presence of any negative predictors.

Generally, the presence of at least 1, 2, 3, 4, or 5 positive predictors, and the absence of all of the negative predictors is predictive of AD activity. In some embodiments, a panel of from 5 to about 100, or from about 5 to about 60 sRNA predictors are detected in the sample. While not each experimental sample will be positive for each positive predictor, the panel is large enough to provide at least about 75%, at least about 80%, at least about 85%, at least about 90%, at least about 95%, or about 100% coverage for the condition in an AD cohort. By selecting a panel in which a plurality of sRNA predictors are present in each sample of the experimental cohort, the panel will be tuned to provide for 100 Sensitivity and 100 Specificity for the training samples (the experimental cohort and the comparator cohort).

In various embodiments, detection of the sRNA predictors involves one of various detection platforms, which can employ reverse-transcription, amplification, and/or hybridization of a probe, including quantitative or qualitative PCR, or RealTime PCR. PCR detection formats can employ stem-loop primers for RT-PCR in some embodiments, and optionally in connection with fluorescently-labeled probes. In some embodiments, sRNAs are detected by RNA sequencing, with computational trimming of the 3′ sequencing adaptor. Sequencing can employ reverse-transcription and/or amplification using at most one specific primer for the binary predictor.

Generally, a real-time polymerase chain reaction (qPCR) monitors the amplification of a targeted DNA molecule during the PCR, i.e. in real-time. Real-time PCR can be used quantitatively, and semi-quantitatively. Two common methods for the detection of PCR products in real-time PCR are: (1) non-specific fluorescent dyes that intercalate with any double-stranded DNA (e.g., SYBR Green (I or II), or ethidium bromide), and (2) sequence-specific DNA probes consisting of oligonucleotides that are labelled with a fluorescent reporter which permits detection only after hybridization of the probe with its complementary sequence (e.g. TAQMAN).

In some embodiments, the assay format is TAQMAN real-time PCR. TAQMAN probes are hydrolysis probes that are designed to increase the Specificity of quantitative PCR. The TAQMAN probe principle relies on the 5′ to 3′ exonuclease activity of Taq polymerase to cleave a dual-labeled probe during hybridization to the complementary target sequence, with fluorophore-based detection. TAQMAN probes are dual labeled with a fluorophore and a quencher, and when the fluorophore is cleaved from the oligonucleotide probe by the Taq exonuclease activity, the fluorophore signal is detected (e.g., the signal is no longer quenched by the proximity of the labels). As in other quantitative PCR methods, the resulting fluorescence signal permits quantitative measurements of the accumulation of the product during the exponential stages of the PCR. The TAQMAN probe format provides high Sensitivity and Specificity of the detection.

In some embodiments, sRNA predictors present in the sample are converted to cDNA using specific primers, e.g., stem-loop primers to interrogate one or both ends of the sRNA. Amplification of the cDNA may then be quantified in real time, for example, by detecting the signal from a fluorescent reporting molecule, where the signal intensity correlates with the level of DNA at each amplification cycle.

Alternatively, sRNA predictors in the panel, or their amplicons, are detected by hybridization. Exemplary platforms include surface plasmon resonance (SPR) and microarray technology. Detection platforms can use microfluidics in some embodiments, for convenient sample processing and sRNA detection.

Generally, any method for determining the presence of sRNAs in samples can be employed. Such methods further include nucleic acid sequence based amplification (NASBA), flap endonuclease-based assays, as well as direct RNA capture with branched DNA (QuantiGene™), Hybrid Capture™ (Digene), or nCounter™ miRNA detection (nanostring). The assay format, in addition to determining the presence of miRNAs and other sRNAs may also provide for the control of, inter alia, intrinsic signal intensity variation. Such controls may include, for example, controls for background signal intensity and/or sample processing, and/or hybridization efficiency, as well as other desirable controls for detecting sRNAs in patient samples (e.g., collectively referred to as “normalization controls”).

In some embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In some embodiments, RNA is extracted from the sample prior to sRNA processing for detection. RNA may be purified using a variety of standard procedures as described, for example, in RNA Methodologies, A laboratory guide for isolation and characterization, 2nd edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNAs may be isolated by organic extraction followed by purification on a glass fiber filter. Alternative methods for isolating miRNAs include hybridization to magnetic beads. Alternatively, miRNA processing for detection (e.g., cDNA synthesis) may be conducted in the biofluid sample, that is, without an RNA extraction step.

In some embodiments, the presence or absence of the sRNAs are determined in a subject sample by nucleic acid sequencing, and individual sRNAs are identified by a process that comprises computational trimming a 3′ sequencing adaptor from individual sRNA sequences. See U.S. 2018/0258486, filed on Jan. 23, 2018, and PCT/US2018/014856, filed on Jan. 23, 2018, which are hereby incorporated by reference in their entireties. In some embodiments, the sequencing process can reverse-transcribe and/or amplify the sRNA predictors using primers specific for the biomarker.

Generally, assays can be constructed such that each assay is at least 80%, or at least 85%, or at least 90%, or at least 95%, or at least 98% specific for the sRNA (e.g., iso-miR) over an annotated sequence and/or other non-predictive iso-miRs and sRNAs. Annotated sequences can be determined with reference to miRBase. For example, in preparing sRNA predictor-specific real-time PCR assays, PCR primers and fluorescent probes can be prepared and tested for their level of Specificity. Bicyclic nucleotides or other modifications involving the 2′ position (e.g., LNA, cET, and MOE), or other nucleotide modifications (including base modifications) can be employed in probes to increase the Sensitivity or Specificity of detection. Specific detection of isomiRs and sRNAs is disclosed in US 2018/0258486, which is hereby incorporated by reference in its entirety.

sRNA predictors can be identified in any biological samples, including solid tissues and/or biological fluids. sRNA predictors can be identified in animals (e.g., vertebrate and invertebrate subjects), or in some embodiments, cultured cells or media from cultured cells. For example, the sample is a biological fluid sample from human or animal subjects (e.g., a mammalian subject), such as blood, serum, plasma, urine, saliva, or cerebrospinal fluid. miRNAs can be found in biological fluid, as a result of a secretory mechanism that may play an important role in cell-to-cell signaling. See, Kosaka N, et al., Circulating microRNA in body fluid: a new potential biomarker for cancer diagnosis and prognosis, Cancer Sci. 2010; 101:2087-2092). miRs from cerebrospinal fluid and serum have been profiled according to conventional methods with the goal of stratifying patients for disease status and pathology features. Burgos K, et al., Profiles of Extracellular miRNA in Cerebrospinal Fluid and Serum from Patients with Alzheimer's and Parkinson's Diseases Correlate with Disease Status and Features of Pathology, PLOS ONE Vol. 9, Issue 5 (2014). In some embodiments, the sample is a solid tissue sample, which may comprise neurons. In some embodiments, the tissue sample is a brain tissue sample, such as from the frontal cortex region. In some embodiments, sRNA predictors are identified in at least two different types of samples, including brain tissue and a biological fluid such as blood. In some embodiments, sRNA predictors are identified in at least three different types of samples, including brain tissue, cerebrospinal fluid (CSF), and blood.

The invention involves detection of sRNA predictors in cells or animals that exhibit an Alzheimer's disease genotype or phenotype. In some embodiments, the sRNA predictor is indicative of AD biological processes in patients or subjects that are otherwise considered non-Alzheimer's patients or subjects. In some embodiments, the sRNA predictor is indicative of specific Braak stage of AD.

In some embodiments, the sRNA predictors are indicative of Braak Stage I and/or II of Alzheimer's disease processes. Braak Stage I/II refers to the transentorhinal (temporal lobe) area of the brain that develops argyrophilic neurofibrillary tangles and neurophil threads over the course of AD progression. Braak Stage I/II is known to be clinically silent at this point in the AD processes.

In some embodiments, the sRNA predictors are indicative of Braak Stage III and/or IV of Alzheimer's disease processes. Braak Stage III/IV refers to the limbic area of the brain that develops argyrophilic neurofibrillary tangles and neurophil threads over the course of AD progression. Braak Stage III/IV is known to be incipient Alzheimer's disease at this point in the AD processes.

In some embodiments, the sRNA predictors are indicative of Braak Stage V and/or VI of Alzheimer's disease processes. Braak Stage V/VI refers to the neocortical area of the brain that develops argyrophilic neurofibrillary tangles and neurophil threads over the course of AD progression. Braak Stage V/VI is known to be full developed Alzheimer's disease at this point in the AD processes.

In some embodiments, the method is repeated to determine the sRNA predictor profile over time, for example, to determine the impact of a therapeutic regimen, or a candidate therapeutic regimen. For example, a subject or patient may be evaluated at a frequency of at least about once per year, or at least about once every six months, or at least once per month, or at least once per week. In some embodiments, a decline in the number of predictors present over time, or a slower increase in the number of predictors detected over time, is indicative of slower disease progression or milder disease symptoms. Embodiments of the invention are useful for constructing animal models for AD treatment, as well as useful as biomarkers in human clinical trials.

In some aspects, the invention provides kits for evaluating samples for Alzheimer's disease activity. In various embodiments, the kits comprise sRNA-specific probes and/or primers configured for detecting a plurality of sRNAs listed in Tables 2A, 4A, and or 7A (SEQ ID NOS: 1-403). In some embodiments, the kit comprises sRNA-specific probes and/or primers configured for detecting at least 2, at least 5, or at least 10, or at least 20, or at least 40 sRNAs listed in Tables 2A, 4A, and or 7A (SEQ ID NOS: 1-403). In some embodiments, the kit comprises sRNA-specific probes and/or primers configured for detecting at least 2, 3, 4, 5, or at least 10, or at least 20 sRNAs listed in Table 2A (SEQ ID NOS: 1-46). In some embodiments, the kit comprises sRNA-specific probes and/or primers configured for detecting at least 2, 3, 4, 5, or at least 10, or at least 20, or at least 40 sRNAs listed in Table 4A (SEQ ID NOS: 47-254). In some embodiments, the kit comprises sRNA-specific probes and/or primers configured for detecting at least 2, 3, 4, 5, or at least 10, or at least 20 sRNAs listed in Table 7A (SEQ ID NOS: 255-403).

The kits may comprise probes and/or primers suitable for a quantitative or qualitative PCR assay, that is, for specific sRNA predictors. In some embodiments, the kits comprise a fluorescent dye or fluorescent-labeled probe, which may optionally comprise a quencher moiety. In some embodiments, the kit comprises a stem-loop RT primer, and in some embodiments may include a stem-loop primer to interrogate each of the sRNA ends. In some embodiments, the kit may comprise an array of sRNA-specific hybridization probes.

In some embodiments, the invention provides a kit comprising reagents for detecting a panel of from 5 to about 100 sRNA predictors, or from about 5 to about 50 sRNA predictors, or from 5 to about 20 sRNAs. In these embodiments, the kit may comprise at least 5, at least 10, at least 20 sRNA predictor assays (e.g., reagents for such assays). In various embodiments, the kit comprises at least 10 positive predictors and at least 5 negative predictors. In some embodiments, the kit comprises a panel of at least 5, or at least 10, or at least 20, or at least 40 sRNA predictor assays, the sRNA predictors being selected from Table 2A, Table 4A, and/or Table 7A. In some embodiments, at least 1 sRNA predictor is selected from Table 4B or Table 7B. Such assays may comprise reverse transcription (RT) primers, amplification primers and probes (such as fluorescent probes or dual labeled probes) specific for the sRNA predictors over annotated sequences as well as other (non-predictive) variations. In some embodiments, the kit is in the form of an array or other substrate containing probes for detection of sRNA predictors by hybridization.

In still other embodiments, the invention involves constructing disease classifiers that classify samples based on the presence or absence of particular sRNA molecules. These disease classifiers are powerful tools for discriminating disease conditions that present with similar symptoms, as well as determining disease subtypes, including predicting the course of the disease, predicting response to treatment, and disease monitoring. Generally, sRNA panels (e.g., panels of distinct sRNA variants) will be determined from sequence data in one or more training sets representing one or more disease conditions of interest. sRNA panels and the classifier algorithm can be constructed using, for example, one or more of supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naïve Bayes Classifier, Perceptron, Quadratic classifiers, Kernel Estimation, k-Nearest Neighbor, Learning Vector Quantization, and Principal Components Analysis. Once the classifier is trained, independent subjects can be evaluated for the disease conditions by detecting the presence or absence, in a biological sample from the subject, of the sRNA markers in the panel, and applying the classification algorithm. Classifiers can be binary classifiers (i.e., classify among two conditions), or may classify among three, four, five, or more disease conditions. In some embodiments, the classifier can classify among at least ten disease conditions.

For example, in some embodiments, the invention provides a method for evaluating a subject for one or more disease conditions. The method comprises providing a biological sample of the subject, and determining the presence or absence of a plurality of sRNAs in the sRNA panel. This profile of “present and absent” sRNAs (binary markers) is used to classify the condition of the subject among two or more disease conditions using the disease classifier. The disease classifier will have been trained based on the presence and absence of the sRNAs in the sRNA panel in a set of training samples. For example, the training samples are annotated as positive or negative for the one or more disease conditions, as well as the presence or absence (or level) of the sRNAs in the panel. In some embodiments, samples are annotated for one or more of disease grade or stage, disease subtype, therapeutic regimen, and drug sensitivity or resistance.

The presence or absence of the sRNAs in the panel is determined in the training set from sRNA sequence data. That is, individual sRNA sequences are identified in the sRNA sequence data by trimming the 5′ and/or 3′ sequencing adaptors and without consolidating sRNA sequence variants to a reference sequence or genetic locus. For example, after trimming, the unique sequence reads within each sample and disease condition or comparator condition are each compiled. Thus, the presence or absence of specific sRNA sequences, such as isoforms, are determined in each sample and for each disease condition, and these variants are not consolidated to reference sequences. These sequences can be used as “binary” markers, that is, evaluated based on their presence or absence in samples, as opposed to discriminating normal and abnormal levels.

In some embodiments, during construction of the classifier, sRNAs are preselected for training. For example, sRNA families can be identified in which variation increases in a disease condition and/or increases with severity of a disease condition, and/or which variation may normalize or be ameliorated in response to a therapeutic regimen. For example, sRNA pre-selection can involve grouping sRNA isoforms (such as isomiRs) into families' based on biologically relevant sequence hyper-features (e.g. ‘seed sequence’ nucleotides 2-8 from the 5′ end of the sRNA isoform, and/or single nucleotide polymorphisms) outside of a lower and upper bound threshold where the lower bound threshold is 0 to 100 trimmed reads per million reads, and the upper bound threshold is 0 to 100 trimmed reads per million reads. These families are evaluated for variation that is correlative with disease activity, and these entire families, or variations with a read count above or below the threshold are selected as candidates for inclusion in the classifier. In some embodiments, these families include at least one sRNA predictor that is unique in at least one of the disease conditions.

Once identified in the sequence data, and selected for inclusion in the computational classifier, molecular detection reagents for the sRNAs in the panel can be prepared. Such detection platforms include quantitative RT-PCR assays, including those employing stem loop primers and fluorescent probes, as described herein. In some embodiments, independent samples are evaluated by sRNA sequencing, rather than migrating to a molecular detection platform.

sRNA panels (e.g., binary sRNA markers used for classification) may contain from about 4 to about 200 sRNAs, or in some embodiments, from about 4 to about 100 sRNAs. In some embodiments, the sRNA panel contains from about 10 to about 100 sRNAs, or from about 10 to about 50 sRNAs.

Classifiers can be trained on various types of samples, including solid tissue samples, biological fluid samples, or cultured cells in some embodiments. When evaluating the subject, biological samples from which sRNAs are evaluated can include biological fluids such as blood, serum, plasma, urine, saliva, or cerebrospinal fluid. Alternatively, the biological sample of the subject is a solid tissue biopsy.

In various embodiments, the training set has at least 50 samples, or at least 100 samples, or at least 200 samples. In some embodiments, the training set includes at least 10 samples for each disease condition or at least 20 or at least 50 samples for each disease condition. A higher number of samples can provide for better statistical powering.

Disease classifiers in accordance with this disclosure can be constructed for various types of disease conditions. For example, in some embodiments, the disease conditions are diseases of the central nervous system. Such diseases can include at least two neurodegenerative diseases involving symptoms of dementia. In some embodiments, at least two disease conditions are selected from Alzheimer's Disease, Parkinson's Disease, Huntington's Disease, Mild Cognitive Impairment, Progressive Supranuclear Palsy, Frontotemporal Dementia, Lewy Body Dementia, and Vascular Dementia. Alternatively, at least two disease conditions are neurodegenerative diseases involving symptoms of loss of movement control, such as Parkinson's Disease, Amyotrophic Lateral Sclerosis, Huntington's Disease, Multiple Sclerosis, and Spinal Muscular Atrophy. In still other embodiments, at least two disease conditions are demyelinating diseases, optionally including multiple sclerosis, optic neuritis, transverse myelitis, and neuromyelitis optica.

Accordingly, in some embodiments, at least one disease condition is selected from Alzheimer's Disease, Parkinson's Disease, Huntington's Disease, Multiple Sclerosis, Amyotrophic Lateral Sclerosis, and Spinal Muscular Atrophy; and training samples are annotated for disease stage, disease severity, drug responsiveness, or course of disease progression.

In still other embodiments, the disease conditions are cancers of different tissue or cell origin. In some embodiments, the disease conditions are drug sensitive versus drug resistant cancer, or sensitivity across two or more therapeutic agents. In such embodiments, the biological sample from the subject can be a tumor or cancer cell biopsy.

In some embodiments, the disease conditions are inflammatory or immunological diseases, and optionally including one or more of Systemic Lupus Erythematosus (SLE), scleroderma, autoimmune vasculitis, diabetes mellitus (type 1 or type 2), Grave's disease, Addison's disease, Sjögren's syndrome, thyroiditis, rheumatoid arthritis, myasthenia gravis, multiple sclerosis, fibromyalgia, psoriasis, Crohn's disease, ulcerative colitis, diverticular disease and celiac disease. For example, the classifier can distinguish gastrointestinal inflammatory conditions such as, but not limited to, Crohn's disease, ulcerative colitis, and diverticular disease. In such embodiments, the biological samples from the subject to be tested can be biological fluid samples such as blood, serum, or plasma, or can be biopsy tissue such as colon epithelial tissue.

In some embodiments, the disease conditions are cardiovascular diseases, optionally including stratification for risk of acute event. In some embodiments, the cardiovascular diseases include one or more of coronary artery disease (CAD), myocardial infarction, stroke, congestive heart failure, hypertensive heart disease, cardiomyopathy, heart arrhythmia, congenital heart disease, valvular heart disease, carditis, aortic aneurysms, peripheral artery disease, and venous thrombosis.

In various embodiments, at least one, or at least two, or at least five, or at least ten sRNAs in the panel are positive sRNA predictors. That is, the positive sRNA predictors were identified as present in a plurality of samples annotated as positive for a disease condition in the training set, and absent in all samples annotated as negative for the disease condition in the training set. In some embodiments, with respect to a disease classifier including Alzheimer's Disease as a disease condition, the sRNA panel may include one or more, or two or more, or five or more, or ten or more, sRNAs from Table 2A, Table 4A, and/or Table 7A (SEQ ID NOS: 1-403).

In some embodiments, the sRNA panel includes one or more sRNA predictors from Table 2A (SEQ ID NOS: 1 to 46). In some embodiments, the sRNA panel includes one or more sRNA predictors from Table 4A (SEQ ID NOS: 47-254). In some embodiments, the sRNA panel includes one or more sRNA predictors from Table 4A (SEQ ID NOS: 255-403). In some embodiments, the sRNA panel includes one or more sRNA predictors from Table 5 (SEQ ID NOS: 58, 189, 78, 172, 193, 97, 122, 215, 248, 164, 120, 93, 126, 253, 112, 144, 213, 244, 123, 222, 150, 240, 52, 220, 221, 169, 165, and 212), which correlate with Braak stages of AD progression in CSF. In some embodiments, the sRNA panel include one or more sRNAs from Table 8 (SEQ ID NOS: 257, 270, 272, 273, 279, 286, 288, 314, 319, 325, 332, 341, 374, 391, and 393), which correlate with Braak stages of AD progression in serum.

Other aspects and embodiments of the invention will be apparent from the following examples.

### EXAMPLES

### Example 1: Binary Classifiers for Alzheimer's Disease were Identified in Either an Experimental or Comparator Group of Brain Tissue, Cerebrospinal Fluid, or Serum

To identify binary small RNA predictors for Alzheimer's Disease, small RNA sequencing data was downloaded from the GEO and dbGaP Databases and used as a Discovery Set (Table 1A-1B: Brain Samples, Table 3A-3B CSF Samples, and Table 6A-6B SER Samples). All samples, regardless of material, were derived from postmortem-verified Alzheimer's or non-Alzheimer's samples (healthy controls or other non-Alzheimer's related neurological diseases such as Parkinson's, Parkinson's with Dementia, Huntington's, etc.).

The overall process is described below:

| | | Number of |
|---|---|---|
| | Sample | Samples |
| Diagnosis | Material | (N) |
| Alzheimer's Disease | brain tissue | 17 |
| Controls | brain tissue | 123 |
| Healthy | | 51 |
| other non-Alzheimer's Neurological Disease | | 72 |
| Alzheimer's Disease | CSF | 64 |
| Controls | CSF | 109 |
| Healthy | | 68 |
| other non-Alzheimer's Neurological Disease | | 41 |
| Alzheimer's Disease | SER | 51 |
| Controls | SER | 130 |
| Healthy | | 70 |
| other non-Alzheimer's Neurological Disease | | 60 |

CSF = cerebrospinal fluid, SER = serum.

Files were converted from a .sra to .fastq format using the SRA Tool Kit v2.8.0 for Centos, and .fastq formatted files were processed as described in U.S. 2018/0258486 and International Application No. PCT/US2018/014856, filed on Jan. 23, 2018 (which are hereby incorporated by reference in their entireties). Specifically, all .fastq data files were processed by trimming adapter sequences using the (Regex) regular expression-based search and trim algorithm, where 5′ TGGAATTCTCGGGTGCCAAGGAA 3′ (SEQ ID NO: 404) (containing up to a 15 nucleotide 3′-end truncation) was input to identify the 3′ adapter sequence, and a Levenshtein Distance of 2 or a Hamming Distance of 5. Parameters for Regex searching requires that the 1^{st} nucleotide of the user-specified search term to be unaltered with respect to nucleotide insertions, deletions, and/or swaps.

Samples are compiled in 1 of 2 groups, either an Experimental Group or a Comparator Group. sRNA-Split identifies small RNAs that are unique to either the Experimental Group or Comparator Group, as well as small RNAs that are present in both the Experimental Group and Comparator Group. Small RNAs that are unique to either the Experimental Group or Comparator Group have 100% Specificity (by definition). Unique (binary) small RNAs serve as classifiers for the Group in which they were identified. Binary small RNA classifiers can be used in non-bootstrapped and/or bootstrapped computational classification algorithms (e.g. supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naïve Bayes Classifier, Perceptron, Quadratic classifiers, Kernel Estimation, k-Nearest Neighbor, Learning Vector Quantization, and Principal Components Analysis, etc.), and they can also be used as targets for Quantitative Reverse-Transcription Polymerase Chain Reaction (RT-qPCR).

Binary small RNA classifiers were identified by analyzing trimmed, small RNA reads with sRNA-Split. Trimmed reads were converted to trimmed-reads per million reads. Biomarkers were filtered such that each sample needed to have a minimum of 1 marker providing coverage. To identify biomarkers correlated with Braak Stage, small RNAs had to be present in a minimum of 3 consecutive Braak Stages and have a Pearson Correlation Coefficient of ≥0.75.

Specific biomarker panels containing binary small RNA predictors (present in samples of the Experimental Group, but not present in any samples of the Comparator Group) were identified as follows:

(1) AD vs non-AD
(A) Brain Tissue (Table 2)
(B) CSF (Table 4)
(C) Serum (Table 7)

(2) Alzheimer's Disease Monitoring
(A) CSF (Table 5)
(B) Serum (Table 8)

Probability scores (p-values) were calculated for each individual binary small RNA predictor using a Chi-Square 2×2 Contingency Table and one-tailed Fisher's Exact Probability Test.

Probability scores (p-values) were calculated for panels of binary small RNA predictor for each Experimental Group using a Chi-Square 2×2 Contingency Table and one-tailed Fisher's Exact Probability Test (all giving 100% Specificity and 100% Sensitivity).

### Example 2: Construction of Multi-Class Disease Classifiers of Inflammatory Bowel Disease (IBD)

To construct disease classifiers that classify IBD samples based on the presence or absence of particular sRNA molecules, sRNA panels were determined from sequence data in various training sets representing different disease conditions of interest, such as Crohn's disease, ulcerative colitis, and diverticular disease.

### Samples

All samples were collected according to their respective Institutional Review Board (IRB) approval and have patient consent for unrestricted use. Data was collected from electronic medical records and chart review. Clinical Data includes information such as: age, gender, race, ethnicity, weight, body mass index, smoking history, alcohol use history, family history of disease. Disease-related data includes information such as: diagnosis, age at Inflammatory Bowel Disease (IBD) diagnosis, current and prior medications, comorbidities, age at proctocolectomy and Ileal Pouch Anal Anastomosis (IPAA), as well as pouch age, time from closure of ileostomy, or from pouch surgery (where applicable from patients undergoing these procedures).

Biopsies were taken from the colon epithelium. Inoperable Ulcerative Colitis (IUC), Operable Ulcerative Colitis (OUC), Crohn's Disease (CD), Diverticular Disease (DD), Polyps/Polyposis (PP), Serrated Polyps/Polyposis (SPP), colon cancer, (CC), rectal cancer (RC) were defined according to clinical, endoscopic, histologic, and imaging studies. Further inclusion criteria were the presence of ileitis for CD patients and having a normal terminal ileum as seen by endoscopy and confirmed by histology for IUC patients. Individuals who required a colonoscopy for routine screening and were verified as having non-diseased bowel tissue by endoscopy and/or histology were labeled as normal controls.

All biopsies were assessed by a minimum of two (2) institutional IBD-trained pathologists and consensus scores and diagnoses were provided according to clinical and industry standard diagnostic protocols. Briefly, active inflammatory characteristics were scored according to neutrophil infiltration (0-3) and area of ulceration (0-3), each sample was classified into inactive, cryptitis, crypt abscess, numerous crypt abscesses (>3/high power field) and ulceration. Original Geboes Score (OGS) or Simplified Geboes Score (SGS) was used to classify UC. Chron's Disease Activity Index (CDAI) and Crohn's Disease Endoscopic Index of Severity (CDEIS) was used to classify CD. Hinchey Classification was used to characterize DD. Colorectal cancers, polyps and serrated polyps were classified according to the most recent recommendations of the Multi-Society Task Force on Colorectal Cancer (CRC).

An overview of the IBD samples used is displayed below:

| | | Crohn's | Ulcerative | Diverticular |
|---|---|---|---|---|
| Diagnosis | Normal | disease | Colitis | Disease |
| Tissue Type | Colon | Colon | Colon | Colon |
| | Epithelium | Epithelium | Epithelium | Epithelium |
| N | 64 | 35 | 139 | 20 |
| Gender (F:M) | 26:38 | 14:21 | 50:89 | 6:14 |
| Age at sampling, years, | 56.4 ± 13.5 | 36.6 ± 15.8 | 45.5 ± 14.1 | 44.9 ± 10.6 |
| mean ± SD (range) | (26-82) | (15-76) | (32-57) | (31-69) |
| Age at IBD diagnosis, | NA | 30.4 ± 12.1 | 32.1 ± 11.6 | 26.2 ± 8.7 |
| years, mean ± SD (range) | | (18-48) | (16-51) | (21-55) |
| IBD duration, years, | NA | 13.3 | 10.5 | 12.6 |
| mean ± SD (range) | | (3-53) | (3-28) | (25-53) |
| Ashkenazi origin | 5 | 2 | 9 | 1 |
| Non-Ashkenazi origin | 53 | 31 | 120 | 17 |
| Mixed origin | 6 | 2 | 10 | 2 |
| Never smoker | 56 | 28 | 122 | 19 |
| Past smokers | 5 | 2 | 10 | 1 |
| Current smokers | 3 | 5 | 7 | 0 |
| Body mass index, | 25.5 ± 2.9 | 27.1 ± 5.3 | 25.8 ± 6.1 | 23.3 ± 5.2 |
| mean ± SD (range) | (17-30) | (18-31) | (15-41) | (18-40) |
| Family history of IBD | 2 | 3 | 8 | 1 |
| Steroid exposure | NA | NA | 110 | NA |
| Severity Score (B1:B2:B3) | NA | 7:6:8 | NA | NA |

To identify small RNA predictors for disease classes associated with IBD, small RNA sequencing data was downloaded from the GEO Database and used as a Discovery Set. small RNA sequencing data was downloaded from the Geodatabase studies for Crohn's disease (GSE66208), Ulcerative colitis (GSE114591), Diverticular disease (GSE89667), and Normal/Control (GSE118504).

Files were converted from a .sra to .fastq format using the SRA Tool Kit v2.8.0 for Centos, and .fastq formatted files were processed as described in U.S. 2018/0258486 and International Application No. PCT/US2018/014856, filed on Jan. 23, 2018 (which are hereby incorporated by reference in their entireties). Specifically, all .fastq data files were processed by trimming adapter sequences using the (Regex) regular expression-based search and trim algorithm, where 5′ TGGAATTCTCGGGTGCCAAGGAA 3′ (SEQ ID NO: 404) (containing up to a 15 nucleotide 3′-end truncation) was input to identify the 3′ adapter sequence, and a Levenshtein Distance of 2 or a Hamming Distance of 5. Parameters for Regex searching requires that the 1st nucleotide of the user-specified search term to be unaltered with respect to nucleotide insertions, deletions, and/or swaps.

Samples are compiled in 1 of 2 groups, either an Experimental Group or a Comparator Group. sRNA-Split identifies small RNAs that are unique to either the Experimental Group or Comparator Group, as well as small RNAs that are present in both the Experimental Group and Comparator Group. Small RNAs that are unique to either the Experimental Group or Comparator Group have 100% Specificity (by definition). Unique (binary) small RNAs serve as classifiers for the Group in which they were identified. Binary small RNA classifiers can be used in non-bootstrapped and/or bootstrapped computational classification algorithms (e.g. supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naïve Bayes Classifier, Perceptron, Quadratic classifiers, Kernel Estimation, k-Nearest Neighbor, Learning Vector Quantization, and Principal Components Analysis, etc.), and they can also be used as targets for Quantitative Reverse-Transcription Polymerase Chain Reaction (RT-qPCR).

Binary small RNA classifiers were identified by analyzing trimmed, small RNA reads with sRNA-Split. Trimmed reads were converted to trimmed-reads per million reads. Biomarkers were filtered such that each sample needed to have a minimum of 1 marker providing coverage.

### Per-Class Metrics

Per-class metrics were determined for each class in order to identify markers that are most important for identifying the disease class. sRNA panels were determined from sequence data in various training sets representing different disease conditions of interest. Specific biomarker panels containing small RNA predictors of disease class were identified as follows:
Controls (Healthy individuals/“Normal” individuals): Table 9;
Crohn's disease: Table 10;
Ulcerative colitis: Table 11; and
Diverticular disease: Table 12.

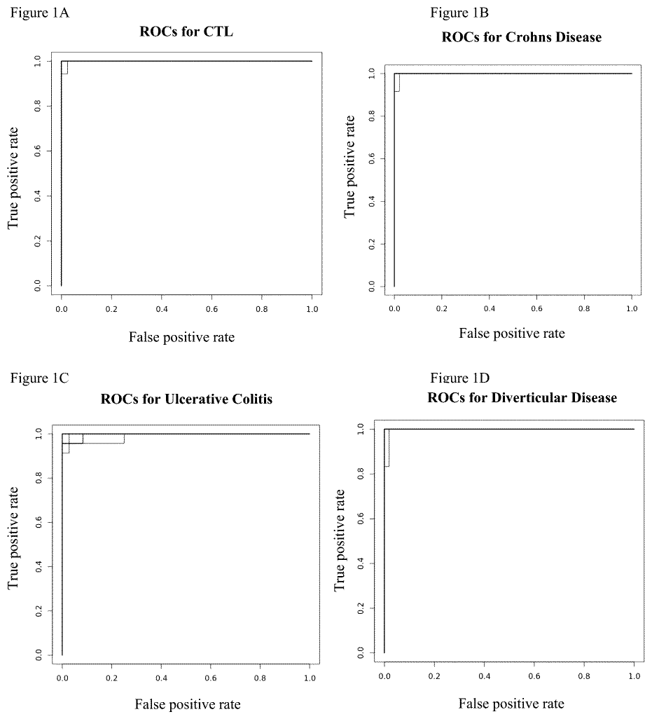

By using a supervised, non-parametric, logistical regression machine learning model, the final selection marker count was reduced from 128 to 100 maximum. In order to assess the classification model's performance, ROC/AUC curves were obtained for each set of markers identified per class, where ROC is a probability curve and AUC represents the degree or measure of separability. The ROC curve is plotted with true positive rate against the false positive rate. ROC/AUC curves were established for the various IBD classes and controls, as discussed above, and these are depicted in FIG. 1.

### Multi-Class Disease Classification

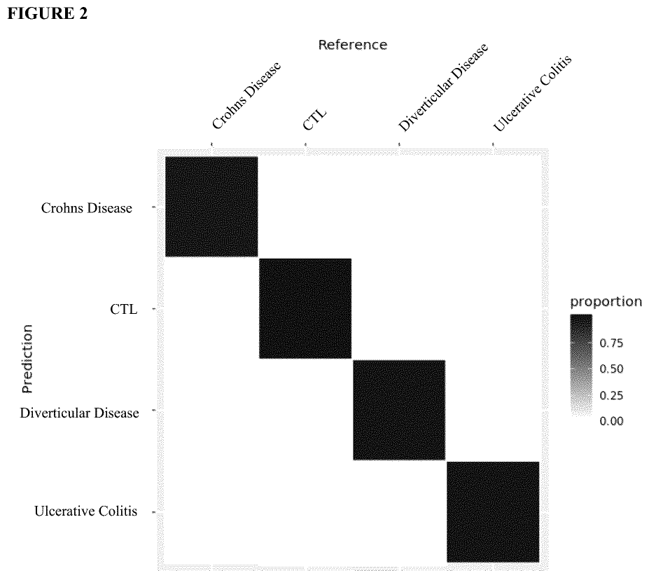

The disease classifier was trained based on the positive or negative markers of the sRNA panels, as well as the presence or absence of the sRNAs in the panels identified above for Controls, Crohn's disease, ulcerative colitis, and diverticular disease. In order to assess the accuracy of the computational model when the class metrics were all combined, a test was run to evaluate the model's identification predictive power against reference samples of each class. It was found that the model had an accuracy rate of 98%. FIG. 2 depicts a heat map showing the proportion of accurate predictions of disease class against their true reference identies. These results are also shown in the matrix below:

| | Reference | | | |
|---|---|---|---|---|
| | Crohn's | | Diverticular | Ulcerative |
| Prediction | Disease | Control | Disease | Colitis |
| Crohn's | 116 | 0 | 0 | 0 |
| Disease | | | | |
| Control | 0 | 179 | 0 | 0 |
| Diverticular | 0 | 0 | 59 | 4 |
| Disease | | | | |
| Ulcerative | 4 | 1 | 1 | 226 |
| Colitis | | | | |

### REFERENCES

1. Santa-Maria I, Alaniz M E, Renwick N, Cela C et al. Dysregulation of microRNA-219 promotes neurodegeneration through post-transcriptional regulation of tau. J Clin Invest 2015 February; 125 (2): 681-6. PMID: 25574843
2. Lau P, Bossers K, Janky R, Salta E et al. Alteration of the microRNA network during the progression of Alzheimer's disease. EMBO Mol Med 2013 Oct.;5 (10): 1613-34. PMID: 24014289
3. Hébert S S, Wang W X, Zhu Q, Nelson P T. A study of small RNAs from cerebral neocortex of pathology-verified Alzheimer's disease, dementia with lewy bodies, hippocampal sclerosis, frontotemporal lobar dementia, and non-demented human controls. J Alzheimers Dis 2013; 35 (2): 335-48. PMID: 23403535
4. Hoss A G, Labadorf A, Beach T G, Latourelle J C et al. microRNA Profiles in Parkinson's Disease Prefrontal Cortex. Front Aging Neurosci 2016; 8:36. PMID: 26973511
5. Hoss A G, Labadorf A, Latourelle J C, Kartha V K et al. miR-10b-5p expression in Huntington's disease brain relates to age of onset and the extent of striatal involvement. BMC Med Genomics 2015 Mar. 1; 8:10. PMID: 25889241
6. Burgos K, Malenica I, Metpally R, Courtright A, et al. Profiles of extracellular miRNA in cerebrospinal fluid and serum from patients with Alzheimer's and Parkinson's diseases correlate with disease status and features of pathology. PLOS One. 2014; 9 (5): e94839. PMID: 24797360

TABLE 1A
Experimental Alzheimer's disease cohort for biomarker discovery, taken from brain samples.

| | | | | | Age at | Braak |
|---|---|---|---|---|---|---|
| Group | Sample ID | Study Number | Disease Type | Gender | Death | score |
| Experimental | SRR1658350 | GSE63501 | Alzheimer's | F | 90 | III-IV |
| Experimental | SRR1658353 | GSE63501 | Alzheimer's | F | 90 | III-IV |
| Experimental | SRR1103943 | GSE48552 | Alzheimer's | M | 79 | V |
| Experimental | SRR828723 | GSE46131 | Alzheimer's | F | 83 | V |
| Experimental | SRR1658347 | GSE63501 | Alzheimer's | F | 92 | V-VI |
| Experimental | SRR1658348 | GSE63501 | Alzheimer's | F | 91 | V-VI |
| Experimental | SRR1658349 | GSE63501 | Alzheimer's | M | 86 | V-VI |
| Experimental | SRR1658351 | GSE63501 | Alzheimer's | M | 98 | V-VI |
| Experimental | SRR1103944 | GSE48552 | Alzheimer's | F | 80 | VI |
| Experimental | SRR1103945 | GSE48552 | Alzheimer's | M | 67 | VI |
| Experimental | SRR1103946 | GSE48552 | Alzheimer's | F | 67 | VI |
| Experimental | SRR1103947 | GSE48552 | Alzheimer's | F | 68 | VI |
| Experimental | SRR1103948 | GSE48552 | Alzheimer's | F | 72 | VI |
| Experimental | SRR828724 | GSE46131 | Alzheimer's | F | 86 | VI |
| Experimental | SRR828725 | GSE46131 | Alzheimer's | F | 67 | VI |
| Experimental | SRR828726 | GSE46131 | Alzheimer's | F | 75 | VI |
| Experimental | SRR828727 | GSE46131 | Alzheimer's | F | 86 | VI |
| AVERAGE | NA | NA | NA | NA | 81.00 ± 10.1 | NA |

TABLE 1B
Comparator cohort for AD biomarker discovery, taken from brain samples, including
healthy controls and various other non-Alzheimer's neurological disorders.

| | | Study | | | Age at | Braak |
|---|---|---|---|---|---|---|
| Group | Sample ID | Number | Disease Type | Gender | Death | score |
| Comparator | SRR828715 | GSE46131 | Bilateral hippocampal | F | 84 | 0 |
| | | | sclerosis | | | |
| Comparator | SRR828716 | GSE46131 | Bilateral hippocampal | F | 84 | 0 |
| | | | sclerosis | | | |
| Comparator | SRR828718 | GSE46131 | Bilateral hippocampal | F | 101 | 0 |
| | | | sclerosis | | | |
| Comparator | SRR1658356 | GSE72962 | Control | M | 93 | 0 |
| Comparator | SRR1658357 | GSE72962 | Control | M | 92 | 0 |
| Comparator | SRR1658359 | GSE72962 | Control | F | 84 | 0 |
| Comparator | SRR1658360 | GSE72962 | Control | F | 85 | 0 |
| Comparator | SRR1103937 | GSE48552 | Control | M | 80 | 0 |
| Comparator | SRR1103938 | GSE48552 | Control | M | 78 | 0 |
| Comparator | SRR1103939 | GSE48552 | Control | F | 52 | 0 |
| Comparator | SRR1103940 | GSE48552 | Control | F | 74 | 0 |
| Comparator | SRR828708 | GSE46131 | Control | F | 75 | 0 |
| Comparator | SRR828709 | GSE46131 | Control | F | 84 | 0 |
| Comparator | SRR828719 | GSE46131 | Dementia with Lewy | M | 78 | 0 |
| | | | bodies | | | |
| Comparator | SRR828720 | GSE46131 | Dementia with Lewy | M | 78 | 0 |
| | | | bodies | | | |
| Comparator | SRR828721 | GSE46131 | Dementia with Lewy | F | 85 | 0 |
| | | | bodies | | | |
| Comparator | SRR828722 | GSE46131 | Dementia with Lewy | M | 68 | 0 |
| | | | bodies | | | |
| Comparator | SRR828710 | GSE46131 | FTLD (TDP43 negative) | F | 37 | 0 |
| Comparator | SRR828711 | GSE46131 | FTLD (TDP43 positive) | F | 53 | 0 |
| Comparator | SRR828712 | GSE46131 | FTLD (TDP43 positive) | M | 48 | 0 |
| Comparator | SRR828713 | GSE46131 | FTLD (TDP43 positive) | F | 87 | 0 |
| Comparator | SRR828714 | GSE46131 | Progressive supranuclear | M | 70 | 0 |
| | | | palsy | | | |
| Comparator | SRR1103941 | GSE48552 | Control | M | 83 | I |
| Comparator | SRR1103942 | GSE48552 | Control | F | 78 | I |
| Comparator | SRR1658345 | GSE63501 | Control | F | 82 | I-II |
| Comparator | SRR1658355 | GSE63501 | Control | M | 90 | I-II |
| Comparator | SRR1658346 | GSE63501 | Control | M | 94 | III-IV |
| Comparator | SRR1658352 | GSE63501 | TPD | F | 93 | III-IV |
| Comparator | SRR1658354 | GSE63501 | TPD | F | 88 | III-IV |
| Comparator | SRR1658358 | GSE63501 | TPD | F | 96 | III-IV |
| Comparator | SRR1759212 | GSE72962 | Control | M | 73 | NA |
| Comparator | SRR1759213 | GSE72962 | Control | M | 91 | NA |
| Comparator | SRR1759214 | GSE72962 | Control | M | 82 | NA |
| Comparator | SRR1759215 | GSE72962 | Control | M | 97 | NA |
| Comparator | SRR1759216 | GSE72962 | Control | M | 86 | NA |
| Comparator | SRR1759217 | GSE72962 | Control | M | 91 | NA |
| Comparator | SRR1759218 | GSE72962 | Control | M | 81 | NA |
| Comparator | SRR1759219 | GSE72962 | Control | M | 79 | NA |
| Comparator | SRR1759220 | GSE72962 | Control | M | 63 | NA |
| Comparator | SRR1759221 | GSE72962 | Control | M | 66 | NA |
| Comparator | SRR1759222 | GSE72962 | Control | M | 69 | NA |
| Comparator | SRR1759223 | GSE72962 | Control | M | 79 | NA |
| Comparator | SRR1759224 | GSE72962 | Control | M | 61 | NA |
| Comparator | SRR1759225 | GSE72962 | Control | M | 58 | NA |
| Comparator | SRR1759226 | GSE72962 | Control | M | 70 | NA |
| Comparator | SRR1759227 | GSE72962 | Control | M | 66 | NA |
| Comparator | SRR1759228 | GSE72962 | Control | M | 60 | NA |
| Comparator | SRR1759229 | GSE72962 | Control | M | 76 | NA |
| Comparator | SRR1759230 | GSE72962 | Control | M | 61 | NA |
| Comparator | SRR1759231 | GSE72962 | Control | M | 62 | NA |
| Comparator | SRR1759232 | GSE72962 | Control | M | 69 | NA |
| Comparator | SRR1759233 | GSE72962 | Control | M | 61 | NA |
| Comparator | SRR1759234 | GSE72962 | Control | M | 93 | NA |
| Comparator | SRR1759235 | GSE72962 | Control | M | 53 | NA |
| Comparator | SRR1759236 | GSE72962 | Control | M | 57 | NA |
| Comparator | SRR1759237 | GSE72962 | Control | M | 43 | NA |
| Comparator | SRR1759238 | GSE72962 | Control | F | 71 | NA |
| Comparator | SRR1759239 | GSE72962 | Control | M | 46 | NA |
| Comparator | SRR1759240 | GSE72962 | Control | M | 40 | NA |
| Comparator | SRR1759241 | GSE72962 | Control | M | 44 | NA |
| Comparator | SRR1759242 | GSE72962 | Control | M | 57 | NA |
| Comparator | SRR1759243 | GSE72962 | Control | M | 80 | NA |
| Comparator | SRR1759244 | GSE72962 | Control | F | 75 | NA |
| Comparator | SRR1759245 | GSE72962 | Control | F | 76 | NA |
| Comparator | SRR1759246 | GSE72962 | Control | M | 68 | NA |
| Comparator | SRR1759247 | GSE72962 | Control | M | 64 | NA |
| Comparator | SRR1759248 | GSE64977 | Huntington's Disease | M | 55 | NA |
| Comparator | SRR1759249 | GSE64977 | Huntington's Disease | M | 69 | NA |
| Comparator | SRR1759250 | GSE64977 | Huntington's Disease | M | 71 | NA |
| Comparator | SRR1759251 | GSE64977 | Huntington's Disease | M | 48 | NA |
| Comparator | SRR1759252 | GSE64977 | Huntington's Disease | M | 40 | NA |
| Comparator | SRR1759253 | GSE64977 | Huntington's Disease | M | 72 | NA |
| Comparator | SRR1759254 | GSE64977 | Huntington's Disease | M | 43 | NA |
| Comparator | SRR1759255 | GSE64977 | Huntington's Disease | M | 68 | NA |
| Comparator | SRR1759256 | GSE64977 | Huntington's Disease | M | 59 | NA |
| Comparator | SRR1759257 | GSE64977 | Huntington's Disease | M | 68 | NA |
| Comparator | SRR1759258 | GSE64977 | Huntington's Disease | M | 57 | NA |
| Comparator | SRR1759259 | GSE64977 | Huntington's Disease | M | 48 | NA |
| Comparator | SRR1759260 | GSE64977 | Huntington's Disease | M | 68 | NA |
| Comparator | SRR1759261 | GSE64977 | Huntington's Disease | M | 54 | NA |
| Comparator | SRR1759262 | GSE64977 | Huntington's Disease | M | 68 | NA |
| Comparator | SRR1759263 | GSE64977 | Huntington's Disease | M | 61 | NA |
| Comparator | SRR1759264 | GSE64977 | Huntington's Disease | M | 48 | NA |
| Comparator | SRR1759265 | GSE64977 | Huntington's Disease | M | 69 | NA |
| Comparator | SRR1759266 | GSE64977 | Huntington's Disease | F | 68 | NA |
| Comparator | SRR1759267 | GSE64977 | Huntington's Disease | M | 55 | NA |
| Comparator | SRR1759268 | GSE64977 | Huntington's Disease | M | 50 | NA |
| Comparator | SRR1759269 | GSE64977 | Huntington's Disease | M | 51 | NA |
| Comparator | SRR1759270 | GSE64977 | Huntington's Disease | M | 79 | NA |
| Comparator | SRR1759271 | GSE64977 | Huntington's Disease | M | 50 | NA |
| Comparator | SRR1759272 | GSE64977 | Huntington's Disease | M | 75 | NA |
| Comparator | SRR1759273 | GSE64977 | Huntington's Disease | M | 53 | NA |
| Comparator | SRR2353419 | GSE72962 | Parkinson's Disease | M | 80 | NA |
| Comparator | SRR2353421 | GSE72962 | Parkinson's Disease | M | 80 | NA |
| Comparator | SRR2353424 | GSE72962 | Parkinson's Disease | M | 81 | NA |
| Comparator | SRR2353425 | GSE72962 | Parkinson's Disease | M | 77 | NA |
| Comparator | SRR2353426 | GSE72962 | Parkinson's Disease | M | 64 | NA |
| Comparator | SRR2353428 | GSE72962 | Parkinson's Disease | M | 94 | NA |
| Comparator | SRR2353430 | GSE72962 | Parkinson's Disease | M | 85 | NA |
| Comparator | SRR2353431 | GSE72962 | Parkinson's Disease | M | 75 | NA |
| Comparator | SRR2353432 | GSE72962 | Parkinson's Disease | M | 74 | NA |
| Comparator | SRR2353433 | GSE72962 | Parkinson's Disease | M | 89 | NA |
| Comparator | SRR2353434 | GSE72962 | Parkinson's Disease | M | 66 | NA |
| Comparator | SRR2353435 | GSE72962 | Parkinson's Disease | M | 65 | NA |
| Comparator | SRR2353436 | GSE72962 | Parkinson's Disease | M | 85 | NA |
| Comparator | SRR2353438 | GSE72962 | Parkinson's Disease | M | 64 | NA |
| Comparator | SRR2353442 | GSE72962 | Parkinson's Disease | M | 74 | NA |
| Comparator | SRR2353443 | GSE72962 | Parkinson's Disease | M | 68 | NA |
| Comparator | SRR2353444 | GSE72962 | Parkinson's Disease | M | 79 | NA |
| Comparator | SRR2353445 | GSE72962 | Parkinson's Disease | M | 70 | NA |
| Comparator | SRR2353417 | GSE72962 | Parkinson's Disease with | M | 74 | NA |
| | | | Dementia | | | |
| Comparator | SRR2353418 | GSE72962 | Parkinson's Disease with | M | 83 | NA |
| | | | Dementia | | | |
| Comparator | SRR2353420 | GSE72962 | Parkinson's Disease with | M | 83 | NA |
| | | | Dementia | | | |
| Comparator | SRR2353422 | GSE72962 | Parkinson's Disease with | M | 84 | NA |
| | | | Dementia | | | |
| Comparator | SRR2353423 | GSE72962 | Parkinson's Disease with | M | 88 | NA |
| | | | Dementia | | | |
| Comparator | SRR2353427 | GSE72962 | Parkinson's Disease with | M | 85 | NA |
| | | | Dementia | | | |
| Comparator | SRR2353429 | GSE72962 | Parkinson's Disease with | M | 80 | NA |
| | | | Dementia | | | |
| Comparator | SRR2353437 | GSE72962 | Parkinson's Disease with | M | 64 | NA |
| | | | Dementia | | | |
| Comparator | SRR2353439 | GSE72962 | Parkinson's Disease with | M | 75 | NA |
| | | | Dementia | | | |
| Comparator | SRR2353440 | GSE72962 | Parkinson's Disease with | M | 68 | NA |
| | | | Dementia | | | |
| Comparator | SRR2353441 | GSE72962 | Parkinson's Disease with | M | 95 | NA |
| | | | Dementia | | | |
| Comparator | SRR1759274 | GSE64977 | Pre-AD | F | 86 | NA |
| Comparator | SRR1759275 | GSE64977 | Pre-AD | M | 49 | NA |
| AVERAGE | NA | NA | NA | NA | 71.32 ± 14.7 | NA |

TABLE 2A
Disease Specific Biomarkers for Alzheimer's Disease Identified in Brain Tissue

| Seq. | | Total | Frequency | | p-value in |
|---|---|---|---|---|---|
| ID | Sequence | Reads | (Sensitivity) | Specificity | Discovery set |
| 1 | CAGGCAGTTACAGATCGAACTCC | 45 | 47.06% | 100% | 8.142E−09 |
| 2 | GGTCAGTTACAGATCGAAC | 31 | 47.06% | 100% | 8.142E−09 |
| 3 | CTGGCTGGGTTGTTCGAGACCCGC | 38 | 41.18% | 100% | 1.083E−07 |
| 4 | TTATGTGATGACTTACA | 78 | 35.29% | 100% | 1.319E−06 |
| 5 | TTCTGTGATGACTTACA | 48 | 35.29% | 100% | 1.319E−06 |
| 6 | AGGTTATGGGTTCGTGTCCCACC | 40 | 35.29% | 100% | 1.319E−06 |
| 7 | TCTTGCTCCGTCCACTCC | 38 | 35.29% | 100% | 1.319E−06 |
| 8 | GGTAGAGCATGGGACTCTTAATCGC | 35 | 35.29% | 100% | 1.319E−06 |
| 9 | TCGTGCTGGGCCCATAACC | 28 | 35.29% | 100% | 1.319E−06 |
| 10 | GGGTTGTGGGTTCGGGTCCCACC | 24 | 35.29% | 100% | 1.319E−06 |
| 11 | TTTATCACGTTCGCCTC | 23 | 35.29% | 100% | 1.319E−06 |
| 12 | AGGTTCCGGGCTCGGGACCCGGC | 23 | 35.29% | 100% | 1.319E−06 |
| 13 | CATATGTGGTGAATACGTGTT | 22 | 35.29% | 100% | 1.319E−06 |
| 14 | GCGGTAGAGCATGGGACTCTTAATCCC | 22 | 35.29% | 100% | 1.319E−06 |
| 15 | GATCCATTGGGGTTTCCCCGCGCAGGT | 21 | 35.29% | 100% | 1.319E−06 |
| 16 | CCATGGGACTCTTAATCC | 20 | 35.29% | 100% | 1.319E−06 |
| 17 | GGTAAACATCTCCGACTGGAA | 20 | 35.29% | 100% | 1.319E−06 |
| 18 | AGGGTGTGGGTTCGAATCCCACC | 73 | 29.41% | 100% | 1.484E−05 |
| 19 | AAGGTTCCGGGTTCGTGTCGCGGC | 62 | 29.41% | 100% | 1.484E−05 |
| 20 | AAGTTTCCGGGTTCGGGCCCCGGC | 62 | 29.41% | 100% | 1.484E−05 |
| 21 | AGGTTGTGGATTCGTGTCCCACC | 55 | 29.41% | 100% | 1.484E−05 |
| 22 | GAAGTTCCGGGTTCGGGTCCCGGC | 52 | 29.41% | 100% | 1.484E−05 |
| 23 | AGGCTGTGGGTTCGAATCCCACC | 39 | 29.41% | 100% | 1.484E−05 |
| 24 | GGGTGTGATGACTTACA | 37 | 29.41% | 100% | 1.484E−05 |
| 25 | AAGTTTCCGGGTTCGGGACCCGGC | 35 | 29.41% | 100% | 1.484E−05 |
| 26 | AAGGTTCCGGGTTCGGTTCCCGGC | 34 | 29.41% | 100% | 1.484E−05 |
| 27 | ACTGTGGACTCTGAATCCA | 31 | 29.41% | 100% | 1.484E−05 |
| 28 | AAGGTTCCGGGTTCGGGTACCGGC | 31 | 29.41% | 100% | 1.484E−05 |
| 29 | GCACGGGACTCTTAATCCC | 30 | 29.41% | 100% | 1.484E−05 |
| 30 | AAGTTTGTGGGTTCGTATCCCACC | 28 | 29.41% | 100% | 1.484E−05 |
| 31 | GGAGTGTGGGTTCGTGTCCCATC | 27 | 29.41% | 100% | 1.484E−05 |
| 32 | AGGTTGTGGGTTCGAGGCCCACC | 26 | 29.41% | 100% | 1.484E−05 |
| 33 | AGAGTTTCCGGGTTCGTGTCCCGGC | 25 | 29.41% | 100% | 1.484E−05 |
| 34 | TTGAGGGTGCGTGTCCCT | 24 | 29.41% | 100% | 1.484E−05 |
| 35 | AGAGGTTCCGGGGTCGGGTCCCGGC | 24 | 29.41% | 100% | 1.484E−05 |
| 36 | AGTGTGAGGGTTCGTGTCCCT | 23 | 29.41% | 100% | 1.484E−05 |
| 37 | CACCCGTAGTACCGACCTCGCG | 23 | 29.41% | 100% | 1.484E−05 |
| 38 | AGAGGTTCCGAGTTCGGGTCCCGGC | 23 | 29.41% | 100% | 1.484E−05 |
| 39 | TCCCCGGTGGTCTAGTGGTTAGGATTCCGCGCT | 23 | 29.41% | 100% | 1.484E−05 |
| 40 | GACGTCGGATCAGAAGA | 22 | 29.41% | 100% | 1.484E−05 |
| 41 | TTTTGGGATGACTTACA | 22 | 29.41% | 100% | 1.484E−05 |
| 42 | TTCACGTAATCCAGGAAAAGCT | 22 | 29.41% | 100% | 1.484E−05 |
| 43 | GAGGTTACGGGTTCGTGTCCCGGC | 22 | 29.41% | 100% | 1.484E−05 |
| 44 | ATGTGACTCTTAATCTC | 21 | 29.41% | 100% | 1.484E−05 |
| 45 | AGGGTGTGGGTTCGTCCCACC | 21 | 29.41% | 100% | 1.484E−05 |
| 46 | TATAGCACTCTGGACTCTGAATCCAGC | 20 | 29.41% | 100% | 1.484E−05 |

TABLE 2B
Disease Specific Biomarkers for Alzheimer's Disease Identified in Brain Tissue

| | Stage | | | | | | |
|---|---|---|---|---|---|---|---|
| | NA | NA | NA | NA | NA | NA | Braak V |
| Seq. ID | SRR1658347 | SRR1658348 | SRR1658349 | SRR1658350 | SRR1658351 | SRR1658353 | SRR828723 |
| 1 | | 0.549 | 0.225 | 2.012 | | | |
| 2 | | 0.549 | | 0.063 | | | |
| 3 | | | 0.674 | 0.44 | | | |
| 4 | | | | | | | |
| 5 | | | | | | | |
| 6 | 0.092 | 2.563 | 0.075 | 1.383 | 0.085 | 0.146 | |
| 7 | | | | | | | 0.181 |
| 8 | | | | | | | |
| 9 | | | | | | | |
| 10 | | 1.464 | | 0.754 | 0.085 | | |
| 11 | | 0.183 | | | | | |
| 12 | 0.092 | 0.732 | 0.15 | 0.88 | 0.085 | 0.146 | |
| 13 | | | | | | | |
| 14 | | | | | | | |
| 15 | | | | | | | |
| 16 | | | | | | | |
| 17 | | | | | | | |
| 18 | 0.277 | 2.014 | 0.075 | 3.583 | 0.085 | | |
| 19 | 0.277 | 6.407 | 0.449 | 1.006 | 0.17 | | |
| 20 | 0.277 | 3.844 | 0.15 | 2.2 | 0.085 | | |
| 21 | | 3.295 | 0.075 | 2.075 | 0.17 | | |
| 22 | 0.185 | 5.858 | 0.075 | 0.943 | 0.17 | | |
| 23 | 0.092 | 1.098 | 0.15 | 1.823 | 0.085 | | |
| 24 | | | | | | | |
| 25 | 0.092 | 3.478 | 0.3 | 0.503 | 0.255 | | |
| 26 | 0.185 | 2.929 | 0.075 | 0.88 | 0.085 | | |
| 27 | | | 0.075 | 1.634 | 0.17 | | |
| 28 | 0.277 | 2.014 | 0.524 | 0.566 | 0.085 | | |
| 29 | 0.185 | 0.366 | 0.15 | 1.257 | 0.34 | | |
| 30 | | 0.732 | 0.15 | 1.194 | 0.17 | | |
| 31 | 0.092 | 2.929 | 0.075 | 0.377 | 0.255 | | |
| 32 | | 1.098 | 0.075 | 1.006 | 0.17 | | |
| 33 | 0.092 | 3.112 | 0.3 | 0.126 | | | |
| 34 | 0.831 | 0.366 | 0.075 | 0.629 | 0.17 | | |
| 35 | 0.554 | 2.197 | 0.075 | 0.126 | 0.255 | | |
| 36 | 0.554 | 0.915 | 0.075 | 0.44 | 0.34 | | |
| 37 | | | | | | | 1.268 |
| 38 | 0.092 | 2.929 | 0.15 | 0.189 | 0.085 | | |
| 39 | | | | | | | 0.906 |
| 40 | 0.554 | 2.197 | 0.15 | 0.063 | | | |
| 41 | | | | | | | |
| 42 | | | 0.15 | | | | 1.087 |
| 43 | 0.092 | 2.929 | 0.075 | 0.189 | 0.085 | | |
| 44 | 0.092 | 0.549 | | 0.943 | | | |
| 45 | | 1.647 | 0.075 | 0.566 | 0.085 | | |
| 46 | | | | | | | |
| # Biomarkers Per | 20 | 28 | 27 | 29 | 23 | 2 | 4 |
| Sample | | | | | | | |
| % Coverage | 43% | 61% | 59% | 63% | 50% | 4% | 9% |
| | Stage | | | | | | |
| | Braak VI | Braak VI | Braak VI | Braak VI | Braak VI | Braak VI | Braak VI |
| Seq. ID | SRR1103943 | SRR1103944 | SRR1103945 | SRR1103946 | SRR1103947 | SRR1103948 | SRR828724 |
| 1 | 0.074 | 0.199 | 0.111 | 0.139 | 0.108 | | |
| 2 | 0.074 | 0.598 | 0.445 | 0.278 | 0.867 | 0.378 | |
| 3 | | 0.299 | 0.445 | 0.417 | 0.65 | 0.284 | |
| 4 | 0.222 | 0.498 | 0.668 | 1.252 | 0.867 | 3.595 | |
| 5 | 0.296 | 0.299 | 0.223 | 0.626 | 0.433 | 2.46 | |
| 6 | | | | | | | |
| 7 | 0.37 | 0.598 | | 1.183 | 0.433 | 0.473 | |
| 8 | 0.296 | 0.498 | 0.223 | 0.765 | 0.542 | 0.757 | |
| 9 | 0.37 | 0.199 | 0.223 | 0.835 | 0.433 | 0.284 | |
| 10 | 0.074 | | 0.111 | 0.07 | | | |
| 11 | | 0.199 | 0.445 | 0.905 | 0.217 | 0.095 | |
| 12 | | | | | | | |
| 13 | 0.074 | 0.299 | 0.334 | 0.348 | 0.65 | 0.378 | |
| 14 | 0.074 | | 0.111 | 0.557 | 0.758 | 0.378 | 0.211 |
| 15 | 0.148 | 0.199 | 0.334 | 0.278 | 0.325 | 0.662 | |
| 16 | 0.222 | 0.299 | 0.111 | 0.626 | 0.217 | 0.189 | |
| 17 | 0.222 | 0.199 | 0.668 | 0.209 | 0.108 | 0.473 | |
| 18 | | | | | | | |
| 19 | | | | | | | |
| 20 | | | | | | | |
| 21 | | | | | | | 0.211 |
| 22 | | | | | | | |
| 23 | | | | | | | |
| 24 | 0.296 | 0.1 | | 0.835 | 0.325 | 1.608 | |
| 25 | | | | | | | |
| 26 | | | | | | | |
| 27 | | | 0.111 | 0.07 | | | |
| 28 | | | | | | | |
| 29 | | | | | | | |
| 30 | | 0.1 | | | | | |
| 31 | | | | | | | |
| 32 | | | 0.111 | | | | |
| 33 | | | | | | | 0.211 |
| 34 | | | | | | | |
| 35 | | | | | | | |
| 36 | | | | | | | |
| 37 | | | | | | | 0.634 |
| 38 | | | | | | | |
| 39 | | | | | | | 2.747 |
| 40 | | | | | 0.108 | | |
| 41 | | 0.199 | 0.111 | 0.696 | 0.758 | 0.189 | |
| 42 | | | | | | | 2.536 |
| 43 | | | | | | | |
| 44 | | | | 0.07 | 0.108 | | |
| 45 | | | | 0.07 | | | |
| 46 | | 0.199 | 0.78 | 0.278 | 0.217 | 0.473 | |
| # Biomarkers Per | 14 | 17 | 18 | 21 | 19 | 16 | 6 |
| Sample | | | | | | | |
| % Coverage | 30% | 37% | 39% | 46% | 41% | 35% | 13% |

| | Stage | | |
|---|---|---|---|
| | Braak VI | Braak VI | Braak VI |
| Seq. ID | SRR828725 | SRR828726 | SRR828727 |
| 1 | | | |
| 2 | | | |
| 3 | | | |
| 4 | | | |
| 5 | | | |
| 6 | | | |
| 7 | | | |
| 8 | | | |
| 9 | | | |
| 10 | | | |
| 11 | | | |
| 12 | | | |
| 13 | | | |
| 14 | | | |
| 15 | | | |
| 16 | | | |
| 17 | | | |
| 18 | | | |
| 19 | | | |
| 20 | | | |
| 21 | | | |
| 22 | | | |
| 23 | | | |
| 24 | | | |
| 25 | | | |
| 26 | | | |
| 27 | | | |
| 28 | | | |
| 29 | | | |
| 30 | | | |
| 31 | | | |
| 32 | | | |
| 33 | | | |
| 34 | | | |
| 35 | | | |
| 36 | | | |
| 37 | 4.334 | 6.641 | 30.067 |
| 38 | | | |
| 39 | 4.334 | 1.811 | 30.067 |
| 40 | | | |
| 41 | | | |
| 42 | 4.334 | 0.604 | |
| 43 | | | |
| 44 | | | |
| 45 | | | |
| 46 | | | |
| # Biomarkers Per | 3 | 3 | 2 |
| Sample | | | |
| % Coverage | 7% | 7% | 4% |

TABLE 3A
Experimental Alzheimer's disease cohort for biomarker discovery,
taken from CSF samples

| | | | | Age | Disease | |
|---|---|---|---|---|---|---|
| | | Disease | Gen- | at | Dura- | Braak |
| | Sample ID | Type | der | Death | tion | Score |
| Experimental | SRR1568546 | Alzheimer's | F | 91 | 19 | II |
| Experimental | SRR1568552 | Alzheimer's | M | 79 | 5 | II |
| Experimental | SRR1568556 | Alzheimer's | M | 90 | 1 | III |
| Experimental | SRR1568685 | Alzheimer's | M | 85 | 1 | III |
| Experimental | SRR1568693 | Alzheimer's | F | 91 | 4 | III |
| Experimental | SRR1568751 | Alzheimer's | M | 83 | 3 | III |
| Experimental | SRR1568420 | Alzheimer's | F | 77 | 3 | IV |
| Experimental | SRR1568436 | Alzheimer's | F | 88 | 3 | IV |
| Experimental | SRR1568488 | Alzheimer's | M | 82 | 9 | IV |
| Experimental | SRR1568533 | Alzheimer's | F | 86 | NA | IV |
| Experimental | SRR1568540 | Alzheimer's | F | 91 | 10 | IV |
| Experimental | SRR1568585 | Alzheimer's | F | 89 | 9 | IV |
| Experimental | SRR1568644 | Alzheimer's | F | 79 | 14 | IV |
| Experimental | SRR1568651 | Alzheimer's | M | 88 | 5 | IV |
| Experimental | SRR1568655 | Alzheimer's | M | 87 | 9 | IV |
| Experimental | SRR1568733 | Alzheimer's | M | 80 | 3 | IV |
| Experimental | SRR1568743 | Alzheimer's | F | 85 | 5 | IV |
| Experimental | SRR1568368 | Alzheimer's | M | 87 | 12 | V |
| Experimental | SRR1568370 | Alzheimer's | M | 86 | 21 | V |
| Experimental | SRR1568397 | Alzheimer's | M | 83 | 8 | V |
| Experimental | SRR1568406 | Alzheimer's | M | 75 | 10 | V |
| Experimental | SRR1568408 | Alzheimer's | M | 76 | 2 | V |
| Experimental | SRR1568445 | Alzheimer's | M | 76 | 4 | V |
| Experimental | SRR1568454 | Alzheimer's | M | 80 | 8 | V |
| Experimental | SRR1568467 | Alzheimer's | M | 75 | 7 | V |
| Experimental | SRR1568474 | Alzheimer's | F | 86 | 9 | V |
| Experimental | SRR1568480 | Alzheimer's | F | 75 | 5 | V |
| Experimental | SRR1568514 | Alzheimer's | F | 78 | 8 | V |
| Experimental | SRR1568522 | Alzheimer's | F | 87 | 5 | V |
| Experimental | SRR1568573 | Alzheimer's | F | 86 | 17 | V |
| Experimental | SRR1568638 | Alzheimer's | M | 75 | 6 | V |
| Experimental | SRR1568642 | Alzheimer's | F | 86 | 10 | V |
| Experimental | SRR1568665 | Alzheimer's | F | 81 | 7 | V |
| Experimental | SRR1568667 | Alzheimer's | F | 85 | 1 | V |
| Experimental | SRR1568673 | Alzheimer's | M | 75 | 8 | V |
| Experimental | SRR1568687 | Alzheimer's | M | 82 | 7 | V |
| Experimental | SRR1568704 | Alzheimer's | F | 86 | 5 | V |
| Experimental | SRR1568718 | Alzheimer's | F | 74 | 7 | V |
| Experimental | SRR1568388 | Alzheimer's | F | 97 | 5 | VI |
| Experimental | SRR1568422 | Alzheimer's | F | 84 | 15 | VI |
| Experimental | SRR1568432 | Alzheimer's | F | 60 | 5 | VI |
| Experimental | SRR1568434 | Alzheimer's | F | 74 | 12 | VI |
| Experimental | SRR1568440 | Alzheimer's | F | 84 | 14 | VI |
| Experimental | SRR1568456 | Alzheimer's | M | 78 | 8 | VI |
| Experimental | SRR1568489 | Alzheimer's | F | 70 | 4 | VI |
| Experimental | SRR1568495 | Alzheimer's | F | 74 | 8 | VI |
| Experimental | SRR1568524 | Alzheimer's | F | 70 | 5 | VI |
| Experimental | SRR1568529 | Alzheimer's | F | 57 | 10 | VI |
| Experimental | SRR1568537 | Alzheimer's | F | 65 | 3 | VI |
| Experimental | SRR1568539 | Alzheimer's | F | 82 | 11 | VI |
| Experimental | SRR1568561 | Alzheimer's | M | 87 | 6 | VI |
| Experimental | SRR1568565 | Alzheimer's | M | 78 | 5 | VI |
| Experimental | SRR1568599 | Alzheimer's | M | 85 | 5 | VI |
| Experimental | SRR1568610 | Alzheimer's | F | 68 | 8 | VI |
| Experimental | SRR1568640 | Alzheimer's | M | 83 | 6 | VI |
| Experimental | SRR1568647 | Alzheimer's | M | 77 | 1 | VI |
| Experimental | SRR1568661 | Alzheimer's | F | 93 | 3 | VI |
| Experimental | SRR1568663 | Alzheimer's | M | 81 | 7 | VI |
| Experimental | SRR1568672 | Alzheimer's | F | 78 | 7 | VI |
| Experimental | SRR1568677 | Alzheimer's | F | 90 | 12 | VI |
| Experimental | SRR1568722 | Alzheimer's | M | 83 | 8 | VI |
| Experimental | SRR1568740 | Alzheimer's | M | 80 | 10 | VI |
| Experimental | SRR1568747 | Alzheimer's | F | 89 | 9 | VI |
| Experimental | SRR1568755 | Alzheimer's | F | 79 | 10 | VI |
| AVERGAGE | NA | NA | NA | 81.00 ± | NA | NA |
| | | | | 10.1 | | |

TABLE 3B
Comparator cohort for AD biomarker discovery, taken from CSF samples, including
healthy controls and various other non-Alzheimer's neurological disorders

| | | | | Age at | Braak |
|---|---|---|---|---|---|
| Group | Sample ID | Disease Type | Gender | Death | Score |
| Comparator | SRR1568380 | Control | F | 88 | II |
| Comparator | SRR1568384 | Control | F | 78 | III |
| Comparator | SRR1568386 | Control | F | 90 | III |
| Comparator | SRR1568393 | Control | F | 80 | III |
| Comparator | SRR1568404 | Control | M | 85 | III |
| Comparator | SRR1568413 | Control | M | 89 | IV |
| Comparator | SRR1568415 | Control | F | 88 | III |
| Comparator | SRR1568417 | Control | M | 80 | II |
| Comparator | SRR1568428 | Control | M | 80 | I |
| Comparator | SRR1568441 | Control | M | 86 | II |
| Comparator | SRR1568447 | Control | F | 85 | III |
| Comparator | SRR1568459 | Control | F | 78 | IV |
| Comparator | SRR1568461 | Control | M | 82 | IV |
| Comparator | SRR1568463 | Control | F | 83 | II |
| Comparator | SRR1568469 | Control | F | 86 | IV |
| Comparator | SRR1568476 | Control | M | 82 | III |
| Comparator | SRR1568482 | Control | M | 75 | IV |
| Comparator | SRR1568484 | Control | M | 91 | IV |
| Comparator | SRR1568491 | Control | F | 88 | III |
| Comparator | SRR1568493 | Control | M | 84 | II |
| Comparator | SRR1568497 | Control | F | 87 | III |
| Comparator | SRR1568499 | Control | M | 84 | II |
| Comparator | SRR1568501 | Control | M | 73 | II |
| Comparator | SRR1568505 | Control | M | 78 | II |
| Comparator | SRR1568508 | Control | M | 89 | III |
| Comparator | SRR1568520 | Control | F | 84 | III |
| Comparator | SRR1568526 | Control | F | 90 | III |
| Comparator | SRR1568527 | Control | F | 75 | III |
| Comparator | SRR1568542 | Control | F | 88 | III |
| Comparator | SRR1568544 | Control | F | 87 | IV |
| Comparator | SRR1568550 | Control | F | 76 | I |
| Comparator | SRR1568559 | Control | M | 87 | IV |
| Comparator | SRR1568563 | Control | M | 76 | I |
| Comparator | SRR1568567 | Control | M | 94 | IV |
| Comparator | SRR1568569 | Control | M | 71 | I |
| Comparator | SRR1568578 | Control | F | 91 | IV |
| Comparator | SRR1568581 | Control | M | 82 | III |
| Comparator | SRR1568583 | Control | M | 65 | I |
| Comparator | SRR1568589 | Control | F | 99 | III |
| Comparator | SRR1568591 | Control | M | 92 | IV |
| Comparator | SRR1568593 | Control | M | 38 | 0 |
| Comparator | SRR1568601 | Control | M | 97 | III |
| Comparator | SRR1568602 | Control | M | 53 | I |
| Comparator | SRR1568605 | Control | M | 80 | III |
| Comparator | SRR1568608 | Control | M | 85 | III |
| Comparator | SRR1568612 | Control | F | 59 | I |
| Comparator | SRR1568614 | Control | F | 95 | III |
| Comparator | SRR1568620 | Control | F | 84 | IV |
| Comparator | SRR1568626 | Control | M | 93 | I |
| Comparator | SRR1568632 | Control | F | 92 | III |
| Comparator | SRR1568635 | Control | M | 74 | II |
| Comparator | SRR1568649 | Control | M | 90 | III |
| Comparator | SRR1568653 | Control | M | 84 | III |
| Comparator | SRR1568659 | Control | M | 78 | II |
| Comparator | SRR1568670 | Control | M | 83 | I |
| Comparator | SRR1568675 | Control | M | 79 | I |
| Comparator | SRR1568681 | Control | M | 84 | III |
| Comparator | SRR1568695 | Control | F | 87 | III |
| Comparator | SRR1568697 | Control | M | 90 | III |
| Comparator | SRR1568706 | Control | F | 73 | I |
| Comparator | SRR1568708 | Control | M | 78 | III |
| Comparator | SRR1568712 | Control | F | 70 | I |
| Comparator | SRR1568720 | Control | M | 86 | II |
| Comparator | SRR1568727 | Control | F | 76 | I |
| Comparator | SRR1568731 | Control | F | 88 | III |
| Comparator | SRR1568735 | Control | M | 81 | IV |
| Comparator | SRR1568741 | Control | M | 69 | I |
| Comparator | SRR1568749 | Control | F | 91 | III |
| Comparator | SRR1568366 | Parkinson's Disease | M | 70 | III |
| Comparator | SRR1568382 | Parkinson's Disease | M | 85 | II |
| Comparator | SRR1568424 | Parkinson's Disease | F | 86 | IV |
| Comparator | SRR1568450 | Parkinson's Disease | M | 89 | III |
| Comparator | SRR1568457 | Parkinson's Disease | F | 79 | IV |
| Comparator | SRR1568486 | Parkinson's Disease | M | 73 | I |
| Comparator | SRR1568512 | Parkinson's Disease | F | 87 | I |
| Comparator | SRR1568531 | Parkinson's Disease | F | 81 | III |
| Comparator | SRR1568554 | Parkinson's Disease | M | 86 | III |
| Comparator | SRR1568576 | Parkinson's Disease | F | 79 | II |
| Comparator | SRR1568630 | Parkinson's Disease | M | 80 | II |
| Comparator | SRR1568700 | Parkinson's Disease | M | 81 | I |
| Comparator | SRR1568702 | Parkinson's Disease | M | 77 | III |
| Comparator | SRR1568716 | Parkinson's Disease | F | 77 | II |
| Comparator | SRR1568724 | Parkinson's Disease | F | 83 | III |
| Comparator | SRR1568726 | Parkinson's Disease | F | 89 | IV |
| Comparator | SRR1568738 | Parkinson's Disease | F | 78 | III |
| Comparator | SRR1568364 | Parkinson's Disease | F | 73 | III |
| | | with Dementia | | | |
| Comparator | SRR1568372 | Parkinson's Disease | F | 87 | IV |
| | | with Dementia | | | |
| Comparator | SRR1568400 | Parkinson's Disease | F | 78 | III |
| | | with Dementia | | | |
| Comparator | SRR1568402 | Parkinson's Disease | F | 82 | III |
| | | with Dementia | | | |
| Comparator | SRR1568412 | Parkinson's Disease | M | 74 | I |
| | | with Dementia | | | |
| Comparator | SRR1568426 | Parkinson's Disease | M | 78 | III |
| | | with Dementia | | | |
| Comparator | SRR1568430 | Parkinson's Disease | M | 79 | II |
| | | with Dementia | | | |
| Comparator | SRR1568443 | Parkinson's Disease | M | 70 | II |
| | | with Dementia | | | |
| Comparator | SRR1568452 | Parkinson's Disease | M | 83 | III |
| | | with Dementia | | | |
| Comparator | SRR1568478 | Parkinson's Disease | F | 84 | II |
| | | with Dementia | | | |
| Comparator | SRR1568516 | Parkinson's Disease | M | 83 | 0 |
| | | with Dementia | | | |
| Comparator | SRR1568518 | Parkinson's Disease | F | 82 | III |
| | | with Dementia | | | |
| Comparator | SRR1568548 | Parkinson's Disease | M | 75 | III |
| | | with Dementia | | | |
| Comparator | SRR1568571 | Parkinson's Disease | M | 74 | III |
| | | with Dementia | | | |
| Comparator | SRR1568575 | Parkinson's Disease | M | 75 | IV |
| | | with Dementia | | | |
| Comparator | SRR1568616 | Parkinson's Disease | F | 85 | III |
| | | with Dementia | | | |
| Comparator | SRR1568624 | Parkinson's Disease | F | 84 | IV |
| | | with Dementia | | | |
| Comparator | SRR1568628 | Parkinson's Disease | M | 83 | III |
| | | with Dementia | | | |
| Comparator | SRR1568657 | Parkinson's Disease | F | 87 | II |
| | | with Dementia | | | |
| Comparator | SRR1568683 | Parkinson's Disease | M | 72 | I |
| | | with Dementia | | | |
| Comparator | SRR1568689 | Parkinson's Disease | M | 76 | III |
| | | with Dementia | | | |
| Comparator | SRR1568710 | Parkinson's Disease | M | 83 | III |
| | | with Dementia | | | |
| Comparator | SRR1568729 | Parkinson's Disease | F | 79 | II |
| | | with Dementia | | | |
| Comparator | SRR1568753 | Parkinson's Disease | M | 85 | III |
| | | with Dementia | | | |
| AVERGAGE | NA | NA | NA | 81.41 ± 8.5 | NA |

TABLE 4A
Disease Specific Biomarkers for Alzheimer's Disease Identified in CSF

| Seq. | | Total | Frequency | Speci- | p-value in |
|---|---|---|---|---|---|
| ID | Sequence | Reads | (Sensitivity) | ficity | Discovery set |
| 47 | CCACGGACTCCCAAAAGCAGCTT | 16 | 9.38% | 100% | 2.20E−03 |
| 48 | ACCCCGTAGATCCGACCTTGTGA | 14 | 9.38% | 100% | 2.20E−03 |
| 49 | TCACCGGGTGTACATCAAGC | 9 | 9.38% | 100% | 2.20E−03 |
| 50 | CAACGGAATCTCCAAAGCAGCT | 9 | 9.38% | 100% | 2.20E−03 |
| 51 | TCTTGCACTCGTCCCGGCCTCAT | 9 | 9.38% | 100% | 2.20E−03 |
| 52 | TTTCGGCACTGAGGCCT | 8 | 9.38% | 100% | 2.20E−03 |
| 53 | TCACCCGGGTGTCAATCAGCTG | 8 | 9.38% | 100% | 2.20E−03 |
| 54 | CCCCCGTCGAACCGCCCTTGCGA | 8 | 9.38% | 100% | 2.20E−03 |
| 55 | GTTAAAATTCCTGAACCGGGACGCGGC | 33 | 9.38% | 100% | 2.20E−03 |
| 56 | GGTTCGTGCTGACGGCCTGTATCCTAGGCTACA | 31 | 9.38% | 100% | 2.20E−03 |
| | CCCTGAGGACT | | | | |
| 57 | CCCCCGTCGAACCGACCTTG | 27 | 9.38% | 100% | 2.20E−03 |
| 58 | TTCACAGTGGCTCAGTTCTGCC | 21 | 9.38% | 100% | 2.20E−03 |
| 59 | TTAAACTCTGTCGTGCTGG | 19 | 9.38% | 100% | 2.20E−03 |
| 60 | GCTAATACCGGATAAGAAAGC | 18 | 9.38% | 100% | 2.20E−03 |
| 61 | TCCCTGGTGGTCTGGTGGTTAGGAGTCGGCGC | 18 | 9.38% | 100% | 2.20E−03 |
| 62 | TAAAGTGCTGACCGTGCAGAT | 16 | 9.38% | 100% | 2.20E−03 |
| 63 | TCCTCTGTAGTTCAGTCGGTAGAAC | 13 | 9.38% | 100% | 2.20E−03 |
| 64 | TCCCTGTGGTCTAATGGTTAGGATCCGGCGCT | 13 | 9.38% | 100% | 2.20E−03 |
| 65 | CCTTGGCTGGGAGAACGCCTGGGAATACCGGG | 12 | 9.38% | 100% | 2.20E−03 |
| | TGCTGTAGGCTT | | | | |
| 66 | CAACATAGCGAGCCCCCGTCTCT | 11 | 9.38% | 100% | 2.20E−03 |
| 67 | CAGTTGCCACGTTCCCGTGG | 10 | 9.38% | 100% | 2.20E−03 |
| 68 | TGTAAACCTCCTGGCCTGGAAGCT | 10 | 9.38% | 100% | 2.20E−03 |
| 69 | CGCATTGCCGAGTAGCTATGTTCGGATG | 10 | 9.38% | 100% | 2.20E−03 |
| 70 | GACGGAAAGACCCCATGAACCTTTACTGTAGCT | 10 | 9.38% | 100% | 2.20E−03 |
| | TTGTATTGGAC | | | | |
| 71 | GGCTAATACCTGGGACTC | 9 | 9.38% | 100% | 2.20E−03 |
| 72 | CGCGGGGTGGAGCAGCCTGGTAGCT | 9 | 9.38% | 100% | 2.20E−03 |
| 73 | CGGGTCGTGGGTTCGCCCCACGTTGGGCGC | 9 | 9.38% | 100% | 2.20E−03 |
| 74 | TCTACAGTCCGACGATACGACTCTTAGCGG | 9 | 9.38% | 100% | 2.20E−03 |
| 75 | GGGCCCCTACCCGGCCGTCGCCGGCAGTCGAG | 9 | 9.38% | 100% | 2.20E−03 |
| 76 | TCTTCCGTAGTGTAGTGGTTATGACGTTCGCCT | 9 | 9.38% | 100% | 2.20E−03 |
| 77 | TCAAGGCTAAAACTCAAA | 8 | 9.38% | 100% | 2.20E−03 |
| 78 | TACAGTACTGTGCTAACTGAAAA | 8 | 9.38% | 100% | 2.20E−03 |
| 79 | GCCACGGTGGCCGAGTGGTTAAGGC | 8 | 9.38% | 100% | 2.20E−03 |
| 80 | CCCCCACTGCTACATTTGACTGTCTT | 8 | 9.38% | 100% | 2.20E−03 |
| 81 | ACGGATAAAAGGTACCTCGGGGATAAC | 8 | 9.38% | 100% | 2.20E−03 |
| 82 | CTTCTAGAAATTTCTGAAAATGCTCTG | 8 | 9.38% | 100% | 2.20E−03 |
| 83 | CCCCCCACTGCTAAATTTGACTGGCTACT | 8 | 9.38% | 100% | 2.20E−03 |
| 84 | GGCCGCGTGCCTAATGGATAAGGCGTCTGAT | 8 | 9.38% | 100% | 2.20E−03 |
| 85 | CTGTGAGGGTGAGCGAATCGCTGAAAGCCGGC | 8 | 9.38% | 100% | 2.20E−03 |
| | C | | | | |
| 86 | GCTTGCGGAGTGTAGTGGTTATCACGTTCGCCT | 8 | 9.38% | 100% | 2.20E−03 |
| 87 | CAACGGATAAAAGGTACTCTAGGGATAACAGG | 8 | 9.38% | 100% | 2.20E−03 |
| | CT | | | | |
| 88 | CATTGGTGGTTCCGTGGTAGAATTCTCGCCTGC | 8 | 9.38% | 100% | 2.20E−03 |
| | C | | | | |
| 89 | GGCTGGTCCGATGGTAGTGGGGTATCAGAACT | 8 | 9.38% | 100% | 2.20E−03 |
| | TG | | | | |
| 90 | TTGACCTTACCGGATGGCACAAAGAGAAGTGG | 8 | 9.38% | 100% | 2.20E−03 |
| | GCAAGTTC | | | | |
| 91 | TCCCTAGTTCGTTTCTGGGAGCGGAGACCA | 49 | 9.38% | 100% | 2.20E−03 |
| 92 | TCCCATGTGGTCTAGCGGTTAGGATTCCT | 29 | 9.38% | 100% | 2.20E−03 |
| 93 | CGGGCCTTTCGGGGCCTCTTCCCCGGGC | 22 | 9.38% | 100% | 2.20E−03 |
| 94 | GTGGTTCCGGCTTTGGAC | 18 | 9.38% | 100% | 2.20E−03 |
| 95 | GTGCTAATCTGCGATAAGCGTCGGT | 16 | 9.38% | 100% | 2.20E−03 |
| 96 | TCAGTGCATCACCGACCTTTGTT | 15 | 9.38% | 100% | 2.20E−03 |
| 97 | TCCCTGAGACCCTTTAAACCTGT | 15 | 9.38% | 100% | 2.20E−03 |
| 98 | CTAGTACGAGAGGACCGGAGTGGACGCATC | 15 | 9.38% | 100% | 2.20E−03 |
| 99 | GAGGCAGCAGTAGGGAATAT | 14 | 9.38% | 100% | 2.20E−03 |
| 100 | TAGCACCATTTGCAATCGGTTG | 14 | 9.38% | 100% | 2.20E−03 |
| 101 | TTAGACAGTTCGGTCCCTATCTGCC | 14 | 9.38% | 100% | 2.20E−03 |
| 102 | TGATGTCGGCTCATCTCATCCTGGGGCT | 14 | 9.38% | 100% | 2.20E−03 |
| 103 | AATCCTGGTCGGACATCA | 13 | 9.38% | 100% | 2.20E−03 |
| 104 | TGCACCATGGTTCTCTGAGCATG | 13 | 9.38% | 100% | 2.20E−03 |
| 105 | TGGGGAGTTCGAGTCTCTCCGCCCCTGCCA | 13 | 9.38% | 100% | 2.20E−03 |
| 106 | CCAAGGGGTCGTGGGTTCGAATCCTGCCAGCC | 13 | 9.38% | 100% | 2.20E−03 |
| | GCACCA | | | | |
| 107 | TCGTGATACAGTTCGGTC | 12 | 9.38% | 100% | 2.20E−03 |
| 108 | TCCGGGGAGCACGCCTGTTCGAGTATCGT | 12 | 9.38% | 100% | 2.20E−03 |
| 109 | GCCCCGTTCGTCTAGCGGCCTAGGACGCC | 12 | 9.38% | 100% | 2.20E−03 |
| | GGCCTCT | | | | |
| 110 | CTTCCACAACGTTCCCG | 11 | 9.38% | 100% | 2.20E−03 |
| 111 | TTCGATCCCGTCATCACC | 11 | 9.38% | 100% | 2.20E−03 |
| 112 | AAAGAGGAGGAGAGGAGAAC | 11 | 9.38% | 100% | 2.20E−03 |
| 113 | TCCACCACGTTCCCGTGGTAAATCAGCTTG | 11 | 9.38% | 100% | 2.20E−03 |
| 114 | GCAAGCAGGGGTCGTCGGTTCGATCCCGTC | 11 | 9.38% | 100% | 2.20E−03 |
| | ATCCTCCACCA | | | | |
| 115 | CCCCCACGTTCCCGTTGG | 10 | 9.38% | 100% | 2.20E−03 |
| 116 | TTTGGTATCTGCGCTCTGC | 10 | 9.38% | 100% | 2.20E−03 |
| 117 | CACCTTGCGCAATCAGGACTGA | 10 | 9.38% | 100% | 2.20E−03 |
| 118 | GGGATAGTAGGTCGTTGCCAACC | 10 | 9.38% | 100% | 2.20E−03 |
| 119 | GGAAGAACGGGTGCTGTAGGCTTT | 10 | 9.38% | 100% | 2.20E−03 |
| 120 | CGAGACCAGGACTTTGATAGGCTGGGTG | 10 | 9.38% | 100% | 2.20E−03 |
| 121 | AAGCAGCAATGCGACGTATAGGGTCTGACGCC | 10 | 9.38% | 100% | 2.20E−03 |
| | T | | | | |
| 122 | TCAAATGGTAGAGCGCTCGCTTGGCTTGCGAG | 10 | 9.38% | 100% | 2.20E−03 |
| | A | | | | |
| 123 | GACCCAGTTGCCTAATTGGATAAGGCATCAGCC | 10 | 9.38% | 100% | 2.20E−03 |
| | T | | | | |
| 124 | TCCCTGGTGGTCTGGTGGTTAGGAGTCGGCGCT | 10 | 9.38% | 100% | 2.20E−03 |
| | CT | | | | |
| 125 | ATAGATCCTGAAACCGC | 9 | 9.38% | 100% | 2.20E−03 |
| 126 | CTCTTCGAGGCCCTGTAAT | 9 | 9.38% | 100% | 2.20E−03 |
| 127 | AGGTCCTCAATACGTATTTG | 9 | 9.38% | 100% | 2.20E−03 |
| 128 | CAAGGCAAAGACGCGTAGCT | 9 | 9.38% | 100% | 2.20E−03 |
| 129 | AACTGGAGAGTTTGATTCTGGCT | 9 | 9.38% | 100% | 2.20E−03 |
| 130 | CGGTGAATACGTTCCCGGGCCTT | 9 | 9.38% | 100% | 2.20E−03 |
| 131 | TTCCCTTTTTAATCCTATGCCTG | 9 | 9.38% | 100% | 2.20E−03 |
| 132 | AGCACGCGCGCACGTGTTAGGACC | 9 | 9.38% | 100% | 2.20E−03 |
| 133 | CAGATGGCGGAATTGGTAGACGCGCT | 9 | 9.38% | 100% | 2.20E−03 |
| 134 | CGTGGTTCATTTCCCCCTTTCGGGCG | 9 | 9.38% | 100% | 2.20E−03 |
| 135 | GGTCGATGATGATTGGTAAAAGGTCTG | 9 | 9.38% | 100% | 2.20E−03 |
| 136 | GTCGCCGGTTCAAGTCCGGCAGTCGGCTCCA | 9 | 9.38% | 100% | 2.20E−03 |
| 137 | AACACCGTGGAAGTTCGAGTCTTCTCCTG | 9 | 9.38% | 100% | 2.20E−03 |
| | GGCACCA | | | | |
| 138 | AGGGATGTCGCTCAACG | 8 | 9.38% | 100% | 2.20E−03 |
| 139 | GCCTGTAGTCGTGCCCG | 8 | 9.38% | 100% | 2.20E−03 |
| 140 | AATCGATCGAGGGCTTAAC | 8 | 9.38% | 100% | 2.20E−03 |
| 141 | GCAACCATCCTCTGCTACC | 8 | 9.38% | 100% | 2.20E−03 |
| 142 | TCAACTTCGGAACTGCCTT | 8 | 9.38% | 100% | 2.20E−03 |
| 143 | ACATTGGGACTGAGCCACGGC | 8 | 9.38% | 100% | 2.20E−03 |
| 144 | GGAGGGGAGTGAAATAGAACC | 8 | 9.38% | 100% | 2.20E−03 |
| 145 | TGAATACCGTGCTGTAGGCTT | 8 | 9.38% | 100% | 2.20E−03 |
| 146 | CTAATCGATCGAGGGCTTAACC | 8 | 9.38% | 100% | 2.20E−03 |
| 147 | TGACCGGGAGTCAATCAGCTTG | 8 | 9.38% | 100% | 2.20E−03 |
| 148 | TGAGGGGCAGAGCGCGAGACTA | 8 | 9.38% | 100% | 2.20E−03 |
| 149 | TGCGGACAAGGGGAATCTGACT | 8 | 9.38% | 100% | 2.20E−03 |
| 150 | TTATGTAGTAGATTGTTATAGT | 8 | 9.38% | 100% | 2.20E−03 |
| 151 | CCCCGTCCGCCCCCCGTTCCCCC | 8 | 9.38% | 100% | 2.20E−03 |
| 152 | GGAGGGGCAGAGAGCGAGCCTTT | 8 | 9.38% | 100% | 2.20E−03 |
| 153 | TAGGGGTGAAAGGCTAAACAAAC | 8 | 9.38% | 100% | 2.20E−03 |
| 154 | TGTCTGAACATGGGGGGACCACC | 8 | 9.38% | 100% | 2.20E−03 |
| 155 | TTCATTCGGCTGTCCGAGATGTA | 8 | 9.38% | 100% | 2.20E−03 |
| 156 | AGCTAGACAGCAGGACGGTGGCCA | 8 | 9.38% | 100% | 2.20E−03 |
| 157 | TTATGGCCAGGCTGTCTCCACCCGA | 8 | 9.38% | 100% | 2.20E−03 |
| 158 | AATAGAACCTGAAACCGGATGCCTAC | 8 | 9.38% | 100% | 2.20E−03 |
| 159 | CGCGCTCGCCGGCCGAGGTGGGATCCC | 8 | 9.38% | 100% | 2.20E−03 |
| 160 | GCGGATGTGGCTCAGCTGGTAGAGCATC | 8 | 9.38% | 100% | 2.20E−03 |
| 161 | CTCGTACCAAACGAGAACTTTGAAGGCCGAAG | 8 | 9.38% | 100% | 2.20E−03 |
| 162 | GCGGCTGTAGTGTAGTGGTGATCACGTTCGCCC | 8 | 9.38% | 100% | 2.20E−03 |
| 163 | ACGTAGAGGCCGGAGGTTCGAATCCTCTCACCC | 8 | 9.38% | 100% | 2.20E−03 |
| | C | | | | |
| 164 | TCATTGGTGGTTCAGTGGTAGACTTCTCGCCTG | 8 | 9.38% | 100% | 2.20E−03 |
| | CC | | | | |
| 165 | ACGATGTGGGATTGCATTGACAATCAGGAGGT | 8 | 9.38% | 100% | 2.20E−03 |
| | TGGCT | | | | |
| 166 | AACCTATCTGTGTAGGATAGGTGGGAGGCTTT | 8 | 9.38% | 100% | 2.20E−03 |
| | GAAGTC | | | | |
| 167 | CTAAATACTCGTACATGACC | 16 | 10.94% | 100% | 7.63E−04 |
| 168 | CCCTAGCTTGTGCGCTCCTGGA | 15 | 10.94% | 100% | 7.63E−04 |
| 169 | TGCAACTCGACTCCATGAAGTC | 10 | 10.94% | 100% | 7.63E−04 |
| 170 | TCCCCGTAATCTTCATAATCCGGAG | 8 | 10.94% | 100% | 7.63E−04 |
| 171 | GCATTGGTGGTTCGGTGGTAGAATGCTCGCCTG | 17 | 10.94% | 100% | 7.63E−04 |
| 172 | TTCGAGCCCCGCGGGTGCTTACTGACCCTTT | 15 | 10.94% | 100% | 7.63E−04 |
| 173 | ACTTGGCTGGGAGACCGCCTGGGAATACCGGG | 14 | 10.94% | 100% | 7.63E−04 |
| | TGCTGTATGCT | | | | |
| 174 | CCCCATGAAGTCGGAGTCGCTAGTAATCGCAG | 13 | 10.94% | 100% | 7.63E−04 |
| | AT | | | | |
| 175 | AATTGGCATGAGTCCACTTTAAATCCTTTAACG | 12 | 10.94% | 100% | 7.63E−04 |
| | AGGATCCAT | | | | |
| 176 | CAAAACTCCCGTGCTGATC | 10 | 10.94% | 100% | 7.63E−04 |
| 177 | TGCCCGTTGGTCTAGGGGGATGATTCTCGCTT | 10 | 10.94% | 100% | 7.63E−04 |
| 178 | TCCTCGATAGCTCAGTTGGTAGAGCGCCGGACT | 10 | 10.94% | 100% | 7.63E−04 |
| 179 | CGAGCCCAGGTTGGAGAGCCA | 9 | 10.94% | 100% | 7.63E−04 |
| 180 | GATCAGCTACCGTCGTAGTTC | 9 | 10.94% | 100% | 7.63E−04 |
| 181 | GTCTTTTTGTCCTCCTATGCCTG | 9 | 10.94% | 100% | 7.63E−04 |
| 182 | ATGGTTCGCACTCTGGACTCTGAAT | 9 | 10.94% | 100% | 7.63E−04 |
| 183 | CCACGTTCCCGTGGATTCCACCACGTTCCCGGG | 9 | 10.94% | 100% | 7.63E−04 |
| | G | | | | |
| 184 | CCTAAAAAGACGGATGTTGCTGAGTGTGGACC | 9 | 10.94% | 100% | 7.63E−04 |
| | TGG | | | | |
| 185 | TAGAAACCGGGCGGAAACA | 8 | 10.94% | 100% | 7.63E−04 |
| 186 | CTGGAGACCGGGGTTCGATTTCCCGACGGGGA | 8 | 10.94% | 100% | 7.63E−04 |
| | GCC | | | | |
| 187 | TCTGCTGAGGCTAAGCCCGTGTTCTAAAGATTT | 8 | 10.94% | 100% | 7.63E−04 |
| | GT | | | | |
| 188 | CCATGTGTCGTAGGTTCGAATCCTATCGGGGCC | 8 | 10.94% | 100% | 7.63E−04 |
| | GCCA | | | | |
| 189 | TCAGTGCATGACCGAACTTGT | 26 | 10.94% | 100% | 7.63E−04 |
| 190 | TAGTTGGTTTTCGGAACTGAGGCCA | 20 | 10.94% | 100% | 7.63E−04 |
| 191 | GGACAGTGTCTGGTGGGTAGTTTGACTGGGGC | 16 | 10.94% | 100% | 7.63E−04 |
| | GGTCTCCT | | | | |
| 192 | TGCCCTTTGTCATCCTCTTCCTG | 14 | 10.94% | 100% | 7.63E−04 |
| 193 | CGCTACCTCAGATCAGGACGTGGCGACCCGCT | 14 | 10.94% | 100% | 7.63E−04 |
| | GAAT | | | | |
| 194 | GTTGTCGTGGGTTCGAGCCCCATCAGCCACCCC | 13 | 10.94% | 100% | 7.63E−04 |
| | A | | | | |
| 195 | GCGGAAGTAGTTCAGTGGTAGAACATCA | 12 | 10.94% | 100% | 7.63E−04 |
| 196 | CGCGACCTCAGATCAGACGTGGCGACCCGCTG | 12 | 10.94% | 100% | 7.63E−04 |
| | AGTGTAAGC | | | | |
| 197 | GCAGGTTCAGTCCTGCCGCGGTCGC | 11 | 10.94% | 100% | 7.63E−04 |
| 198 | GTGATATAGACAGCAGGACGGTGGCCA | 11 | 10.94% | 100% | 7.63E−04 |
| 199 | CCAGTGTGAAAGTAGGTTATCTTCAGGCT | 11 | 10.94% | 100% | 7.63E−04 |
| 200 | GTACCGGGTGTAAATCAGCTG | 10 | 10.94% | 100% | 7.63E−04 |
| 201 | CACCGAAATCGCGGATATGAGCGTTCCT | 10 | 10.94% | 100% | 7.63E−04 |
| 202 | AGTCTGGCACGGTGAAGAGACATGAGAGGGG | 10 | 10.94% | 100% | 7.63E−04 |
| 203 | GTAACCGGGGTTCGAATCCCCGTAGGGACGCC | 10 | 10.94% | 100% | 7.63E−04 |
| | A | | | | |
| 204 | GCTGCATGGCCGTCGTC | 9 | 10.94% | 100% | 7.63E−04 |
| 205 | CGGGCGCTGTAGGCTTTT | 9 | 10.94% | 100% | 7.63E−04 |
| 206 | GTCCTCTCGGCCGCACCA | 9 | 10.94% | 100% | 7.63E−04 |
| 207 | CGCAGAGTCGCGCAGCGGAAG | 9 | 10.94% | 100% | 7.63E−04 |
| 208 | CGGGGTGTAGCTTAGCCTGGTA | 9 | 10.94% | 100% | 7.63E−04 |
| 209 | GCCGGCTAGCTCAGTCGGTAGAG | 9 | 10.94% | 100% | 7.63E−04 |
| 210 | TTCCGTTTGTCATCCTATGGCTG | 9 | 10.94% | 100% | 7.63E−04 |
| 211 | ATCCTGTCTGAATATGGGGGGACC | 9 | 10.94% | 100% | 7.63E−04 |
| 212 | GGCTCATAACCCGAAGGTCGTCGGT | 9 | 10.94% | 100% | 7.63E−04 |
| 213 | TCCAGGGTTCAGTTCCCTGTTCGGGCG | 9 | 10.94% | 100% | 7.63E−04 |
| 214 | ACGGATAAAAGGTACCTCGGGGATAACAG | 9 | 10.94% | 100% | 7.63E−04 |
| 215 | GCATTTGTGGTGCAGTGGTAGAATTCTAGCCT | 9 | 10.94% | 100% | 7.63E−04 |
| 216 | CACAACGAGATCACCTCTGGGTCGTCTGCCGGT | 9 | 10.94% | 100% | 7.63E−04 |
| | CTCCACC | | | | |
| 217 | CTGCACTACAGCCTGGGCAACATAGCGAGACCC | 9 | 10.94% | 100% | 7.63E−04 |
| | CGTCTCTA | | | | |
| 218 | ATTGACCGATTGAGAGCT | 8 | 10.94% | 100% | 7.63E−04 |
| 219 | CCGGGGCCACGTGCCCGTGG | 8 | 10.94% | 100% | 7.63E−04 |
| 220 | GTTCAGATCCCGGACGAGCCA | 8 | 10.94% | 100% | 7.63E−04 |
| 221 | TCAAACAGAACTTTGAAGGCCGAAG | 8 | 10.94% | 100% | 7.63E−04 |
| 222 | CGTGTTCAGGTGACGTCGGGGTCACC | 8 | 10.94% | 100% | 7.63E−04 |
| 223 | TGTCGGGCTGGGGCGCGAAGCGGGGC | 8 | 10.94% | 100% | 7.63E−04 |
| 224 | GCCCGGCTAGCTCAGTCGGTAGATCATGAGAC | 8 | 10.94% | 100% | 7.63E−04 |
| | A | | | | |
| 225 | TCCCACATCGTCCAGCGGTTAGGATTCCTGGTT | 8 | 10.94% | 100% | 7.63E−04 |
| 226 | TCCCTGGTGGTCTAGTGACTAGGATTCGGCGCT | 8 | 10.94% | 100% | 7.63E−04 |
| | T | | | | |
| 227 | ACAAACCGGAGGAAGGT | 9 | 12.50% | 100% | 2.62E−04 |
| 228 | CTCGACCCTTCGAACGCACTTGCGGCCCCGGGT | 26 | 12.50% | 100% | 2.62E−04 |
| | T | | | | |
| 229 | GTAGTACCGCCATGTCTGT | 9 | 12.50% | 100% | 2.62E−04 |
| 230 | CGGTGGCACCACGTTCCCGGGG | 9 | 12.50% | 100% | 2.62E−04 |
| 231 | GCCACGATCGACTGAGATTCAGCCTTTGTTCTG | 9 | 12.50% | 100% | 2.62E−04 |
| | TAGATTTGT | | | | |
| 232 | TAGAGGTTATCACGTCTGCTT | 8 | 12.50% | 100% | 2.62E−04 |
| 233 | CAGATGGTAGTGGGTTATCAGAACTT | 8 | 12.50% | 100% | 2.62E−04 |
| 234 | GCTTGCGTAGGGTAGTGGTTATCACGTTCGCCT | 8 | 12.50% | 100% | 2.62E−04 |
| 235 | TAGACCGCCTGGGAATACCGGTTGCTGTAGGCT | 24 | 12.50% | 100% | 2.62E−04 |
| | T | | | | |
| 236 | GGGAGGCTTTGAAGTGTGGACGCCAGTCTGC | 16 | 12.50% | 100% | 2.62E−04 |
| 237 | GGGATGAACCGACCGCCGGGTT | 15 | 12.50% | 100% | 2.62E−04 |
| 238 | GTCGGCAGTTCAATCCTGCCCATGGGCACCA | 13 | 12.50% | 100% | 2.62E−04 |
| 239 | ATAGTGCGTGTTCCCGTGTGAAAGTAGGTCATC | 10 | 12.50% | 100% | 2.62E−04 |
| | GTCAGGCT | | | | |
| 240 | GGTCATCTCGGGGGAACCT | 9 | 12.50% | 100% | 2.62E−04 |
| 241 | CACTCCAGCCTGGGCAACATAGCGCGACCCCGT | 9 | 12.50% | 100% | 2.62E−04 |
| | CTCTTA | | | | |
| 242 | TACGCCTGTCTGGGCGTCGC | 8 | 12.50% | 100% | 2.62E−04 |
| 243 | TGACCGGGGTAAATAAGCTTG | 8 | 12.50% | 100% | 2.62E−04 |
| 244 | CAGCGATCCGAGGTCAAATCTCGGTGGAACCTC | 8 | 12.50% | 100% | 2.62E−04 |
| | C | | | | |
| 245 | GGCTGGTCCGATGGGAGGGGGTTATCAGAACT | 10 | 14.06% | 100% | 8.90E−05 |
| | TAT | | | | |
| 246 | CAGTTCGGTCCCTATCTGCCGTGG | 17 | 14.06% | 100% | 8.90E−05 |
| 247 | TCAGTGCACTAAAGCACTTTGT | 10 | 14.06% | 100% | 8.90E−05 |
| 248 | GACGGATTGCGTAACTTGTTCAGACT | 15 | 14.06% | 100% | 8.90E−05 |
| 249 | TGGGAGAGTAGGTCGCCGCCGGACA | 14 | 14.06% | 100% | 8.90E−05 |
| 250 | GACGAAGACTGACGCTCAGGTGCGAAAGC | 14 | 14.06% | 100% | 8.90E−05 |
| 251 | GGGGTAGAGCACTGTTTAG | 10 | 14.06% | 100% | 8.90E−05 |
| 252 | GAAGTAGAAAAGAGCACATGGTGGATG | 13 | 15.62% | 100% | 2.98E−05 |
| 253 | TATTACACTCGTCCCGGCCTC | 13 | 17.19% | 100% | 9.88E−06 |
| 254 | TACCTGGTGGTATAGTGGTTAGGATTCGGCGCT | 22 | 18.75% | 100% | 3.23E−06 |
| | CT | | | | |

TABLE 4B
Disease Specific Biomarkers for Alzheimer's Disease Identified in CSF

| Stage | Braak II | Braak II | Braak III | Braak III | Braak III | Braak III | Braak IV |
|---|---|---|---|---|---|---|---|
| Seq. ID | SRR1568546 | SRR1568552 | SRR1568556 | SRR1568685 | SRR1568693 | SRR1568751 | SRR1568420 |
| 47 | 1.126 | | 0.9 | | | | |
| 48 | 1.126 | | 0.257 | | | | |
| 49 | 1.126 | | 0.257 | | | | |
| 50 | 1.126 | | 0.386 | | | | |
| 51 | 1.126 | 0.16 | 0.386 | | | | |
| 52 | 1.126 | | | | 1.74 | | |
| 53 | 2.252 | | 0.257 | | | | |
| 54 | 1.126 | | 0.129 | | | | |
| 55 | | | 0.129 | | | | |
| 56 | 2.252 | | 2.058 | | | | |
| 57 | | | 1.544 | | | | |
| 58 | | | 0.386 | | | | |
| 59 | | 0.16 | | | | 0.114 | |
| 60 | | 0.16 | | | | | |
| 61 | | | | 0.454 | | | |
| 62 | | | 1.286 | | | | |
| 63 | | | | | 0.58 | | |
| 64 | | | | 0.303 | | | |
| 65 | | | | | 2.899 | | |
| 66 | | | 0.129 | | | | |
| 67 | | | | 0.151 | | | |
| 68 | | | 0.129 | | | | |
| 69 | | 0.32 | 0.257 | | | | |
| 70 | | 0.16 | | | | | |
| 71 | | | 0.129 | | | | |
| 72 | | 0.16 | | | | | |
| 73 | | | | | 0.58 | | |
| 74 | | | | 0.151 | | 0.114 | |
| 75 | | | | | 2.319 | | |
| 76 | | | | 0.151 | | | |
| 77 | | 0.32 | | 0.151 | | | |
| 78 | | | 0.129 | | | | |
| 79 | | 0.32 | | | | | |
| 80 | 1.126 | | 0.386 | | | | |
| 81 | | 0.48 | | | | | |
| 82 | | | | 0.151 | | | |
| 83 | | | 0.257 | | | | |
| 84 | | | | | 0.58 | | |
| 85 | | 0.48 | | 0.151 | | | |
| 86 | | | | 0.151 | | | |
| 87 | | 0.16 | | 0.454 | | | |
| 88 | 1.126 | | 0.257 | | | | |
| 89 | | 0.32 | | | | | |
| 90 | | | | 0.151 | | | |
| 91 | | | | 0.151 | | | |
| 92 | | | | | | | |
| 93 | | | | | | | |
| 94 | | | | | | | |
| 95 | | | 0.129 | | | | |
| 96 | | | | | | | |
| 97 | | | | | | | |
| 98 | | | | | | 0.228 | |
| 99 | | | | | | | |
| 100 | | | 0.515 | | | | |
| 101 | | | | | | | |
| 102 | | | | | | | |
| 103 | | | | | | 0.228 | |
| 104 | | | | | | | |
| 105 | | | | | | | |
| 106 | | | | 0.303 | | | |
| 107 | | | | | | | |
| 108 | | | | | | | |
| 109 | | 0.16 | | | | | |
| 110 | | | | | | | |
| 111 | | | | | | | |
| 112 | | | | | | | 3.673 |
| 113 | | | | | | | |
| 114 | | | | | | | |
| 115 | | | | | | 0.114 | |
| 116 | | | 0.386 | | | | |
| 117 | | | | | | | |
| 118 | | | | | | | |
| 119 | | | | | | | |
| 120 | | | | | | | |
| 121 | | | | | | | |
| 122 | | | | | | | |
| 123 | | | | | | | |
| 124 | | | | | | | |
| 125 | | | | | | | |
| 126 | | | | | | | |
| 127 | | | | | | | |
| 128 | | 0.48 | | | | | |
| 129 | | | 0.129 | 0.151 | | | |
| 130 | | 0.32 | | | | | |
| 131 | | | | | | | |
| 132 | | | | | | | |
| 133 | | | | | | | |
| 134 | | | | | | | |
| 135 | 2.252 | | | | | | |
| 136 | | | | | | | |
| 137 | | | | | | | |
| 138 | | | | | | 0.228 | |
| 139 | | | 0.129 | | | | |
| 140 | | | | | | | |
| 141 | | | | | | | |
| 142 | | | 0.129 | | | | |
| 143 | | | | | | | |
| 144 | | | | | | | |
| 145 | | | | | | | |
| 146 | | | | | | | |
| 147 | | 0.16 | | | | | |
| 148 | | | 0.257 | | | | |
| 149 | | | | | | | |
| 150 | | | | | | | |
| 151 | | | | | | | |
| 152 | | | | | | | |
| 153 | | 0.32 | | | | | |
| 154 | | | | | | | |
| 155 | | | | | | | |
| 156 | | | | | | 0.114 | |
| 157 | | | | | | | |
| 158 | | | | | | | |
| 159 | | | | | | | |
| 160 | | | | | | | |
| 161 | | | | | | | |
| 162 | | | | | | | |
| 163 | | | | | | | |
| 164 | | | | | | | |
| 165 | | | | | | | |
| 166 | | | | | | | |
| 167 | | 1.12 | | 0.303 | | | |
| 168 | 2.252 | | 0.9 | | | | |
| 169 | 1.126 | | | | 0.58 | | |
| 170 | 1.126 | | 0.257 | | | | |
| 171 | | | | 0.303 | | | |
| 172 | | | | | 2.319 | | |
| 173 | | | | | 3.479 | | |
| 174 | | 0.48 | | | | | |
| 175 | | | | | 1.16 | | |
| 176 | | | | | 1.16 | | |
| 177 | | | | 0.151 | | | |
| 178 | | 0.16 | | | | | |
| 179 | | 0.16 | | | | | |
| 180 | | | | | 1.16 | | |
| 181 | | | | 0.303 | | 0.114 | |
| 182 | | | | 0.151 | | | |
| 183 | | 0.16 | | | 0.58 | | |
| 184 | | | 0.129 | | | 0.114 | |
| 185 | | | | 0.303 | | | |
| 186 | | | | | | 0.114 | 1.469 |
| 187 | | 0.16 | | | | | |
| 188 | | | | | 0.58 | | |
| 189 | | | | | | | |
| 190 | | | | | | | |
| 191 | | 0.16 | | | | | |
| 192 | | | | | | | |
| 193 | | | | | | | |
| 194 | | | | 0.303 | | | |
| 195 | | | | | | 0.114 | |
| 196 | | | | | | | |
| 197 | | | | | | | |
| 198 | | | | | | | |
| 199 | | | | | | | |
| 200 | | | | | | | |
| 201 | | | | 0.151 | | | |
| 202 | | | | | | | |
| 203 | | 0.16 | | | | | |
| 204 | | | | | | | |
| 205 | | | | 0.151 | | | |
| 206 | | | | 0.151 | | | |
| 207 | | | | | | | |
| 208 | | | | | | | |
| 209 | | | | | | | |
| 210 | | | | 0.151 | | | |
| 211 | | | | | | | |
| 212 | | | | | | | |
| 213 | | | | | | | |
| 214 | | 0.16 | | | | | |
| 215 | | | | | | | 0.735 |
| 216 | | | | 0.303 | | | |
| 217 | | | | | | | |
| 218 | 1.126 | | | | | | |
| 219 | | | | | | | |
| 220 | | | | | | | |
| 221 | | | | | | | |
| 222 | | | | | | | |
| 223 | | 0.32 | | | | | |
| 224 | | | | | | | |
| 225 | | | | | | | |
| 226 | | | | | | | |
| 227 | | 0.16 | | | | 0.114 | |
| 228 | | | | | 3.479 | | |
| 229 | | | | | | 0.114 | |
| 230 | | | | | | 0.114 | |
| 231 | | | | | | 0.114 | |
| 232 | | | | | | 0.114 | |
| 233 | | | | 0.151 | | | |
| 234 | | | | 0.151 | | 0.114 | |
| 235 | | | | | | | |
| 236 | | | | | | | |
| 237 | | | | | | | |
| 238 | | | | | | | |
| 239 | | | | | | | |
| 240 | | | | | | | 0.735 |
| 241 | | | | | | | |
| 242 | | | | | | | |
| 243 | | | | | | 0.114 | |
| 244 | | | | | | | |
| 245 | | 0.32 | | | | 0.114 | |
| 246 | | 0.16 | | | | | |
| 247 | | | | 0.151 | | | |
| 248 | | | | | | | |
| 249 | | | 0.129 | | 0.58 | | |
| 250 | | | | | | | |
| 251 | | | | | | | |
| 252 | | 0.16 | | | | | |
| 253 | | | | | | | |
| 254 | | | | 0.303 | | 0.114 | |
| # Bio- | 16 | 31 | 31 | 30 | 16 | 20 | 4 |
| markers | | | | | | | |
| Per | | | | | | | |
| Sample | | | | | | | |
| % | 5% | 10% | 10% | 10% | 5% | 6% | 1% |
| Coverage | | | | | | | |

TABLE 4B
Disease Specific Biomarkers for Alzheimer's Disease Identified in CSF

| Stage | Braak IV | Braak IV | Braak IV | Braak IV | Braak IV | Braak IV | Braak IV |
|---|---|---|---|---|---|---|---|
| Seq. ID | SRR1568436 | SRR1568488 | SRR1568533 | SRR1568540 | SRR1568585 | SRR1568644 | SRR1568651 |
| 47 | | | | 0.298 | | | |
| 48 | | | | | | | |
| 49 | | 0.489 | | | | | |
| 50 | | 0.245 | | 0.298 | | | |
| 51 | | | | 0.595 | | | |
| 52 | | | | | 0.584 | | |
| 53 | | 0.245 | | 0.298 | | | |
| 54 | | | | 0.298 | | | |
| 55 | | | | 0.298 | | | |
| 56 | | | | 1.191 | | | |
| 57 | | | | 0.298 | | | |
| 58 | | 0.489 | | 0.595 | | | |
| 59 | | | | | | | |
| 60 | | | | | | | 0.646 |
| 61 | 0.391 | | | | | | |
| 62 | | | | 0.298 | | | |
| 63 | | | 0.377 | | | | |
| 64 | 0.391 | | 0.377 | | | | |
| 65 | | 0.489 | | | | | |
| 66 | | 0.489 | | | | | |
| 67 | | | 0.377 | | | | |
| 68 | | | | 0.595 | | | |
| 69 | | | | | | | |
| 70 | | | | | | 0.286 | |
| 71 | | | | | | | 0.646 |
| 72 | | | | | | | 0.215 |
| 73 | | 0.245 | | | | | |
| 74 | 0.391 | | | | | | |
| 75 | | | | 0.298 | | | |
| 76 | 0.391 | | | | | | |
| 77 | | | | | | | |
| 78 | | 0.489 | | 0.298 | | | |
| 79 | | | | | | | 0.215 |
| 80 | | | | 0.298 | | | |
| 81 | 0.195 | | | | | | |
| 82 | 0.195 | | | | | | |
| 83 | | | | | | | |
| 84 | | | | | | | 0.43 |
| 85 | | | | | | | |
| 86 | 0.391 | | | | | | |
| 87 | | | | | | | |
| 88 | | 0.245 | | 0.595 | | | |
| 89 | | | | | | | |
| 90 | 0.195 | | | | | 0.143 | |
| 91 | | | | | | | |
| 92 | 0.195 | | 0.377 | | | | |
| 93 | | 3.913 | | | | | |
| 94 | | 0.978 | | | | | |
| 95 | | | | | | | |
| 96 | | 2.201 | | | | | |
| 97 | | | | | | 0.143 | |
| 98 | | | | | | | |
| 99 | | | | | | 0.143 | |
| 100 | | 0.978 | | 0.298 | | 0.286 | |
| 101 | 0.195 | | | | | | |
| 102 | | | | | | | |
| 103 | | | | | | | |
| 104 | | | | | | 0.429 | |
| 105 | | | | | | | 0.215 |
| 106 | | | | | | | |
| 107 | | | | | | | 0.215 |
| 108 | 0.195 | | 0.377 | | | 0.286 | 0.646 |
| 109 | | | | | | | |
| 110 | | 0.245 | | | | | |
| 111 | | | | | | | 0.215 |
| 112 | | | | | | | |
| 113 | 0.391 | | | | | | |
| 114 | | | | | | | |
| 115 | | | | | | | |
| 116 | | | | | | | |
| 117 | | | 0.377 | | | | |
| 118 | | | 0.377 | | | | |
| 119 | | | | | | 0.143 | |
| 120 | | | | | | | |
| 121 | | | | | 0.584 | 0.143 | |
| 122 | 0.195 | | | | | | |
| 123 | | | | | | 0.572 | |
| 124 | 0.391 | | 0.377 | | | | |
| 125 | | 0.245 | | | | | |
| 126 | | | | | | | |
| 127 | | | | 0.595 | | | |
| 128 | | | | | | | |
| 129 | | | | | | | |
| 130 | | | | | | | |
| 131 | | 0.489 | | | | | |
| 132 | | | | | 0.584 | | |
| 133 | | | | | | | 0.43 |
| 134 | | | | | 0.584 | | |
| 135 | | | | | | | |
| 136 | | | | | | | |
| 137 | | | | | | 0.143 | |
| 138 | | | | | | | |
| 139 | | | | | | | |
| 140 | | | | | | | 0.215 |
| 141 | 0.195 | | | | | | |
| 142 | | | | | | | |
| 143 | | | | | | 0.286 | |
| 144 | | | | 0.298 | | | |
| 145 | | | | | | 0.143 | |
| 146 | | | | | | 0.286 | |
| 147 | | | | | | | |
| 148 | | 0.489 | | 0.298 | | | |
| 149 | | | | | | 0.143 | |
| 150 | | | | | | 0.286 | |
| 151 | 0.195 | | | | | | |
| 152 | | | | | | | 0.215 |
| 153 | | | | | | | |
| 154 | | | | | | | |
| 155 | | | | | | 0.286 | |
| 156 | | | | | | | |
| 157 | | | | | | | |
| 158 | 0.195 | | | | | | |
| 159 | | | 0.377 | | 0.584 | | |
| 160 | | | | | | 0.143 | |
| 161 | | | | | | | 0.215 |
| 162 | | | | | | 0.143 | |
| 163 | | | | | | 0.286 | |
| 164 | | | 0.377 | | | | |
| 165 | | | 1.132 | | | | |
| 166 | 0.195 | | | | | 0.286 | |
| 167 | | | | | | | |
| 168 | | | | 0.298 | | | |
| 169 | | | | | | | 0.43 |
| 170 | | | | | | | |
| 171 | 0.781 | | 0.377 | | | | |
| 172 | | | | | 1.752 | | |
| 173 | | | | | 0.584 | | |
| 174 | | | | | | | 0.646 |
| 175 | 0.195 | | | | 2.336 | | |
| 176 | 0.391 | | | | | | |
| 177 | 0.391 | | | | | | |
| 178 | 0.195 | | | 0.298 | | | |
| 179 | | | | | | | |
| 180 | | 0.245 | | | 1.168 | | |
| 181 | 0.195 | | | | | | |
| 182 | | 0.245 | | | | | |
| 183 | | | | | | | |
| 184 | 0.391 | | | | | | |
| 185 | | | | | | | |
| 186 | 0.195 | | | | | | |
| 187 | | | | | | | |
| 188 | | | | | | | 0.43 |
| 189 | | 0.978 | | | | | |
| 190 | 0.195 | | | | | | |
| 191 | | | | | | | |
| 192 | | | | | | 0.143 | |
| 193 | | | | | | 0.143 | |
| 194 | | | | | | | |
| 195 | | | | | | | |
| 196 | | | | | | 0.143 | |
| 197 | | | | | | 0.143 | |
| 198 | | | | | | | |
| 199 | | | 0.377 | | | | 0.215 |
| 200 | | | | | | | 0.215 |
| 201 | | | | | | | |
| 202 | | | | | | | |
| 203 | | | | | | | |
| 204 | | | | | | | 0.215 |
| 205 | | | | | | | |
| 206 | | | | | | | |
| 207 | | 0.245 | | | | | |
| 208 | | 0.245 | | | | | |
| 209 | | | | | 0.584 | | |
| 210 | | | | | | 0.143 | |
| 211 | | | | | | | |
| 212 | 0.195 | | | | | | |
| 213 | | | | | | 0.286 | |
| 214 | | | | | | | |
| 215 | | | | | | | 0.215 |
| 216 | | | | | | | |
| 217 | | | | | 1.752 | | |
| 218 | | | | 0.298 | | | |
| 219 | | | | | | 0.143 | |
| 220 | 0.391 | | | | | 0.143 | |
| 221 | | | | | 0.584 | | |
| 222 | 0.195 | | | | | | |
| 223 | | | | | | | |
| 224 | | | | | | | |
| 225 | 0.195 | | | | | | |
| 226 | 0.195 | | | | | | |
| 227 | | | | | | 0.143 | |
| 228 | | | | | 2.336 | | |
| 229 | | | | | | 0.143 | |
| 230 | | | | | | | 0.215 |
| 231 | | | | | | | |
| 232 | | | | | | | 0.215 |
| 233 | 0.195 | | | | | | |
| 234 | 0.195 | | | | | | |
| 235 | | | | | | | |
| 236 | | | | | | | 1.076 |
| 237 | | 0.978 | | 0.298 | | 0.143 | |
| 238 | | | | | | | |
| 239 | | | | | | | |
| 240 | | | | | | | |
| 241 | | 0.489 | | | | | |
| 242 | | | | | | 0.143 | |
| 243 | | | | | | | |
| 244 | 0.195 | | | | | | |
| 245 | | | | | | 0.143 | |
| 246 | | | | | | | |
| 247 | | | | | | | |
| 248 | | | | | | 0.143 | |
| 249 | | | | | | | |
| 250 | 0.195 | | | | | | |
| 251 | | | 0.377 | | | | |
| 252 | | | | | | | 0.215 |
| 253 | | | 0.377 | | | | |
| 254 | 0.976 | | 0.377 | | | | |
| # Bio- | 37 | 24 | 16 | 23 | 13 | 35 | 24 |
| markers | | | | | | | |
| Per | | | | | | | |
| Sample | | | | | | | |
| % Cover- | 12% | 8% | 5% | 7% | 4% | 11% | 8% |
| age | | | | | | | |

TABLE 4B
Disease Specific Biomarkers for Alzheimer's Disease Identified in CSF

| | Stage | | | | | | |
|---|---|---|---|---|---|---|---|
| | Braak IV | Braak IV | Braak IV | Braak V | Braak V | Braak V | Braak V |
| Seq. ID | SRR1568655 | SRR1568733 | SRR1568743 | SRR1568368 | SRR1568370 | SRR1568397 | SRR1568406 |
| 47 | | 0.614 | | | | | |
| 48 | | 0.614 | | | | | |
| 49 | | | | | | 0.928 | |
| 50 | | | | | | | |
| 51 | | | | | | | |
| 52 | 0.503 | | | | | | |
| 53 | | | | | | 0.464 | |
| 54 | | 0.307 | | | | | |
| 55 | | | | 0.093 | | | |
| 56 | | 0.614 | | | | | |
| 57 | | 0.921 | | | | | |
| 58 | | | | | | 0.928 | |
| 59 | | | | | 1.549 | | |
| 60 | | | | | | | |
| 61 | | | | | | | |
| 62 | | 0.614 | | | | | |
| 63 | | | | 0.186 | | | |
| 64 | | | | | | | |
| 65 | | | | | | | |
| 66 | 0.503 | | | | | 1.391 | |
| 67 | | | | | | | |
| 68 | | | | | | | |
| 69 | | | | | | | |
| 70 | | | | | | | |
| 71 | | | | | | | |
| 72 | | | | | | | |
| 73 | | | | | | | |
| 74 | | | | | 0.282 | | |
| 75 | | | | | 0.141 | 0.464 | 0.075 |
| 76 | | | | | | | |
| 77 | | | | | | | |
| 78 | | 0.307 | | | | | |
| 79 | | | | 0.093 | | | |
| 80 | | 0.307 | | | | | |
| 81 | | | | | | | |
| 82 | | | | | | | |
| 83 | | | 0.145 | | | | |
| 84 | | | | | | | |
| 85 | | | | | | | |
| 86 | | | | | | | |
| 87 | | | | | 0.141 | | |
| 88 | | | | | | | |
| 89 | | 0.307 | | | | | |
| 90 | | | | | | | |
| 91 | | | | | | | |
| 92 | | | | | 0.141 | | |
| 93 | 0.503 | | | | | | |
| 94 | 2.517 | | | 0.279 | 0.563 | | |
| 95 | | | | | 0.422 | | |
| 96 | | 0.307 | | | | 0.464 | |
| 97 | | | | | | | |
| 98 | | | | 0.093 | | | |
| 99 | | | | 0.372 | 0.422 | | |
| 100 | | 0.307 | | | | | |
| 101 | | | | | 0.141 | | |
| 102 | 0.503 | | | | | | |
| 103 | | | | | | | |
| 104 | | 0.307 | | | | | |
| 105 | | | | | 0.141 | | |
| 106 | | | | | | | |
| 107 | | | | | 0.845 | | |
| 108 | | | | | | | |
| 109 | | | | | | | 0.075 |
| 110 | 0.503 | | | | 0.282 | | |
| 111 | | | | | 0.141 | | |
| 112 | | | | | | 0.464 | |
| 113 | | | | | | | |
| 114 | | | 0.145 | | | | |
| 115 | | | | | | | |
| 116 | | | | | 0.282 | | |
| 117 | | | | | 0.563 | | |
| 118 | | | | | | | |
| 119 | | 1.535 | | | | | |
| 120 | | | 0.145 | | | | |
| 121 | | | | | | | |
| 122 | | | | | | | |
| 123 | | | | | | | |
| 124 | | | | | | | |
| 125 | 0.503 | | | | | | |
| 126 | | 0.614 | | | | | |
| 127 | | | | | | 0.928 | |
| 128 | | | | | | | |
| 129 | | | | | 0.282 | | |
| 130 | | | | | | | 0.075 |
| 131 | | | | | | | |
| 132 | 0.503 | | | | | | |
| 133 | | | | | | | |
| 134 | | | | 0.186 | 0.141 | | |
| 135 | | | | | | | |
| 136 | | | 0.145 | | | | 0.075 |
| 137 | | | | | | | |
| 138 | | | | | | | |
| 139 | | | | | | | |
| 140 | | | | 0.186 | | | |
| 141 | | | | | | | |
| 142 | | | | | | | 0.075 |
| 143 | | | | 0.186 | | | |
| 144 | | | | | | | |
| 145 | | | | | | | |
| 146 | | | | | | | |
| 147 | | | | 0.093 | | | |
| 148 | | | | | | | |
| 149 | | | | | 0.422 | | 0.075 |
| 150 | | | | | | | |
| 151 | | | | | | | |
| 152 | | | | | | | |
| 153 | | | | | | | 0.075 |
| 154 | | 0.307 | | | | | 0.075 |
| 155 | | | | | | | |
| 156 | | | | 0.093 | 0.141 | | |
| 157 | | | 0.145 | | 0.282 | | |
| 158 | | | | | | | 0.075 |
| 159 | | | | | | | |
| 160 | | 0.614 | | | | | |
| 161 | | | | | | | |
| 162 | | | | | | | |
| 163 | | | | | | | |
| 164 | | | | | | | |
| 165 | | | | | | | |
| 166 | | | | 0.186 | | | |
| 167 | | 0.307 | | | | | |
| 168 | | 0.307 | | | | | |
| 169 | | | | | 0.282 | | |
| 170 | | 0.307 | | | | | |
| 171 | | | | | | | |
| 172 | | | | | | 0.464 | |
| 173 | | | | | | | 0.075 |
| 174 | | | | | | | |
| 175 | | 0.307 | | | | | |
| 176 | | | | | | | |
| 177 | | | | | | | |
| 178 | | | | | | | 0.075 |
| 179 | | | 0.145 | | | 0.928 | |
| 180 | 0.503 | | | | 0.141 | | |
| 181 | | | | | | | |
| 182 | | | | | | | |
| 183 | | | | | | 0.464 | |
| 184 | | | | | | | |
| 185 | | 0.307 | | | | | |
| 186 | | | | | | | |
| 187 | 0.503 | | | | 0.282 | 0.464 | |
| 188 | | | | | | 0.464 | |
| 189 | | 0.307 | | | | | |
| 190 | | | | | | | |
| 191 | | | | | 0.422 | | |
| 192 | | | | | | | |
| 193 | | | | | | | |
| 194 | | | | | 0.282 | | |
| 195 | | | | | 0.141 | | |
| 196 | | | | | | | |
| 197 | | | | | 0.282 | | |
| 198 | | 0.921 | | | | | |
| 199 | | | | | | | |
| 200 | | | | 0.093 | | | |
| 201 | | | | | | | 0.149 |
| 202 | | 0.307 | | | 0.141 | | |
| 203 | | | | | 0.282 | | |
| 204 | | | | | 0.282 | | |
| 205 | | | | | | | |
| 206 | | | | | | | |
| 207 | | | | 0.186 | 0.282 | | |
| 208 | 1.007 | | | | | | |
| 209 | | | | 0.279 | | | |
| 210 | | | | | | | |
| 211 | | | 0.145 | | 0.141 | | 0.075 |
| 212 | | | | 0.093 | 0.422 | | |
| 213 | | | | | 0.141 | | |
| 214 | | | | | | | |
| 215 | | | | | | | |
| 216 | | | | | | | |
| 217 | | 0.307 | | | | | |
| 218 | 0.503 | | | 0.093 | 0.282 | | |
| 219 | | | | 0.093 | | | |
| 220 | | | | | | | |
| 221 | | | | | | | |
| 222 | | | | | | | |
| 223 | | | | | | | |
| 224 | | | 0.145 | | | | |
| 225 | | | | | 0.141 | | |
| 226 | | | | | | | |
| 227 | | | | | | | |
| 228 | | | | 0.093 | | | |
| 229 | | | | | | | |
| 230 | | | | | | | 0.075 |
| 231 | | | 0.145 | | 0.282 | | |
| 232 | | | | | | | |
| 233 | | 0.307 | | | | | |
| 234 | | | | | | | |
| 235 | | 3.991 | | | | | |
| 236 | | | | 0.279 | 0.141 | | |
| 237 | | | | | | 0.464 | |
| 238 | | | 0.145 | | 0.141 | | 0.149 |
| 239 | | | 0.145 | | | | |
| 240 | | | | | 0.141 | | |
| 241 | | | 0.145 | | | | |
| 242 | | | | | | | |
| 243 | | | | | 0.141 | | 0.075 |
| 244 | | | | | | | |
| 245 | | | | | | | |
| 246 | | | 0.289 | | 0.563 | | |
| 247 | | | 0.145 | | | | 0.075 |
| 248 | | | | | | | |
| 249 | | | | | 0.141 | 0.464 | |
| 250 | | | | | 0.141 | | |
| 251 | | | | | | | |
| 252 | | | 0.145 | 0.279 | | | |
| 253 | | | | | | | |
| 254 | | | | | | | |
| # Biomarkers Per | 12 | 27 | 15 | 21 | 44 | 15 | 17 |
| Sample | | | | | | | |
| % Coverage | 4% | 9% | 5% | 7% | 14% | 5% | 5% |
| | Stage | | | | | | |
| | Braak V | Braak V | Braak V | Braak V | Braak V | Braak V | Braak V |
| Seq. ID | SRR1568408 | SRR1568445 | SRR1568454 | SRR1568467 | SRR1568474 | SRR1568480 | SRR1568514 |
| 47 | | | | | | | |
| 48 | | | | | 0.366 | | |
| 49 | | | | | | | |
| 50 | | | | | 0.731 | | |
| 51 | | | | | 0.366 | | |
| 52 | | | 0.277 | | | | |
| 53 | | | | | | | |
| 54 | | | | | | | |
| 55 | | | | | | 0.188 | |
| 56 | | | | | 1.097 | | |
| 57 | | | | | 1.462 | | |
| 58 | | | | | | | |
| 59 | | | | | | | |
| 60 | | | | | | 0.188 | |
| 61 | | 0.227 | | | | | |
| 62 | | | | | 0.366 | | |
| 63 | | | 0.832 | | | | |
| 64 | | | | | | | |
| 65 | | | | | | | |
| 66 | | | | | 0.731 | | |
| 67 | | 0.227 | | | | | |
| 68 | | | | | | | 0.128 |
| 69 | | | | | | | |
| 70 | | | | | | | |
| 71 | | | | | 0.366 | | |
| 72 | | | | | | | |
| 73 | 0.46 | | | | | | |
| 74 | | | | 0.391 | | | |
| 75 | | | | | | | |
| 76 | | 0.34 | | | | | |
| 77 | | | | | | | |
| 78 | | | | | 0.366 | | |
| 79 | | | | | | | |
| 80 | | | | | 0.366 | | |
| 81 | | | | | 0.366 | | |
| 82 | | | | | 0.366 | | |
| 83 | | | | | | | |
| 84 | | | | 0.195 | | | |
| 85 | | | | | | | |
| 86 | | 0.113 | | | | | |
| 87 | | | | 0.195 | | | |
| 88 | | | | | | | |
| 89 | | | | | | | |
| 90 | | | | | | | |
| 91 | | | | | | | |
| 92 | 0.115 | | | | | | |
| 93 | | | | | 0.366 | | |
| 94 | | | | | | | |
| 95 | | | | | | | |
| 96 | | | | | 0.366 | | |
| 97 | | | | | | | 0.064 |
| 98 | | | | | | | |
| 99 | | | | | | | |
| 100 | | | | | | | |
| 101 | | | | | | | |
| 102 | | | | | | | |
| 103 | | | | | | | |
| 104 | | | | | | | 0.191 |
| 105 | | | | | | | |
| 106 | | | | | | | |
| 107 | | | | | | | |
| 108 | | 0.113 | | | | | |
| 109 | | | | | | | |
| 110 | | | | | | | |
| 111 | | | | | | | |
| 112 | | 0.113 | | 0.391 | | | |
| 113 | | | | 0.195 | | | |
| 114 | | | | 0.195 | | | |
| 115 | | | | 0.195 | | | |
| 116 | 0.23 | | | | | | |
| 117 | | | | | | | |
| 118 | | | | | | | |
| 119 | 0.115 | | | | | | |
| 120 | | | | | | | |
| 121 | | | | | | | |
| 122 | 0.46 | | | | | | 0.064 |
| 123 | | | | | | | 0.128 |
| 124 | | 0.227 | | | | | |
| 125 | | | | | | | |
| 126 | | | | | | | |
| 127 | | | | | 0.731 | | |
| 128 | | | | | | | |
| 129 | | | | | | | |
| 130 | | | | 0.391 | | | |
| 131 | | | | | | | 0.064 |
| 132 | | | | | | | |
| 133 | | 0.113 | | | | | |
| 134 | 0.345 | | | | | | |
| 135 | | | | | | | 0.064 |
| 136 | | | | | | | |
| 137 | | | | | 0.366 | | |
| 138 | | | | | | | |
| 139 | 0.115 | | | | | | |
| 140 | | | | | | | |
| 141 | | | | | | | 0.064 |
| 142 | | | | | | | |
| 143 | 0.115 | | | | | | |
| 144 | | | | | | | |
| 145 | 0.115 | | | | | | |
| 146 | | | | | | | |
| 147 | | | | | | | |
| 148 | | | | | | | |
| 149 | 0.115 | | | | | | |
| 150 | | | | | | | |
| 151 | 0.115 | | | | | | |
| 152 | | | | 0.195 | | | |
| 153 | | | | | | | |
| 154 | | | | | | | |
| 155 | | | | | | | 0.064 |
| 156 | | | | | | | |
| 157 | | | | | | | |
| 158 | | | | | | | |
| 159 | | | | | | | 0.064 |
| 160 | | | | | | | |
| 161 | | | | | | | |
| 162 | | | | | | | 0.191 |
| 163 | | | | | | | |
| 164 | | 0.113 | | | | | |
| 165 | | | | | | 0.188 | |
| 166 | | | | | | | |
| 167 | | | | | 0.731 | | |
| 168 | | | | | 0.731 | | |
| 169 | | | | | | | |
| 170 | | | | | | | |
| 171 | | 0.227 | | | | 0.188 | |
| 172 | | 0.113 | | | | | |
| 173 | | | | | | | |
| 174 | | 0.113 | | | | | |
| 175 | | | | | | | |
| 176 | | | | | | | |
| 177 | | 0.113 | | | | | |
| 178 | | | | 0.195 | | | |
| 179 | | | | | | 0.188 | |
| 180 | | | | | | | |
| 181 | | 0.113 | | | | | |
| 182 | | | | | | | |
| 183 | | | | | | | |
| 184 | | | | | | | |
| 185 | | | | | | | |
| 186 | | | | | | | |
| 187 | | | | | | | |
| 188 | | | | | | | |
| 189 | | | | | 0.731 | | |
| 190 | | | | | | | |
| 191 | | 0.113 | | | | | |
| 192 | | | | | | | 0.064 |
| 193 | | | | | | | 0.191 |
| 194 | | | | | | | |
| 195 | 0.23 | | | | | | |
| 196 | | | | | | | 0.064 |
| 197 | 0.345 | | | | | | |
| 198 | 0.115 | 0.113 | | | | | |
| 199 | | | | | | | |
| 200 | | | | | | | |
| 201 | | | | | | | |
| 202 | | | | | | | |
| 203 | | | | | 1.097 | 0.188 | |
| 204 | | | | | | | |
| 205 | | | | 0.195 | | | |
| 206 | | | | | | | |
| 207 | 0.115 | | | | | | |
| 208 | | | | | | | |
| 209 | | | | | | | |
| 210 | | | | | | | |
| 211 | | | | | | | |
| 212 | | | | | | | |
| 213 | | | | | | | 0.064 |
| 214 | | 0.113 | | | | | |
| 215 | | | | | | 0.188 | |
| 216 | | | | | | | |
| 217 | 0.115 | | | | | | |
| 218 | | | | | | | |
| 219 | | | | | | | 0.064 |
| 220 | | 0.113 | | | | | |
| 221 | 0.115 | | | | | | |
| 222 | 0.23 | 0.113 | | | | | |
| 223 | 0.115 | | | | 0.366 | | |
| 224 | | | | | | | 0.128 |
| 225 | | | | | | | |
| 226 | 0.115 | | | | | | |
| 227 | | | | 0.195 | | | |
| 228 | 0.115 | | | | | | |
| 229 | | | | | | | |
| 230 | | | | | | | |
| 231 | | | | | | | |
| 232 | 0.115 | | | | | | |
| 233 | | | | 0.195 | | 0.188 | |
| 234 | | 0.113 | | | | | |
| 235 | | | | | | | |
| 236 | | | | | | 0.188 | |
| 237 | | | | | | | |
| 238 | | | | | | | |
| 239 | | | | | | | |
| 240 | 0.23 | | | | | | 0.064 |
| 241 | | | | | | | |
| 242 | | | | | | | 0.064 |
| 243 | | | | | | | 0.064 |
| 244 | 0.115 | | | 0.195 | | | |
| 245 | | | | | | | |
| 246 | | | | | | | |
| 247 | | | | | | | |
| 248 | | | | | | | |
| 249 | | | | | 1.097 | | |
| 250 | | | | | | | |
| 251 | | | | | | | |
| 252 | | | | | | | |
| 253 | | 0.227 | | | 0.366 | | 0.064 |
| 254 | | 0.227 | | | | | |
| # Biomarkers Per | 24 | 22 | 2 | 14 | 23 | 9 | 21 |
| Sample | | | | | | | |
| % Coverage | 8% | 7% | 1% | 4% | 7% | 3% | 7% |
| | Stage | | | | | | |
| | Braak V | Braak V | Braak V | Braak V | Braak V | Braak V | Braak V |
| Seq. ID | SRR1568522 | SRR1568573 | SRR1568638 | SRR1568642 | SRR1568665 | SRR1568667 | SRR1568673 |
| 47 | | | | 0.391 | | | |
| 48 | | | | 0.391 | | | |
| 49 | | | | 0.391 | | | |
| 50 | | | | | | | |
| 51 | | | | | | | |
| 52 | | | | | | | |
| 53 | | | | | | | |
| 54 | | | | 0.783 | | | |
| 55 | | | | | | | |
| 56 | | | | 1.566 | | | |
| 57 | | | | 0.783 | | | |
| 58 | | | | 0.391 | | | |
| 59 | | | 0.335 | | | | |
| 60 | | | | | | | |
| 61 | | | | | | | |
| 62 | | | 0.112 | | | | |
| 63 | | | | | | 0.418 | |
| 64 | | | | | | | |
| 65 | | | | | | | |
| 66 | | | | | | | |
| 67 | | | | | | | |
| 68 | 0.26 | | | | | | |
| 69 | 0.26 | | | | | | |
| 70 | | 0.081 | | | | | |
| 71 | | | | | | | |
| 72 | | 0.162 | | | | 0.084 | |
| 73 | | | 0.112 | | | 0.084 | |
| 74 | | | | | | | |
| 75 | | | | | | | |
| 76 | | | | | | | |
| 77 | | | 0.112 | | | | |
| 78 | | | | | | | |
| 79 | 0.13 | | | | | | |
| 80 | | | | 0.391 | | | |
| 81 | | | | | 0.074 | | |
| 82 | | | | 0.391 | | | |
| 83 | | | | | 0.148 | | |
| 84 | | | | | | | |
| 85 | | | | 0.391 | | | |
| 86 | | | | | | | |
| 87 | | | | | | | |
| 88 | | | | | | | |
| 89 | | | | | | | 0.127 |
| 90 | | | | 0.783 | 0.074 | | |
| 91 | 0.13 | | 4.798 | | | | |
| 92 | | | | | | | |
| 93 | | | | | | | |
| 94 | | | | | | | |
| 95 | | 0.081 | | | | 0.251 | |
| 96 | | | | | | | |
| 97 | 0.52 | | | | | | |
| 98 | | 0.081 | | | | 0.585 | |
| 99 | | 0.162 | | | | | |
| 100 | | | | | | | |
| 101 | | | | | | 0.418 | |
| 102 | | | | | | 0.251 | 0.127 |
| 103 | | 0.081 | | | | 0.251 | |
| 104 | 0.26 | | | | | | |
| 105 | | | | | | 0.335 | |
| 106 | | 0.162 | | | | 0.502 | |
| 107 | | 0.081 | | | | 0.167 | 0.127 |
| 108 | | | | | | | |
| 109 | 0.26 | | 0.335 | | | 0.167 | |
| 110 | | | 0.335 | | | | |
| 111 | | 0.081 | | | | 0.418 | |
| 112 | | | | | | | |
| 113 | | 0.081 | | | | 0.167 | |
| 114 | | | | | | 0.167 | |
| 115 | | | | | | | 0.381 |
| 116 | | | 0.112 | | | | |
| 117 | | | | | | | |
| 118 | | 0.162 | | | | | 0.381 |
| 119 | | | | | | | |
| 120 | | | | | | 0.251 | 0.127 |
| 121 | | | | | 0.148 | 0.084 | 0.127 |
| 122 | | | | | | | |
| 123 | 0.13 | | | | | | |
| 124 | | | | | | | |
| 125 | | 0.081 | | | | 0.167 | |
| 126 | | | | | | | 0.254 |
| 127 | | | | 0.391 | | | |
| 128 | 0.13 | | | | | | 0.254 |
| 129 | | | | | | 0.084 | 0.381 |
| 130 | | | | | | 0.167 | |
| 131 | 0.13 | | | | | | |
| 132 | | | | | | | |
| 133 | | | | | | | 0.127 |
| 134 | | | | | | 0.084 | |
| 135 | | | | | | | |
| 136 | | 0.081 | | | | 0.167 | |
| 137 | | | | | | 0.335 | 0.127 |
| 138 | | 0.081 | | | | 0.084 | |
| 139 | | | | | | | 0.254 |
| 140 | | | | | | | 0.127 |
| 141 | | | | | | | |
| 142 | | | | | | | |
| 143 | | | | | | 0.084 | |
| 144 | | 0.081 | | | | | 0.254 |
| 145 | | | | | | | |
| 146 | | | | | 0.074 | | |
| 147 | | | | | | 0.084 | |
| 148 | | | | | | | |
| 149 | | | 0.112 | | | | |
| 150 | 0.26 | | | | | | |
| 151 | | | | | | | |
| 152 | | | | | | | |
| 153 | | 0.081 | | | | | |
| 154 | | | | | | | |
| 155 | 0.13 | | | | | | |
| 156 | | | 0.335 | | | | |
| 157 | | 0.081 | | | | 0.167 | |
| 158 | | | | | | | |
| 159 | | | | | | | |
| 160 | | | | | | | |
| 161 | | 0.081 | | | | | |
| 162 | 0.13 | | | | | | |
| 163 | | 0.162 | | | 0.074 | | |
| 164 | | | | 0.391 | | | |
| 165 | | 0.081 | | | | | |
| 166 | 0.13 | 0.081 | | | | | |
| 167 | | | | | | | |
| 168 | | | | 0.391 | | | |
| 169 | | 0.081 | | | | 0.167 | |
| 170 | | | | 0.391 | | | |
| 171 | | | | | | | |
| 172 | | | | | | | |
| 173 | | | | | | | |
| 174 | | | | | | | |
| 175 | | | | | | | |
| 176 | 0.26 | | | 0.391 | | | |
| 177 | | | | | | 0.084 | |
| 178 | | | | | | 0.335 | |
| 179 | | | | | | | |
| 180 | | | | | | | |
| 181 | | | | | | | |
| 182 | | | | | | | |
| 183 | | | 0.112 | | | | |
| 184 | | | | | | 0.167 | |
| 185 | | | | | | | |
| 186 | | | | | | 0.084 | |
| 187 | | | 0.112 | | | | |
| 188 | | | | | | | 0.127 |
| 189 | | | | 1.566 | | | |
| 190 | | | | 0.391 | | | 0.127 |
| 191 | | 0.243 | | | | 0.418 | |
| 192 | 0.13 | | | | | | |
| 193 | 0.13 | | | | | | |
| 194 | | | | | | 0.167 | 0.127 |
| 195 | | | | | 0.074 | | |
| 196 | 0.13 | | | | | | |
| 197 | | | | | | | |
| 198 | | | | | | | |
| 199 | | | 0.112 | | | 0.084 | |
| 200 | | | | | | | 0.127 |
| 201 | | | | | | 0.084 | |
| 202 | | 0.243 | | | | | |
| 203 | | | | | | | |
| 204 | | | | | | 0.167 | |
| 205 | | | | | | 0.084 | |
| 206 | | | | | | | |
| 207 | | | 0.112 | | | 0.084 | |
| 208 | | | | | 0.074 | 0.084 | |
| 209 | | 0.081 | | | | | |
| 210 | | | | | | | |
| 211 | | | | | | | |
| 212 | | 0.081 | | | | 0.084 | |
| 213 | 0.26 | | | | | | |
| 214 | | | | | 0.074 | | 0.254 |
| 215 | | | | 0.783 | | 0.084 | |
| 216 | | | | | | 0.084 | |
| 217 | | | | | | | |
| 218 | | | | | | 0.084 | |
| 219 | 0.13 | | | | | | |
| 220 | | | | | | | |
| 221 | | 0.081 | | | | | 0.254 |
| 222 | | | | | | | |
| 223 | | 0.081 | | | | | |
| 224 | | | | | | | |
| 225 | | | | | | | |
| 226 | | 0.081 | | | | 0.084 | |
| 227 | | 0.081 | | | | | |
| 228 | | | | | | | |
| 229 | 0.13 | | | | | | 0.254 |
| 230 | 0.13 | | | | | | |
| 231 | | | | | | | |
| 232 | | | | | | | |
| 233 | | | | | | | |
| 234 | | | | | | | 0.127 |
| 235 | | 0.081 | | | | | |
| 236 | | 0.081 | | | | 0.084 | |
| 237 | | | 0.223 | | | | |
| 238 | | 0.081 | | | | | |
| 239 | | | | | | 0.084 | |
| 240 | 0.13 | 0.081 | | | | | |
| 241 | | | | | | | |
| 242 | 0.13 | 0.081 | | | | 0.084 | |
| 243 | | | | | | 0.084 | 0.127 |
| 244 | | | | | | | |
| 245 | | 0.081 | | | 0.074 | 0.084 | |
| 246 | | 0.162 | | | | 0.167 | |
| 247 | | | | | | | |
| 248 | 0.26 | | | | | 0.167 | |
| 249 | | | | | | | |
| 250 | | 0.081 | | | | 0.084 | |
| 251 | | 0.081 | | | | 0.084 | 0.127 |
| 252 | | | | | | 0.084 | 0.127 |
| 253 | 0.13 | | | | | | |
| 254 | | | | | | | |
| # of biomarkers | 26 | 39 | 15 | 19 | 10 | 55 | 26 |
| per sample | | | | | | | |
| % Coverage | 8% | 12% | 5% | 6% | 3% | 18% | 8% |
| | Stage | | | | | | |
| | Braak V | Braak V | Braak V | Braak VI | Braak VI | Braak VI | Braak VI |
| Seq. ID | SRR1568687 | SRR1568704 | SRR1568718 | SRR1568388 | SRR1568422 | SRR1568432 | SRR1568434 |
| 47 | | | | | | | |
| 48 | | | | | | | |
| 49 | | | | | | | |
| 50 | | | | | | | |
| 51 | | | | | | | |
| 52 | | | | | | | |
| 53 | | | | | 0.197 | | |
| 54 | | | | | | | |
| 55 | | | | | | | |
| 56 | | | | | | | |
| 57 | | | | | | | |
| 58 | | | | | | | |
| 59 | | | | | | | |
| 60 | | | | | | | |
| 61 | | 0.64 | | | | | |
| 62 | | | | | | | |
| 63 | | | | | | | |
| 64 | | 0.512 | | | | | |
| 65 | 0.311 | | | 0.597 | | | |
| 66 | | | | | 0.395 | | |
| 67 | | | | | | | |
| 68 | | | | | | | 0.098 |
| 69 | | | | | | | |
| 70 | | | | | | 0.274 | |
| 71 | | | 0.494 | | 0.395 | | |
| 72 | | | | | | | |
| 73 | | | | | | | |
| 74 | | | | | | | |
| 75 | | | | | | | |
| 76 | | | | | | | |
| 77 | | | | | | | |
| 78 | | | | | | | |
| 79 | | | | | | | |
| 80 | | | | | | | |
| 81 | | | | | | | |
| 82 | | | | | | | |
| 83 | | 0.128 | | | | | |
| 84 | | | | | | | |
| 85 | | | | | | | |
| 86 | | | | | | | |
| 87 | | | | | | | |
| 88 | | | | | | | |
| 89 | | | | | | | 0.098 |
| 90 | | | | | | | |
| 91 | | | | | | 0.137 | |
| 92 | | | | | | | |
| 93 | | | | | | | |
| 94 | 0.311 | | | | | | |
| 95 | | | | | | | |
| 96 | | | | 0.597 | | | |
| 97 | | | | | | 0.411 | 0.295 |
| 98 | | | | | | | |
| 99 | | | | | | | |
| 100 | | | | | | | |
| 101 | | | | | | | |
| 102 | | | 1.975 | | | | |
| 103 | | | | | | | |
| 104 | | | | | | 0.274 | 0.196 |
| 105 | | | | | | | |
| 106 | | | | | | | |
| 107 | | | | | | | |
| 108 | | | | | | | 0.393 |
| 109 | | | | | | | 0.295 |
| 110 | | | | 1.194 | | | |
| 111 | | | | | | | |
| 112 | | | | | 0.197 | | 0.098 |
| 113 | | | | | | | |
| 114 | | | | | | | |
| 115 | | | | | 0.197 | | |
| 116 | | | | | | | |
| 117 | | | | | | 0.137 | |
| 118 | | | | | | 0.137 | |
| 119 | | | | | | | |
| 120 | | | | | | | |
| 121 | | | | | | | |
| 122 | | | | | | | |
| 123 | | | | | | 0.137 | 0.098 |
| 124 | | 0.128 | | | | | |
| 125 | | | | | | | |
| 126 | | | | | 0.395 | 0.137 | |
| 127 | | | | 0.597 | | | |
| 128 | | | | | | | |
| 129 | | | | | | | |
| 130 | | | | | | | |
| 131 | | | | | | 0.274 | 0.098 |
| 132 | 0.311 | | | | | | |
| 133 | | | | | | | |
| 134 | | | | | | | |
| 135 | | 0.128 | | | | | |
| 136 | | | | | | | 0.196 |
| 137 | | | | | | | |
| 138 | | | | | | | |
| 139 | | | | | 0.197 | | 0.098 |
| 140 | | | | | | | 0.098 |
| 141 | | | | | 0.592 | | |
| 142 | | | | | | 0.137 | |
| 143 | | | | | | | |
| 144 | | | | | | | |
| 145 | | | | | | | |
| 146 | | | | | | | |
| 147 | | | | | | | |
| 148 | | | | | | | |
| 149 | | | | | | | |
| 150 | | | | | | 0.137 | 0.098 |
| 151 | | | | | 0.395 | 0.274 | 0.098 |
| 152 | | | | | | | |
| 153 | | | | | | | |
| 154 | | | | | | | |
| 155 | | | | | | 0.274 | 0.098 |
| 156 | | | | | | | 0.098 |
| 157 | | | | | | | |
| 158 | | | | | | 0.137 | |
| 159 | | | | | 0.395 | | |
| 160 | | 0.128 | | | | | |
| 161 | | 0.256 | | | | | |
| 162 | | | | | | 0.137 | |
| 163 | | | | | | | |
| 164 | | 0.256 | | | | | |
| 165 | | | | | | | 0.098 |
| 166 | | | | | | | |
| 167 | | | | | | | |
| 168 | | | | | | | |
| 169 | | | | | | | |
| 170 | | | | | | | |
| 171 | | | | | | | |
| 172 | 1.243 | | | | | | |
| 173 | 0.311 | | | | | | |
| 174 | | | | | | | |
| 175 | 0.311 | | | | | | |
| 176 | | | | 0.597 | | | |
| 177 | | | | | | | |
| 178 | | | | | | 0.137 | |
| 179 | | | | | | | |
| 180 | 0.311 | | | | | | |
| 181 | | 0.128 | | | | | 0.098 |
| 182 | 0.622 | | | | | | |
| 183 | | | | 0.597 | | | |
| 184 | 0.311 | | | | | | |
| 185 | | 0.128 | | | | 0.137 | |
| 186 | | 0.128 | | | | | |
| 187 | | | | | | | |
| 188 | | | | 0.597 | | | |
| 189 | | | | 0.597 | | | |
| 190 | | | | | | | |
| 191 | | | | | | | |
| 192 | | | | | | 0.411 | 0.491 |
| 193 | | | | | 0.986 | 0.137 | 0.196 |
| 194 | | | | | | | |
| 195 | | | | | | | |
| 196 | | | | | 0.197 | 0.274 | |
| 197 | | | | | 0.395 | | |
| 198 | | | | | | | |
| 199 | | | | | | | |
| 200 | | | | | | | |
| 201 | | | | | | | |
| 202 | | | | | | 0.137 | |
| 203 | | | | | | | |
| 204 | | | | | | | 0.098 |
| 205 | | 0.256 | | | | | 0.098 |
| 206 | 0.311 | | | | 0.592 | | |
| 207 | | | | 0.597 | | | |
| 208 | | 0.128 | | | | | |
| 209 | 0.311 | | | | | | |
| 210 | | | | | | 0.411 | 0.098 |
| 211 | | | | | | | |
| 212 | | | | | | | |
| 213 | | | | | | | |
| 214 | | | | 0.597 | | | |
| 215 | | | | | | | |
| 216 | | | | | | | 0.098 |
| 217 | 0.311 | | | | | | |
| 218 | 0.311 | | | | | | |
| 219 | | | | | | | |
| 220 | | 0.128 | | | | | |
| 221 | | 0.128 | | | | | |
| 222 | | | | | | | |
| 223 | | | | | | 0.137 | |
| 224 | | | | | 0.197 | 0.137 | 0.098 |
| 225 | | | | | 0.197 | 0.137 | 0.098 |
| 226 | | | | | | | |
| 227 | | | 0.494 | | | | |
| 228 | 1.554 | | | | | | |
| 229 | | | | | | 0.137 | 0.098 |
| 230 | | | 0.988 | | 0.197 | | |
| 231 | | 0.128 | | | | | 0.098 |
| 232 | | | | | | | 0.098 |
| 233 | | | | | | | |
| 234 | | | | | | | |
| 235 | | | | | 0.197 | | |
| 236 | | | | | | | |
| 237 | | | | 1.791 | | 0.137 | |
| 238 | | | | | | | |
| 239 | | | | | | | |
| 240 | | | | | | 0.137 | |
| 241 | | 0.128 | | 0.597 | | | |
| 242 | | | | | | | 0.098 |
| 243 | 0.311 | | | | | | |
| 244 | | 0.128 | | | 0.197 | | |
| 245 | | | | | | | |
| 246 | | | | | | 0.274 | |
| 247 | | 0.128 | | | | | |
| 248 | | | | | | 0.137 | 0.098 |
| 249 | | | 0.988 | | | | |
| 250 | | | 0.494 | | | | |
| 251 | | | | | | | 0.098 |
| 252 | | | | | | | |
| 253 | | 0.128 | | | | | |
| 254 | | 0.256 | | | | | |
| # Biomarkers Per | 15 | 21 | 6 | 12 | 19 | 30 | 32 |
| Sample | | | | | | | |
| % Coverage | 5% | 7% | 2% | 4% | 6% | 10% | 10% |
| | Stage | | | | | | |
| | Braak VI | Braak VI | Braak VI | Braak VI | Braak VI | Braak VI | Braak VI |
| Seq. ID | SRR1568440 | SRR1568456 | SRR1568489 | SRR1568495 | SRR1568524 | SRR1568529 | SRR1568537 |
| 47 | | | | | | | 1.539 |
| 48 | | | | | | | 2.694 |
| 49 | | | | | | | |
| 50 | | | | | | | 0.385 |
| 51 | | | | | | | 0.385 |
| 52 | | | | | | | |
| 53 | | | | | | | |
| 54 | | | | | | | 0.77 |
| 55 | | | | 0.189 | | | |
| 56 | | | | | | | |
| 57 | | | | | | | 1.924 |
| 58 | | | | | | | 4.233 |
| 59 | | | | | | | |
| 60 | | | | | | | |
| 61 | 0.177 | | | | 0.665 | | |
| 62 | | | | | | | 0.385 |
| 63 | | | | | | | |
| 64 | 0.53 | | | | 0.133 | | |
| 65 | | | | | | | |
| 66 | | | | | | | |
| 67 | 0.177 | | | | | | |
| 68 | | | | | | | |
| 69 | | | | | | | |
| 70 | | | | | | | |
| 71 | | 0.051 | | | | | |
| 72 | | 0.101 | | | | | |
| 73 | | | | | | | |
| 74 | | | | | | | |
| 75 | | | | | | | |
| 76 | | | | | | | |
| 77 | | 0.101 | | | | | |
| 78 | | | | | | | 0.77 |
| 79 | | 0.101 | | | | | |
| 80 | | | | | | | |
| 81 | | | | | 0.133 | | |
| 82 | | | | | | | 0.385 |
| 83 | | | | | 0.133 | | |
| 84 | | | | | | | |
| 85 | | | | | | | |
| 86 | 0.353 | | | | 0.133 | 0.205 | |
| 87 | | | | | | | |
| 88 | | | | | | | |
| 89 | | | | | | | |
| 90 | | | | | | | |
| 91 | 0.177 | | | | | | |
| 92 | | 1.216 | | | | | |
| 93 | | | | | | | |
| 94 | | | | | | | |
| 95 | | 0.355 | | | 0.133 | | |
| 96 | | | | | | | 0.77 |
| 97 | | | | | | | |
| 98 | | 0.152 | | | | | |
| 99 | | 0.152 | | | | | |
| 100 | | | | | | | 0.77 |
| 101 | 0.353 | 0.101 | | 0.284 | | | |
| 102 | | | | | | | |
| 103 | 0.177 | 0.253 | | | | | |
| 104 | | | | | | | |
| 105 | 0.177 | 0.203 | | 0.189 | | | |
| 106 | | 0.051 | | | | | |
| 107 | | 0.051 | | | | | |
| 109 | | | | | | | |
| 110 | | | | | | | |
| 111 | | 0.051 | | 0.189 | | | |
| 112 | | | | | | | |
| 113 | | 0.203 | | | | | |
| 114 | | 0.203 | | | | | |
| 115 | | | | | | | |
| 116 | | | | | | | |
| 117 | | | | | | | |
| 118 | | 0.101 | | | | | |
| 119 | | | | 0.095 | | | |
| 120 | | 0.051 | | | 0.266 | | |
| 121 | | | | | | | |
| 122 | | | | | | | |
| 123 | | | | | | | |
| 124 | | | | | 0.399 | 0.205 | |
| 125 | | | | | | | |
| 126 | | 0.051 | | | | | |
| 127 | | | | | | | 0.385 |
| 128 | | | | | 0.133 | | |
| 129 | | | | 0.095 | | | |
| 130 | | | | | | | |
| 131 | | | | | | | |
| 132 | | 0.051 | | | | | |
| 133 | | | | | | | |
| 134 | | | | | | | |
| 135 | | | 0.423 | | | | |
| 136 | | | | | | | |
| 137 | | | | | | | |
| 138 | 0.353 | 0.051 | | 0.095 | | | |
| 139 | | | | | | | |
| 140 | | 0.101 | | | | | |
| 141 | | | | | 0.133 | | |
| 142 | | 0.101 | | | | | |
| 143 | | | | | | | |
| 144 | | 0.051 | | | | | |
| 145 | | | | | | 0.205 | |
| 146 | 0.177 | | | 0.189 | | | |
| 147 | | 0.152 | | | 0.133 | | |
| 148 | | | | 0.095 | | | 0.385 |
| 149 | | | | | | | |
| 150 | | | | | | | |
| 151 | 0.177 | | | | | | |
| 152 | | | | | | | |
| 153 | | | | | | | |
| 154 | | | | | | | |
| 155 | | | | | | | |
| 156 | | | | | | | |
| 157 | 0.177 | | | 0.095 | | | |
| 158 | | 0.101 | | | | | |
| 159 | | | | | | | |
| 160 | | | | | | | |
| 161 | | 0.051 | | | | | |
| 162 | | | | | | | |
| 163 | | | | | | | |
| 164 | | | | | 0.266 | | |
| 165 | | 0.051 | | | | | |
| 166 | | | | | | 0.205 | |
| 167 | | | | | | | |
| 168 | | | | | | | 0.385 |
| 169 | | | | 0.095 | | | |
| 170 | | | | 0.095 | | | 0.385 |
| 171 | 0.353 | | | | 0.665 | | |
| 172 | | | | | | | |
| 173 | | | | | | | |
| 174 | | 0.051 | | | | | |
| 175 | 0.177 | | | | | | |
| 176 | | | | | | | 0.385 |
| 177 | 0.177 | | | | 0.133 | | |
| 178 | | | | | | | |
| 179 | 0.177 | | | 0.095 | | | |
| 180 | | | | | | | |
| 181 | | | | | | | |
| 182 | | | | | 0.133 | | |
| 183 | | | | | | | |
| 184 | 0.177 | | | | | | |
| 185 | | 0.051 | | | 0.133 | | |
| 186 | | | | | | | |
| 187 | | | | | | | |
| 188 | | | | 0.095 | | | |
| 189 | | | | | | | 5.002 |
| 190 | | 0.709 | | 0.095 | | | |
| 191 | 0.177 | 0.101 | | | | | |
| 192 | | | | | | | |
| 193 | | | | | | | |
| 194 | | | | | | | |
| 195 | | | | 0.095 | 0.133 | | |
| 196 | | | | | | | |
| 197 | | | | 0.095 | | 0.205 | |
| 198 | 0.177 | 0.051 | | | | 0.616 | |
| 199 | | | | | | | |
| 200 | | 0.203 | | | | | |
| 201 | | 0.051 | | | | | |
| 202 | | | | | | 0.205 | |
| 203 | | 0.051 | | | | | |
| 204 | | 0.051 | | | | | |
| 205 | | | | 0.095 | | 0.411 | |
| 206 | | 0.051 | | 0.095 | 0.133 | | |
| 207 | | | | | | | |
| 208 | | | | | 0.266 | | |
| 209 | | 0.051 | | | | | |
| 210 | | | | | | | |
| 211 | | 0.101 | | 0.095 | | | |
| 212 | | 0.051 | | 0.095 | | | |
| 213 | | | | | 0.133 | 0.205 | |
| 214 | | | | | | | |
| 215 | | | | | | | |
| 216 | | | | | | | |
| 217 | 0.177 | | | | | | |
| 218 | | | | | | | |
| 219 | | | | 0.189 | | | |
| 220 | | | | 0.095 | 0.133 | | |
| 221 | | | | 0.095 | | | |
| 222 | | | | 0.095 | | 0.205 | |
| 223 | | | | | | | |
| 224 | | | 0.141 | | | | |
| 225 | | | | | | | |
| 226 | 0.177 | | | | 0.266 | 0.205 | |
| 227 | | 0.051 | | 0.189 | | | |
| 228 | | | | | | | |
| 229 | | | | | | | |
| 230 | | | | 0.095 | | | |
| 231 | | | | | | | |
| 232 | | 0.051 | | | | | |
| 233 | 0.177 | | | | | | |
| 234 | 0.177 | | | | | | |
| 235 | | 0.051 | | | | 0.205 | |
| 236 | | 0.152 | | | | | |
| 237 | | | | | | | 0.77 |
| 238 | | | | | | | |
| 239 | | 0.051 | | | | | |
| 240 | | | | 0.095 | | | |
| 241 | 0.177 | | | 0.095 | | 0.205 | |
| 242 | | | | | | | |
| 243 | | 0.051 | | | | | |
| 244 | 0.177 | | | | | | |
| 245 | | | | | | | |
| 246 | | | | 0.095 | | | |
| 247 | | | | | 0.266 | | |
| 248 | | | | | | | |
| 249 | | | | 0.095 | | | |
| 250 | | 0.101 | | 0.473 | | | |
| 251 | | 0.051 | | | | | |
| 252 | | | | | 0.266 | | |
| 253 | | 0.051 | | | 0.133 | | |
| 254 | 0.177 | | | | 0.266 | | |
| # Biomarkers Per | 26 | 50 | 2 | 32 | 26 | 13 | 19 |
| Sample | | | | | | | |
| % Coverage | 8% | 16% | 1% | 10% | 8% | 4% | 6% |
| | Stage | | | | | | |
| | Braak VI | Braak VI | Braak VI | Braak VI | Braak VI | Braak VI | Braak VI |
| Seq. ID | SRR1568539 | SRR1568561 | SRR1568565 | SRR1568599 | SRR1568610 | SRR1568640 | SRR1568647 |
| 47 | | | | | | | |
| 48 | | | | | | | |
| 49 | | | | | | | |
| 50 | | | | | | | |
| 51 | | | | | | | |
| 52 | | | | 0.111 | | | |
| 53 | | | | | | | |
| 54 | | | | | | | |
| 55 | | | | | | | |
| 56 | | | | | | | |
| 57 | | | | | | | |
| 58 | | | | | | | |
| 59 | | | | 0.223 | | | 0.273 |
| 60 | | 0.453 | | | 0.547 | 0.96 | |
| 61 | | | | | | | |
| 62 | | | | | | | |
| 63 | | | | | | | 0.273 |
| 64 | | | | | | | |
| 65 | | | | | | | |
| 66 | | | | | | | |
| 67 | | | 0.654 | | 0.274 | | |
| 68 | | | | | | | |
| 69 | | 0.091 | | | | | 0.547 |
| 70 | | | | | | | |
| 71 | | | | | | | |
| 72 | | 0.181 | | | | | |
| 73 | | | | | | | |
| 74 | | | | | | | 0.273 |
| 75 | | | | | | | |
| 76 | | | 0.164 | | | | |
| 77 | | | | 0.111 | | | |
| 78 | | | | | | | |
| 79 | | | | | | | |
| 80 | | | | | | | |
| 81 | | | | | | 0.16 | |
| 82 | | | | | | | |
| 83 | | | | | | | 0.273 |
| 84 | | | | | | 0.16 | 0.547 |
| 85 | | | | 0.111 | 0.274 | | |
| 86 | | | | | | | |
| 87 | | | | 0.111 | | | |
| 88 | | | | | | | |
| 89 | | | | 0.223 | | | |
| 90 | | | | | | | |
| 91 | | | | | | | |
| 92 | | | | | | | 0.273 |
| 93 | | 0.181 | 0.164 | | 0.274 | | |
| 94 | | | | | | | |
| 95 | | | | | | | |
| 96 | | | | | | | |
| 97 | | | | | | | |
| 98 | | | | | | | |
| 99 | | | | | | | |
| 100 | | | | | | | |
| 101 | | | | | | | |
| 102 | | | | | | 0.64 | |
| 103 | | 0.091 | | | | | |
| 104 | | | | | | | |
| 105 | | | | | | | |
| 106 | | | | 0.111 | | 0.16 | |
| 107 | | | | | | | |
| 108 | | | | | | | |
| 109 | | | | | | | |
| 110 | | | | | | | |
| 111 | | | | | | | |
| 112 | | | | | | | |
| 113 | | 0.091 | | | | | |
| 114 | 0.166 | | | | | | |
| 115 | 0.166 | | | 0.334 | | | |
| 116 | | 0.091 | | | | | 0.273 |
| 117 | | | | 0.223 | | 0.16 | |
| 118 | | | | | | | 0.273 |
| 119 | | | 0.164 | | | 0.16 | |
| 120 | | | | | | 0.32 | |
| 121 | | 0.363 | | | | | |
| 122 | | 0.181 | | 0.111 | | | |
| 123 | | | | | | 0.16 | |
| 124 | | | | | | | |
| 125 | | | | | 0.274 | 0.48 | |
| 126 | | | | | | | |
| 127 | | | | | | | |
| 128 | | 0.091 | | | | 0.16 | |
| 129 | | | | | | | |
| 130 | | 0.091 | | 0.111 | | | |
| 131 | | | 0.327 | | | | |
| 132 | | | | | | | |
| 133 | | 0.091 | | 0.334 | | | 0.273 |
| 134 | | | | | | | |
| 135 | | 0.091 | | | | | |
| 136 | | | | | | 0.32 | |
| 137 | | 0.091 | | 0.111 | | | |
| 138 | | | | | | | |
| 139 | | 0.181 | | | | | |
| 140 | | | | | | 0.16 | |
| 141 | | | | | | | |
| 142 | 0.166 | | | 0.223 | | | |
| 143 | | | | | 0.274 | 0.16 | |
| 144 | | | | | | | |
| 145 | | | | 0.334 | | 0.16 | |
| 146 | | | | 0.111 | | 0.16 | |
| 147 | | 0.091 | | | | | |
| 148 | | | | | | 0.16 | |
| 149 | | | | | | | |
| 150 | | | | | | 0.16 | |
| 151 | | | | | | | |
| 152 | | | 0.327 | 0.111 | 0.547 | | |
| 153 | | 0.091 | | | | | 0.273 |
| 154 | | | | 0.223 | 0.547 | | |
| 155 | | | | | | | |
| 156 | | | | | | 0.16 | |
| 157 | | | | | | | |
| 158 | | | | 0.111 | | 0.32 | |
| 159 | | | | 0.223 | | | |
| 160 | | 0.091 | | | | | 0.273 |
| 161 | | | | | | | |
| 162 | | | | | | 0.16 | |
| 163 | | 0.091 | | | | | |
| 164 | | | 0.164 | | | | |
| 165 | | | | | | | |
| 166 | | | | | | | |
| 167 | | 0.091 | | | 0.547 | | |
| 168 | | | | | | | |
| 169 | | | | | | | |
| 170 | | | | 0.111 | | | |
| 171 | | | | | | | |
| 172 | | | | | | | |
| 173 | | | | | | | |
| 174 | | 0.272 | | | 0.274 | | 0.273 |
| 175 | | | | | | | |
| 176 | | 0.091 | | | | | |
| 177 | | | | 0.334 | | | |
| 178 | | | | | | | |
| 179 | | 0.181 | | | | | |
| 180 | | | | | | | |
| 181 | | | | 0.223 | | | |
| 182 | | | | 0.111 | | | |
| 183 | | 0.272 | | 0.111 | | | |
| 184 | | | | | | | |
| 185 | | | | | | | |
| 186 | | | | | | | |
| 187 | | 0.091 | 0.164 | | | | |
| 188 | | 0.091 | | | | | |
| 189 | | | | | | | |
| 190 | | | | | | | |
| 191 | | | | | | | |
| 192 | | | | | | 0.32 | |
| 193 | | | | | | | |
| 194 | | | | | | | |
| 195 | | | | | | 0.8 | |
| 196 | | | | | | 0.16 | |
| 197 | | | | 0.111 | | | |
| 198 | | | | | 0.274 | | |
| 199 | | | | 0.334 | | | |
| 200 | | | | | 0.274 | | |
| 201 | | | 0.164 | | | 0.48 | 0.273 |
| 202 | | | | | | | |
| 203 | | | 0.164 | | 0.274 | | |
| 204 | 0.166 | | | | | 0.16 | |
| 205 | | | | | | | |
| 206 | | | | | | 0.16 | |
| 207 | | | | | | | |
| 208 | | | | | | | |
| 209 | | | | | | | |
| 210 | 0.166 | | 0.164 | | | | |
| 211 | | | | | | | |
| 212 | | | | | | | |
| 213 | | | | | | | |
| 214 | | | | | 0.274 | | 0.547 |
| 215 | | | 0.327 | | | | |
| 216 | | | | | | 0.32 | 0.273 |
| 217 | | | | | | | |
| 218 | | | | | | | |
| 219 | | | 0.164 | | | 0.16 | |
| 220 | | 0.091 | | | | | |
| 221 | | | | | | | |
| 222 | | 0.091 | | 0.111 | | | |
| 223 | | | | | | | |
| 224 | | | | | | | |
| 225 | | | | | | 0.32 | |
| 226 | | | | | | | |
| 227 | | | | | | | |
| 228 | | | | | | | |
| 229 | | | | | | 0.16 | |
| 230 | | | | 0.111 | | | |
| 231 | 0.166 | | | | | 0.16 | 0.273 |
| 232 | 0.166 | 0.091 | | 0.111 | | | |
| 233 | | | | 0.111 | | | |
| 234 | 0.166 | | | 0.111 | | | |
| 235 | | | 0.491 | | 0.821 | | |
| 236 | | 0.091 | | | | | |
| 237 | | | | | | | |
| 238 | | | | 0.111 | | 0.16 | |
| 239 | | | | 0.111 | | 0.48 | 0.273 |
| 240 | | | | | | | |
| 241 | | | | | | | |
| 242 | | | | | | 0.16 | |
| 243 | | | | | | | |
| 244 | | 0.091 | | | | | |
| 245 | | 0.091 | | 0.111 | | | |
| 246 | | | | | 0.274 | | |
| 247 | | | 0.164 | | | 0.16 | |
| 248 | | 0.091 | | 0.223 | | 0.16 | |
| 249 | | | | 0.223 | | | 0.547 |
| 250 | | 0.091 | | | | | |
| 251 | | | | | | | 0.273 |
| 252 | | | | 0.111 | 0.274 | | |
| 253 | 0.333 | 0.091 | 0.164 | | | | |
| 254 | 0.166 | | 0.164 | 0.334 | | | |
| # Biomarkers Per | 10 | 35 | 17 | 39 | 17 | 37 | 20 |
| Sample | | | | | | | |
| % Coverage | 3% | 11% | 5% | 12% | 5% | 12% | 6% |

| | Stage | | | | | | | |
|---|---|---|---|---|---|---|---|---|
| | Braak VI | Braak VI | Braak VI | Braak VI | Braak VI | Braak VI | Braak VI | Braak VI |
| Seq. ID | SRR1568661 | SRR1568663 | SRR1568672 | SRR1568677 | SRR1568722 | SRR1568740 | SRR1568747 | SRR1568755 |
| 47 | | | | | | | | |
| 48 | | | | | | | | |
| 49 | | | | 0.475 | | | | |
| 50 | | | | | | | | |
| 51 | | | | | | | | |
| 52 | | | | | | | | |
| 53 | | | | | | | | |
| 54 | | | | | | | | |
| 55 | | | | | | | 6.415 | |
| 56 | | | | | | | | |
| 57 | | | | | | | | |
| 58 | | | | | | | | |
| 59 | | | | | | | | |
| 60 | | | | | | | | |
| 61 | | | | | | | | |
| 62 | | | | | | | | |
| 63 | | | | | | | | |
| 64 | | | | | | | | |
| 65 | | | | 0.95 | 0.562 | | | |
| 66 | | | | | | | | |
| 67 | | | | | | | | |
| 68 | | | | | | | | 0.173 |
| 69 | | | | | | | | 0.086 |
| 70 | | | | | 0.562 | | | 0.259 |
| 71 | | | | | | | | |
| 72 | | | | | | | | |
| 73 | | | | 0.475 | | | | |
| 74 | | | | | | | | |
| 75 | 0.672 | | | | | | | |
| 76 | | | 0.11 | | | 0.233 | | |
| 77 | | | | | | | | 0.086 |
| 78 | | | | | | | | |
| 79 | | | 0.11 | | | | | |
| 80 | | | | | | | | |
| 81 | | | | | | | | |
| 82 | | | | | | | | 0.259 |
| 83 | | | | | | | | |
| 84 | | | 0.11 | | | | | |
| 85 | | | | | | | 0.238 | |
| 86 | | | | | | | | |
| 87 | | | | | 0.562 | | | |
| 88 | 0.672 | | | | 0.562 | | | |
| 89 | | | 0.11 | | | | | |
| 90 | | | 0.22 | | | | | |
| 91 | | | | | | | | 0.173 |
| 92 | | | | | | | | |
| 93 | | | | | | | | |
| 94 | | | | | 0.562 | | | |
| 95 | | | | | | | | |
| 96 | | | | | | | | |
| 97 | | | | | | | | 0.259 |
| 98 | | | | | | | | 0.086 |
| 99 | | | | | | | | 0.086 |
| 100 | | | | | | | | |
| 101 | | | | | | | | |
| 102 | | | 0.11 | | | | | |
| 103 | | | | | | | | |
| 104 | | | | | | | | |
| 105 | | | | | | | | |
| 106 | | | | | | | | |
| 107 | | | | | | | | |
| 108 | | | | | | | | |
| 109 | | | | | | | | |
| 110 | | | | 0.95 | | | | |
| 111 | | | | | | | | |
| 112 | | | | | | | | |
| 113 | | | | | | | | |
| 114 | | | | | | | | 0.173 |
| 115 | | | | | | | | |
| 116 | | | | | | | | |
| 117 | | | 0.11 | | | | | |
| 118 | | | | | | | | |
| 119 | | | | | | | | |
| 120 | | | | | | | | |
| 121 | | | | | | | | |
| 122 | 0.672 | | | | | | | |
| 123 | | | | | | | | |
| 124 | | | | | | | | |
| 125 | | | | | | | | |
| 126 | | 0.2 | | | | | | |
| 127 | | | | | | | | |
| 128 | | | | | | | | |
| 129 | | | | | | | | |
| 130 | | | | | | | | |
| 131 | | | | | | | | |
| 132 | 0.672 | | | | 2.248 | | | |
| 133 | | | | | | | | |
| 134 | | | | 0.475 | | | | |
| 135 | 0.672 | | | | | | | |
| 136 | | | | | | | | |
| 137 | | | | | | | | |
| 138 | | | | | | | | |
| 139 | | | | | | | | |
| 140 | | | | | | | | |
| 141 | | | 0.11 | 0.475 | | | | |
| 142 | | | | | | | | |
| 143 | | | | | | | | |
| 144 | | 0.2 | | | | | | 0.173 |
| 145 | | | 0.11 | | | | | |
| 146 | | | | | | | | |
| 147 | | | | | | | | |
| 148 | | | | | | | | |
| 149 | | | | | | | | 0.086 |
| 150 | | | | | | | | 0.086 |
| 151 | | | | | | | | |
| 152 | | | | | | | 0.238 | |
| 153 | | | | | | | 0.475 | |
| 154 | | | 0.11 | | | | | 0.086 |
| 155 | | | | | | | | 0.086 |
| 156 | | | | | | | | |
| 157 | | | | | | | | |
| 158 | | | | | | | | |
| 159 | | | | | 0.562 | | | |
| 160 | | | | | | | | 0.173 |
| 161 | | | 0.22 | | | | | 0.086 |
| 162 | | | | | | | | 0.086 |
| 163 | | | 0.11 | 0.475 | | | | |
| 164 | | | | | | | | |
| 165 | | | 0.11 | | | | | |
| 166 | | | | | | | | |
| 167 | | | | | | 0.233 | | |
| 168 | | | | | | | | |
| 169 | | | | | | | | |
| 170 | | | | | | | | |
| 171 | | | | | | | | |
| 172 | 0.672 | | | 0.475 | | | | |
| 173 | 0.672 | | | 0.95 | 1.124 | | | |
| 174 | | | | | | | | |
| 175 | 1.345 | | | | | | | |
| 176 | | | | | | | | |
| 177 | | | | | | | | |
| 178 | | | | | | | | |
| 179 | | | | | | | | |
| 180 | 0.672 | | | | | | | |
| 181 | | | | | | | | |
| 182 | 1.345 | | | 0.475 | | | | |
| 183 | | | | | | | | |
| 184 | | | | | | | | 0.086 |
| 185 | | | 0.11 | | | | | |
| 186 | | 0.2 | 0.11 | | | | | |
| 187 | | | | | | | | |
| 188 | | | | | | | | |
| 189 | | | | | | | | 0.086 |
| 190 | | | 0.11 | | | | | 0.086 |
| 191 | | | | | | | | |
| 192 | | | | | | | | 0.086 |
| 193 | | | | | | | | 0.086 |
| 194 | | 0.599 | | 0.475 | | | | 0.173 |
| 195 | | | | | | | | |
| 196 | | | | | | | | 0.431 |
| 197 | | | | | | | | |
| 198 | | | | | | | | |
| 199 | | | | | 1.124 | | | 0.173 |
| 200 | | | 0.11 | | | | | 0.086 |
| 201 | | | | | | | | |
| 202 | | | 0.22 | | | | 0.238 | |
| 203 | | | | | | | | |
| 204 | | | | | | | | |
| 205 | | | | | | | | |
| 206 | | | | | | | | |
| 207 | | | | | | | | |
| 208 | | | | | | | | 0.086 |
| 209 | | | | | 0.562 | | 0.238 | |
| 210 | | | | | | | 0.238 | |
| 211 | | | 0.22 | | | | | 0.086 |
| 212 | | | | | | | | |
| 213 | | | | | | | | 0.086 |
| 214 | | | | | | | | |
| 215 | | | | | | 0.233 | | |
| 216 | | 0.2 | | | | | | 0.086 |
| 217 | 0.672 | | | | 0.562 | | | |
| 218 | | | | | | | | |
| 219 | | | | | | | | |
| 220 | | | | | | | | |
| 221 | | | 0.11 | | | | | |
| 222 | | | | | | | | |
| 223 | | | | | 0.562 | | 0.238 | |
| 224 | | | 0.11 | | | | | |
| 225 | | | | | | | | 0.086 |
| 226 | | | | | | | | |
| 227 | | | | | | | | |
| 228 | 4.035 | | | 0.95 | 0.562 | | | |
| 229 | | | | | | 0.233 | | |
| 230 | | | | | | | | |
| 231 | | | | | | | | |
| 232 | | | | | | | | |
| 233 | | | 0.11 | | | | | |
| 234 | | | | | | | | |
| 235 | | | | | | | | 0.086 |
| 236 | | | | | | | | |
| 237 | | | | | | | | |
| 238 | | 0.2 | | | | | | 0.431 |
| 239 | | 0.2 | | | | | | 0.086 |
| 240 | | | | | | | | |
| 241 | | 0.2 | | | | | | |
| 242 | | | | | | 0.233 | | |
| 243 | | | | | | | | |
| 244 | | | | | | | | 0.086 |
| 245 | | | | | | | 0.238 | |
| 246 | | 0.399 | | | | | | |
| 247 | | 0.2 | | | | 0.233 | | |
| 248 | | 0.798 | | | | | | |
| 249 | | | | | | | | |
| 250 | | | | | | | | 0.086 |
| 251 | | | | | | | 0.475 | 0.086 |
| 252 | | | | | | | | 0.086 |
| 253 | | | | | | | | |
| 254 | | | 0.11 | | | | | |
| # Biomarkers | 12 | 11 | 23 | 12 | 13 | 6 | 10 | 39 |
| Per Sample | | | | | | | | |
| % Coverage | 4% | 4% | 7% | 4% | 4% | 2% | 3% | 12% |

TABLE 5
Identified sRNA biomarkers in cerebrospinal
fluid that have a positive correlation with Braak
Stage in order to monitor Alzheimer's Disease

| | | Braak | Braak | Braak | Braak | Braak | | |
|---|---|---|---|---|---|---|---|---|
| Seq. | Total | II | III | IV | V | VI | | Frequency |
| ID | Reads | Avg | Avg | Avg | Avg | Avg | Hits | (Sensitivity) |
| 58 | 21 | 0.000 | 0.386 | 0.542 | 0.660 | 4.233 | 4 | 9.38% |
| 189 | 26 | 0.000 | 0.000 | 0.643 | 1.149 | 1.895 | 3 | 10.94% |
| 78 | 8 | 0.000 | 0.129 | 0.365 | 0.366 | 0.770 | 4 | 9.38% |
| 172 | 15 | 0.000 | 2.319 | 1.752 | 0.607 | 0.574 | 4 | 10.94% |
| 193 | 14 | 0.000 | 0.000 | 0.143 | 0.161 | 0.351 | 3 | 10.94% |
| 97 | 15 | 0.000 | 0.000 | 0.143 | 0.292 | 0.322 | 3 | 9.38% |
| 122 | 10 | 0.000 | 0.000 | 0.195 | 0.262 | 0.321 | 3 | 9.38% |
| 215 | 9 | 0.000 | 0.000 | 0.475 | 0.352 | 0.280 | 3 | 10.94% |
| 248 | 15 | 0.000 | 0.000 | 0.143 | 0.214 | 0.251 | 3 | 14.06% |
| 164 | 8 | 0.000 | 0.000 | 0.377 | 0.253 | 0.215 | 3 | 9.38% |
| 120 | 10 | 0.000 | 0.000 | 0.145 | 0.189 | 0.212 | 3 | 9.38% |
| 93 | 22 | 0.000 | 0.000 | 2.208 | 0.366 | 0.206 | 3 | 9.38% |
| 126 | 9 | 0.000 | 0.000 | 0.614 | 0.254 | 0.196 | 3 | 9.38% |
| 253 | 13 | 0.000 | 0.000 | 0.377 | 0.183 | 0.154 | 3 | 17.19% |
| 112 | 11 | 0.000 | 0.000 | 3.673 | 0.323 | 0.148 | 3 | 9.38% |
| 144 | 8 | 0.000 | 0.000 | 0.298 | 0.168 | 0.141 | 3 | 9.38% |
| 213 | 9 | 0.000 | 0.000 | 0.286 | 0.155 | 0.141 | 3 | 10.94% |
| 244 | 8 | 0.000 | 0.000 | 0.195 | 0.146 | 0.138 | 3 | 12.50% |
| 123 | 10 | 0.000 | 0.000 | 0.572 | 0.129 | 0.132 | 3 | 9.38% |
| 222 | 8 | 0.000 | 0.000 | 0.195 | 0.172 | 0.126 | 3 | 10.94% |
| 150 | 8 | 0.000 | 0.000 | 0.286 | 0.260 | 0.120 | 3 | 9.38% |
| 240 | 9 | 0.000 | 0.000 | 0.735 | 0.129 | 0.116 | 3 | 12.50% |
| 52 | 8 | 1.126 | 1.740 | 0.544 | 0.277 | 0.111 | 5 | 9.38% |
| 220 | 8 | 0.000 | 0.000 | 0.267 | 0.121 | 0.106 | 3 | 10.94% |
| 221 | 8 | 0.000 | 0.000 | 0.584 | 0.145 | 0.103 | 3 | 10.94% |
| 169 | 10 | 1.126 | 0.580 | 0.430 | 0.177 | 0.095 | 5 | 10.94% |
| 165 | 8 | 0.000 | 0.000 | 1.132 | 0.135 | 0.086 | 3 | 9.38% |
| 212 | 9 | 0.000 | 0.000 | 0.195 | 0.170 | 0.073 | 3 | 10.94% |

TABLE 6A
Experimental Alzheimer's disease cohort for biomarker
discovery, taken from serum samples.

| | | Disease | | Age at | Disease | Braak |
|---|---|---|---|---|---|---|
| Group | SRR ID | Type | Gender | Death | Durration | score |
| Experimental | SRR1568369 | Alzheimer's | 1 | 87 | 12 | V |
| Experimental | SRR1568371 | Alzheimer's | 1 | 86 | 21 | V |
| Experimental | SRR1568407 | Alzheimer's | 1 | 75 | 10 | V |
| Experimental | SRR1568409 | Alzheimer's | 1 | 76 | 2 | V |
| Experimental | SRR1568411 | Alzheimer's | 1 | 67 | 9 | V |
| Experimental | SRR1568421 | Alzheimer's | 2 | 77 | 3 | IV |
| Experimental | SRR1568433 | Alzheimer's | 2 | 60 | 5 | VI |
| Experimental | SRR1568435 | Alzheimer's | 2 | 74 | 12 | VI |
| Experimental | SRR1568437 | Alzheimer's | 2 | 88 | 3 | IV |
| Experimental | SRR1568446 | Alzheimer's | 1 | 76 | 4 | V |
| Experimental | SRR1568455 | Alzheimer's | 1 | 80 | 8 | V |
| Experimental | SRR1568468 | Alzheimer's | 1 | 75 | 7 | V |
| Experimental | SRR1568475 | Alzheimer's | 2 | 86 | 9 | V |
| Experimental | SRR1568481 | Alzheimer's | 2 | 75 | 5 | V |
| Experimental | SRR1568490 | Alzheimer's | 2 | 70 | 4 | VI |
| Experimental | SRR1568496 | Alzheimer's | 2 | 74 | 8 | VI |
| Experimental | SRR1568515 | Alzheimer's | 2 | 78 | 8 | V |
| Experimental | SRR1568523 | Alzheimer's | 2 | 87 | 5 | V |
| Experimental | SRR1568525 | Alzheimer's | 2 | 70 | 5 | VI |
| Experimental | SRR1568530 | Alzheimer's | 2 | 57 | 10 | VI |
| Experimental | SRR1568534 | Alzheimer's | 2 | 86 | NA | IV |
| Experimental | SRR1568538 | Alzheimer's | 2 | 65 | 3 | VI |
| Experimental | SRR1568541 | Alzheimer's | 2 | 91 | 10 | IV |
| Experimental | SRR1568547 | Alzheimer's | 2 | 91 | 19 | II |
| Experimental | SRR1568553 | Alzheimer's | 1 | 79 | 5 | II |
| Experimental | SRR1568557 | Alzheimer's | 1 | 90 | 1 | III |
| Experimental | SRR1568562 | Alzheimer's | 1 | 87 | 6 | VI |
| Experimental | SRR1568566 | Alzheimer's | 1 | 78 | 5 | VI |
| Experimental | SRR1568580 | Alzheimer's | 1 | 86 | 4 | II |
| Experimental | SRR1568586 | Alzheimer's | 2 | 89 | 9 | IV |
| Experimental | SRR1568598 | Alzheimer's | 1 | 82 | 12 | VI |
| Experimental | SRR1568600 | Alzheimer's | 1 | 85 | 5 | VI |
| Experimental | SRR1568611 | Alzheimer's | 2 | 68 | 8 | VI |
| Experimental | SRR1568623 | Alzheimer's | 1 | 90 | NA | V |
| Experimental | SRR1568639 | Alzheimer's | 1 | 75 | 6 | V |
| Experimental | SRR1568641 | Alzheimer's | 1 | 83 | 6 | VI |
| Experimental | SRR1568643 | Alzheimer's | 2 | 86 | 10 | V |
| Experimental | SRR1568645 | Alzheimer's | 2 | 79 | 14 | IV |
| Experimental | SRR1568648 | Alzheimer's | 1 | 77 | 1 | VI |
| Experimental | SRR1568652 | Alzheimer's | 1 | 88 | 5 | IV |
| Experimental | SRR1568666 | Alzheimer's | 2 | 81 | 7 | V |
| Experimental | SRR1568669 | Alzheimer's | 2 | 84 | 5 | V |
| Experimental | SRR1568674 | Alzheimer's | 1 | 75 | 8 | V |
| Experimental | SRR1568678 | Alzheimer's | 2 | 90 | 12 | VI |
| Experimental | SRR1568686 | Alzheimer's | 1 | 85 | 1 | III |
| Experimental | SRR1568705 | Alzheimer's | 2 | 86 | 5 | V |
| Experimental | SRR1568719 | Alzheimer's | 2 | 74 | 7 | V |
| Experimental | SRR1568734 | Alzheimer's | 1 | 80 | 3 | IV |
| Experimental | SRR1568744 | Alzheimer's | 2 | 85 | 5 | IV |
| Experimental | SRR1568748 | Alzheimer's | 2 | 89 | 9 | VI |
| Experimental | SRR1568756 | Alzheimer's | 2 | 79 | 10 | VI |
| NA | NA | NA | NA | 80.02 ± 8.1 | 7.16 ± 4.1 | NA |

TABLE 6B
Comparator cohort for AD biomarker discovery, taken from serum samples,
including healthy controls and various other non-Alzheimer's neurological disorders.

| | | Disease | | Age at | Disease | Braak |
|---|---|---|---|---|---|---|
| Group | SRR ID | Type | Gender | Death | Durration | score |
| Comparator | SRR1568594 | Control | 1 | 38 | NA | 0 |
| Comparator | SRR1568429 | Control | 1 | 80 | NA | I |
| Comparator | SRR1568551 | Control | 2 | 76 | NA | I |
| Comparator | SRR1568564 | Control | 1 | 76 | NA | I |
| Comparator | SRR1568570 | Control | 1 | 71 | NA | I |
| Comparator | SRR1568584 | Control | 1 | 65 | NA | I |
| Comparator | SRR1568603 | Control | 1 | 53 | NA | I |
| Comparator | SRR1568613 | Control | 2 | 59 | NA | I |
| Comparator | SRR1568627 | Control | 1 | 93 | NA | I |
| Comparator | SRR1568671 | Control | 1 | 83 | NA | I |
| Comparator | SRR1568676 | Control | 1 | 79 | NA | I |
| Comparator | SRR1568699 | Control | 1 | 68 | NA | I |
| Comparator | SRR1568707 | Control | 2 | 73 | NA | I |
| Comparator | SRR1568713 | Control | 2 | 70 | NA | I |
| Comparator | SRR1568728 | Control | 2 | 76 | NA | I |
| Comparator | SRR1568742 | Control | 1 | 69 | NA | I |
| Comparator | SRR1568381 | Control | 2 | 88 | NA | II |
| Comparator | SRR1568442 | Control | 1 | 86 | NA | II |
| Comparator | SRR1568449 | Control | 2 | 82 | NA | II |
| Comparator | SRR1568464 | Control | 2 | 83 | NA | II |
| Comparator | SRR1568473 | Control | 1 | 91 | NA | II |
| Comparator | SRR1568494 | Control | 1 | 84 | NA | II |
| Comparator | SRR1568500 | Control | 1 | 84 | NA | II |
| Comparator | SRR1568502 | Control | 1 | 73 | NA | II |
| Comparator | SRR1568506 | Control | 1 | 78 | NA | II |
| Comparator | SRR1568507 | Control | 2 | 77 | NA | II |
| Comparator | SRR1568636 | Control | 1 | 74 | NA | II |
| Comparator | SRR1568646 | Control | 1 | 94 | NA | II |
| Comparator | SRR1568660 | Control | 1 | 78 | NA | II |
| Comparator | SRR1568721 | Control | 1 | 86 | NA | II |
| Comparator | SRR1568385 | Control | 2 | 78 | NA | III |
| Comparator | SRR1568387 | Control | 2 | 90 | NA | III |
| Comparator | SRR1568394 | Control | 2 | 80 | NA | III |
| Comparator | SRR1568405 | Control | 1 | 85 | NA | III |
| Comparator | SRR1568416 | Control | 2 | 88 | NA | III |
| Comparator | SRR1568448 | Control | 2 | 85 | NA | III |
| Comparator | SRR1568477 | Control | 1 | 82 | NA | III |
| Comparator | SRR1568492 | Control | 2 | 88 | NA | III |
| Comparator | SRR1568498 | Control | 2 | 87 | NA | III |
| Comparator | SRR1568509 | Control | 1 | 89 | NA | III |
| Comparator | SRR1568521 | Control | 2 | 84 | NA | III |
| Comparator | SRR1568528 | Control | 2 | 75 | NA | III |
| Comparator | SRR1568543 | Control | 2 | 88 | NA | III |
| Comparator | SRR1568582 | Control | 1 | 82 | NA | III |
| Comparator | SRR1568590 | Control | 2 | 99 | NA | III |
| Comparator | SRR1568606 | Control | 1 | 80 | NA | III |
| Comparator | SRR1568609 | Control | 1 | 85 | NA | III |
| Comparator | SRR1568615 | Control | 2 | 95 | 2 | III |
| Comparator | SRR1568633 | Control | 2 | 92 | NA | III |
| Comparator | SRR1568634 | Control | 1 | 68 | NA | III |
| Comparator | SRR1568650 | Control | 1 | 90 | NA | III |
| Comparator | SRR1568654 | Control | 1 | 84 | NA | III |
| Comparator | SRR1568682 | Control | 1 | 84 | NA | III |
| Comparator | SRR1568696 | Control | 2 | 87 | NA | III |
| Comparator | SRR1568698 | Control | 1 | 90 | NA | III |
| Comparator | SRR1568709 | Control | 1 | 78 | NA | III |
| Comparator | SRR1568732 | Control | 2 | 88 | NA | III |
| Comparator | SRR1568750 | Control | 2 | 91 | NA | III |
| Comparator | SRR1568414 | Control | 1 | 89 | 5 | IV |
| Comparator | SRR1568460 | Control | 2 | 78 | NA | IV |
| Comparator | SRR1568462 | Control | 1 | 82 | NA | IV |
| Comparator | SRR1568470 | Control | 2 | 86 | NA | IV |
| Comparator | SRR1568483 | Control | 1 | 75 | 3 | IV |
| Comparator | SRR1568485 | Control | 1 | 91 | 7 | IV |
| Comparator | SRR1568545 | Control | 2 | 87 | NA | IV |
| Comparator | SRR1568560 | Control | 1 | 87 | NA | IV |
| Comparator | SRR1568568 | Control | 1 | 94 | 8 | IV |
| Comparator | SRR1568579 | Control | 2 | 91 | NA | IV |
| Comparator | SRR1568592 | Control | 1 | 92 | NA | IV |
| Comparator | SRR1568621 | Control | 2 | 84 | NA | IV |
| Comparator | SRR1568377 | Parkinson's | 1 | 72 | 9 | I |
| | | Disease | | | | |
| Comparator | SRR1568487 | Parkinson's | 1 | 73 | 18 | I |
| | | Disease | | | | |
| Comparator | SRR1568513 | Parkinson's | 2 | 87 | 9 | I |
| | | Disease | | | | |
| Comparator | SRR1568680 | Parkinson's | 1 | 88 | 0 | I |
| | | Disease | | | | |
| Comparator | SRR1568701 | Parkinson's | 1 | 81 | 8 | I |
| | | Disease | | | | |
| Comparator | SRR1568375 | Parkinson's | 1 | 75 | 8 | II |
| | | Disease | | | | |
| Comparator | SRR1568383 | Parkinson's | 1 | 85 | 15 | II |
| | | Disease | | | | |
| Comparator | SRR1568419 | Parkinson's | 1 | 82 | 13 | II |
| | | Disease | | | | |
| Comparator | SRR1568466 | Parkinson's | 1 | 73 | 13 | II |
| | | Disease | | | | |
| Comparator | SRR1568511 | Parkinson's | 1 | 79 | 4 | II |
| | | Disease | | | | |
| Comparator | SRR1568577 | Parkinson's | 2 | 79 | NA | II |
| | | Disease | | | | |
| Comparator | SRR1568631 | Parkinson's | 1 | 80 | 25 | II |
| | | Disease | | | | |
| Comparator | SRR1568717 | Parkinson's | 2 | 77 | 21 | II |
| | | Disease | | | | |
| Comparator | SRR1568746 | Parkinson's | 1 | 73 | 17 | II |
| | | Disease | | | | |
| Comparator | SRR1568367 | Parkinson's | 1 | 70 | 12 | III |
| | | Disease | | | | |
| Comparator | SRR1568379 | Parkinson's | 1 | 80 | 10 | III |
| | | Disease | | | | |
| Comparator | SRR1568396 | Parkinson's | 1 | 86 | 7 | III |
| | | Disease | | | | |
| Comparator | SRR1568399 | Parkinson's | 1 | 71 | 12 | III |
| | | Disease | | | | |
| Comparator | SRR1568451 | Parkinson's | 1 | 89 | NA | III |
| | | Disease | | | | |
| Comparator | SRR1568532 | Parkinson's | 2 | 81 | 6 | III |
| | | Disease | | | | |
| Comparator | SRR1568555 | Parkinson's | 1 | 86 | 4 | III |
| | | Disease | | | | |
| Comparator | SRR1568692 | Parkinson's | 1 | 88 | 1 | III |
| | | Disease | | | | |
| Comparator | SRR1568703 | Parkinson's | 1 | 77 | 4 | III |
| | | Disease | | | | |
| Comparator | SRR1568725 | Parkinson's | 2 | 83 | 21 | III |
| | | Disease | | | | |
| Comparator | SRR1568739 | Parkinson's | 2 | 78 | 23 | III |
| | | Disease | | | | |
| Comparator | SRR1568363 | Parkinson's | 2 | 82 | 10 | IV |
| | | Disease | | | | |
| Comparator | SRR1568390 | Parkinson's | 2 | 79 | 6 | IV |
| | | Disease | | | | |
| Comparator | SRR1568425 | Parkinson's | 2 | 86 | 11 | IV |
| | | Disease | | | | |
| Comparator | SRR1568439 | Parkinson's | 2 | 85 | 18 | IV |
| | | Disease | | | | |
| Comparator | SRR1568458 | Parkinson's | 2 | 79 | 20 | IV |
| | | Disease | | | | |
| Comparator | SRR1568472 | Parkinson's | 2 | 81 | 4 | IV |
| | | Disease | | | | |
| Comparator | SRR1568504 | Parkinson's | 2 | 77 | 23 | IV |
| | | Disease | | | | |
| Comparator | SRR1568536 | Parkinson's | 1 | 76 | 9 | IV |
| | | Disease | | | | |
| Comparator | SRR1568588 | Parkinson's | 1 | 84 | 17 | IV |
| | | Disease | | | | |
| Comparator | SRR1568596 | Parkinson's | 1 | 80 | 9 | IV |
| | | Disease | | | | |
| Comparator | SRR1568619 | Parkinson's | 1 | 73 | 11 | IV |
| | | Disease | | | | |
| Comparator | SRR1568715 | Parkinson's | 2 | 83 | 1 | IV |
| | | Disease | | | | |
| Comparator | SRR1568737 | Parkinson's | 1 | 76 | 2 | IV |
| | | Disease | | | | |
| Comparator | SRR1568517 | Parkinson's | 1 | 83 | 15 | 0 |
| | | Disease with | | | | |
| | | Dementia | | | | |
| Comparator | SRR1568684 | Parkinson's | 1 | 72 | 27 | I |
| | | Disease with | | | | |
| | | Dementia | | | | |
| Comparator | SRR1568431 | Parkinson's | 1 | 79 | 23 | II |
| | | Disease with | | | | |
| | | Dementia | | | | |
| Comparator | SRR1568444 | Parkinson's | 1 | 70 | 30 | II |
| | | Disease with | | | | |
| | | Dementia | | | | |
| Comparator | SRR1568479 | Parkinson's | 2 | 84 | 23 | II |
| | | Disease with | | | | |
| | | Dementia | | | | |
| Comparator | SRR1568658 | Parkinson's | 2 | 87 | 0 | II |
| | | Disease with | | | | |
| | | Dementia | | | | |
| Comparator | SRR1568730 | Parkinson's | 2 | 79 | 1 | II |
| | | Disease with | | | | |
| | | Dementia | | | | |
| Comparator | SRR1568365 | Parkinson's | 2 | 73 | 29 | III |
| | | Disease with | | | | |
| | | Dementia | | | | |
| Comparator | SRR1568401 | Parkinson's | 2 | 78 | 16 | III |
| | | Disease with | | | | |
| | | Dementia | | | | |
| Comparator | SRR1568403 | Parkinson's | 2 | 82 | 22 | III |
| | | Disease with | | | | |
| | | Dementia | | | | |
| Comparator | SRR1568427 | Parkinson's | 1 | 78 | 19 | III |
| | | Disease with | | | | |
| | | Dementia | | | | |
| Comparator | SRR1568453 | Parkinson's | 1 | 83 | 7 | III |
| | | Disease with | | | | |
| | | Dementia | | | | |
| Comparator | SRR1568519 | Parkinson's | 2 | 82 | 18 | III |
| | | Disease with | | | | |
| | | Dementia | | | | |
| Comparator | SRR1568549 | Parkinson's | 1 | 75 | 21 | III |
| | | Disease with | | | | |
| | | Dementia | | | | |
| Comparator | SRR1568572 | Parkinson's | 1 | 74 | 17 | III |
| | | Disease with | | | | |
| | | Dementia | | | | |
| Comparator | SRR1568617 | Parkinson's | 2 | 85 | 16 | III |
| | | Disease with | | | | |
| | | Dementia | | | | |
| Comparator | SRR1568629 | Parkinson's | 1 | 83 | 4 | III |
| | | Disease with | | | | |
| | | Dementia | | | | |
| Comparator | SRR1568690 | Parkinson's | 1 | 76 | 2 | III |
| | | Disease with | | | | |
| | | Dementia | | | | |
| Comparator | SRR1568711 | Parkinson's | 1 | 83 | 9 | III |
| | | Disease with | | | | |
| | | Dementia | | | | |
| Comparator | SRR1568754 | Parkinson's | 1 | 85 | 0 | III |
| | | Disease with | | | | |
| | | Dementia | | | | |
| Comparator | SRR1568373 | Parkinson's | 2 | 87 | 18 | IV |
| | | Disease with | | | | |
| | | Dementia | | | | |
| Comparator | SRR1568625 | Parkinson's | 2 | 84 | NA | IV |
| | | Disease with | | | | |
| | | Dementia | | | | |
| AVERAGE | NA | NA | 1.4 ± 0.5 | 80.86 ± 8.2 | 11.98 ± 8.1 | NA |

TABLE 7A
Disease Specific Biomarkers for Alzheimer's Disease Identified in Serum

| Seq. | | Total | | | |
|---|---|---|---|---|---|
| ID | Sequence | Reads | Specificity | Sensitivity | p-value |
| 255 | CGTGTTCGGACTGGGGTC | 25 | 100% | 19.61% | 1.58E−06 |
| 256 | TGTGATTAGAGGGCTGGAACTTTCACCCCCACCC | 13 | 100% | 17.65% | 6.48E−06 |
| 257 | TCTGTTACGGAACTGTACTCTCTGAGGGCCTCCCACCTGATTC | 21 | 100% | 15.69% | 2.61E−05 |
| 258 | CACCTGTGCGTGTGGGTGCTGCTGCGGGCTGTCAGATGCTGACC | 19 | 100% | 15.69% | 2.61E−05 |
| 259 | CTCAGATCAGACGTGGCG | 17 | 100% | 15.69% | 2.61E−05 |
| 260 | TTTGAGAGGATGATCAGCCACACTGGGACTG | 27 | 100% | 13.73% | 1.03E−04 |
| 261 | CTGTTTCAACCAACGCTTGACTGAGAACTCTTTC | 23 | 100% | 13.73% | 1.03E−04 |
| 262 | TCAGGGTCAGTCTAAGTGAAGACAAAGAGAGGC | 21 | 100% | 13.73% | 1.03E−04 |
| 263 | AGTGCGAGTTTGAGGGCTGTGACCGGCGCT | 19 | 100% | 13.73% | 1.03E−04 |
| 264 | CATGTTGCTTTATTTATCA | 16 | 100% | 13.73% | 1.03E−04 |
| 265 | TGTGGGAGAGTAGGACGCCGCCGGACA | 15 | 100% | 13.73% | 1.03E−04 |
| 266 | TCTGTTACGGAACTGTACTCTCTGAGGGCCTCCCACCTGACTC | 12 | 100% | 13.73% | 1.03E−04 |
| 267 | AGGACTGGTGGAGCGCTTAGAAG | 75 | 100% | 11.76% | 4.01E−04 |
| 268 | GCCCCAGTGGCCTAATGGATAAGGCATTGGCTTAGGGAC | 23 | 100% | 11.76% | 4.01E−04 |
| 269 | CAGGGCACGGTATTTCTTGTTACTTCCCTGCACACGGACTGTG | 23 | 100% | 11.76% | 4.01E−04 |
| 270 | TACAAGGAAGGTCACTACCGTTCTTTCAC | 19 | 100% | 11.76% | 4.01E−04 |
| 271 | CTGCTTTCTTCTTTGGATCGTCGTTCAACT | 19 | 100% | 11.76% | 4.01E−04 |
| 272 | TTAGCAACAACAGGAAGCCCCTTTTATCCT | 19 | 100% | 11.76% | 4.01E−04 |
| 273 | TCTGAATCAACCCTTATTACTCT | 17 | 100% | 11.76% | 4.01E−04 |
| 274 | TCTCATTTGGGCAGAATATGTCAGAGGGAAGATC | 17 | 100% | 11.76% | 4.01E−04 |
| 275 | CCTCCTAAGTATTACACC | 16 | 100% | 11.76% | 4.01E−04 |
| 276 | CCCATCTTGCTGAGATGAGGCC | 16 | 100% | 11.76% | 4.01E−04 |
| 277 | CCTTGTAATAACCTCTAGTCCTTTCC | 15 | 100% | 11.76% | 4.01E−04 |
| 278 | ATTCATGGTGCTTTCAAGTCAGGTTTTCT | 15 | 100% | 11.76% | 4.01E−04 |
| 279 | CATCAGAGACAGTGGCA | 14 | 100% | 11.76% | 4.01E−04 |
| 280 | CCCTGAAGATGTAACTGTCA | 14 | 100% | 11.76% | 4.01E−04 |
| 281 | CCCTGAAGCATACCAAAATGTGTC | 14 | 100% | 11.76% | 4.01E−04 |
| 282 | TGAAAAGGACTTTGAAAAGAGAGTC | 14 | 100% | 11.76% | 4.01E−04 |
| 283 | CTGTCGGGACCCGAAAGATG | 13 | 100% | 11.76% | 4.01E−04 |
| 284 | TCATCTCATCCTGGGGC | 12 | 100% | 11.76% | 4.01E−04 |
| 285 | CTACTCTGAACGATTGAGACC | 12 | 100% | 11.76% | 4.01E−04 |
| 286 | CGGCGGGCTGTCAGATTCTCACC | 12 | 100% | 11.76% | 4.01E−04 |
| 287 | GGGTGATTAGCTCAGCTGGGAGAGCGTCTGCC | 12 | 100% | 11.76% | 4.01E−04 |
| 288 | CCCTAGTCTTCATTTGTTGTTATGTCATTGCCTGCCTT | 12 | 100% | 11.76% | 4.01E−04 |
| 289 | CCCAGGTTCAAGTGATTCTCCTGCCTCAGCCTCCAGAGTACC | 12 | 100% | 11.76% | 4.01E−04 |
| 290 | CTTCACCTGAGAGTGTC | 11 | 100% | 11.76% | 4.01E−04 |
| 291 | CCCCAGAAGCAGGTGTCAAT | 11 | 100% | 11.76% | 4.01E−04 |
| 292 | CCCATATTTCATAATTTCACGCTTCTGTCTTGCATGCTTC | 11 | 100% | 11.76% | 4.01E−04 |
| 293 | CACTTGTGCTTGTGGGTGCTACTGCGGGCGGTCAGATGCTCACC | 11 | 100% | 11.76% | 4.01E−04 |
| 294 | TGGGCAGTGGCTTATGGGAAGATGACCTCTGATTAAATAATTCC | 11 | 100% | 11.76% | 4.01E−04 |
| 295 | CGCGACCTCAGATCCGACGTGGCGACCCGCTGAATTTAAGCC | 39 | 100% | 9.80% | 1.53E−03 |
| 296 | CGTGAGAGAACTCGGGTGAAGGA | 33 | 100% | 9.80% | 1.53E−03 |
| 297 | AAGCACTGAACCGGGCGACTAGTACTAGAGT | 25 | 100% | 9.80% | 1.53E−03 |
| 298 | CTGTCTGGACTACTTCTTTCTCTGATTAATGCCTTGCT | 24 | 100% | 9.80% | 1.53E−03 |
| 299 | CATTTCCTCCATTGTGTCC | 23 | 100% | 9.80% | 1.53E−03 |
| 300 | TATTTGCGTAGAGGTGTTTGTAGTATTCTCTGATGGTAGTA | 23 | 100% | 9.80% | 1.53E−03 |
| 301 | CCTCCTGGAGAGATCTCTTGAGTTCCTGCCTC | 22 | 100% | 9.80% | 1.53E−03 |
| 302 | CGGGAGAGTAGGTCGCGCCAGGTCC | 21 | 100% | 9.80% | 1.53E−03 |
| 303 | CTGTAAGTGTTTGGAGTTGGAATTTAC | 20 | 100% | 9.80% | 1.53E−03 |
| 304 | CCATGCCTGTGGCACACTTCTGTCCTTCACGCTGTCTTCTC | 20 | 100% | 9.80% | 1.53E−03 |
| 305 | CCCTCTCTCAGCATTTTTGCTGTTCGTGAAATGAGGACATAG | 20 | 100% | 9.80% | 1.53E−03 |
| 306 | CCGAGATGGATCTGGCTGGGACCC | 19 | 100% | 9.80% | 1.53E−03 |
| 307 | TCTGTTACGGAAGTGTACTCTCTGAGGGCCTCCCACCTGAGTC | 19 | 100% | 9.80% | 1.53E−03 |
| 308 | AGAAGAAGAAGAGGAAG | 18 | 100% | 9.80% | 1.53E−03 |
| 309 | CCCAGAGTCCATATCAATGG | 18 | 100% | 9.80% | 1.53E−03 |
| 310 | GAGAGGACCGGGTTGGACGA | 18 | 100% | 9.80% | 1.53E−03 |
| 311 | AAAGGGAAGGCTGAACTGCTG | 18 | 100% | 9.80% | 1.53E−03 |
| 312 | ATGGGGTGCAAGCTCTTGATCGAAGCC | 18 | 100% | 9.80% | 1.53E−03 |
| 313 | ACTGTAGTAACTCCTAC | 17 | 100% | 9.80% | 1.53E−03 |
| 314 | TCTTTAGGATCAATTTCCATTC | 17 | 100% | 9.80% | 1.53E−03 |
| 315 | AAGCGAGTCTGAACAGGGCGACTGAGTTTGA | 17 | 100% | 9.80% | 1.53E−03 |
| 316 | CCTTCCTAATTCTTCTTTCAATAGCTATTTA | 17 | 100% | 9.80% | 1.53E−03 |
| 317 | GGCTGGTCCGATGGGAGTGGGTGATCCGAACT | 17 | 100% | 9.80% | 1.53E−03 |
| 318 | GGCTGGTCCGATGGTAGTGGGTTATAGGGATT | 17 | 100% | 9.80% | 1.53E−03 |
| 319 | GAAAAGACATGGAGGGTGTAGAATAAGTGGGAGCTT | 17 | 100% | 9.80% | 1.53E−03 |
| 320 | CCTGCATCAGAGGACAAACCCGCTAATAACTTGATCC | 17 | 100% | 9.80% | 1.53E−03 |
| 321 | CAGGGAGCTGGAGAGGGTTC | 16 | 100% | 9.80% | 1.53E−03 |
| 322 | TGCGAGTGTAGAGGTGAAATTCG | 16 | 100% | 9.80% | 1.53E−03 |
| 323 | CTGTGTCCCCACCCAAATCTCATC | 16 | 100% | 9.80% | 1.53E−03 |
| 324 | GTGTCCATGTTGAAAACTCGCCTG | 16 | 100% | 9.80% | 1.53E−03 |
| 325 | CCCTTCCCATTTTTAATAGTTGTAGC | 16 | 100% | 9.80% | 1.53E−03 |
| 326 | TGCTGCGGGCTGTCAGGATGCTCACC | 16 | 100% | 9.80% | 1.53E−03 |
| 327 | GTTATTTGGATTCTGGGTATGCTCTGG | 16 | 100% | 9.80% | 1.53E−03 |
| 328 | CAGCCCGGGTTCCCTCTTTCTGCCATCTC | 16 | 100% | 9.80% | 1.53E−03 |
| 329 | TAGGTGGATGGTGGATGGGTGGATGATGGA | 16 | 100% | 9.80% | 1.53E−03 |
| 330 | CACCTGTGCGTGTGGGTGATGCTGCGGGCTGTCAGATGCTGACC | 16 | 100% | 9.80% | 1.53E−03 |
| 331 | CCTATCTCAGAATGCCTGAACCAC | 15 | 100% | 9.80% | 1.53E−03 |
| 332 | TTCTGGTAGAATTCAGCTGTGAATCCGTCTTGTCC | 15 | 100% | 9.80% | 1.53E−03 |
| 333 | CCCATTCATTCATTTCAATATCCTTCAAACATTTCTTTTC | 15 | 100% | 9.80% | 1.53E−03 |
| 334 | AGGACTGTCCTCGGGAA | 14 | 100% | 9.80% | 1.53E−03 |
| 335 | ATTTGAGAGGGGCTGACCTT | 14 | 100% | 9.80% | 1.53E−03 |
| 336 | CCCCAGAATGATCTTGCCTTC | 14 | 100% | 9.80% | 1.53E−03 |
| 337 | ATACATGAGTTGGGCTTACTGAGTG | 14 | 100% | 9.80% | 1.53E−03 |
| 338 | TAAATGGGTAAGAAGCCCGGCTCGCT | 14 | 100% | 9.80% | 1.53E−03 |
| 339 | CAGAACTGGAACTTGAACCCACATTTC | 14 | 100% | 9.80% | 1.53E−03 |
| 340 | GCATTGGTGGTTCAGTGGTAGAATTCTCGCCTGGTGGA | 14 | 100% | 9.80% | 1.53E−03 |
| 341 | CAAAGGTCAAACAACACAAGTGAGTCTCAAACTCTCAAC | 14 | 100% | 9.80% | 1.53E−03 |
| 342 | CCTCGCGTCGCTTCCTCTTCTCCTTCAGGAGCGTTTTATCCC | 14 | 100% | 9.80% | 1.53E−03 |
| 343 | CAAGTGCAAAGGGAATTCATTTTGAAGAGTTTTATGCAACTGTG | 14 | 100% | 9.80% | 1.53E−03 |
| 344 | AGTTCTACAGTCGGCCGATC | 13 | 100% | 9.80% | 1.53E−03 |
| 345 | AATGGAGGAGTGGTCGGAGGA | 13 | 100% | 9.80% | 1.53E−03 |
| 346 | CAAATGACTATCTCACTGCTC | 13 | 100% | 9.80% | 1.53E−03 |
| 347 | CATATTGTTCTGTGATCTTAACTG | 13 | 100% | 9.80% | 1.53E−03 |
| 348 | GGGACGTTAGCTCAGTTGGTAGAGC | 13 | 100% | 9.80% | 1.53E−03 |
| 349 | TTGATCTCTGGACTGAGGCTTTGTGTGTGCC | 13 | 100% | 9.80% | 1.53E−03 |
| 350 | ACACGATCTCGGCTCACTGCAACCTCTGCCTCC | 13 | 100% | 9.80% | 1.53E−03 |
| 351 | CCCTGGCTCCCTGCTGGGCTTGGGGAGCCTCTTC | 13 | 100% | 9.80% | 1.53E−03 |
| 352 | TGCGAGCGGTCCCGGGTTCACATCCCGGACGAGCCC | 13 | 100% | 9.80% | 1.53E−03 |
| 353 | CCCTCAATCCCTGGTCGAGGGAGAGGGACTTCCTGTC | 13 | 100% | 9.80% | 1.53E−03 |
| 354 | GATTAGGATACAAGGTCTTGCTAGAACTCCCTATCTCCC | 13 | 100% | 9.80% | 1.53E−03 |
| 355 | CTGTGGAACGGGGTGAGATGGGATGGGATGGGACAGGATAGGA | 13 | 100% | 9.80% | 1.53E−03 |
| 356 | CTGGAAGGTTTGACTGT | 12 | 100% | 9.80% | 1.53E−03 |
| 357 | TGCCCTTTGTCATCCCTATGCCT | 12 | 100% | 9.80% | 1.53E−03 |
| 358 | CCCCATGACCCTATTCAAGACTTC | 12 | 100% | 9.80% | 1.53E−03 |
| 359 | CGGTAGCTCGTCAGGCTCATAACC | 12 | 100% | 9.80% | 1.53E−03 |
| 360 | TTCCCTTTGTCATCCTTATGCCTG | 12 | 100% | 9.80% | 1.53E−03 |
| 361 | CTTCAACATCACCTGTAGCCATCAC | 12 | 100% | 9.80% | 1.53E−03 |
| 362 | CCTTCCACCTTGGCCTCCCAAAGTGC | 12 | 100% | 9.80% | 1.53E−03 |
| 363 | AGGGGAATGGAATGGAATGGAATGCAA | 12 | 100% | 9.80% | 1.53E−03 |
| 364 | CGCGGGTGAGTAGGTCGCTGCCAGGTCT | 12 | 100% | 9.80% | 1.53E−03 |
| 365 | AGGGACCCTCTGTGGCGGGTAGTTTGACT | 12 | 100% | 9.80% | 1.53E−03 |
| 366 | TATATGGAAGACATAAAAAGAGAAGCTCC | 12 | 100% | 9.80% | 1.53E−03 |
| 367 | AGGAATTTCGGTCCAGATTGTTTCTTGAGTCACT | 12 | 100% | 9.80% | 1.53E−03 |
| 368 | AAAAAGTCTTTAACTCCACCATTAGCACCCAAAGC | 12 | 100% | 9.80% | 1.53E−03 |
| 369 | CTAAGGGGTCGGGAGTTCGAATCTCTCTGAGCGCAC | 12 | 100% | 9.80% | 1.53E−03 |
| 370 | CGTAGTGTCGGTGGTTCGATTCCGCCCCTGGGCACCA | 12 | 100% | 9.80% | 1.53E−03 |
| 371 | GAGCTGATTGGTACTAATCGGTCGTGAGGCTTGACCT | 12 | 100% | 9.80% | 1.53E−03 |
| 372 | GCTCTAAGTTCGAGTCTCTCTTTCACTTCTTCTCTTGG | 12 | 100% | 9.80% | 1.53E−03 |
| 373 | CCCAGGTTGAGTTTATGGGGGTAGTGCTGTAAGGTCATT | 12 | 100% | 9.80% | 1.53E−03 |
| 374 | AATCGGACTGTTCAACTCACCTGGCAACCACTCCCAGAGCCCC | 12 | 100% | 9.80% | 1.53E−03 |
| 375 | TTTCAAGGACTGTGTTTAATTTCCTTTTGGATTTGTTTATTTTG | 12 | 100% | 9.80% | 1.53E−03 |
| 376 | CGAATAAGCTTTGATCCA | 11 | 100% | 9.80% | 1.53E−03 |
| 377 | CACTGGAATTCTGAGCCCCT | 11 | 100% | 9.80% | 1.53E−03 |
| 378 | CAGGAGTCGGGGGTGGGACG | 11 | 100% | 9.80% | 1.53E−03 |
| 379 | AAAAGAGGACCACCACCAAGA | 11 | 100% | 9.80% | 1.53E−03 |
| 380 | GGTGGTGGCGGCGGTGGTGGC | 11 | 100% | 9.80% | 1.53E−03 |
| 381 | GTCTTACTCTGTTGCTCAGGC | 11 | 100% | 9.80% | 1.53E−03 |
| 382 | CCTCCTCTGGATCACATGGGCTC | 11 | 100% | 9.80% | 1.53E−03 |
| 383 | CCTTCGGGCCTGTCCAGAACCTC | 11 | 100% | 9.80% | 1.53E−03 |
| 384 | TTCGAATCTCACCGCTTCCGCCA | 11 | 100% | 9.80% | 1.53E−03 |
| 385 | CCATCACATAGGGGATTAGATTTCAATGC | 11 | 100% | 9.80% | 1.53E−03 |
| 386 | TGTAAGGGCTGGGTCGGTCGGGCTGGGGC | 11 | 100% | 9.80% | 1.53E−03 |
| 387 | CAGCGCCTTTGCACACGCTATTCTCTCTGCC | 11 | 100% | 9.80% | 1.53E−03 |
| 388 | CGCGGAGCCCAGGGTTCGATTCCCTGTACCG | 11 | 100% | 9.80% | 1.53E−03 |
| 389 | CTGATGGGCTGGGCAGGGCTCCCTGGATGGG | 11 | 100% | 9.80% | 1.53E−03 |
| 390 | CCCCACTTCCGTACTGAGTTTCTCACCTGTTTG | 11 | 100% | 9.80% | 1.53E−03 |
| 391 | AGTACTGTTATTTAGCGTGCTAAATATATTGTCC | 11 | 100% | 9.80% | 1.53E−03 |
| 392 | AGTGCATCGCGCGAAAGTAGGTCGTCGCCGGCTT | 11 | 100% | 9.80% | 1.53E−03 |
| 393 | CCTGATTTTTTTTGCAATTTCTTTGTATTGTTTTTA | 11 | 100% | 9.80% | 1.53E−03 |
| 394 | TGATGGAGTGGCCTGGACTCACATTAAAATAAGTACT | 11 | 100% | 9.80% | 1.53E−03 |
| 395 | CCCCTTACCCATCAAATTTTCCTTAAAAACTCCAATCC | 11 | 100% | 9.80% | 1.53E−03 |
| 396 | CTCTTTGGGGGGGGGTGGGGGAGGGGGAGCCTCGCGTCC | 11 | 100% | 9.80% | 1.53E−03 |
| 397 | CCTGAGCTCTTGTTCGATGTCCAAGGATAATGAGGTGGCA | 11 | 100% | 9.80% | 1.53E−03 |
| 398 | TAAGGAGGAGGAACATTGTGAGCAGGAGAAGGATCTGGGG | 11 | 100% | 9.80% | 1.53E−03 |
| 399 | TCCTGTCCGGTTGAGGCCTTTCTCTTGGGGTCTTGCTGTC | 11 | 100% | 9.80% | 1.53E−03 |
| 400 | CCTTTCATATCTTCTCAAATACTGATTTAATTTTATACTGG | 11 | 100% | 9.80% | 1.53E−03 |
| 401 | CCTAGGTTCAAGTGATCCTCCTGCTTCAGCTTCCTGAGTAGC | 11 | 100% | 9.80% | 1.53E−03 |
| 402 | CCTGGCCTCAAGCAATCCTCCCACCTTGGCCTCCACAAGTAC | 11 | 100% | 9.80% | 1.53E−03 |
| 403 | CATCTCAGCTCCAAACCCACAGGTTGGGTTCAGTTCTTGCATCC | 11 | 100% | 9.80% | 1.53E−03 |

TABLE 7B
Disease Specific Biomarkers for Alzheimer's Disease Identified in Serum

| Stage | Braak II | Braak II | Braak II | Braak III | Braak III | Braak IV | Braak IV | Braak IV |
|---|---|---|---|---|---|---|---|---|
| Seq. ID | SRR1568547 | SRR1568553 | SRR1568580 | SRR1568557 | SRR1568686 | SRR1568421 | SRR1568437 | SRR1568534 |
| 255 | | | | | 0.197 | | | |
| 256 | | | | | | 0.92 | | |
| 257 | | | | | | | | |
| 258 | | | | | | | | |
| 259 | | 0.076 | 0.125 | | | | | |
| 260 | | | | | 1.181 | | | |
| 261 | | | | | | 3.678 | | |
| 262 | | | | | | | | |
| 263 | | 0.076 | | | | | | |
| 264 | | | | | | | | |
| 265 | | | 0.125 | | | | 0.301 | |
| 266 | | | | | 0.197 | | | |
| 267 | | | | 0.6 | 11.611 | | | |
| 268 | | | | | 0.787 | | | |
| 269 | | | | | | | | |
| 270 | | | | | | | | |
| 271 | | | | | | 0.92 | | |
| 272 | | | | | | 2.759 | | |
| 273 | | | | | | | | |
| 274 | | | | | 1.574 | | | |
| 275 | | | | | 0.787 | 2.759 | | |
| 276 | | | | | | | | |
| 277 | | | | | | | | |
| 278 | | | | | | | | |
| 279 | | | | | | 3.678 | | |
| 280 | | | | | | | | |
| 281 | | | | | | | | |
| 282 | | 0.229 | | | | | 0.602 | |
| 283 | | 0.305 | | | | 2.759 | 0.602 | |
| 284 | | | | | | | | |
| 285 | | 0.076 | | | | | | |
| 286 | | | | | | | | |
| 287 | 3.825 | 0.153 | | 0.3 | | | 0.301 | |
| 288 | | | | | | 1.839 | | |
| 289 | | | | | | | | |
| 290 | | | | | | | | |
| 291 | | | | | | 0.92 | | |
| 292 | | | | | | | | |
| 293 | | | | | | | | |
| 294 | | | | | | | | |
| 295 | | | | 0.3 | 1.574 | | | |
| 296 | | | | | | | | |
| 297 | | | | | | | | |
| 298 | | | | | | 1.839 | | |
| 299 | | | | | | | | |
| 300 | | | | | | | | |
| 301 | | | | | | | | |
| 302 | | | | | 1.771 | | 2.106 | |
| 303 | | | | | | | | |
| 304 | | | | | | | | |
| 305 | | | | | | | | |
| 306 | | | | | | | 0.301 | |
| 307 | | | | | | | | |
| 308 | | | | | | | | |
| 309 | | | | | | | | |
| 310 | | | | | | | | |
| 311 | | | | | | | | |
| 312 | | | | | 2.362 | | | |
| 313 | | | | | | | | |
| 314 | | | | | | | | |
| 315 | | | | | 1.574 | | | |
| 316 | | | | | | | | |
| 317 | | | | | | | | |
| 318 | | | | | | | | |
| 319 | | | | | | | 0.301 | |
| 320 | | | | | | | | |
| 321 | | | | | | | | |
| 322 | 2.55 | | | 0.9 | 1.771 | | | |
| 323 | | | | | | 5.518 | | |
| 324 | | | | | | 1.839 | | |
| 325 | | | | | | 1.839 | | |
| 326 | | | | | | | | |
| 327 | | | | | | 4.598 | | |
| 328 | | | | | | | | |
| 329 | | | | | | | | |
| 330 | | | | | | | | |
| 331 | | | | | | 0.92 | | |
| 332 | | | | | | 2.759 | | |
| 333 | | | | | | | | |
| 334 | | | 0.25 | | | | | |
| 335 | | | | | | | | |
| 336 | | | | | | | | |
| 337 | | | | | | 0.92 | | |
| 338 | | | | | | | | |
| 339 | | | | | | 1.839 | | |
| 340 | | | 0.25 | | | | | |
| 341 | | | | | | 4.598 | | |
| 342 | | | | | | | | |
| 343 | | | | | | 0.92 | | |
| 344 | | | | | 0.984 | | | |
| 345 | | | | | | | | |
| 346 | | 0.076 | | | | | | |
| 347 | | | | | | | | |
| 348 | | | | | 0.59 | | | |
| 349 | | | | | | | | |
| 350 | | | | | | | | |
| 351 | | | | | | 1.839 | | |
| 352 | | | | | 0.394 | | | |
| 353 | | | | | 0.984 | | | |
| 354 | | | | | | | | |
| 355 | | | | | 0.984 | | | |
| 356 | | | | | | | | |
| 357 | | | | | 0.59 | | | |
| 358 | | | | | | | | |
| 359 | | | 0.125 | | 0.787 | | 0.301 | 0.247 |
| 360 | | | | | 1.378 | | | |
| 361 | | | | | | | | |
| 362 | | | | | | | | |
| 363 | | | | | | 2.759 | | 0.247 |
| 364 | | | | | 1.181 | | 0.602 | 0.247 |
| 365 | | | | | | | | |
| 366 | | | | | | | | |
| 367 | | | | | | 3.678 | | |
| 368 | | | | | | | | 0.494 |
| 369 | | | | | | | | |
| 370 | | | | | 1.378 | | 0.301 | |
| 371 | | | | | | | 0.903 | |
| 372 | | | | | | | | |
| 373 | | | | | | | | |
| 374 | | | | | | 5.518 | | |
| 375 | | | | | | 3.678 | | |
| 376 | | | | | 1.181 | | | |
| 377 | | | | | | | | |
| 378 | | | 0.25 | 0.3 | | | | |
| 379 | | | | | | | | |
| 380 | | 0.076 | | | | | 0.301 | |
| 381 | | 0.076 | | | | | | |
| 382 | | | | | | 0.92 | | |
| 383 | | | | | | | | |
| 384 | | | | | 0.984 | | 0.301 | |
| 385 | | | | | | 1.839 | | |
| 386 | | | | | | | | |
| 387 | | | | | | | | |
| 388 | | 0.076 | 0.125 | | | | | |
| 389 | | | | | 0.59 | | | |
| 390 | | | | | | | | |
| 391 | | | | | | | | |
| 392 | | | | | 0.787 | | | 0.247 |
| 393 | | | | | | 2.759 | | |
| 394 | | | | | | | | |
| 395 | | | | | | | | |
| 396 | | | | | | 2.759 | | |
| 397 | | | | | | | | |
| 398 | | | | | | | | |
| 399 | | | | | | | | |
| 400 | | | | | | | | |
| 401 | | | | | | | | |
| 402 | | | | | | | | |
| 403 | | | | | | | | |
| # Biomarkers | 2 | 10 | 7 | 5 | 26 | 29 | 13 | 5 |
| Per Sample | | | | | | | | |
| % | 1% | 7% | 5% | 3% | 17% | 19% | 9% | 3% |
| Coverage | | | | | | | | |
| Stage | Braak IV | Braak IV | Braak IV | Braak IV | Braak IV | Braak IV | Braak V | Braak V |
| Seq. ID | SRR1568541 | SRR1568586 | SRR1568645 | SRR1568652 | SRR1568734 | SRR1568744 | SRR1568369 | SRR1568371 |
| 255 | | | 9.27 | | | | | |
| 256 | | | | | | | | |
| 257 | | | 7.416 | | | | | 0.067 |
| 258 | | | 1.854 | | | | | |
| 259 | | | | | | | | |
| 260 | | | | | | | | 0.033 |
| 261 | | | | | | | | 0.033 |
| 262 | | | | | | | 0.307 | |
| 263 | | | | | | | | |
| 264 | | | | | | | | 0.033 |
| 265 | | | | | | | | 0.1 |
| 266 | | | 3.708 | | | | | |
| 267 | | | | | | | | |
| 268 | | | | | | | | |
| 269 | | | | | | | | 0.033 |
| 270 | | | | 5.944 | | | | |
| 271 | | | | | | | 0.614 | |
| 272 | | | | | | | | |
| 273 | | | | 1.981 | | | | 0.033 |
| 274 | | | | | | | | |
| 275 | | | | | | | | 0.1 |
| 276 | | | | | | | | |
| 277 | | | | | | | | |
| 278 | | | | | | | 0.307 | |
| 279 | | | | | | | | 0.033 |
| 280 | | | | | | | | 0.033 |
| 281 | | | | | | | | |
| 282 | | | | | | | 0.307 | |
| 283 | | | | | | | | |
| 284 | | | | | | | | |
| 285 | | | | | | | | 0.067 |
| 286 | | | | 0.991 | | | | |
| 287 | | | | | | | | |
| 288 | | | | | | | | |
| 289 | | | | | | | | |
| 290 | | | | | | | | |
| 291 | | | | | | | | |
| 292 | | | | | | | 0.307 | |
| 293 | | | 1.854 | | | | | |
| 294 | | | | | | | | 0.033 |
| 295 | | | | | | | | |
| 296 | | | | 28.73 | | | | 0.033 |
| 297 | | | | | 0.741 | | | 0.134 |
| 298 | | | | | | | | |
| 299 | | | | | | | | |
| 300 | | | 11.124 | | | | | |
| 301 | | | | | | | | |
| 302 | | | | | | | | 0.067 |
| 303 | | | | | | | 0.307 | |
| 304 | | | | | | | | |
| 305 | | | 3.708 | | | | | |
| 306 | | | | | | | | |
| 307 | | | 1.854 | | | | | |
| 308 | | | | | | | | |
| 309 | | | | | | | | |
| 310 | | | | 10.898 | | | | |
| 311 | 0.585 | | | | | | | |
| 312 | | | | | | | 0.307 | |
| 313 | | | | | | | | |
| 314 | | | | 5.944 | | | | 0.067 |
| 315 | | | | | | | | |
| 316 | | | | | | | 0.307 | |
| 317 | | | | | | | | 0.435 |
| 318 | | | | | | | | 0.435 |
| 319 | | | | 7.926 | | | | |
| 320 | | | | 1.981 | | | | |
| 321 | | | | | | | | 0.1 |
| 322 | | | | | | | | 0.033 |
| 323 | | | | | | | | 0.1 |
| 324 | | | | | | | | |
| 325 | | | | | | | | |
| 326 | | | | | | | | |
| 327 | | | 1.854 | | | | | |
| 328 | | | | | | | | |
| 329 | | | | | | | | |
| 330 | | | 1.854 | | | | | |
| 331 | | | | 2.972 | | | | 0.033 |
| 332 | | | | | | | | |
| 333 | | | | | | | | 0.067 |
| 334 | | | | | | | | |
| 335 | | | | 6.935 | | | | |
| 336 | | | | 4.954 | | | | 0.067 |
| 337 | | | | | | | | |
| 338 | | | | | | | | |
| 339 | | | 11.124 | | | | | |
| 340 | | | | | | | | 0.033 |
| 341 | | | | | | | | 0.067 |
| 342 | | | | | | | | 0.1 |
| 343 | | | | | | | | 0.067 |
| 344 | | | 1.854 | | | 1.55 | | |
| 345 | | | | | | | | |
| 346 | | | 1.854 | | | | | |
| 347 | | | | | | | 0.307 | |
| 348 | | | | | | | | 0.167 |
| 349 | | | | | | | | |
| 350 | | | | | | | | 0.134 |
| 351 | | | 7.416 | | | | | |
| 352 | | | | | | | | |
| 353 | | | | | | | | |
| 354 | | | | | | | | |
| 355 | | | | | | | | 0.033 |
| 356 | | | | | | | | |
| 357 | | | | | | | | |
| 358 | | | | 4.954 | | | | 0.1 |
| 359 | | | | | | | | |
| 360 | | | | | | | | |
| 361 | | | | | | | 0.307 | 0.033 |
| 362 | | | | | | | | |
| 363 | | | | | | | | |
| 364 | | | | | | | | |
| 365 | | | | | | | | |
| 366 | | | | | | | | |
| 367 | | | | | | | | |
| 368 | | | | | 2.224 | | | |
| 369 | | | | | | | | |
| 370 | | | | | | | | |
| 371 | | | | | | | 0.307 | 0.033 |
| 372 | | | | | | | | |
| 373 | | | | | | | | 0.201 |
| 374 | | | | | | | | 0.1 |
| 375 | | | | | | | | 0.033 |
| 376 | | | | | | | | |
| 377 | | | | 1.981 | | | | 0.067 |
| 378 | | | | | | | | |
| 379 | | | | | | | | |
| 380 | | | | | | 4.651 | | |
| 381 | | | | | | | | |
| 382 | | | | | | | | |
| 383 | | 2.14 | | | | | | |
| 384 | | | | | | | 0.307 | |
| 385 | | | | | | | | 0.067 |
| 386 | | | | | | | | 0.167 |
| 387 | | | | | | | | |
| 388 | | | | | | | | 0.134 |
| 389 | | | | | | | | 0.033 |
| 390 | | | | | | | | 0.067 |
| 391 | | | 1.854 | | | | | 0.067 |
| 392 | | | | | | | 0.614 | 0.1 |
| 393 | | | | | | | 0.921 | |
| 394 | | | | | | | | 0.033 |
| 395 | | | | 2.972 | | | | 0.1 |
| 396 | | | | | | | | |
| 397 | | | | | | | | |
| 398 | | | | | | | | |
| 399 | | | 5.562 | | | | | 0.067 |
| 400 | | | | | | | | 0.033 |
| 401 | | | | | | | | 0.067 |
| 402 | | | | | | | | |
| 403 | | | 5.562 | 1.981 | | | | 0.1 |
| # Biomarkers | 1 | 1 | 17 | 15 | 2 | 2 | 14 | 51 |
| Per Sample | | | | | | | | |
| % | 1% | 1% | 11% | 10% | 1% | 1% | 9% | 34% |
| Coverage | | | | | | | | |
| Stage | Braak V | Braak V | Braak V | Braak V | Braak V | Braak V | Braak V | Braak V |
| Seq. ID | SRR1568407 | SRR1568409 | SRR1568411 | SRR1568446 | SRR1568455 | SRR1568468 | SRR1568475 | SRR1568481 |
| 255 | | | | | | | | |
| 256 | | 0.243 | | | | | | |
| 257 | | | 1.988 | | | | | |
| 258 | | 0.243 | | | | | | |
| 259 | 0.589 | | | | | | | |
| 260 | | | | | | | | |
| 261 | | | | | | | | |
| 262 | | | 7.952 | | | | | 0.199 |
| 263 | 1.325 | | | | | | 1.032 | |
| 264 | | | | | | | | |
| 265 | | | | | | | | |
| 266 | | | | | | | | |
| 267 | 0.442 | | | | | | | |
| 268 | 0.147 | | | | | | | |
| 269 | | 0.487 | | | | | | |
| 270 | | | | 4.457 | | | | |
| 271 | | | | | | | | |
| 272 | | | | 0.743 | | | | |
| 273 | | | | | | | | |
| 274 | | | | 2.228 | | | | |
| 275 | | | | | | | | |
| 276 | | 0.974 | | | | | | |
| 277 | | 0.243 | | | | | | |
| 278 | | | | | | | 1.548 | |
| 279 | | 0.73 | | 2.228 | | | | 0.199 |
| 280 | | 0.73 | | | | | | |
| 281 | | 0.487 | | | | | | |
| 282 | | | | | | | | |
| 283 | | | | | | | | |
| 284 | 0.442 | | | | | | 1.548 | |
| 285 | | | | | | | | |
| 286 | | | 1.988 | | | | | |
| 287 | | | | | | | | |
| 288 | | 0.243 | | | | | | |
| 289 | | 0.243 | | 3.714 | | | | |
| 290 | | 0.487 | | | | | | |
| 291 | | | | | 0.349 | | | |
| 292 | | 0.243 | | | | | | |
| 293 | | 0.487 | 1.988 | | | | | |
| 294 | | 0.243 | 5.964 | | | | | |
| 295 | | | | | | | | |
| 296 | | | | | | | | |
| 297 | | | | | | | | 0.199 |
| 298 | | 0.487 | | | | | | |
| 299 | | | | 5.199 | 0.349 | 16.967 | | |
| 300 | | 0.487 | | | | | | |
| 301 | | | | 3.714 | | | | |
| 302 | | | | | | | | |
| 303 | | | | | | | | |
| 304 | | 0.243 | | | | | | |
| 305 | | | | | | | | |
| 306 | | | | | | | | 0.199 |
| 307 | | | | | | | | |
| 308 | | | | | | | 1.548 | |
| 309 | | 0.73 | | 3.714 | | | | |
| 310 | | | | | | | | |
| 311 | | | | | | | | |
| 312 | | 0.243 | | | | | | |
| 313 | | | | | | | | |
| 314 | | | | 1.486 | | | | |
| 315 | | | | | | | | |
| 316 | | | | | | | | |
| 317 | 0.147 | | | | 0.349 | | | |
| 318 | 0.147 | | | | 0.349 | | | 0.199 |
| 319 | | | | | | | | |
| 320 | | | | | | | | 0.199 |
| 321 | | | 7.952 | | | | | |
| 322 | | | | | | | | |
| 323 | | | | | | | | |
| 324 | | | | | | | | |
| 325 | | 0.243 | | | 0.349 | | | |
| 326 | | | | | | | | |
| 327 | | | 11.928 | | | | | |
| 328 | | | | 4.457 | | | | |
| 329 | | | | | | | | |
| 330 | | | | | | | | |
| 331 | | | | | | | | |
| 332 | | | | 1.486 | | | | |
| 333 | | 0.73 | | | | | | |
| 334 | 0.736 | | | | | | | |
| 335 | | | | | | | | |
| 336 | | | | | | | | |
| 337 | | 0.487 | | | | | | |
| 338 | | | | | | | | |
| 339 | | | | | | | | |
| 340 | 1.178 | | | | | | | |
| 341 | | | | | | | | |
| 342 | | 0.243 | | | | | | |
| 343 | | | 5.964 | | | | | |
| 344 | | | | | | | | |
| 345 | | 0.73 | | | | | | |
| 346 | | | | 3.714 | 1.047 | | | |
| 347 | | 0.487 | | | 0.698 | | | |
| 348 | | | | | | | | |
| 349 | | | | 4.457 | | | 0.516 | |
| 350 | | | | 0.743 | 0.349 | | | |
| 351 | | | | 0.743 | | | | |
| 352 | 0.147 | | | | 0.349 | | | |
| 353 | | | | | | | | |
| 354 | | | | | | | | |
| 355 | | | | | | | | |
| 356 | | | | | | | | |
| 357 | | | | | | | | |
| 358 | | | | | 0.698 | | | |
| 359 | | | | | | | | |
| 360 | | | | | | | | |
| 361 | | | | 0.743 | | | | |
| 362 | | | | | | | | |
| 363 | | | | | | | | |
| 364 | | | | | | | | |
| 365 | | | | | | | | |
| 366 | | 0.243 | | 2.228 | | | | |
| 367 | | | | | | | | |
| 368 | | | | | | | | 0.199 |
| 369 | | | | | | | | |
| 370 | | | | | | | | |
| 371 | | | | | | | | |
| 372 | | | | 3.714 | | | | |
| 373 | | | | | | | | |
| 374 | | | | 0.743 | | | | |
| 375 | | | | | | | | |
| 376 | | | | | | | | |
| 377 | | | | | | | | |
| 378 | | | | | | | | |
| 379 | | | | | | | 0.516 | |
| 380 | | | | | | | | |
| 381 | | | | | | | | |
| 382 | | | | | | | | |
| 383 | | | | | | | | |
| 384 | | | | | | | | |
| 385 | | | | | | | | |
| 386 | | 0.487 | | | | | | |
| 387 | | 0.487 | | | 1.395 | | | |
| 388 | | | | | | | | |
| 389 | | | | | | | | |
| 390 | | | | | | | | |
| 391 | | | | 2.228 | | | | |
| 392 | | | | | | | | |
| 393 | | | | | | | | |
| 394 | | | | | | | | |
| 395 | | | | | | | | |
| 396 | | | | | | | | |
| 397 | | | | | | | | |
| 398 | | | | | | | | |
| 399 | | 0.243 | | | | | | |
| 400 | | 0.243 | | | | | | 0.199 |
| 401 | | 0.487 | | | | | | |
| 402 | | | | | | | | |
| 403 | | | | 1.486 | | | | |
| | 10 | 31 | 8 | 21 | 11 | 1 | 6 | 8 |
| | 7% | 21% | 5% | 14% | 7% | 1% | 4% | 5% |
| Seq. ID | SRR1568515 | SRR1568523 | SRR1568623 | SRR1568639 | SRR1568643 | SRR1568666 | SRR1568669 | SRR1568674 |
| 255 | 0.466 | 0.824 | | | | | 0.091 | |
| 256 | 0.466 | 0.412 | | 0.31 | | 0.075 | | |
| 257 | | 1.647 | | 0.155 | | | | |
| 258 | 0.466 | 0.824 | | | | | | |
| 259 | | | | 0.466 | | 0.075 | | 0.628 |
| 260 | | | | | 0.05 | | 0.914 | 1.885 |
| 261 | | | | | | | | |
| 262 | | | | | | 0.151 | | |
| 263 | | | | | | | | |
| 264 | | | | | | 0.151 | | |
| 265 | | | | | | | | |
| 266 | | 0.412 | | | | | | |
| 267 | | | | | 0.202 | | | |
| 268 | 0.466 | | | | 0.101 | | 0.457 | |
| 269 | | | | | | | | |
| 270 | | | | | | 0.075 | | |
| 271 | | | | | | | | |
| 272 | | 3.295 | | | | | | |
| 273 | | 3.295 | | | | | | |
| 274 | | | | | | 0.075 | | |
| 275 | | | | | | | | |
| 276 | | 2.883 | | | | 0.151 | | |
| 277 | | 0.824 | | | | 0.151 | | |
| 278 | | | | | | | | |
| 279 | | | | | | | | |
| 280 | | | | | | 0.151 | | |
| 281 | | | | | | 0.151 | | |
| 282 | | | | | | | | |
| 283 | | | | | 0.101 | | | |
| 284 | | | | | 0.05 | | | |
| 285 | | | | | | | | |
| 286 | | 0.412 | | | | | | |
| 287 | | | | | | | | 0.943 |
| 288 | | | | 0.31 | | | | |
| 289 | | | | | | | | |
| 290 | | 0.824 | | | | | | |
| 291 | | | | | | 0.075 | | |
| 292 | | | | | | 0.226 | | |
| 293 | 0.466 | 0.412 | | | | | | |
| 294 | | | | | | | | |
| 295 | | | | 0.155 | 0.555 | | | |
| 296 | | | | | | 0.075 | 0.091 | |
| 297 | | | | | | | | |
| 298 | | | | | | | | |
| 299 | | | | | | | | |
| 300 | | | | 0.155 | | | | |
| 301 | | | | | | 0.151 | | |
| 302 | | | | | | | | |
| 303 | | | | | | | | |
| 304 | | 2.059 | | | | | | |
| 305 | | 0.412 | | | | | | |
| 306 | | | | | | | 0.091 | |
| 307 | | 3.707 | | 0.155 | | | | |
| 308 | 2.328 | | | | | | | |
| 309 | | | | | | 0.226 | | |
| 310 | | | | | | | 0.091 | 0.314 |
| 311 | 6.054 | | | 0.155 | | 0.151 | | |
| 312 | | | | | | | | |
| 313 | | | | 0.931 | | 0.226 | | |
| 314 | | | | | | | | |
| 315 | | | | | 0.202 | | 0.091 | |
| 316 | | | | | | 0.075 | | |
| 317 | | | | | | | | |
| 318 | | | | | | | | |
| 319 | | | | | | | | |
| 320 | 4.191 | | | | | | | |
| 321 | | | | | | | | |
| 322 | | | | | | | | |
| 323 | | | | | | | | |
| 324 | | | | | | | | |
| 325 | | | | | | | | |
| 326 | 0.466 | 0.824 | | | | | | |
| 327 | | | | | | | | |
| 328 | | 1.647 | | | | | | |
| 329 | | 0.412 | | | | | | |
| 330 | | 0.412 | | | | | | |
| 331 | | | | | | | | |
| 332 | | | | | | | | |
| 333 | | | | | | | | |
| 334 | | | | | | | | |
| 335 | | | | | 0.05 | 0.151 | 0.091 | 0.943 |
| 336 | | | | | | 0.075 | | |
| 337 | | | | | | | | |
| 338 | | | 0.584 | | | | | 3.142 |
| 339 | | | | | | 0.075 | | |
| 340 | | | | | 0.101 | | | |
| 341 | | | | | | | | |
| 342 | | 2.059 | | | | | | 0.314 |
| 343 | | | | | | | | |
| 344 | 0.931 | | | | | | | |
| 345 | | 2.059 | | | | | | |
| 346 | | | | | | | | |
| 347 | | | | | | | | |
| 348 | | | | | 0.05 | | | |
| 349 | | | | | | | | |
| 350 | | | | 0.466 | | | | |
| 351 | | | | | | | | |
| 352 | | | | | | | 0.091 | |
| 353 | | | | | | 0.075 | | |
| 354 | | | | | | 0.075 | | |
| 355 | | | | 0.155 | | | | |
| 356 | | | | | | 0.075 | | |
| 357 | 0.466 | | | 0.776 | | | | |
| 358 | | | | | | 0.075 | | |
| 359 | | | | | | | | |
| 360 | | | | 0.155 | | | | |
| 361 | | | | | | | | |
| 362 | 1.863 | | | | | 0.075 | | |
| 363 | | | | | | 0.075 | | |
| 364 | | | | | | | | |
| 365 | | | | | 0.151 | | 0.183 | |
| 366 | | | | | | | | |
| 367 | | | | | | 0.075 | | |
| 368 | | | | | | | 0.091 | |
| 369 | | | | 0.776 | 0.05 | | | 1.257 |
| 370 | | | | | | | | |
| 371 | | | | | | | | |
| 372 | | | | | | | | |
| 373 | | | | | | 0.075 | | |
| 374 | | | | | | | | |
| 375 | | | | | | | | |
| 376 | | 0.824 | | | 0.05 | | | |
| 377 | | | | 0.466 | | | | |
| 378 | | | | 0.621 | | | | |
| 379 | | | | | | | | |
| 380 | | | | | | | | |
| 381 | | | | | | | | |
| 382 | | | | | | | | |
| 383 | | | | | | 0.075 | | 0.943 |
| 384 | | | | | | | | |
| 385 | | | | | | 0.075 | | |
| 386 | | | | 0.31 | | | | 0.314 |
| 387 | | | | | | 0.075 | | |
| 388 | | | | | | | | 0.314 |
| 389 | | | | | | | | |
| 390 | | | | 0.155 | | 0.075 | | |
| 391 | | | | | | | | |
| 392 | | | | | | | | |
| 393 | | | | | | 0.226 | | |
| 394 | | | | | | | | |
| 395 | | | | | | | | |
| 396 | | | | | | | | |
| 397 | | 1.236 | | | | | | |
| 398 | | | | | | 0.075 | | |
| 399 | | | | | | | | |
| 400 | | | | | | | | |
| 401 | | 1.647 | | | | 0.075 | | |
| 402 | | | | | | | | 0.628 |
| 403 | | | | | 0.05 | | | |
| # Biomarkers | 12 | 24 | 1 | 18 | 14 | 36 | 11 | 12 |
| Per Sample | | | | | | | | |
| % Coverage | 8% | 16% | 1% | 12% | 9% | 24% | 7% | 8% |
| Stage | Braak V | Braak V | Braak VI | Braak VI | Braak VI | Braak VI | Braak VI | Braak VI |
| Seq. ID | SRR1568705 | SRR1568719 | SRR1568433 | SRR1568435 | SRR1568490 | SRR1568496 | SRR1568525 | SRR1568530 |
| 255 | | | 0.405 | 1.713 | | | 0.398 | 0.39 |
| 256 | | | | 0.514 | 0.572 | | | |
| 257 | | | | 1.028 | | | | |
| 258 | | | | 1.884 | | | | |
| 259 | | | | 0.685 | 0.572 | | | |
| 260 | | | 0.203 | | | | | |
| 261 | | 0.618 | | | | | 0.796 | |
| 262 | | 0.618 | | 1.884 | | | | |
| 263 | | | 0.608 | | | 0.765 | | |
| 264 | | 2.472 | | 0.343 | | 2.296 | | 0.78 |
| 265 | | | | | | | | |
| 266 | 0.671 | | 0.203 | 0.856 | | | | |
| 267 | | 1.854 | | | | | | |
| 268 | | | | | | | | |
| 269 | | | 0.608 | 2.055 | 1.144 | | | |
| 270 | | | | 0.343 | | 0.765 | | |
| 271 | | 0.618 | | | | 0.765 | | |
| 272 | | | | 0.514 | | 2.296 | | |
| 273 | | | | | 0.572 | | 0.796 | 1.171 |
| 274 | | 0.618 | | | | 0.765 | | |
| 275 | | | | | 1.144 | 0.765 | | |
| 276 | | | | | | | 0.398 | |
| 277 | | | | | | 2.296 | | |
| 278 | | | | 1.028 | | 2.296 | 0.398 | 0.39 |
| 279 | | | | | | | | |
| 280 | | | | 0.856 | | | | |
| 281 | | | 0.203 | 0.685 | | | | 0.39 |
| 282 | | | | 0.514 | | 0.765 | | |
| 283 | | | | | | | | |
| 284 | 2.013 | | | | | | | |
| 285 | | 0.618 | | | 1.144 | | | |
| 286 | | | | 0.171 | 0.572 | | | |
| 287 | | | | | | | | |
| 288 | | | | 0.514 | 1.717 | | | |
| 289 | | | | | 1.144 | 0.765 | | |
| 290 | | 0.618 | | 0.685 | | | | |
| 291 | | | | 0.685 | | 0.765 | | |
| 292 | | | | | | 0.765 | | |
| 293 | | | | 0.856 | | | | |
| 294 | | 0.618 | | | 0.572 | | | |
| 295 | | | | | | | | |
| 296 | | | | | | | | |
| 297 | | | | | | | | |
| 298 | | | | 1.37 | | | | |
| 299 | | | | | | 1.531 | | |
| 300 | | | | 2.227 | | | | |
| 301 | | | | 0.343 | | | | |
| 302 | | | 0.405 | | | | | |
| 303 | | | 0.203 | | | | | |
| 304 | | | | 1.028 | | | | 1.171 |
| 305 | | | | 2.398 | | | | |
| 306 | | | | | | | | |
| 307 | | | | 1.199 | | | | |
| 308 | | | | 0.171 | 1.144 | | | |
| 309 | | | | 0.685 | | 2.296 | | |
| 310 | | | | | 1.717 | | | |
| 311 | | | 0.203 | | | | | |
| 312 | | | | | | | | |
| 313 | | 2.472 | | | | 0.765 | | |
| 314 | | 3.708 | 0.203 | | | | | |
| 315 | | | 0.203 | | 1.717 | | | |
| 316 | | | 0.811 | 0.514 | | | | |
| 317 | | | | | | | | |
| 318 | | | | | | | | |
| 319 | | 1.854 | | | | | | |
| 320 | | | 0.203 | | | | | |
| 321 | | | 0.608 | 0.514 | | | | |
| 322 | | | | | | | | |
| 323 | | 1.236 | | | | | | 0.39 |
| 324 | | | 0.203 | 0.343 | | 0.765 | | |
| 325 | | 3.708 | | 1.028 | | | | |
| 326 | | | 0.608 | 1.199 | | | | |
| 327 | | 1.236 | | | | | | 0.78 |
| 328 | | 1.854 | 0.203 | | | | | |
| 329 | | | | 1.37 | | | | |
| 330 | 0.671 | | 0.203 | 2.055 | | | | |
| 331 | | | | | | | | |
| 332 | | | | 1.199 | | | | |
| 333 | 4.025 | | 0.203 | | | 2.296 | | |
| 334 | 3.355 | | | | 0.572 | | | |
| 335 | | | | | | | | |
| 336 | | | | | | | | |
| 337 | | 2.472 | | 1.028 | | | | |
| 338 | | | | | | | | 0.39 |
| 339 | | | | | 1.717 | 1.531 | | |
| 340 | | | | | | | | |
| 341 | | 2.472 | | 0.343 | 0.572 | | | |
| 342 | | | | | | | | |
| 343 | | 0.618 | | 1.199 | | | | |
| 344 | 2.684 | | | | | | | |
| 345 | | | 0.203 | | | | 0.796 | |
| 346 | | | | | | 2.296 | | |
| 347 | | 1.236 | | 1.028 | | | | |
| 348 | | | | | | | | |
| 349 | | | | 0.685 | | | | |
| 350 | | | | | | | | |
| 351 | | | | | | | | |
| 352 | | | | | | | | |
| 353 | | | | 0.514 | 1.717 | 0.765 | | |
| 354 | | | | | 2.289 | 1.531 | | |
| 355 | | | 0.405 | 0.685 | | | | |
| 356 | | 1.854 | 0.608 | 0.343 | | | | |
| 357 | | | 0.405 | 0.171 | | | | |
| 358 | | 0.618 | | | | | | |
| 359 | | | | | | | | |
| 360 | | | 0.405 | 0.171 | | | | |
| 361 | | | | | | 2.296 | | |
| 362 | | | | 0.514 | | 2.296 | | 0.39 |
| 363 | | | | | | | | 0.78 |
| 364 | | | | | | | | |
| 365 | | | 0.608 | | | | | |
| 366 | | | | | | 1.531 | | |
| 367 | | | | 0.685 | | 1.531 | | |
| 368 | | | | | | | | |
| 369 | | 0.618 | | | | | | |
| 370 | | | 0.203 | | | | | |
| 371 | | | 0.203 | | | | | |
| 372 | | | | 0.171 | | | | 1.561 |
| 373 | | 0.618 | | | | | | 0.78 |
| 374 | | | | 0.171 | | | | |
| 375 | | 0.618 | 0.203 | 0.856 | | | | |
| 376 | | | | | | | | |
| 377 | | | | | 1.144 | 1.531 | | |
| 378 | | | 0.608 | | | | | |
| 379 | | | 0.608 | | | | 1.989 | |
| 380 | 2.684 | | | | | | | |
| 381 | | | | | | | | |
| 382 | | 1.854 | | | | | | 0.39 |
| 383 | | | | 0.856 | | | | |
| 384 | | 0.618 | | | | | | |
| 385 | | | | 0.856 | | | | |
| 386 | | | | | | | | |
| 387 | | | | | | 0.765 | | |
| 388 | | | | | | | | |
| 389 | | 1.854 | | | | | | |
| 390 | | | | 1.028 | | | | |
| 391 | | | | 0.514 | | | | |
| 392 | | | | | | | | 0.39 |
| 393 | | 0.618 | | | | | | |
| 394 | | 0.618 | | 1.028 | | 0.765 | | |
| 395 | | 0.618 | | | | 1.531 | | |
| 396 | | | | | 0.572 | | | |
| 397 | | | 0.203 | 0.685 | | | 0.398 | |
| 398 | | | | 0.856 | 0.572 | | | |
| 399 | | | | | | | | |
| 400 | | | | | 1.144 | | | |
| 401 | | | | | 1.144 | | | |
| 402 | | 0.618 | | 0.856 | | 1.531 | 0.398 | |
| 403 | | | | | | | | |
| # Biomarkers | 7 | 32 | 31 | 59 | 23 | 31 | 9 | 15 |
| Per Sample | | | | | | | | |
| % Coverage | 5% | 21% | 21% | 40% | 15% | 21% | 6% | 10% |
| Stage | Braak VI | Braak VI | Braak VI | Braak VI | Braak VI | Braak VI | Braak VI | Braak VI |
| Seq. ID | SRR156853 | SRR1568562 | SRR1568566 | SRR1568598 | SRR1568600 | SRR1568611 | SRR1568641 | SRR1568648 |
| 255 | | | | | | | 0.674 | |
| 256 | | | | | | | 1.348 | |
| 257 | | 0.227 | | | | | 1.348 | |
| 258 | | 0.227 | | | | | 0.674 | 0.414 |
| 259 | | | | | | | | |
| 260 | | | | | | | | 0.828 |
| 261 | | | | | 3.88 | | 3.37 | |
| 262 | | | | | | | | |
| 263 | | 0.455 | | | | | | |
| 264 | | | | 0.236 | | | | |
| 265 | | 0.455 | 12.099 | | | | 3.37 | |
| 266 | | | | | | | 0.674 | |
| 267 | | | | | | | | |
| 268 | | | | | | | | |
| 269 | | | | | | | 2.022 | |
| 270 | | 0.682 | | | | | | |
| 271 | | | | | 7.76 | | | |
| 272 | | 0.227 | | | | | | |
| 273 | | | | | | | | |
| 274 | | 0.682 | | | | | | |
| 275 | | 0.682 | | | | | | |
| 276 | 0.633 | 0.227 | | | | | | |
| 277 | | 1.137 | | | | | 1.348 | |
| 278 | | | | | | | | |
| 279 | | 0.455 | | | | | | |
| 280 | | 0.455 | | | | | | |
| 281 | | | | | | | 2.696 | |
| 282 | | 0.91 | | | | | | |
| 283 | | | | | | | | |
| 284 | | 0.227 | | | | | | |
| 285 | | 1.137 | | | | | 0.674 | |
| 286 | | | | | | | | 2.899 |
| 287 | | | | | | | | 0.828 |
| 288 | | | | | | | | |
| 289 | | | | 0.236 | | | 0.674 | |
| 290 | | 0.227 | | | | | 0.674 | |
| 291 | | | | | | | 2.022 | |
| 292 | | 0.91 | | | | | | |
| 293 | | | | | | | | |
| 294 | | | | | | | 2.696 | |
| 295 | | | | | | | | |
| 296 | | | | | | | | 0.414 |
| 297 | | | | | | | | 7.04 |
| 298 | | | | 0.236 | | | 6.741 | |
| 299 | | | | | | | | |
| 300 | | | | | | | 0.674 | |
| 301 | | 0.227 | | | | | 8.089 | |
| 302 | | | | | | | | |
| 303 | | | | | 9.053 | | 1.348 | |
| 304 | | | | | | | 3.37 | |
| 305 | | | | 0.118 | | | | |
| 306 | | | | 0.707 | | | 6.741 | |
| 307 | | | | | | | 0.674 | |
| 308 | | | | | 4.526 | | | |
| 309 | | | | | | | | |
| 310 | | | | | | | | 0.828 |
| 311 | | | | | | | | |
| 312 | | | | 0.353 | | | | |
| 313 | | | | | | | 2.022 | |
| 314 | | | | | | | | |
| 315 | | | | | | | | |
| 316 | | | | | | | 5.393 | |
| 317 | | | | | | | | 0.414 |
| 318 | | | | | | 0.265 | | |
| 319 | | | | 0.236 | | | | 1.242 |
| 320 | | | | | | | | 1.656 |
| 321 | | | | | | | 2.022 | |
| 322 | | | | 0.118 | | | | |
| 323 | | | | | | | 2.696 | |
| 324 | | | | | 6.466 | | | |
| 325 | | | | | | | | |
| 326 | | | | | | | 2.022 | |
| 327 | | | | | | | | |
| 328 | | 0.455 | | | | | | |
| 329 | | 0.227 | | | | | 1.348 | |
| 330 | | | | | | | | |
| 331 | | | | | | | 5.393 | |
| 332 | | 0.455 | | | | | | |
| 333 | | | | | | | | |
| 334 | | 0.227 | | | | | | |
| 335 | | | | | | | | |
| 336 | | 0.682 | | | | | 2.022 | |
| 337 | | | | | | | | |
| 338 | | | | | | | | |
| 339 | | | | | | | | |
| 340 | | | | | | | | |
| 341 | | | | | | | | |
| 342 | | 0.91 | | | | | | |
| 343 | | | | | | | | |
| 344 | | | | | | | | |
| 345 | | 0.455 | | | | | | |
| 346 | | | | | | | | |
| 347 | | | | | | | | |
| 348 | | 0.455 | | | | | | |
| 349 | | 0.227 | | | | | | |
| 350 | | | | | 2.587 | | | |
| 351 | | | | | | | 1.348 | 1.656 |
| 352 | | | | | | | | |
| 353 | | | | | | | | |
| 354 | | 0.455 | | | 2.587 | | | |
| 355 | | | | | | | | |
| 356 | | | | | | | 2.022 | |
| 357 | | | | | | | | |
| 358 | | | | | | | | |
| 359 | | | | | | | | |
| 360 | | | | | | | 0.674 | |
| 361 | | | | | | | | |
| 362 | | | | | | | | |
| 363 | | | | | | | | |
| 364 | | | | 0.118 | | | | |
| 365 | | | | | | | | 0.414 |
| 366 | | | | | | | | |
| 367 | | 0.227 | | | | | | |
| 368 | | | | | | | | |
| 369 | | | | | | | | |
| 370 | | 0.227 | | | | | | |
| 371 | | | | | | | | |
| 372 | | 0.227 | | 0.118 | | | | |
| 373 | | 0.455 | | | | | | |
| 374 | | 0.227 | | | | | | |
| 375 | | | | | | | | |
| 376 | | | | 0.118 | | | | |
| 377 | | | | | | | | |
| 378 | | | | | | | | |
| 379 | 0.633 | | | | | | | |
| 380 | | 0.455 | | | | | | |
| 381 | | 0.227 | | 0.118 | | | 4.719 | |
| 382 | | 0.227 | | | | | 3.37 | |
| 383 | | 0.227 | | | | | | |
| 384 | | | | | | | | |
| 385 | | | | 0.118 | | | | |
| 386 | | | | 0.118 | | | | |
| 387 | | | | | | | | |
| 388 | | | | | | | | |
| 389 | | 0.682 | | | | | | |
| 390 | | | | 0.118 | | | | |
| 391 | | | | | | | | 0.828 |
| 392 | | | | | | | | |
| 393 | | 0.227 | | | | | | |
| 394 | | 0.455 | | | | | | |
| 395 | | 0.455 | | | | | | |
| 396 | | 0.455 | | 0.118 | | | 2.696 | |
| 397 | | | | 0.236 | | | | |
| 398 | | 0.455 | | | | | | 0.828 |
| 399 | | 0.227 | | | | | 2.696 | |
| 400 | | | | | 3.88 | | | |
| 401 | | | | | | | | |
| 402 | | | | | | | | |
| 403 | | | | | | | | |
| # Biomarkers | 2 | 44 | 1 | 17 | 8 | 1 | 37 | 14 |
| Per Sample | | | | | | | | |
| % Coverage | 1% | 30% | 1% | 11% | 5% | 1% | 25% | 9% |

| Stage | Braak VI | Braak VI | Braak VI |
|---|---|---|---|
| Seq. ID | SRR1568678 | SRR1568748 | SRR1568756 |
| 255 | | | |
| 256 | | | |
| 257 | | | |
| 258 | | | |
| 259 | | | |
| 260 | | | |
| 261 | | | 0.313 |
| 262 | | 0.244 | |
| 263 | | | 0.078 |
| 264 | | | |
| 265 | | | 0.078 |
| 266 | | | |
| 267 | | | 0.313 |
| 268 | | | 0.782 |
| 269 | | | |
| 270 | | | |
| 271 | | 0.489 | |
| 272 | | | |
| 273 | | | |
| 274 | | | |
| 275 | | | |
| 276 | | | |
| 277 | | | |
| 278 | | | |
| 279 | | | |
| 280 | | 0.244 | |
| 281 | | | |
| 282 | | | |
| 283 | | 0.244 | 0.078 |
| 284 | | | 0.078 |
| 285 | | | |
| 286 | | | |
| 287 | | | |
| 288 | | 0.244 | |
| 289 | | | |
| 290 | | | |
| 291 | | | |
| 292 | | 0.244 | |
| 293 | | | |
| 294 | | | |
| 295 | | | 1.408 |
| 296 | | | |
| 297 | | | 0.156 |
| 298 | | | |
| 299 | | 0.244 | |
| 300 | | | |
| 301 | | | |
| 302 | | | 0.078 |
| 303 | | 0.489 | |
| 304 | | | |
| 305 | | 0.489 | |
| 306 | | | |
| 307 | | | |
| 308 | | | |
| 309 | | | |
| 310 | | | |
| 311 | | | |
| 312 | | | 0.078 |
| 313 | | | |
| 314 | | | |
| 315 | | | |
| 316 | | | |
| 317 | | 0.244 | |
| 318 | | | |
| 319 | | | |
| 320 | | | |
| 321 | | | |
| 322 | | | |
| 323 | | | |
| 324 | | | |
| 325 | | | |
| 326 | | | |
| 327 | | | |
| 328 | | | |
| 329 | | | 0.313 |
| 330 | | | |
| 331 | | 0.489 | |
| 332 | | 0.244 | |
| 333 | | | |
| 334 | | | |
| 335 | | | |
| 336 | | | |
| 337 | | | 0.078 |
| 338 | 0.14 | 0.244 | |
| 339 | | | |
| 340 | | 0.244 | |
| 341 | | | |
| 342 | | | |
| 343 | | | |
| 344 | | | |
| 345 | | | |
| 346 | | | |
| 347 | | | |
| 348 | | | 0.156 |
| 349 | | 0.244 | |
| 350 | | | |
| 351 | | | |
| 352 | | | 0.626 |
| 353 | | | |
| 354 | | | |
| 355 | | | |
| 356 | | | |
| 357 | | | |
| 358 | | | |
| 359 | | | 0.391 |
| 360 | | | |
| 361 | | | 0.469 |
| 362 | | | |
| 363 | | | 0.391 |
| 364 | | | 0.156 |
| 365 | | | 0.235 |
| 366 | | 0.244 | 0.391 |
| 367 | | | |
| 368 | | | 0.391 |
| 369 | | | 0.078 |
| 370 | | | 0.156 |
| 371 | | | 0.469 |
| 372 | | | |
| 373 | | | |
| 374 | | | |
| 375 | | | |
| 376 | | | 0.078 |
| 377 | | | |
| 378 | | | 0.078 |
| 379 | | | 0.078 |
| 380 | | | |
| 381 | | 0.244 | |
| 382 | | | |
| 383 | | | |
| 384 | | | 0.235 |
| 385 | | | |
| 386 | | | |
| 387 | | 0.733 | |
| 388 | | | 0.313 |
| 389 | | 0.244 | |
| 390 | | | |
| 391 | | | |
| 392 | | | |
| 393 | | | |
| 394 | | | |
| 395 | | | |
| 396 | | | |
| 397 | | | |
| 398 | | | |
| 399 | | | |
| 400 | | | |
| 401 | | | |
| 402 | | | |
| 403 | | | |
| # Biomarkers | 1 | 19 | 30 |
| Per Sample | | | |
| % Coverage | 1% | 13% | 20% |

TABLE 8
Identified sRNA biomarkers in serum that have a positive correlation
with Braak Stage in order to monitor Alzheimer's Disease

| | | | | | | | | | | |
|---|---|---|---|---|---|---|---|---|---|---|
| | | | | | Braak II | Braak III | Braak IV | Braak V | Braak VI | |
| Seq. ID | Total Reads | Specificity | Sensitivity | p-value | Avg | Avg | Avg | Avg | Avg | # Hits |

| | | | | | | | | |
|---|---|---|---|---|---|---|---|---|
| 257 | 21 | 100% | 15.69% | 2.61E−05 | 7.416 | 0.964 | 0.868 | 3 |
| 270 | 19 | 100% | 11.76% | 4.01E−04 | 5.944 | 2.266 | 0.597 | 3 |
| 272 | 19 | 100% | 11.76% | 4.01E−04 | 2.759 | 2.019 | 1.012 | 3 |
| 273 | 17 | 100% | 11.76% | 4.01E−04 | 1.981 | 1.664 | 0.846 | 3 |
| 279 | 14 | 100% | 11.76% | 4.01E−04 | 3.678 | 0.798 | 0.455 | 3 |
| 286 | 12 | 100% | 11.76% | 4.01E−04 | 0.991 | 1.200 | 1.214 | 3 |
| 288 | 12 | 100% | 11.76% | 4.01E−04 | 1.839 | 0.277 | 0.825 | 3 |
| 314 | 17 | 100% | 9.80% | 1.53E−03 | 5.944 | 1.754 | 0.203 | 3 |
| 319 | 17 | 100% | 9.80% | 1.53E−03 | 4.114 | 1.854 | 0.739 | 3 |
| 325 | 16 | 100% | 9.80% | 1.53E−03 | 1.839 | 1.433 | 1.028 | 3 |
| 332 | 15 | 100% | 9.80% | 1.53E−03 | 2.759 | 1.486 | 0.633 | 3 |
| 341 | 14 | 100% | 9.80% | 1.53E−03 | 4.598 | 1.270 | 0.458 | 3 |
| 374 | 12 | 100% | 9.80% | 1.53E−03 | 5.518 | 0.422 | 0.199 | 3 |
| 391 | 11 | 100% | 9.80% | 1.53E−03 | 1.854 | 1.148 | 0.671 | 3 |
| 393 | 11 | 100% | 9.80% | 1.53E−03 | 2.759 | 0.588 | 0.227 | 3 |

TABLE 9
Identified sRNA biomarkers in colon epithelium tissue that are associated with Normal individuals.

| SEQ | | | | | | | | | | |
|---|---|---|---|---|---|---|---|---|---|---|
| ID | | import- | | | | | | | | |
| NO: | Marker | ance | imp_SE | sRNA_name | ref | ext | swaps | chosen | thislbl | otherlbl |
| 405 | GCTGATTGTCACGTTCTGATT | 0.61173 | 0.11392 | hsa-mir- | (0:0) | (GC:) | (1: | 0.9 | 2.305 | 0.767 |
| | | | | 5701 | | | T > C) | | | |
| 406 | GCCCCTGGGCCTATCCTAGA | −0.50514 | 0.07172 | hsa-mir- | (0:−1) | (:) | ( ) | 1 | 1.473 | 2.614 |
| | | | | 331-3p | | | | | | |
| 407 | AGTTCTTCAGTGGCAAGCT | −0.43217 | 0.12976 | hsa-mir- | (0:−3) | (:) | ( ) | 0.7 | −0.639 | 0.822 |
| | | | | 22-5p | | | | | | |
| 408 | ACCCTGTAGAACCGAATTTGTA | 0.23477 | 0.08481 | hsa-mir- | (1:−1) | (:A) | ( ) | 0.5 | 3.3 | 1.212 |
| | | | | 10b-5p | | | | | | |
| 409 | TAGGTAGTTTCCTGTTGTTGGAT | 0.17757 | 0.0569 | hsa-mir- | (0:−1) | (:AT) | (11: | 0.8 | 0.15 | −0.592 |
| | | | | 196a-5p | | | A > C) | | | |
| 410 | ACCCTGTAGATCTGAATTTGT | 0.16483 | 0.10074 | hsa-mir- | (1:−1) | (:) | (10: | 0.3 | 0.782 | −0.34 |
| | | | | 10b-5p | | | A > T, 12: | | | |
| | | | | | | | C > T) | | | |
| 411 | TGAGATGAAGCTGTAGCTC | 0.16362 | 0.03238 | hsa-mir- | (0:0) | (:C) | (8: | 0.8 | 0.779 | −0.308 |
| | | | | 4770 | | | C > A, 9: | | | |
| | | | | | | | A > G) | | | |
| 412 | TACCCTGTAGAACCGAATTGGT | 0.15816 | 0.04547 | hsa-mir- | (0:−1) | (:) | (19: | 0.7 | 1.483 | −0.398 |
| | | | | 10b-5p | | | T > G) | | | |
| 413 | ACCCTGTAGAACCGAATTTGG | 0.1312 | 0.04783 | hsa-mir- | (1:−2) | (:G) | (10: | 0.5 | 0.875 | −0.605 |
| | | | | 10a-5p | | | T > A) | | | |
| 414 | TAACAGTCTACAGCCATGGTCG | −0.12465 | 0.06087 | hsa-mir- | (0:0) | (:) | ( ) | 0.6 | 3.56 | 4.436 |
| | | | | 132-3p | | | | | | |
| 415 | AGTTCTTCAGTGGCAAGCTT | −0.11012 | 0.05699 | hsa-mir- | (0:−2) | (:) | ( ) | 0.3 | −0.394 | 1.187 |
| | | | | 22-5p | | | | | | |
| 416 | TACCCTGTAGAACCGAATTTGG | 0.09977 | 0.03596 | hsa-mir- | (0:−2) | (:G) | ( ) | 0.5 | 4.121 | 1.664 |
| | | | | 10b-5p | | | | | | |
| 417 | CAGTGCAATGATGAAAGGGCAT | −0.08933 | 0.05037 | hsa-mir- | (0:0) | (:) | (10: | 0.3 | 0.717 | 2.623 |
| | | | | 130a-3p | | | T > A, | | | |
| | | | | | | | 12: | | | |
| | | | | | | | A > G) | | | |
| 418 | TACCCTGTAGAACCGAATTTA | 0.07544 | 0.04788 | hsa-mir- | (0:−3) | (:A) | ( ) | 0.4 | 2.698 | 0.845 |
| | | | | 10b-5p | | | | | | |
| 419 | TACAGTTGTTCAACCAGTTACT | −0.07464 | 0.05019 | hsa-mir- | (1:0) | (:) | ( ) | 0.2 | −0.358 | 0.671 |
| | | | | 582-5p | | | | | | |
| 420 | ACCCTGTAGAACCGAATTTGGG | 0.06375 | 0.06375 | hsa-mir- | (1:0) | (:) | (10: | 0.1 | 0.747 | −0.188 |
| | | | | 10a-5p | | | T > A, | | | |
| | | | | | | | 20: | | | |
| | | | | | | | T > G) | | | |
| 421 | TACCCTGTAGGACCGAATTTGT | 0.05883 | 0.03032 | hsa-mir- | (0:−1) | (:) | (10: | 0.4 | 1.962 | −0.355 |
| | | | | 10b-5p | | | A > G) | | | |
| 422 | TGGCAGTGTCTTAGCTGGTT | −0.05794 | 0.04762 | hsa-mir- | (0:−2) | (:) | ( ) | 0.2 | −0.482 | 1.044 |
| | | | | 34a-5p | | | | | | |
| 423 | ACCCTGTAGAACCGAATTTA | 0.04848 | 0.03233 | hsa-mir- | (1:−3) | (:A) | (10: | 0.2 | 0.32 | −0.63 |
| | | | | 10a-5p | | | T > A) | | | |
| 424 | ACCCTGTAGAACCGAATTTGTT | 0.04605 | 0.04605 | hsa-mir- | (1:−1) | (:T) | ( ) | 0.1 | 1.076 | −0.146 |
| | | | | 10b-5p | | | | | | |
| 425 | TACCCTGTAGATCCGATTTTGT | 0.04078 | 0.01861 | hsa-mir- | (0:−1) | (:) | (11: | 0.4 | 1.192 | −0.283 |
| | | | | 10b-5p | | | A > T, 16: | | | |
| | | | | | | | A > T) | | | |
| 426 | TACCCTGTAGAACCGAGTTTGT | 0.03972 | 0.03306 | hsa-mir- | (0:−1) | (:) | (16:A > G) | 0.2 | 2.752 | 0.399 |
| | | | | 10b-5p | | | | | | |
| 427 | TTCAAGTAATCCAGGATAGGCC | 0.03965 | 0.03658 | hsa-mir- | (0:−1) | (:CT) | ( ) | 0.2 | 0.841 | −0.548 |
| | T | | | 26a-5p | | | | | | |
| 428 | TACCCTGTAGAACCGAATTTAT | 0.03939 | 0.03051 | hsa-mir- | (0:−1) | (:) | (20: | 0.2 | 1.886 | 0.183 |
| | | | | 10b-5p | | | G> A) | | | |
| 429 | TACCCTGTAGAACCGGATTTG | 0.03714 | 0.02781 | hsa-mir- | (0:−2) | (:) | (15: | 0.2 | 0.166 | −0.663 |
| | | | | 10b-5p | | | A > G) | | | |
| 430 | TATTGCACTTGTCCCGGCCTGTA | 0.03206 | 0.03206 | hsa-mir- | (0:2) | (:C) | (22: | 0.1 | 0.533 | −0.546 |
| | GC | | | 92a-3p | | | G > A) | | | |
| 431 | ACCCTGTAGATCTGAATTTGTGA | 0.02789 | 0.02789 | hsa-mir- | (1:0) | (:A) | (12: | 0.1 | 0.267 | −0.681 |
| | | | | 10a-5p | | | C > T) | | | |
| 432 | CACTAGATTGTGAGCTCCT | 0.02652 | 0.02652 | hsa-mir- | (0:−3) | (:) | ( ) | 0.1 | 2.028 | 0.439 |
| | | | | 28-3p | | | | | | |
| 433 | TACCCTGTAGTACCGAATTTGT | 0.02641 | 0.02641 | hsa-mir- | (0:−1) | (:) | (10: | 0.1 | 1.227 | −0.21 |
| | | | | 10b-5p | | | A > T) | | | |
| 434 | CAGTGCAATGTTAAAAGGGCAA | −0.026 | 0.01733 | hsa-mir- | (0:−1) | (:A) | (10: | 0.2 | −0.212 | 1.183 |
| | | | | 130b-3p | | | A > T, 12: | | | |
| | | | | | | | G > A) | | | |
| 435 | CTGACCTATGATTTGACAGCC | 0.02413 | 0.01324 | hsa-mir- | (0:0) | (:) | (11: | 0.3 | 1.746 | 0.096 |
| | | | | 192-5p | | | A > T) | | | |
| 436 | CTGACCTATGAATTGACAGCCCT | 0.02306 | 0.01562 | hsa-mir- | (0:0) | (:CT) | ( ) | 0.2 | 2.004 | 0.427 |
| | | | | 192-5p | | | | | | |
| 437 | CCACTGCCCCAGGTGCTGCTGG | −0.02248 | 0.02248 | hsa-mir- | (−2:0) | (:) | ( ) | 0.1 | −0.481 | 0.945 |
| | | | | 324-3p | | | | | | |
| 438 | TGAGGTAGTAGGTTGTGTGGGT | 0.02215 | 0.02215 | hsa-let- | (0:0) | (:) | (16: | 0.1 | 0.975 | 0.325 |
| | | | | 7c-5p | | | A > G, 20: | | | |
| | | | | | | | T > G) | | | |
| 439 | ACTGTGCGTGTGACAGCGGCT | −0.02097 | 0.01562 | hsa-mir- | (−1:−2) | (:) | ( ) | 0.2 | −0.666 | 0.215 |
| | | | | 210-3p | | | | | | |
| 440 | CTGCGCAAGCTACTGCCTTG | −0.0202 | 0.0202 | hsa-let- | (0:−2) | (:) | ( ) | 0.1 | 1.199 | 2.896 |
| | | | | 7i-3p | | | | | | |
| 441 | CACCCGTAGAACCGACCTTGCG | −0.02011 | 0.01097 | hsa-mir- | (0:0) | (:A) | ( ) | 0.3 | 3.612 | 4.648 |
| | A | | | 99b-5p | | | | | | |
| 442 | CTGACCTATGTATTGACAGCC | 0.01839 | 0.01249 | hsa-mir- | (0:0) | (:) | (10: | 0.2 | 2.279 | 0.663 |
| | | | | 192-5p | | | A > T) | | | |
| 443 | TACCCTGTAGAACCGAATTTGC | 0.01577 | 0.01577 | hsa-mir- | (0:−2) | (:C) | ( ) | 0.1 | 4.555 | 1.079 |
| | | | | 10b-5p | | | | | | |
| 444 | TGAGAACTGAATTCCATAGGCT | −0.01551 | 0.01551 | hsa-mir- | (0:1) | (:AA) | (17: | 0.1 | −0.359 | 0.464 |
| | GAA | | | 146a-5p | | | G > A, 20: | | | |
| | | | | | | | T > C) | | | |
| 445 | TGACCTATGAATTGACAGCCAAT | 0.01402 | 0.01402 | hsa-mir- | (1:3) | (:T) | (18: | 0.1 | 0.754 | 0.46 |
| | T | | | 215-5p | | | A > C) | | | |
| 446 | TACCCTGTAGAACCGAATTTGTA | 0.01382 | 0.01382 | hsa-mir- | (0:−1) | (:A) | ( ) | 0.1 | 5.669 | 4.122 |
| | | | | 10b-5p | | | | | | |
| 447 | TGAGATGAAGCACTGTAGATC | 0.01158 | 0.01158 | hsa-mir- | (0:0) | (:) | (18: | 0.1 | 2.526 | 1.048 |
| | | | | 143-3p | | | C > A) | | | |
| 448 | TACCCTGTAGAACCGAACTTGT | 0.0115 | 0.00939 | hsa-mir- | (0:−1) | (:) | (17: | 0.2 | 1.946 | 0.086 |
| | | | | 10b-5p | | | T >C) | | | |
| 449 | CTGACCTATGAACTGACAGCC | 0.01068 | 0.0088 | hsa-mir- | (0:0) | (:) | (12: | 0.2 | 2.713 | 0.568 |
| | | | | 192-5p | | | T > C) | | | |
| 450 | GATTGTCACGTTCTGATT | 0.00994 | 0.00994 | hsa-mir- | (2:0) | (G:) | ( ) | 0.1 | 0.926 | −0.013 |
| | | | | 5701 | | | | | | |
| 451 | TTACAGTCTACAGCCATGGTCG | −0.007 | 0.007 | hsa-mir- | (0:0) | (:) | (1: | 0.1 | −0.541 | 0.325 |
| | | | | 132-3p | | | A > T) | | | |
| 452 | CATTGCACTTGTCTCGGTCTGAA | 0.00642 | 0.00642 | hsa-mir- | (0:0) | (:AT) | ( ) | 0.1 | 2.02 | 0.798 |
| | T | | | 25-3p | | | | | | |
| 453 | TACCCTGTTGAACCGAATTTGT | 0.00629 | 0.00629 | hsa-mir- | (0:−1) | (:) | (8: | 0.1 | 0.959 | −0.227 |
| | | | | 10b-5p | | | A > T) | | | |
| 45 | CAAAGTGCTGTTCGTGCAGGTA | −0.00623 | 0.00623 | hsa-mir- | (0:−1) | (:) | ( ) | 0.1 | 2.94 | 3.614 |
| | | | | 93-5p | | | | | | |
| 455 | CTCGCTTCTGGCGCCAAGCGCC | −0.00413 | 0.00413 | <NA > | (NA:NA | (NA:NA) | ( ) | 0.1 | −0.552 | 0.651 |
| | CGGC | | | | 1 | | | | | |
| 456 | AACTGGCCCTCAAAGTCCCG | −0.00368 | 0.00368 | hsa-mir- | (0:−2) | (:) | ( ) | 0.1 | 0.083 | 1.702 |
| | | | | 193b-3p | | | | | | |
| 457 | TGAGAACTGAATTCCATAGGCA | −0.00364 | 0.00364 | hsa-mir- | (0:−1) | (:AA) | ( ) | 0.1 | 0.256 | 1.187 |
| | A | | | 146b-5p | | | | | | |
| 458 | TGAGGTAGTAGATTGTATAGTT | 0.00325 | 0.00325 | hsa-let- | (0:2) | (:) | (11: | 0.1 | 0.75 | −0.212 |
| | TT | | | 7a-5p | | | G > A) | | | |
| 459 | ACCCTGTAGATCCGAAT | 0.00148 | 0.00148 | hsa-mir- | (1:−5) | (:) | ( ) | 0.1 | 0.215 | −0.459 |
| | | | | 10a-5p | | | | | | |
| 460 | AGGCTGTGATGCTCTCCTGAGC | 0.00039 | 0.00039 | hsa-mir- | (0:−1) | (:CT) | ( ) | 0.1 | 0.595 | −0.142 |
| | CCT | | | 7974 | | | | | | |
| 461 | TAACACTGTCTGGTAAC | 0.00027 | 0.00027 | hsa-mir- | (0:−5) | (:) | ( ) | 0.1 | 1.631 | −0.336 |
| | | | | 200a-3p | | | | | | |
| 462 | TACCCTGTAGATCCGAATTCGT | 0.00024 | 0.00024 | hsa-mir- | (0:−1) | (:) | (11: | 0.1 | 1.832 | −0.081 |
| | | | | | | | A > T, 19: | | | |
| | | | | 10b-5p | | | T > C) | | | |

TABLE 10
Identified sRNA biomarkers in colon epithelium tissue that are associated with Crohn's disease.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislb | otherlb |
| 463 | CCGCCCCACCCCGCGCGCGCCGC | 0.74618 | 0.16463 | <NA > | (NA:NA) | (NA:NA) | ( ) | 0.8 | 1.72 | −0.59 |
| 464 | CGCTTCTGGCGCCAAGCGCCCGGC | 0.25545 | 0.08406 | <NA > | (NA:NA) | (NA:NA) | ( ) | 0.7 | 1.39 | −0.62 |
| | CGC | | | | | | | | | |
| 465 | AGATTGAGGGTTCGTCCCTTCGTG | 0.25408 | 0.05563 | <NA > | (NA:NA) | (NA:NA) | ( ) | 0.8 | 2.73 | 0.37 |
| | GTCGCC | | | | | | | | | |
| 466 | GGCTTGGTCTAGGGGTATGATTCT | 0.21881 | 0.06902 | <NA > | (NA:NA) | (NA:NA) | ( ) | 0.7 | 2.2 | −0.46 |
| | CGCTTT | | | | | | | | | |
| 467 | GGCTTTGTCTAGGGGTATGATTCT | 0.18401 | 0.12882 | <NA > | (NA:NA) | (NA:NA) | ( ) | 0.4 | 1.34 | −0.65 |
| | CGCTT | | | | | | | | | |
| 468 | CCCGCCCCACCCCGCGCGCGCCGC | 0.15615 | 0.09596 | <NA > | (NA:NA) | (NA:NA) | ( ) | 0.3 | 1.5 | −0.64 |
| | T | | | | | | | | | |
| 469 | CGTACGGAAGACCCGCTCCCCGGC | 0.11296 | 0.05941 | <NA > | (NA:NA) | (NA:NA) | ( ) | 0.3 | 1.26 | 0.61 |
| | GCCGCT | | | | | | | | | |
| 470 | GTACGGAAGACCCGCTCCCCGGCG | 0.10944 | 0.10944 | <NA > | (NA:NA) | (NA:NA) | ( ) | 0.1 | 1.36 | 0.59 |
| | CCG | | | | | | | | | |
| 471 | TGGTCTAGCGGTTAGGATTCCTGG | 0.09687 | 0.06389 | <NA > | (NA:NA) | (NA:NA) | ( ) | 0.3 | 1.02 | −0.66 |
| | TTTT | | | | | | | | | |
| 472 | CGCCCCACCCCGCGCGCGCCGC | 0.09422 | 0.03815 | <NA > | (NA:NA) | (NA:NA) | ( ) | 0.5 | 1.64 | 0.61 |
| 473 | CCCGCGAGGGGGGCCCGGGCAC | 0.07217 | 0.05546 | <NA > | (NA:NA) | (NA:NA) | ( ) | 0.2 | 1.03 | −0.58 |
| 474 | GCGCCGCCGCCCCCCCCACGCCCG | 0.06871 | 0.04611 | <NA > | (NA:NA) | (NA:NA) | ( ) | 0.2 | 1.64 | −0.67 |
| | GGGC | | | | | | | | | |
| 475 | GCTCCCCGTCCTCCCCCCTCCCC | 0.06762 | 0.06762 | <NA > | (NA:NA) | (NA:NA) | ( ) | 0.1 | 1.58 | −0.67 |
| 476 | GCGCAATGAAGGTGAAGGCCGGC | 0.06288 | 0.03999 | <NA > | (NA:NA) | (NA:NA) | ( ) | 0.4 | 1.03 | −0.6 |
| | GC | | | | | | | | | |
| 477 | ACGCTGCCAGTTGAAGAACTGT | 0.05063 | 0.05063 | hsa-mir- | (0:0) | (:) | (1: | 0.1 | 0.86 | −0.46 |
| | | | | 22-3p | | | A > C) | | | |
| 478 | GCCCCTGGGCCTATCCTAGAAAA | 0.04958 | 0.03308 | hsa-mir- | (0:0) | (:AA) | ( ) | 0.2 | 0.68 | −0.65 |
| | | | | 331-3p | | | | | | |
| 479 | GCGGGTCCGGCCGTGTCGGCGGC | 0.04831 | 0.04831 | <NA > | (NA:NA) | (NA:NA) | ( ) | 0.1 | 0.65 | −0.67 |
| 480 | GGCTTGGTCTAGGGGTATGATTCT | 0.04437 | 0.04437 | <NA > | (NA:NA) | (NA:NA) | ( ) | 0.1 | 3.5 | 0.65 |
| | CGCT | | | | | | | | | |
| 481 | CCACCTCCCCTGCAAACGTCC | 0.03994 | 0.02586 | hsa-mir- | (0:−1) | (:) | ( ) | 0.4 | 0.46 | −0.6 |
| | | | | 1306-5p | | | | | | |
| 482 | GGTTAGGATTCCTGGTTTT | 0.03829 | 0.03829 | <NA > | (NA:NA) | (NA:NA) | ( ) | 0.1 | 1.08 | −0.57 |
| 483 | TCTGGCATGCTAACTAGTTACGCG | 0.03622 | 0.03622 | <NA > | (NA:NA) | (NA:NA) | ( ) | 0.1 | 0.84 | −0.67 |
| | ACCCCC | | | | | | | | | |
| 484 | CGCGTCCCCCGAAGAGGGGGACG | 0.03391 | 0.03391 | <NA > | (NA:NA) | (NA:NA) | ( ) | 0.1 | 1.08 | −0.68 |
| | GCGGAGC | | | | | | | | | |
| 485 | GCGGAGCGAGCGCACGGGGTCGG | 0.0323 | 0.0323 | <NA > | (NA:NA) | (NA:NA) | ( ) | 0.1 | 0.79 | −0.52 |
| | CGGCGAC | | | | | | | | | |
| 486 | CCCCCGCCCCACCCCGCGCGCGCC | 0.02563 | 0.02563 | <NA > | (NA:NA) | (NA:NA) | ( ) | 0.1 | 1.3 | −0.68 |
| | GCTCGC | | | | | | | | | |
| 487 | CCGTAGGTGAACCTGCGGAAGGAT | 0.02433 | 0.01963 | <NA > | (NA:NA) | (NA:NA) | ( ) | 0.2 | 2.36 | −0.5 |
| | CATTA | | | | | | | | | |
| 488 | GGGCTACGCCTGTCTGAGCGTCGC | 0.02206 | 0.02206 | <NA > | (NA:NA) | (NA:NA) | ( ) | 0.1 | 2.74 | 0.07 |
| | TT | | | | | | | | | |
| 489 | GCTACGCCTGTCTGAGCGTCGCTT | 0.02103 | 0.02103 | <NA > | (NA:NA) | (NA:NA) | ( ) | 0.1 | 1.48 | −0.46 |
| 490 | CCCCCACAACCGCGCTTGACTAGCT | 0.0204 | 0.0204 | <NA > | (NA:NA) | (NA:NA) | ( ) | 0.1 | 1.43 | −0.36 |
| | T | | | | | | | | | |
| 491 | CCCTACCCCCCCGGCCCCGTC | 0.01307 | 0.01307 | <NA > | (NA:NA) | (NA:NA) | ( ) | 0.1 | 1.25 | −0.56 |
| 492 | CCCGCCCCACCCCGCGCGCGCCGC | 0.01108 | 0.01108 | <NA > | (NA:NA) | (NA:NA) | ( ) | 0.1 | 1.7 | −0.59 |
| | TCGC | | | | | | | | | |
| 493 | GGGGGTATAGCTCAGTGGTAGAG | 0.01022 | 0.01022 | <NA > | (NA:NA) | (NA:NA) | ( ) | 0.1 | 1.12 | −0.58 |
| | CGTGCTT | | | | | | | | | |
| 494 | GTCGGTCGGGCTGGGGCGCGAAG | 0.00996 | 0.00996 | <NA > | (NA:NA) | (NA:NA) | ( ) | 0.1 | 2.53 | −0.51 |
| | CGGGGCT | | | | | | | | | |
| 495 | TCAGTGGAGAGCATTTGACT | 0.00991 | 0.00991 | <NA > | (NA:NA) | (NA:NA) | ( ) | 0.1 | 0.54 | −0.66 |
| 496 | CACCCCTAGAACCGACCTTGCG | 0.0095 | 0.0095 | hsa-mir- | (0:0) | (:) | (5: | 0.1 | 0.17 | −0.66 |
| | | | | 99b-5p | | | G > C) | | | |
| 497 | CCTCACCATCCCTTCTGCCTGCA | 0.00892 | 0.00892 | hsa-mir- | (0:1) | (:) | ( ) | 0.1 | 0.2 | −0.65 |
| | | | | 6511a-3p | | | | | | |
| 498 | GTCAGGATGGCCGAGCGGTCT | 0.00647 | 0.00647 | <NA > | (NA:NA) | (NA:NA) | ( ) | 0.1 | 2.13 | 0.36 |
| 499 | TCCCTGGTCTAGTGGTTAGGATTC | 0.00644 | 0.00644 | <NA > | (NA:NA) | (NA:NA) | ( ) | 0.1 | 1.6 | −0.27 |
| | GGCGCG | | | | | | | | | |
| 500 | TGAGATGAAGCACTGTAGATC | −0.00555 | 0.00555 | hsa-mir- | (0:0) | (:) | (18: | 0.1 | −0.07 | 1.91 |
| | | | | 143-3p | | | C > A) | | | |
| 501 | GGATCGGCCCCGCCGGGGTCGGC | 0.00523 | 0.00523 | <NA > | (NA:NA) | (NA:NA) | ( ) | 0.1 | 1.04 | −0.68 |
| 502 | GGAACCTGCGGAAGGATCATTA | 0.00215 | 0.00215 | <NA > | (NA:NA) | (NA:NA) | ( ) | 0.1 | 2.24 | 0.33 |
| 503 | TGAGGTAGTAGGTTGTATGGTTG | 0.00179 | 0.00179 | hsa-mir- | (0:1) | (:) | (5: | 0.1 | 0.92 | 0.53 |
| | | | | 4510 | | | G > T, | | | |
| | | | | | | | 12: | | | |
| | | | | | | | A > T) | | | |
| 504 | GTCTAGTGGTTAGGATTCGGCGCT | 0.00093 | 0.00093 | <NA > | (NA:NA) | (NA:NA) | ( ) | 0.1 | 1.61 | −0.38 |
| 505 | TCCCTGGTCTAGTGGCTAGGATTC | 0.00085 | 0.00085 | <NA > | (NA:NA) | (NA:NA) | ( ) | 0.1 | 0.72 | −0.64 |
| | GGCGCT | | | | | | | | | |
| 506 | GCCGCCCCCCCCACGCCCGGGGC | 0.0002 | 0.0002 | <NA > | (NA:NA) | (NA:NA) | ( ) | 0.1 | 0.59 | −0.68 |

TABLE 11
Identified sRNA biomarkers in colon epithelium tissue
that are associated with Ulcerative colitis.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislbl | otherlbl |
| 507 | TGTCAGTTTGTCAAATACCC | 0.46706 | 0.1009 | hsa-mir- | (0:2) | (:) | ( ) | 0.9 | 1.892 | 0.1084 |
| | CAAG | | | 223-3p | | | | | | |
| 508 | CAGCAGCAATTCATGTTTTG | 0.29749 | 0.09883 | hsa-mir- | (0:0) | (:T) | ( ) | 0.6 | 0.578 | 0.613 |
| | AAT | | | 424-5p | | | | | | |
| 509 | GTGGTTGTAGTCCGTGCGA | −0.22154 | 0.09667 | <NA > | (NA:NA) | (NA:NA) | ( ) | 0.5 | −0.373 | 1.2368 |
| | GAATACC | | | | | | | | | |
| 510 | GGATATCATCATATACTGTA | 0.1973 | 0.11602 | hsa-mir- | (0:1) | (:) | ( ) | 0.4 | 2.428 | 0.8535 |
| | AGT | | | 144-5p | | | | | | |
| 511 | TAACAGTCTCCAGTCACGG | 0.14329 | 0.07797 | hsa-mir- | (0:−1) | (:) | ( ) | 0.6 | 1.215 | −0.5329 |
| | C | | | 212-3p | | | | | | |
| 512 | TCAGTGCACTACAGAACTTT | 0.13604 | 0.06626 | hsa-mir- | (0:0) | (:T) | (20: | 0.5 | 0.643 | −0.6209 |
| | TTT | | | 148a-3p | | | G > T) | | | |
| 513 | CCAGTGGGGCTGCTGTTAT | −0.13318 | 0.07284 | hsa-mir- | (0:0) | (:T) | ( ) | 0.3 | 0.857 | 2.7111 |
| | CTGT | | | 194-3p | | | | | | |
| 514 | GATAAAGTAGAAAGCACTA | 0.13252 | 0.06175 | hsa-mir- | (1:0) | (G:) | ( ) | 0.4 | 1.653 | −0.6021 |
| | CT | | | 142-5p | | | | | | |
| 515 | TAGGTAGTTTCCTGTTGTTG | −0.1183 | 0.04091 | hsa-mir- | (0:−1) | (:AT) | (11: | 0.6 | −0.676 | −0.1724 |
| | GAT | | | 196a-5p | | | A > C) | | | |
| 516 | ATGCTTATCAGACTGATGTT | 0.11425 | 0.07239 | hsa-mir- | (2:0) | (AT:) | ( ) | 0.3 | 1.241 | −0.512 |
| | GA | | | 21-5p | | | | | | |
| 517 | TAGTGCAATATTGCTTATAG | 0.10893 | 0.0759 | hsa-mir- | (0:−1) | (:) | ( ) | 0.3 | 0.82 | 0.0483 |
| | GG | | | 454-3p | | | | | | |
| 518 | CCCATAAAGTAGAAAGCAC | 0.10582 | 0.05342 | hsa-mir- | (−2:0) | (:) | ( ) | 0.5 | 1.414 | −0.294 |
| | TACT | | | 142-5p | | | | | | |
| 519 | TACCCATTGCATATCGGAGT | 0.097 | 0.07557 | hsa-mir- | (0:−1) | (:) | ( ) | 0.3 | 0.876 | −0.4505 |
| | T | | | 660-5p | | | | | | |
| 520 | ACTGGACTTGGAGTCAGAA | −0.09333 | 0.05017 | hsa-mir- | (0:3) | (:A) | (13: | 0.3 | 2.232 | 4.1887 |
| | GGAA | | | 378b | | | G > T, | | | |
| | | | | | | | 19: | | | |
| | | | | | | | A > G) | | | |
| 521 | AAGCAGCAATTCATGTTTTG | 0.09165 | 0.06219 | hsa-mir- | (1:−1) | (A:) | ( ) | 0.2 | 0.263 | −0.6458 |
| | A | | | 424-5p | | | | | | |
| 522 | CTGCAGCACGTAAATATTG | 0.0866 | 0.05794 | hsa-mir- | (2:0) | (CT:) | ( ) | 0.2 | 0.882 | −0.5753 |
| | GCG | | | 16-5p | | | | | | |
| 523 | TGGCAGTGTCTTAGCTGGT | 0.07815 | 0.06409 | hsa-mir- | (0:−2) | (:) | ( ) | 0.3 | 1.71 | −0.1242 |
| | T | | | 34a-5p | | | | | | |
| 524 | ACTGGACTTGGAGTCAGAA | −0.07752 | 0.052 | hsa-mir- | (0:−2) | (:) | (20:A > | 0.2 | −0.284 | 1.3769 |
| | GGTT | | | 378c | | | G, 21: | | | |
| | | | | | | | G > T) | | | |
| 525 | TGAGAACTGAATTCCATAG | 0.07149 | 0.03423 | hsa-mir- | (0:4) | (:) | (24: | 0.6 | 2.372 | 0.6917 |
| | GCTGTAA | | | 146b-5p | | | G > A) | | | |
| 526 | ACTGGACTTGGAGTCAGAA | −0.0679 | 0.04539 | hsa-mir- | (0:−2) | (:) | (20:A > | 0.2 | 0.289 | 2.0819 |
| | GGAT | | | 378c | | | G, 21: | | | |
| | | | | | | | G > A) | | | |
| 527 | TGAGAACTGAATTCCATAG | 0.06566 | 0.04343 | hsa-mir- | (0:4) | (:T) | (24: | 0.3 | 0.687 | −0.4488 |
| | GCTGTAAT | | | 146b-5p | | | G > A) | | | |
| 528 | GTTGAGACTCTGAAATCTG | −0.06461 | 0.05023 | hsa-mir- | (−2:−7) | (G:GATT) | (3: | 0.2 | −0.649 | 0.1771 |
| | ATT | | | 4431 | | | C > G, | | | |
| | | | | | | | 14: | | | |
| | | | | | | | A > A) | | | |
| 529 | TTAATGCTAATCGTGATAG | 0.06346 | 0.02758 | hsa-mir- | (0:−4) | (:) | ( ) | 0.4 | 2.46 | 0.3365 |
| | | | | 155-5p | | | | | | |
| 530 | TGAGAACTGAATTCCATAG | 0.06095 | 0.0468 | hsa-mir- | (0:−2) | (:AA) | (17: | 0.2 | 1.103 | 0.1217 |
| | GAA | | | 146a-5p | | | G > A) | | | |
| 531 | CTATACGACCTGCTGCCTTT | −0.05799 | 0.05799 | hsa-let- | (0:−1) | (:A) | ( ) | 0.1 | 0.725 | 1.845 |
| | CA | | | 7d-3p | | | | | | |
| 532 | TACCCTGTAGAACCGAATTT | −0.05773 | 0.04012 | hsa-mir- | (0:0) | (:) | (11: | 0.2 | −0.445 | 0.5034 |
| | GCG | | | 10a-5p | | | T > A, | | | |
| | | | | | | | 21: | | | |
| | | | | | | | T > C) | | | |
| 533 | TGGCAGTGTCTTAGCTGGT | 0.05695 | 0.04073 | hsa-mir- | (0:−3) | (:) | ( ) | 0.2 | 0.721 | −0.5822 |
| | | | | 34a-5p | | | | | | |
| 534 | CCAGTGGGGCTGCTGTTAT | −0.05534 | 0.03762 | hsa-mir- | (0:−1) | (:) | ( ) | 0.3 | 1.163 | 2.2638 |
| | CT | | | 194-3p | | | | | | |
| 535 | TTGAGAACTGAATTCCATG | 0.05453 | 0.04544 | hsa-mir- | (−1:0) | (:) | ( ) | 0.2 | 2.563 | 0.8429 |
| | GGTT | | | 146a-5p | | | | | | |
| 536 | TTACAGTCTACAGCCATGGT | 0.04999 | 0.04437 | hsa-mir- | (0:0) | (:) | (1: | 0.2 | 0.833 | −0.4181 |
| | CG | | | 132-3p | | | A > T) | | | |
| 537 | ACTGGACTTGGAGTCAGAA | −0.04834 | 0.0324 | hsa-mir- | (0:3) | (:) | (19: | 0.2 | 5.356 | 6.5699 |
| | GGCT | | | 378d | | | A > G, | | | |
| | | | | | | | 20: | | | |
| | | | | | | | A > G) | | | |
| 538 | TGAGAACTGAATTCCATAG | 0.04829 | 0.0337 | hsa-mir- | (0:2) | (:AG) | ( ) | 0.2 | 0.761 | −0.2346 |
| | GCTGTAG | | | 146b-5p | | | | | | |
| 539 | CCCATAAAGTAGAAAGCAC | 0.04703 | 0.03279 | hsa-mir- | (−2:−1) | (:A) | ( ) | 0.2 | 2.327 | 0.2258 |
| | TACA | | | 142-5p | | | | | | |
| 540 | TGAGGTAGTAGTTTGTGCT | 0.04637 | 0.04637 | hsa-let- | (0:−3) | (:) | ( ) | 0.1 | 3.668 | 2.5754 |
| | | | | 7i-5p | | | | | | |
| 541 | CGGCGCAAGCTACTGCCTT | 0.04625 | 0.04625 | hsa-let- | (0:−2) | (:) | (1: | 0.1 | 0.127 | −0.6692 |
| | G | | | 7i-3p | | | T > G) | | | |
| 542 | AGTTCTTCAGTGGCAAGCT | 0.04577 | 0.04577 | hsa-mir- | (0:−3) | (:) | ( ) | 0.1 | 1.084 | −0.0644 |
| | | | | 22-5p | | | | | | |
| 543 | TCCCCTGTAGAACCGAATTT | −0.04267 | 0.02897 | hsa-mir- | (0:−1) | (:) | (1: | 0.2 | −0.655 | 0.1801 |
| | GT | | | 10b-5p | | | A > C) | | | |
| 544 | ACTGGACTTGGAGTCAGAA | −0.04209 | 0.02716 | hsa-mir- | (0:0) | (:ATT) | (9: | 0.3 | 1.615 | 3.1346 |
| | GGCATT | | | 422a | | | A > G, | | | |
| | | | | | | | 11: | | | |
| | | | | | | | G > A) | | | |
| 545 | AAGCTCGGTCTGAGGCCCC | −0.04032 | 0.03266 | hsa-mir- | (−1:−2) | (:) | ( ) | 0.2 | 0.598 | 1.7929 |
| | TCA | | | 423-3p | | | | | | |
| 546 | CCAGTGGGGCTGCTGTTAT | −0.03971 | 0.03971 | hsa-mir- | (0:0) | (:A) | ( ) | 0.1 | −0.383 | 1.5327 |
| | CTGA | | | 194-3p | | | | | | |
| 547 | TGAGGGAGTAGTTTGTGCT | 0.03743 | 0.02474 | hsa-let- | (0:0) | (:A) | (5: | 0.3 | 0.516 | −0.5159 |
| | GTTA | | | 7i-5p | | | T > G) | | | |
| 548 | AAGAAAGTAGAAAGCACTA | 0.03726 | 0.03726 | hsa-mir- | (1:0) | (A:) | (1: | 0.1 | 0.759 | −0.6659 |
| | CT | | | 142-5p | | | T > A) | | | |
| 549 | CGCTGCCAGTTGAAGAACT | 0.03671 | 0.03671 | hsa-mir- | (2:0) | (C:) | ( ) | 0.1 | 1.055 | −0.5449 |
| | GT | | | 22-3p | | | | | | |
| 550 | GGCTGGTCCGATGGTAGT | −0.03534 | 0.03534 | hsa-mir- | (0:−1) | (:) | (8: | 0.1 | 0.079 | 1.378 |
| | | | | 6131 | | | A > C, | | | |
| | | | | | | | 14: | | | |
| | | | | | | | G > T) | | | |
| 551 | CTGGGAGAAGGCTGTTTAC | −0.03467 | 0.03467 | hsa-mir- | (0:0) | (:) | ( ) | 0.1 | 0.783 | 1.6525 |
| | TCT | | | 30C-2-3p | | | | | | |
| 552 | AAGCAATTCTCAAAGGAGC | 0.03329 | 0.01693 | hsa-mir- | (−3:−5) | (:) | ( ) | 0.4 | 0.38 | −0.6931 |
| | | | | 5571-5p | | | | | | |
| 553 | CTCGGCGCCCCCTCGATGCT | −0.03132 | 0.02602 | <NA > | (NA:NA) | (NA:NA) | ( ) | 0.2 | −0.37 | 0.6322 |
| | CT | | | | | | | | | |
| 554 | TGTCTTGCAGGCCGTCATGC | 0.02612 | 0.01998 | hsa-mir- | (0:−1) | (:) | ( ) | 0.2 | 0.613 | −0.6042 |
| | | | | 431-5p | | | | | | |
| 555 | CGAATCATTATTTGCTGCT | 0.02532 | 0.02532 | hsa-mir- | (0:−3) | (:) | ( ) | 0.1 | 1.521 | −0.0129 |
| | | | | 15b-3p | | | | | | |
| 556 | CAGCAGCAATTCATGTTTTG | 0.02138 | 0.02138 | hsa-mir- | (0:0) | (:A) | ( ) | 0.1 | 0.241 | −0.3669 |
| | AAA | | | 424-5p | | | | | | |
| 557 | ACCAATATTACTGTGCTGCT | 0.0205 | 0.01422 | hsa-mir- | (−1:−3) | (:) | ( ) | 0.2 | 3.128 | 1.1757 |
| | | | | 16-2-3p | | | | | | |
| 558 | TTCAAGTAATCCAGGATAG | −0.02004 | 0.02004 | hsa-mir- | (0:2) | (:) | (22: | 0.1 | 3.007 | 4.1471 |
| | GCTTT | | | 26a-5p | | | G > T) | | | |
| 559 | TTGAGAACTGAATTCCATG | 0.01968 | 0.01968 | hsa-mir- | (−1:−1) | (:) | ( ) | 0.1 | 1.968 | 0.5389 |
| | GGT | | | 146a-5p | | | | | | |
| 560 | TATTGCACATTACTAAGTTG | 0.01865 | 0.01865 | hsa-mir- | (0:−2) | (:) | ( ) | 0.1 | 3.749 | 1.603 |
| | | | | 32-5p | | | | | | |
| 561 | TGACCTATGAATTGACAGC | −0.01793 | 0.01793 | hsa-mir- | (1:2) | (:) | (18: | 0.1 | −0.659 | 0.189 |
| | CTA | | | 215-5p | | | A > C, | | | |
| | | | | | | | 20: | | | |
| | | | | | | | A > T) | | | |
| 562 | ACTGTAAACGCTTTCTGATG | −0.01783 | 0.01783 | hsa-mir- | (0:0) | (:) | ( ) | 0.1 | 1.014 | 1.2253 |
| | | | | 3607-3p | | | | | | |
| 563 | CATTGCACTTGTCTCGGTCT | −0.01738 | 0.01738 | hsa-mir- | (0:0) | (:AT) | ( ) | 0.1 | 0.719 | 1.4522 |
| | GAAT | | | 25-3p | | | | | | |
| 564 | ATAAAGTAGAAAGCACTAC | 0.01695 | 0.01695 | hsa-mir- | (1:0) | (:) | ( ) | 0.1 | 2.536 | 0.3764 |
| | T | | | 142-5p | | | | | | |
| 565 | AAGTGCAATGATGAAAGGG | 0.01537 | 0.01537 | hsa-mir- | (1:−1) | (A:) | (9: | 0.1 | 0.631 | −0.6633 |
| | CA | | | 130a-3p | | | T > G, | | | |
| | | | | | | | 11: | | | |
| | | | | | | | A > T) | | | |
| 566 | ACCATAAAGTAGAAAGCAC | 0.01523 | 0.01523 | hsa-mir- | (−1:−2) | (A:) | ( ) | 0.1 | 1.096 | −0.3697 |
| | TA | | | 142-5p | | | | | | |
| 567 | CCCCACTGCTAAATTTGACT | −0.01424 | 0.01424 | <NA > | (NA:NA) | (NA:NA) | ( ) | 0.1 | −0.076 | 1.0335 |
| | GGCTTT | | | | | | | | | |
| 568 | TGTCAGTTTGTCAAATACCC | 0.01423 | 0.01423 | hsa-mir- | (0:2) | (:A) | ( ) | 0.1 | 0.507 | −0.6124 |
| | CAAGA | | | 223-3p | | | | | | |
| 569 | TACCCAGTAGAACCGAATTT | −0.01326 | 0.01326 | hsa-mir- | (0:−1) | (:) | (5: | 0.1 | −0.197 | 0.5859 |
| | GT | | | 10b-5p | | | T > A) | | | |
| 570 | TTTGTTCGTTCGGCTCGCGT | −0.01282 | 0.01282 | hsa-mir- | (0:0) | (:) | (20: | 0.1 | −0.245 | 1.5709 |
| | AA | | | 375 | | | G > A) | | | |
| 571 | ATGCTGCCAGTTGAAGAAC | 0.01218 | 0.01218 | hsa-mir- | (0:0) | (:A) | (1: | 0.1 | 0.462 | −0.555 |
| | TGTA | | | 22-3p | | | A > T) | | | |
| 572 | TGAGAACCACGTCTGCTCT | 0.01124 | 0.01124 | hsa-mir- | (0:−2) | (:) | ( ) | 0.1 | 0.523 | −0.2778 |
| | G | | | 589-5p | | | | | | |
| 573 | CTGCCAATTCCATAGGTCAC | −0.0098 | 0.0098 | hsa-mir- | (0:0) | (:T) | ( ) | 0.1 | 0.349 | 1.5762 |
| | AGT | | | 192-3p | | | | | | |
| 574 | TAGCTTATCAGACTGATGTT | 0.00974 | 0.00974 | hsa-mir- | (0:0) | (:GA) | ( ) | 0.1 | 0.626 | 0.2759 |
| | GAGA | | | 21-5p | | | | | | |
| 575 | GTAGCTTATCAGACTGATGT | 0.00953 | 0.00953 | hsa-mir- | (−1:2) | (:) | ( ) | 0.1 | 1.628 | 0.0433 |
| | TGACT | | | 21-5p | | | | | | |
| 576 | TTTGGTCCCCTTCAACCAGC | −0.00945 | 0.00945 | hsa-mir- | (0:0) | (:A) | ( ) | 0.1 | −0.62 | −0.0075 |
| | TGA | | | 133a-3p | | | | | | |
| 577 | TGTAATAGCAACTCCATGTG | −0.00844 | 0.00844 | hsa-mir- | (0:1) | (:) | (5: | 0.1 | −0.638 | 0.24 |
| | GAA | | | 194-5p | | | C > T) | | | |
| 578 | GGGACCTATGAATTGACAG | 0.00774 | 0.00774 | hsa-mir- | (2:0) | (GG:) | (17: | 0.1 | 0.989 | −0.4886 |
| | AC | | | 192-5p | | | C > A) | | | |
| 579 | TAAGGTGCATCTAGTGCAG | 0.00772 | 0.00772 | hsa-mir- | (0:−1) | (:) | (19: | 0.1 | 2.295 | 0.6414 |
| | ATA | | | 18b-5p | | | T > A) | | | |
| 580 | GTACTGGAAAGTGCACTTG | −0.00721 | 0.00721 | <NA > | (NA:NA) | (NA:NA) | ( ) | 0.1 | −0.395 | 1.7345 |
| | GACGAACA | | | | | | | | | |
| 581 | CCCGGGGCTACGCCTGTCT | −0.00713 | 0.00713 | <NA > | (NA:NA) | (NA:NA) | ( ) | 0.1 | 1.856 | 2.7329 |
| | GAGCGTCGCT | | | | | | | | | |
| 582 | AAAGCTGGGTTGAGAGGG | −0.00655 | 0.00655 | hsa-mir- | (1:2) | (:) | ( ) | 0.1 | 0.172 | 0.9853 |
| | CGAAA | | | 320a | | | | | | |
| 583 | CATAAAGTAGAAAGCACTA | 0.00604 | 0.00537 | hsa-mir- | (0:−2) | (:) | ( ) | 0.2 | 2.95 | 1.1211 |
| | | | | 142-5p | | | | | | |
| 584 | TGTCAGTTTGTCAAATAC | 0.00602 | 0.00602 | hsa-mir- | (0:-4) | (:) | ( ) | 0.1 | 2.716 | −0.261 |
| | | | | 223-3p | | | | | | |
| 585 | TCCGGTGAGCTCTCGCTGG | 0.00578 | 0.00578 | hsa-mir- | (−1:1) | (T:) | (9: | 0.1 | 0.207 | −0.4932 |
| | CC | | | 4792 | | | G > C) | | | |
| 586 | TATAAAGTAGAAAGCACTA | 0.00555 | 0.00555 | hsa-mir- | (1:−1) | (T:) | ( ) | 0.1 | 0.13 | −0.6931 |
| | C | | | 142-5p | | | | | | |
| 587 | TGCTGCCAGTTGAAGAACT | 0.00546 | 0.00546 | hsa-mir- | (2:0) | (T:) | ( ) | 0.1 | 0.158 | −0.6517 |
| | GT | | | 22-3p | | | | | | |
| 588 | AGCTCGGTCTGAGGCCCCT | −0.00518 | 0.00518 | hsa-mir- | (0:2 | (:) | (23: | 0.1 | 0.091 | 1.3932 |
| | CAGTTT | | | 423-3p | | | C > T) | | | |
| 589 | TGTCAGTTTGTCAAATACCC | 0.00464 | 0.00464 | hsa-mir- | (0:2) | (:) | (22: | 0.1 | 0.204 | −0.642 |
| | CATG | | | 223-3p | | | A > T) | | | |
| 590 | ATCACAGTGGCTAAGTTCC | 0.00413 | 0.00413 | hsa-mir- | (1:−2) | (A:) | ( ) | 0.1 | 0.487 | −0.6176 |
| | | | | 27a-3p | | | | | | |
| 591 | TGAGAACTGAATTCCATAG | 0.0039 | 0.0039 | hsa-mir- | (0:−1) | (:AA) | ( ) | 0.1 | 1.542 | 0.5058 |
| | GCAA | | | 146b-5p | | | | | | |
| 592 | TGGGTCTTTGCGGGCGAGA | −0.00383 | 0.00383 | hsa-mir- | (0:0) | (:) | ( ) | 0.1 | 1.582 | 2.1855 |
| | TGA | | | 193a-5p | | | | | | |
| 593 | TACCCTGTAGAACCGGATTT | −0.00313 | 0.00313 | hsa-mir- | (0:−2) | (:) | (15: | 0.1 | −0.657 | −0.2565 |
| | G | | | 10b-5p | | | A > G) | | | |
| 594 | TGAGGGAGTAGATTGTATA | 0.00301 | 0.00301 | hsa-let- | (0:−1) | (:) | (5: | 0.1 | 1.916 | 0.1598 |
| | GT | | | 7a-5p | | | T > G, | | | |
| | | | | | | | 11: | | | |
| | | | | | | | G > A) | | | |
| 595 | TACCCTGTTGAACCGAATTT | −0.00297 | 0.00297 | hsa-mir- | (0:−1) | (:) | (8: | 0.1 | −0.159 | 0.3187 |
| | GT | | | 10b-5p | | | A > T) | | | |
| 596 | TAAGGTGCATCTAGTGCAG | 0.00245 | 0.00245 | hsa-mir- | (0:−2) | (:) | ( ) | 0.1 | 2.559 | 0.72 |
| | AT | | | 18a-5p | | | | | | |
| 597 | GAGAACTGAATTCCATAGG | 0.0021 | 0.0021 | hsa-mir- | (1:2) | (:) | ( ) | 0.1 | 0.549 | −0.328 |
| | CTGT | | | 146b-5p | | | | | | |
| 598 | TAGCAGCACGCAAATATTG | 0.00209 | 0.00209 | hsa-mir- | (0:0) | (:) | (10: | 0.1 | 0.28 | −0.5687 |
| | GCG | | | 16-5p | | | T > C) | | | |
| 599 | GGCTCGTTGGTCTAGGGG | −0.0019 | 0.0019 | hsa-mir- | (0:−2) | (:) | (5: | 0.1 | −0.534 | 0.0195 |
| | | | | 4448 | | | C > G) | | | |
| 600 | CAGCAGCAATTCATGTTTTG | 0.00173 | 0.00173 | hsa-mir- | (0:−2) | (:) | ( ) | 0.1 | 0.987 | −0.0245 |
| | | | | 424-5p | | | | | | |
| 601 | AACATTCAACGCTGTCGGT | −0.00169 | 0.00169 | hsa-mir- | (0:−3) | (:) | (8:T > | 0.1 | 3.67 | 3.8391 |
| | G | | | 181b-5p | | | A, 9: | | | |
| | | | | | | | T > C) | | | |
| 602 | ATGCAGCACGTAAATATTG | 0.00169 | 0.00169 | hsa-mir- | (2:0) | (AT:) | ( ) | 0.1 | 0.338 | −0.6428 |
| | GCG | | | 16-5p | | | | | | |
| 603 | TGCCGACGGGCGCTGACCC | −0.00159 | 0.00159 | <NA > | (NA:NA) | (NA:NA) | ( ) | 0.1 | −0.369 | 0.6898 |
| | CCTT | | | | | | | | | |
| 604 | ATTGGTCGTGGTTGTAGTC | −0.00106 | 0.00106 | <NA > | (NA:NA) | (NA:NA) | ( ) | 0.1 | −0.405 | 0.4592 |
| | CGTGCGAGAA | | | | | | | | | |
| 605 | TGGCAGTGTCTTAGCTGGT | 0.001 | 0.001 | hsa-mir- | (0:−1) | (:) | ( ) | 0.1 | 1.828 | 0.6251 |
| | TG | | | 34a-5p | | | | | | |
| 606 | TGTCAGTTTGTCAAATA | 0.00095 | 0.00095 | hsa-mir- | (0:−5) | (:) | ( ) | 0.1 | 0.047 | −0.6931 |
| | | | | 223-3p | | | | | | |
| 607 | ACCCTGAGACCCTAACTTGT | 0.00016 | 0.00016 | hsa-mir- | (1:0) | (A:) | ( ) | 0.1 | 0.322 | −0.5771 |
| | GA | | | 125b-5p | | | | | | |
| 608 | TGGCAGTTTGTCAAATACC | 0.00011 | 0.00011 | hsa-mir- | (0:−3) | (:) | (2: | 0.1 | 1.467 | −0.5979 |
| | | | | 223-3p | | | T > G) | | | |

TABLE 12
Identified sRNA biomarkers in colon epithelium tissue
that are associated with Diverticular disease.

| SEQ | | | | | | | | | | |
|---|---|---|---|---|---|---|---|---|---|---|
| ID | | Import- | imp_ | sRNA_ | | | | | | |
| NO: | Marker | ance | SE | name | ref | ext | swaps | chosen | thislbl | otherlbl |
| 609 | ACTGGACTTGGAGTCAGAAGGCA | 1.3057 | 0.12197 | hsa-mir- | (0:0) | (:ATAT) | (9: | 1 | 1.458 | −0.67 |
| | TAT | | | 422a | | | A > G, | | | |
| | | | | | | | 11: | | | |
| | | | | | | | G > A) | | | |
| 610 | TCGACCGGACCTCGACCGGCTAG | 0.23143 | 0.11311 | hsa-mir- | (0:2) | (:A) | (21: | 0.4 | 1.008 | −0.59 |
| | A | | | 1307-5p | | | C > A) | | | |
| 611 | TCAGCACCAGGATATTGTTGGA | 0.11606 | 0.05936 | hsa-mir- | (0:−1) | (:) | ( ) | 0.4 | 1.535 | −0.58 |
| | | | | 3065-3p | | | | | | |
| 612 | TGTAACCGCAACTCCATGTGGA | 0.09378 | 0.05427 | hsa-mir- | (0:0) | (:) | (6: | 0.3 | 1.788 | −0.39 |
| | | | | 194-5p | | | A > C) | | | |
| 613 | ACTGGACTTGGAGTCAGAAGGCA | 0.08715 | 0.04571 | hsa-mir- | (0:0) | (:ATTA) | (9: | 0.3 | 1.098 | −0.67 |
| | TTA | | | 422a | | | A > G, | | | |
| | | | | | | | 11: | | | |
| | | | | | | | G > A) | | | |
| 614 | AACACTGTCTGGTAAAGATGGC | 0.08212 | 0.0662 | hsa-mir- | (1:1) | (:) | ( ) | 0.2 | 1.265 | −0.63 |
| | | | | 141-3p | | | | | | |
| 615 | TGTAAACATCCTACACTCTCAG | 0.08206 | 0.03761 | hsa-mir- | (0:1) | (:TA) | ( ) | 0.5 | 0.138 | −0.69 |
| | CTTA | | | 30c-5p | | | | | | |
| 616 | ACTGGACTTTGAGTCAGAAGGCA | 0.06028 | 0.04522 | hsa-mir- | (0:0) | (:A) | (9: | 0.3 | 0.671 | −0.65 |
| | | | | 422a | | | A > T, | | | |
| | | | | | | | 11: | | | |
| | | | | | | | G > A) | | | |
| 617 | ACTGGACTTGGAGCCAGAAGGCA | 0.05242 | 0.04482 | hsa-mir- | (0:2) | (:AA) | (20: | 0.2 | 0.921 | −0.65 |
| | A | | | 378f | | | T > G) | | | |
| 618 | GTAACAGCAACTCCATGTGGAAA | 0.04186 | 0.02857 | hsa-mir- | (1:1) | (:A) | ( ) | 0.2 | 0.92 | −0.67 |
| | | | | 194-5p | | | | | | |
| 619 | ACTGGACTTGGAGTCAGAAGGCA | 0.03645 | 0.01948 | hsa-mir- | (0:0) | (:AATA) | (9: | 0.5 | −0.038 | −0.69 |
| | ATA | | | 422a | | | A > G, | | | |
| | | | | | | | 11: | | | |
| | | | | | | | G > A) | | | |
| 620 | CTGGACTTGGAGTCAGAAGGCAG | 0.0346 | 0.02916 | hsa-mir- | (1:2) | (:AGA) | (12: | 0.2 | 0.159 | −0.68 |
| | A | | | 378f | | | C > T, | | | |
| | | | | | | | 19: | | | |
| | | | | | | | T > G) | | | |
| 621 | TGATATGTTTGATATATTAGG | 0.03153 | 0.02537 | hsa-mir- | (0:1) | (:A) | ( ) | 0.2 | 1.842 | −0.53 |
| | TTA | | | 190a-5p | | | | | | |
| 622 | TGAAATGTTTAGGACCACTAGAA | 0.02779 | 0.02185 | hsa-mir- | (1:1) | (:AT) | ( ) | 0.2 | 0.309 | −0.68 |
| | T | | | 203a-3p | | | | | | |
| 623 | TGGACTTGGAGTCAGAAGGCAT | 0.02407 | 0.01645 | hsa-mir- | (2:0) | (:AT) | ( ) | 0.2 | 0.622 | −0.66 |
| | | | | 378a-3p | | | | | | |
| 624 | TGTAACAGCAACTCCATGTGGAC | 0.01862 | 0.01862 | hsa-mir- | (0:2) | (:A) | ( ) | 0.1 | 0.327 | −0.58 |
| | TA | | | 194-5p | | | | | | |
| 625 | TCGACCGGACCTCGACCGGCTA | 0.01749 | 0.01519 | hsa-mir- | (0:0) | (:A) | ( ) | 0.2 | 1.518 | −0.6 |
| | | | | 1307-5p | | | | | | |
| 626 | TGAGATGAAGCACTGTAGCTCAT | 0.01455 | 0.01455 | hsa-mir- | (0:1) | (:TA) | ( ) | 0.1 | 0.975 | −0.61 |
| | A | | | 143-3p | | | | | | |
| 627 | TTTCAGTCGGATGTTTGCAGCAA | 0.01444 | 0.01444 | hsa-mir- | (1:0) | (:AA) | (16: | 0.1 | 0.141 | −0.69 |
| | | | | 30e-3p | | | A > G) | | | |
| 628 | GACCTATGAATTGACAGCCAT | 0.01188 | 0.00963 | hsa-mir- | (2:1) | (:T) | (17: | 0.2 | 1.014 | −0.58 |
| | | | | 215-5p | | | A > C) | | | |
| 629 | CCACTGCCCCAGGTGCTGCTGGA | 0.01092 | 0.01092 | hsa-mir- | (−2:0) | (:A) | ( ) | 0.1 | 0.692 | −0.6 |
| | | | | 324-3p | | | | | | |
| 630 | CTGACCTATGAATTGACAGCCAT | 0.0102 | 0.0102 | hsa-mir- | (0:1) | (:TGA) | ( ) | 0.1 | 0.583 | −0.63 |
| | GA | | | 192-5p | | | | | | |
| 631 | ACCACAGGGTAGAACCACGGACG | 0.00927 | 0.00927 | hsa-mir- | (1:2) | (:GA) | ( ) | 0.1 | 0.682 | −0.58 |
| | A | | | 140-3p | | | | | | |
| 632 | TCGACCGGACCTCGACCGGCTGA | 0.00896 | 0.00896 | hsa-mir- | (0:0) | (:GA) | ( ) | 0.1 | −0.463 | −0.68 |
| | | | | 1307-5p | | | | | | |
| 633 | TGGCTCAGTTCAGCAGGAACAGG | 0.00641 | 0.00641 | hsa-mir- | (0:2) | (:) | ( ) | 0.1 | 0.543 | −0.6 |
| | A | | | 24-3p | | | | | | |
| 634 | AGCTTATCAGACTGATGTTGAAA | 0.00487 | 0.00487 | hsa-mir- | (1:0) | (:AA) | ( ) | 0.1 | 0.052 | −0.66 |
| | | | | 21-5p | | | | | | |
| 635 | ATCACATTGCCAGGGATAAAA | 0.00469 | 0.00469 | hsa-mir- | (0:−3) | (:AA) | (13: | 0.1 | 0.333 | −0.66 |
| | | | | 23c | | | T > G, | | | |
| | | | | | | | 17: | | | |
| | | | | | | | T > A) | | | |
| 636 | TCAACAAAATCACTGATGCTGGA | 0.0018 | 0.0018 | hsa-mir- | (0:0) | (:) | ( ) | 0.1 | 0.71 | −0.53 |
| | | | | 3065-5p | | | | | | |
| 637 | ACATTGCCAGGGATTTCCA | 0.00084 | 0.00084 | hsa-mir- | (3:1) | (:) | ( ) | 0.1 | 1.31 | −0.57 |
| | | | | 23a-3p | | | | | | |
| 638 | AACACTGTCTGGTAAAGATG | 0.00065 | 0.00065 | hsa-mir- | (1:−1) | (:) | ( ) | 0.1 | −0.094 | −0.69 |
| | | | | 141-3p | | | | | | |

1.-90. (canceled)

91. A method for constructing a disease classifier, comprising:
providing small RNA (sRNA) sequence data for one or more training sets representing one or more disease conditions of interest,
determining the presence or absence of sRNA sequences in samples of the training sets, and constructing a classifier algorithm using supervised, semi-supervised, or unsupervised machine learning that discriminates the one or more disease conditions of interest based on the presence or absence of sRNA sequences in a panel;
validating the classifier algorithm in an independent testing set of biological samples from subjects having the one or more disease conditions of interest by detecting the presence or absence of sRNAs sequences in the panel.

92. The method of claim 91, wherein the classifier algorithm is constructed using one or more of Logistic Regression, Support Vector Machines, Decision Trees, Random Forests, Neural Networks, Probit Regression, Fisher's Linear Discriminant, Naïve Bayes Classifier, Perceptron, Quadratic classifiers, Kernel Estimation, k-Nearest Neighbor, Learning Vector Quantization, and Principal Components Analysis.

93. The method of claim 91, wherein the classifier algorithm comprises a non-parametric, logistical regression, and supervised machine learning.

94. The method of claim 91, wherein the machine learning is supervised machine learning, and the training samples are labeled as positive or negative for the one or more disease conditions.

95. The method of claim 91, wherein individual sRNA sequences are identified in the sRNA sequence data by trimming 3′ sequencing adaptors and without consolidating sRNA sequence variants to a reference sequence or genetic locus.

96. The method of claim 91, wherein the presence or absence of sRNAs in the panel are determined in the independent testing set by quantitative RT-PCR.

97. The method of claim 91, wherein the disease classifier classifies samples among at least three disease conditions.

98. The method of claim 91, wherein the panel contains from about 4 to about 200 sRNAs.

99. The method of claim 91, wherein the training and testing samples are blood, serum, plasma, urine, saliva, or cerebrospinal fluid.

100. The method of claim 91, wherein the training set has at least 100 samples, including at least 10 samples for each disease condition.

101. The method of claim 100, wherein the disease conditions are diseases of the central nervous system.

102. The method of claim 101, wherein at least two disease conditions are neurodegenerative diseases involving symptoms of dementia.

103. The method of claim 101, wherein at least two disease conditions are selected from Alzheimer's Disease, Parkinson's Disease, Huntington's Disease, Mild Cognitive Impairment, Progressive Supranuclear Palsy, Frontotemporal Dementia, Lewy Body Dementia, and Vascular Dementia.

104. The method of claim 101, wherein at least two disease conditions are neurodegenerative diseases involving symptoms of loss of movement control.

105. The method of claim 101, wherein at least one disease condition is selected from Alzheimer's Disease, Parkinson's Disease, Huntington's Disease, Multiple Sclerosis, Amyotrophic Lateral Sclerosis, and Spinal Muscular Atrophy; and training samples are annotated for disease stage, disease severity, drug responsiveness, or course of disease progression.

106. The method of claim 100, wherein the disease conditions are cancers of different tissue or cell origin.

107. The method of claim 100, wherein the disease conditions are inflammatory or immunological diseases, and optionally including one or more of Systemic Lupus Erythematosus (SLE), scleroderma, autoimmune vasculitis, diabetes mellitus (type 1 or type 2), Grave's disease, Addison's disease, Sjögren's syndrome, thyroiditis, rheumatoid arthritis, myasthenia gravis, multiple sclerosis, fibromyalgia, psoriasis, Crohn's disease, ulcerative colitis, and celiac disease.

108. The method of claim 107, wherein the biological samples are blood, serum, or plasma.

109. The method of claim 100, wherein the disease conditions are cardiovascular diseases, optionally including stratification for risk of acute event.

110. The method of claim 109, wherein the cardiovascular diseases include one or more of coronary artery disease (CAD), myocardial infarction, stroke, congestive heart failure, hypertensive heart disease, cardiomyopathy, heart arrhythmia, congenital heart disease, valvular heart disease, carditis, aortic aneurysms, peripheral artery disease, and venous thrombosis.

111. The method of claim 91, wherein at least one, or at least two, or at least five, or at least 10 sRNAs in the panel are positive sRNA predictors, which were identified as present in a plurality of samples labeled as positive for a disease condition in the training set, and absent in all samples labeled as negative for the disease condition in the training set.


SEQUENCE LISTING (638 sequences)

SEQ ID NO: 1 - DNA, 23 bp, synthetic construct
  source 1..23: mol_type = other DNA
caggcagttacagatcgaactcc

SEQ ID NO: 2 - DNA, 19 bp, synthetic construct
  source 1..19: mol_type = other DNA
ggtcagttacagatcgaac

SEQ ID NO: 3 - DNA, 24 bp, synthetic construct
  source 1..24: mol_type = other DNA
ctggctgggttgttcgagacccgc

SEQ ID NO: 4 - DNA, 17 bp, synthetic construct
  source 1..17: mol_type = other DNA
ttatgtgatgacttaca

SEQ ID NO: 5 - DNA, 17 bp, synthetic construct
  source 1..17: mol_type = other DNA
ttctgtgatgacttaca

SEQ ID NO: 6 - DNA, 23 bp, synthetic construct
  source 1..23: mol_type = other DNA
aggttatgggttcgtgtcccacc

SEQ ID NO: 7 - DNA, 18 bp, synthetic construct
  source 1..18: mol_type = other DNA
tcttgctccgtccactcc

SEQ ID NO: 8 - DNA, 25 bp, synthetic construct
  source 1..25: mol_type = other DNA
ggtagagcatgggactcttaatcgc

SEQ ID NO: 9 - DNA, 19 bp, synthetic construct
  source 1..19: mol_type = other DNA
tcgtgctgggcccataacc

SEQ ID NO: 10 - DNA, 23 bp, synthetic construct
  source 1..23: mol_type = other DNA
gggttgtgggttcgggtcccacc

SEQ ID NO: 11 - DNA, 17 bp, synthetic construct
  source 1..17: mol_type = other DNA
tttatcacgttcgcctc

SEQ ID NO: 12 - DNA, 23 bp, synthetic construct
  source 1..23: mol_type = other DNA
aggttccgggctcgggacccggc

SEQ ID NO: 13 - DNA, 21 bp, synthetic construct
  source 1..21: mol_type = other DNA
catatgtggtgaatacgtgtt

SEQ ID NO: 14 - DNA, 27 bp, synthetic construct
  source 1..27: mol_type = other DNA
gcggtagagcatgggactcttaatccc

SEQ ID NO: 15 - DNA, 27 bp, synthetic construct
  source 1..27: mol_type = other DNA
gatccattggggtttccccgcgcaggt

SEQ ID NO: 16 - DNA, 18 bp, synthetic construct
  source 1..18: mol_type = other DNA
ccatgggactcttaatcc

SEQ ID NO: 17 - DNA, 21 bp, synthetic construct
  source 1..21: mol_type = other DNA
ggtaaacatctccgactggaa

SEQ ID NO: 18 - DNA, 23 bp, synthetic construct
  source 1..23: mol_type = other DNA
agggtgtgggttcgaatcccacc

SEQ ID NO: 19 - DNA, 24 bp, synthetic construct
  source 1..24: mol_type = other DNA
aaggttccgggttcgtgtcgcggc

SEQ ID NO: 20 - DNA, 24 bp, synthetic construct
  source 1..24: mol_type = other DNA
aagtttccgggttcgggccccggc

SEQ ID NO: 21 - DNA, 23 bp, synthetic construct
  source 1..23: mol_type = other DNA
aggttgtggattcgtgtcccacc

SEQ ID NO: 22 - DNA, 24 bp, synthetic construct
  source 1..24: mol_type = other DNA
gaagttccgggttcgggtcccggc

SEQ ID NO: 23 - DNA, 23 bp, synthetic construct
  source 1..23: mol_type = other DNA
aggctgtgggttcgaatcccacc

SEQ ID NO: 24 - DNA, 17 bp, synthetic construct
  source 1..17: mol_type = other DNA
gggtgtgatgacttaca

SEQ ID NO: 25 - DNA, 24 bp, synthetic construct
  source 1..24: mol_type = other DNA
aagtttccgggttcgggacccggc

SEQ ID NO: 26 - DNA, 24 bp, synthetic construct
  source 1..24: mol_type = other DNA
aaggttccgggttcggttcccggc

SEQ ID NO: 27 - DNA, 19 bp, synthetic construct
  source 1..19: mol_type = other DNA
actgtggactctgaatcca

SEQ ID NO: 28 - DNA, 24 bp, synthetic construct
  source 1..24: mol_type = other DNA
aaggttccgggttcgggtaccggc

SEQ ID NO: 29 - DNA, 19 bp, synthetic construct
  source 1..19: mol_type = other DNA
gcacgggactcttaatccc

SEQ ID NO: 30 - DNA, 24 bp, synthetic construct
  source 1..24: mol_type = other DNA
aagtttgtgggttcgtatcccacc

SEQ ID NO: 31 - DNA, 23 bp, synthetic construct
  source 1..23: mol_type = other DNA
ggagtgtgggttcgtgtcccatc

SEQ ID NO: 32 - DNA, 23 bp, synthetic construct
  source 1..23: mol_type = other DNA
aggttgtgggttcgaggcccacc

SEQ ID NO: 33 - DNA, 25 bp, synthetic construct
  source 1..25: mol_type = other DNA
agagtttccgggttcgtgtcccggc

SEQ ID NO: 34 - DNA, 18 bp, synthetic construct
  source 1..18: mol_type = other DNA
ttgagggtgcgtgtccct

SEQ ID NO: 35 - DNA, 25 bp, synthetic construct
  source 1..25: mol_type = other DNA
agaggttccggggtcgggtcccggc

SEQ ID NO: 36 - DNA, 21 bp, synthetic construct
  source 1..21: mol_type = other DNA
agtgtgagggttcgtgtccct

SEQ ID NO: 37 - DNA, 22 bp, synthetic construct
  source 1..22: mol_type = other DNA
cacccgtagtaccgacctcgcg

SEQ ID NO: 38 - DNA, 25 bp, synthetic construct
  source 1..25: mol_type = other DNA
agaggttccgagttcgggtcccggc

SEQ ID NO: 39 - DNA, 33 bp, synthetic construct
  source 1..33: mol_type = other DNA
tccccggtggtctagtggttaggattccgcgct

SEQ ID NO: 40 - DNA, 17 bp, synthetic construct
  source 1..17: mol_type = other DNA
gacgtcggatcagaaga

SEQ ID NO: 41 - DNA, 17 bp, synthetic construct
  source 1..17: mol_type = other DNA
ttttgggatgacttaca

SEQ ID NO: 42 - DNA, 22 bp, synthetic construct
  source 1..22: mol_type = other DNA
ttcacgtaatccaggaaaagct

SEQ ID NO: 43 - DNA, 24 bp, synthetic construct
  source 1..24: mol_type = other DNA
gaggttacgggttcgtgtcccggc

SEQ ID NO: 44 - DNA, 17 bp, synthetic construct
  source 1..17: mol_type = other DNA
atgtgactcttaatctc

SEQ ID NO: 45 - DNA, 21 bp, synthetic construct
  source 1..21: mol_type = other DNA
agggtgtgggttcgtcccacc

SEQ ID NO: 46 - DNA, 27 bp, synthetic construct
  source 1..27: mol_type = other DNA
tatagcactctggactctgaatccagc

SEQ ID NO: 47 - DNA, 23 bp, synthetic construct
  source 1..23: mol_type = other DNA
ccacggactcccaaaagcagctt

SEQ ID NO: 48 - DNA, 23 bp, synthetic construct
  source 1..23: mol_type = other DNA
accccgtagatccgaccttgtga

SEQ ID NO: 49 - DNA, 20 bp, synthetic construct
  source 1..20: mol_type = other DNA
tcaccgggtgtacatcaagc

SEQ ID NO: 50 - DNA, 22 bp, synthetic construct
  source 1..22: mol_type = other DNA
caacggaatctccaaagcagct

SEQ ID NO: 51 - DNA, 23 bp, synthetic construct
  source 1..23: mol_type = other DNA
tcttgcactcgtcccggcctcat

SEQ ID NO: 52 - DNA, 17 bp, synthetic construct
  source 1..17: mol_type = other DNA
tttcggcactgaggcct

SEQ ID NO: 53 - DNA, 22 bp, synthetic construct
  source 1..22: mol_type = other DNA
tcacccgggtgtcaatcagctg

SEQ ID NO: 54 - DNA, 23 bp, synthetic construct
  source 1..23: mol_type = other DNA
cccccgtcgaaccgcccttgcga

SEQ ID NO: 55 - DNA, 27 bp, synthetic construct
  source 1..27: mol_type = other DNA
gttaaaattcctgaaccgggacgcggc

SEQ ID NO: 56 - DNA, 44 bp, synthetic construct
  source 1..44: mol_type = other DNA
ggttcgtgctgacggcctgtatcctaggctacaccctgaggact

SEQ ID NO: 57 - DNA, 20 bp, synthetic construct
  source 1..20: mol_type = other DNA
cccccgtcgaaccgaccttg

SEQ ID NO: 58 - DNA, 22 bp, synthetic construct
  source 1..22: mol_type = other DNA
ttcacagtggctcagttctgcc

SEQ ID NO: 59 - DNA, 19 bp, synthetic construct
  source 1..19: mol_type = other DNA
ttaaactctgtcgtgctgg

SEQ ID NO: 60 - DNA, 21 bp, synthetic construct
  source 1..21: mol_type = other DNA
gctaataccggataagaaagc

SEQ ID NO: 61 - DNA, 32 bp, synthetic construct
  source 1..32: mol_type = other DNA
tccctggtggtctggtggttaggagtcggcgc

SEQ ID NO: 62 - DNA, 21 bp, synthetic construct
  source 1..21: mol_type = other DNA
taaagtgctgaccgtgcagat

SEQ ID NO: 63 - DNA, 25 bp, synthetic construct
  source 1..25: mol_type = other DNA
tcctctgtagttcagtcggtagaac

SEQ ID NO: 64 - DNA, 32 bp, synthetic construct
  source 1..32: mol_type = other DNA
tccctgtggtctaatggttaggatccggcgct

SEQ ID NO: 65 - DNA, 44 bp, synthetic construct
  source 1..44: mol_type = other DNA
ccttggctgggagaacgcctgggaataccgggtgctgtaggctt

SEQ ID NO: 66 - DNA, 23 bp, synthetic construct
  source 1..23: mol_type = other DNA
caacatagcgagcccccgtctct

SEQ ID NO: 67 - DNA, 20 bp, synthetic construct
  source 1..20: mol_type = other DNA
cagttgccacgttcccgtgg

SEQ ID NO: 68 - DNA, 24 bp, synthetic construct
  source 1..24: mol_type = other DNA
tgtaaacctcctggcctggaagct

SEQ ID NO: 69 - DNA, 28 bp, synthetic construct
  source 1..28: mol_type = other DNA
cgcattgccgagtagctatgttcggatg

SEQ ID NO: 70 - DNA, 44 bp, synthetic construct
  source 1..44: mol_type = other DNA
gacggaaagaccccatgaacctttactgtagctttgtattggac

SEQ ID NO: 71 - DNA, 18 bp, synthetic construct
  source 1..18: mol_type = other DNA
ggctaatacctgggactc

SEQ ID NO: 72 - DNA, 25 bp, synthetic construct
  source 1..25: mol_type = other DNA
cgcggggtggagcagcctggtagct

SEQ ID NO: 73 - DNA, 30 bp, synthetic construct
  source 1..30: mol_type = other DNA
cgggtcgtgggttcgccccacgttgggcgc

SEQ ID NO: 74 - DNA, 30 bp, synthetic construct
  source 1..30: mol_type = other DNA
tctacagtccgacgatacgactcttagcgg

SEQ ID NO: 75 - DNA, 32 bp, synthetic construct
  source 1..32: mol_type = other DNA
gggcccctacccggccgtcgccggcagtcgag

SEQ ID NO: 76 - DNA, 33 bp, synthetic construct
  source 1..33: mol_type = other DNA
tcttccgtagtgtagtggttatgacgttcgcct

SEQ ID NO: 77 - DNA, 18 bp, synthetic construct
  source 1..18: mol_type = other DNA
tcaaggctaaaactcaaa

SEQ ID NO: 78 - DNA, 23 bp, synthetic construct
  source 1..23: mol_type = other DNA
tacagtactgtgctaactgaaaa

SEQ ID NO: 79 - DNA, 25 bp, synthetic construct
  source 1..25: mol_type = other DNA
gccacggtggccgagtggttaaggc

SEQ ID NO: 80 - DNA, 26 bp, synthetic construct
  source 1..26: mol_type = other DNA
cccccactgctacatttgactgtctt

SEQ ID NO: 81 - DNA, 27 bp, synthetic construct
  source 1..27: mol_type = other DNA
acggataaaaggtacctcggggataac

SEQ ID NO: 82 - DNA, 27 bp, synthetic construct
  source 1..27: mol_type = other DNA
cttctagaaatttctgaaaatgctctg

SEQ ID NO: 83 - DNA, 29 bp, synthetic construct
  source 1..29: mol_type = other DNA
ccccccactgctaaatttgactggctact

SEQ ID NO: 84 - DNA, 31 bp, synthetic construct
  source 1..31: mol_type = other DNA
ggccgcgtgcctaatggataaggcgtctgat

SEQ ID NO: 85 - DNA, 33 bp, synthetic construct
  source 1..33: mol_type = other DNA
ctgtgagggtgagcgaatcgctgaaagccggcc

SEQ ID NO: 86 - DNA, 33 bp, synthetic construct
  source 1..33: mol_type = other DNA
gcttgcggagtgtagtggttatcacgttcgcct

SEQ ID NO: 87 - DNA, 34 bp, synthetic construct
  source 1..34: mol_type = other DNA
caacggataaaaggtactctagggataacaggct

SEQ ID NO: 88 - DNA, 34 bp, synthetic construct
  source 1..34: mol_type = other DNA
cattggtggttccgtggtagaattctcgcctgcc

SEQ ID NO: 89 - DNA, 34 bp, synthetic construct
  source 1..34: mol_type = other DNA
ggctggtccgatggtagtggggtatcagaacttg

SEQ ID NO: 90 - DNA, 40 bp, synthetic construct
  source 1..40: mol_type = other DNA
ttgaccttaccggatggcacaaagagaagtgggcaagttc

SEQ ID NO: 91 - DNA, 30 bp, synthetic construct
  source 1..30: mol_type = other DNA
tccctagttcgtttctgggagcggagacca

SEQ ID NO: 92 - DNA, 29 bp, synthetic construct
  source 1..29: mol_type = other DNA
tcccatgtggtctagcggttaggattcct

SEQ ID NO: 93 - DNA, 28 bp, synthetic construct
  source 1..28: mol_type = other DNA
cgggcctttcggggcctcttccccgggc

SEQ ID NO: 94 - DNA, 18 bp, synthetic construct
  source 1..18: mol_type = other DNA
gtggttccggctttggac

SEQ ID NO: 95 - DNA, 25 bp, synthetic construct
  source 1..25: mol_type = other DNA
gtgctaatctgcgataagcgtcggt

SEQ ID NO: 96 - DNA, 23 bp, synthetic construct
  source 1..23: mol_type = other DNA
tcagtgcatcaccgacctttgtt

SEQ ID NO: 97 - DNA, 23 bp, synthetic construct
  source 1..23: mol_type = other DNA
tccctgagaccctttaaacctgt

SEQ ID NO: 98 - DNA, 30 bp, synthetic construct
  source 1..30: mol_type = other DNA
ctagtacgagaggaccggagtggacgcatc

SEQ ID NO: 99 - DNA, 20 bp, synthetic construct
  source 1..20: mol_type = other DNA
gaggcagcagtagggaatat

SEQ ID NO: 100 - DNA, 22 bp, synthetic construct
  source 1..22: mol_type = other DNA
tagcaccatttgcaatcggttg

SEQ ID NO: 101 - DNA, 25 bp, synthetic construct
  source 1..25: mol_type = other DNA
ttagacagttcggtccctatctgcc

SEQ ID NO: 102 - DNA, 28 bp, synthetic construct
  source 1..28: mol_type = other DNA
tgatgtcggctcatctcatcctggggct

SEQ ID NO: 103 - DNA, 18 bp, synthetic construct
  source 1..18: mol_type = other DNA
aatcctggtcggacatca

SEQ ID NO: 104 - DNA, 23 bp, synthetic construct
  source 1..23: mol_type = other DNA
tgcaccatggttctctgagcatg

SEQ ID NO: 105 - DNA, 30 bp, synthetic construct
  source 1..30: mol_type = other DNA
tggggagttcgagtctctccgcccctgcca

SEQ ID NO: 106 - DNA, 38 bp, synthetic construct
  source 1..38: mol_type = other DNA
ccaaggggtcgtgggttcgaatcctgccagccgcacca

SEQ ID NO: 107 - DNA, 18 bp, synthetic construct
  source 1..18: mol_type = other DNA
tcgtgatacagttcggtc

SEQ ID NO: 108 - DNA, 29 bp, synthetic construct
  source 1..29: mol_type = other DNA
tccggggagcacgcctgttcgagtatcgt

SEQ ID NO: 109 - DNA, 36 bp, synthetic construct
  source 1..36: mol_type = other DNA
gccccgttcgtctagcggcctaggacgccggcctct

SEQ ID NO: 110 - DNA, 17 bp, synthetic construct
  source 1..17: mol_type = other DNA
cttccacaacgttcccg

SEQ ID NO: 111 - DNA, 18 bp, synthetic construct
  source 1..18: mol_type = other DNA
ttcgatcccgtcatcacc

SEQ ID NO: 112 - DNA, 20 bp, synthetic construct
  source 1..20: mol_type = other DNA
aaagaggaggagaggagaac

SEQ ID NO: 113 - DNA, 30 bp, synthetic construct
  source 1..30: mol_type = other DNA
tccaccacgttcccgtggtaaatcagcttg

SEQ ID NO: 114 - DNA, 41 bp, synthetic construct
  source 1..41: mol_type = other DNA
gcaagcaggggtcgtcggttcgatcccgtcatcctccacca

SEQ ID NO: 115 - DNA, 18 bp, synthetic construct
  source 1..18: mol_type = other DNA
cccccacgttcccgttgg

SEQ ID NO: 116 - DNA, 19 bp, synthetic construct
  source 1..19: mol_type = other DNA
tttggtatctgcgctctgc

SEQ ID NO: 117 - DNA, 22 bp, synthetic construct
  source 1..22: mol_type = other DNA
caccttgcgcaatcaggactga

SEQ ID NO: 118 - DNA, 23 bp, synthetic construct
  source 1..23: mol_type = other DNA
gggatagtaggtcgttgccaacc

SEQ ID NO: 119 - DNA, 24 bp, synthetic construct
  source 1..24: mol_type = other DNA
ggaagaacgggtgctgtaggcttt

SEQ ID NO: 120 - DNA, 28 bp, synthetic construct
  source 1..28: mol_type = other DNA
cgagaccaggactttgataggctgggtg

SEQ ID NO: 121 - DNA, 33 bp, synthetic construct
  source 1..33: mol_type = other DNA
aagcagcaatgcgacgtatagggtctgacgcct

SEQ ID NO: 122 - DNA, 33 bp, synthetic construct
  source 1..33: mol_type = other DNA
tcaaatggtagagcgctcgcttggcttgcgaga

SEQ ID NO: 123 - DNA, 34 bp, synthetic construct
  source 1..34: mol_type = other DNA
gacccagttgcctaattggataaggcatcagcct

SEQ ID NO: 124 - DNA, 35 bp, synthetic construct
  source 1..35: mol_type = other DNA
tccctggtggtctggtggttaggagtcggcgctct

SEQ ID NO: 125 - DNA, 17 bp, synthetic construct
  source 1..17: mol_type = other DNA
atagatcctgaaaccgc

SEQ ID NO: 126 - DNA, 19 bp, synthetic construct
  source 1..19: mol_type = other DNA
ctcttcgaggccctgtaat

SEQ ID NO: 127 - DNA, 20 bp, synthetic construct
  source 1..20: mol_type = other DNA
aggtcctcaatacgtatttg

SEQ ID NO: 128 - DNA, 20 bp, synthetic construct
  source 1..20: mol_type = other DNA
caaggcaaagacgcgtagct

SEQ ID NO: 129 - DNA, 23 bp, synthetic construct
  source 1..23: mol_type = other DNA
aactggagagtttgattctggct

SEQ ID NO: 130 - DNA, 23 bp, synthetic construct
  source 1..23: mol_type = other DNA
cggtgaatacgttcccgggcctt

SEQ ID NO: 131 - DNA, 23 bp, synthetic construct
  source 1..23: mol_type = other DNA
ttccctttttaatcctatgcctg

SEQ ID NO: 132 - DNA, 24 bp, synthetic construct
  source 1..24: mol_type = other DNA
agcacgcgcgcacgtgttaggacc

SEQ ID NO: 133 - DNA, 26 bp, synthetic construct
  source 1..26: mol_type = other DNA
cagatggcggaattggtagacgcgct

SEQ ID NO: 134 - DNA, 26 bp, synthetic construct
  source 1..26: mol_type = other DNA
cgtggttcatttccccctttcgggcg

SEQ ID NO: 135 - DNA, 27 bp, synthetic construct
  source 1..27: mol_type = other DNA
ggtcgatgatgattggtaaaaggtctg

SEQ ID NO: 136 - DNA, 31 bp, synthetic construct
  source 1..31: mol_type = other DNA
gtcgccggttcaagtccggcagtcggctcca

SEQ ID NO: 137 - DNA, 36 bp, synthetic construct
  source 1..36: mol_type = other DNA
aacaccgtggaagttcgagtcttctcctgggcacca

SEQ ID NO: 138 - DNA, 17 bp, synthetic construct
  source 1..17: mol_type = other DNA
agggatgtcgctcaacg

SEQ ID NO: 139 - DNA, 17 bp, synthetic construct
  source 1..17: mol_type = other DNA
gcctgtagtcgtgcccg

SEQ ID NO: 140 - DNA, 19 bp, synthetic construct
  source 1..19: mol_type = other DNA
aatcgatcgagggcttaac

SEQ ID NO: 141 - DNA, 19 bp, synthetic construct
  source 1..19: mol_type = other DNA
gcaaccatcctctgctacc

SEQ ID NO: 142 - DNA, 19 bp, synthetic construct
  source 1..19: mol_type = other DNA
tcaacttcggaactgcctt

SEQ ID NO: 143 - DNA, 21 bp, synthetic construct
  source 1..21: mol_type = other DNA
acattgggactgagccacggc

SEQ ID NO: 144 - DNA, 21 bp, synthetic construct
  source 1..21: mol_type = other DNA
ggaggggagtgaaatagaacc

SEQ ID NO: 145 - DNA, 21 bp, synthetic construct
  source 1..21: mol_type = other DNA
tgaataccgtgctgtaggctt

SEQ ID NO: 146 - DNA, 22 bp, synthetic construct
  source 1..22: mol_type = other DNA
ctaatcgatcgagggcttaacc

SEQ ID NO: 147 - DNA, 22 bp, synthetic construct
  source 1..22: mol_type = other DNA
tgaccgggagtcaatcagcttg

SEQ ID NO: 148 - DNA, 22 bp, synthetic construct
  source 1..22: mol_type = other DNA
tgaggggcagagcgcgagacta

SEQ ID NO: 149 - DNA, 22 bp, synthetic construct
  source 1..22: mol_type = other DNA
tgcggacaaggggaatctgact

SEQ ID NO: 150 - DNA, 22 bp, synthetic construct
  source 1..22: mol_type = other DNA
ttatgtagtagattgttatagt

SEQ ID NO: 151 - DNA, 23 bp, synthetic construct
  source 1..23: mol_type = other DNA
ccccgtccgccccccgttccccc

SEQ ID NO: 152 - DNA, 23 bp, synthetic construct
  source 1..23: mol_type = other DNA
ggaggggcagagagcgagccttt

SEQ ID NO: 153 - DNA, 23 bp, synthetic construct
  source 1..23: mol_type = other DNA
taggggtgaaaggctaaacaaac

SEQ ID NO: 154 - DNA, 23 bp, synthetic construct
  source 1..23: mol_type = other DNA
tgtctgaacatggggggaccacc

SEQ ID NO: 155 - DNA, 23 bp, synthetic construct
  source 1..23: mol_type = other DNA
ttcattcggctgtccgagatgta

SEQ ID NO: 156 - DNA, 24 bp, synthetic construct
  source 1..24: mol_type = other DNA
agctagacagcaggacggtggcca

SEQ ID NO: 157 - DNA, 25 bp, synthetic construct
  source 1..25: mol_type = other DNA
ttatggccaggctgtctccacccga

SEQ ID NO: 158 - DNA, 26 bp, synthetic construct
  source 1..26: mol_type = other DNA
aatagaacctgaaaccggatgcctac

SEQ ID NO: 159 - DNA, 27 bp, synthetic construct
  source 1..27: mol_type = other DNA
cgcgctcgccggccgaggtgggatccc

SEQ ID NO: 160 - DNA, 28 bp, synthetic construct
  source 1..28: mol_type = other DNA
gcggatgtggctcagctggtagagcatc

SEQ ID NO: 161 - DNA, 32 bp, synthetic construct
  source 1..32: mol_type = other DNA
ctcgtaccaaacgagaactttgaaggccgaag

SEQ ID NO: 162 - DNA, 33 bp, synthetic construct
  source 1..33: mol_type = other DNA
gcggctgtagtgtagtggtgatcacgttcgccc

SEQ ID NO: 163 - DNA, 34 bp, synthetic construct
  source 1..34: mol_type = other DNA
acgtagaggccggaggttcgaatcctctcacccc

SEQ ID NO: 164 - DNA, 35 bp, synthetic construct
  source 1..35: mol_type = other DNA
tcattggtggttcagtggtagacttctcgcctgcc

SEQ ID NO: 165 - DNA, 37 bp, synthetic construct
  source 1..37: mol_type = other DNA
acgatgtgggattgcattgacaatcaggaggttggct

SEQ ID NO: 166 - DNA, 38 bp, synthetic construct
  source 1..38: mol_type = other DNA
aacctatctgtgtaggataggtgggaggctttgaagtc

SEQ ID NO: 167 - DNA, 20 bp, synthetic construct
  source 1..20: mol_type = other DNA
ctaaatactcgtacatgacc

SEQ ID NO: 168 - DNA, 22 bp, synthetic construct
  source 1..22: mol_type = other DNA
ccctagcttgtgcgctcctgga

SEQ ID NO: 169 - DNA, 22 bp, synthetic construct
  source 1..22: mol_type = other DNA
tgcaactcgactccatgaagtc

SEQ ID NO: 170 - DNA, 25 bp, synthetic construct
  source 1..25: mol_type = other DNA
tccccgtaatcttcataatccggag

SEQ ID NO: 171 - DNA, 33 bp, synthetic construct
  source 1..33: mol_type = other DNA
gcattggtggttcggtggtagaatgctcgcctg

SEQ ID NO: 172 - DNA, 31 bp, synthetic construct
  source 1..31: mol_type = other DNA
ttcgagccccgcgggtgcttactgacccttt

SEQ ID NO: 173 - DNA, 43 bp, synthetic construct
  source 1..43: mol_type = other DNA
acttggctgggagaccgcctgggaataccgggtgctgtatgct

SEQ ID NO: 174 - DNA, 34 bp, synthetic construct
  source 1..34: mol_type = other DNA
ccccatgaagtcggagtcgctagtaatcgcagat

SEQ ID NO: 175 - DNA, 42 bp, synthetic construct
  source 1..42: mol_type = other DNA
aattggcatgagtccactttaaatcctttaacgaggatccat

SEQ ID NO: 176 - DNA, 19 bp, synthetic construct
  source 1..19: mol_type = other DNA
caaaactcccgtgctgatc

SEQ ID NO: 177 - DNA, 32 bp, synthetic construct
  source 1..32: mol_type = other DNA
tgcccgttggtctagggggatgattctcgctt

SEQ ID NO: 178 - DNA, 33 bp, synthetic construct
  source 1..33: mol_type = other DNA
tcctcgatagctcagttggtagagcgccggact

SEQ ID NO: 179 - DNA, 21 bp, synthetic construct
  source 1..21: mol_type = other DNA
cgagcccaggttggagagcca

SEQ ID NO: 180 - DNA, 21 bp, synthetic construct
  source 1..21: mol_type = other DNA
gatcagctaccgtcgtagttc

SEQ ID NO: 181 - DNA, 23 bp, synthetic construct
  source 1..23: mol_type = other DNA
gtctttttgtcctcctatgcctg

SEQ ID NO: 182 - DNA, 25 bp, synthetic construct
  source 1..25: mol_type = other DNA
atggttcgcactctggactctgaat

SEQ ID NO: 183 - DNA, 34 bp, synthetic construct
  source 1..34: mol_type = other DNA
ccacgttcccgtggattccaccacgttcccgggg

SEQ ID NO: 184 - DNA, 35 bp, synthetic construct
  source 1..35: mol_type = other DNA
cctaaaaagacggatgttgctgagtgtggacctgg

SEQ ID NO: 185 - DNA, 19 bp, synthetic construct
  source 1..19: mol_type = other DNA
tagaaaccgggcggaaaca

SEQ ID NO: 186 - DNA, 35 bp, synthetic construct
  source 1..35: mol_type = other DNA
ctggagaccggggttcgatttcccgacggggagcc

SEQ ID NO: 187 - DNA, 35 bp, synthetic construct
  source 1..35: mol_type = other DNA
tctgctgaggctaagcccgtgttctaaagatttgt

SEQ ID NO: 188 - DNA, 37 bp, synthetic construct
  source 1..37: mol_type = other DNA
ccatgtgtcgtaggttcgaatcctatcggggccgcca

SEQ ID NO: 189 - DNA, 21 bp, synthetic construct
  source 1..21: mol_type = other DNA
tcagtgcatgaccgaacttgt

SEQ ID NO: 190 - DNA, 25 bp, synthetic construct
  source 1..25: mol_type = other DNA
tagttggttttcggaactgaggcca

SEQ ID NO: 191 - DNA, 40 bp, synthetic construct
  source 1..40: mol_type = other DNA
ggacagtgtctggtgggtagtttgactggggcggtctcct

SEQ ID NO: 192 - DNA, 23 bp, synthetic construct
  source 1..23: mol_type = other DNA
tgccctttgtcatcctcttcctg

SEQ ID NO: 193 - DNA, 36 bp, synthetic construct
  source 1..36: mol_type = other DNA
cgctacctcagatcaggacgtggcgacccgctgaat

SEQ ID NO: 194 - DNA, 34 bp, synthetic construct
  source 1..34: mol_type = other DNA
gttgtcgtgggttcgagccccatcagccacccca

SEQ ID NO: 195 - DNA, 28 bp, synthetic construct
  source 1..28: mol_type = other DNA
gcggaagtagttcagtggtagaacatca

SEQ ID NO: 196 - DNA, 41 bp, synthetic construct
  source 1..41: mol_type = other DNA
cgcgacctcagatcagacgtggcgacccgctgagtgtaagc

SEQ ID NO: 197 - DNA, 25 bp, synthetic construct
  source 1..25: mol_type = other DNA
gcaggttcagtcctgccgcggtcgc

SEQ ID NO: 198 - DNA, 27 bp, synthetic construct
  source 1..27: mol_type = other DNA
gtgatatagacagcaggacggtggcca

SEQ ID NO: 199 - DNA, 29 bp, synthetic construct
  source 1..29: mol_type = other DNA
ccagtgtgaaagtaggttatcttcaggct

SEQ ID NO: 200 - DNA, 21 bp, synthetic construct
  source 1..21: mol_type = other DNA
gtaccgggtgtaaatcagctg

SEQ ID NO: 201 - DNA, 28 bp, synthetic construct
  source 1..28: mol_type = other DNA
caccgaaatcgcggatatgagcgttcct

SEQ ID NO: 202 - DNA, 31 bp, synthetic construct
  source 1..31: mol_type = other DNA
agtctggcacggtgaagagacatgagagggg

SEQ ID NO: 203 - DNA, 33 bp, synthetic construct
  source 1..33: mol_type = other DNA
gtaaccggggttcgaatccccgtagggacgcca

SEQ ID NO: 204 - DNA, 17 bp, synthetic construct
  source 1..17: mol_type = other DNA
gctgcatggccgtcgtc

SEQ ID NO: 205 - DNA, 18 bp, synthetic construct
  source 1..18: mol_type = other DNA
cgggcgctgtaggctttt

SEQ ID NO: 206 - DNA, 18 bp, synthetic construct
  source 1..18: mol_type = other DNA
gtcctctcggccgcacca

SEQ ID NO: 207 - DNA, 21 bp, synthetic construct
  source 1..21: mol_type = other DNA
cgcagagtcgcgcagcggaag

SEQ ID NO: 208 - DNA, 22 bp, synthetic construct
  source 1..22: mol_type = other DNA
cggggtgtagcttagcctggta

SEQ ID NO: 209 - DNA, 23 bp, synthetic construct
  source 1..23: mol_type = other DNA
gccggctagctcagtcggtagag

SEQ ID NO: 210 - DNA, 23 bp, synthetic construct
  source 1..23: mol_type = other DNA
ttccgtttgtcatcctatggctg

SEQ ID NO: 211 - DNA, 24 bp, synthetic construct
  source 1..24: mol_type = other DNA
atcctgtctgaatatggggggacc

SEQ ID NO: 212 - DNA, 25 bp, synthetic construct
  source 1..25: mol_type = other DNA
ggctcataacccgaaggtcgtcggt

SEQ ID NO: 213 - DNA, 27 bp, synthetic construct
  source 1..27: mol_type = other DNA
tccagggttcagttccctgttcgggcg

SEQ ID NO: 214 - DNA, 29 bp, synthetic construct
  source 1..29: mol_type = other DNA
acggataaaaggtacctcggggataacag

SEQ ID NO: 215 - DNA, 32 bp, synthetic construct
  source 1..32: mol_type = other DNA
gcatttgtggtgcagtggtagaattctagcct

SEQ ID NO: 216 - DNA, 40 bp, synthetic construct
  source 1..40: mol_type = other DNA
cacaacgagatcacctctgggtcgtctgccggtctccacc

SEQ ID NO: 217 - DNA, 41 bp, synthetic construct
  source 1..41: mol_type = other DNA
ctgcactacagcctgggcaacatagcgagaccccgtctcta

SEQ ID NO: 218 - DNA, 18 bp, synthetic construct
  source 1..18: mol_type = other DNA
attgaccgattgagagct

SEQ ID NO: 219 - DNA, 20 bp, synthetic construct
  source 1..20: mol_type = other DNA
ccggggccacgtgcccgtgg

SEQ ID NO: 220 - DNA, 21 bp, synthetic construct
  source 1..21: mol_type = other DNA
gttcagatcccggacgagcca

SEQ ID NO: 221 - DNA, 25 bp, synthetic construct
  source 1..25: mol_type = other DNA
tcaaacagaactttgaaggccgaag

SEQ ID NO: 222 - DNA, 26 bp, synthetic construct
  source 1..26: mol_type = other DNA
cgtgttcaggtgacgtcggggtcacc

SEQ ID NO: 223 - DNA, 26 bp, synthetic construct
  source 1..26: mol_type = other DNA
tgtcgggctggggcgcgaagcggggc

SEQ ID NO: 224 - DNA, 33 bp, synthetic construct
  source 1..33: mol_type = other DNA
gcccggctagctcagtcggtagatcatgagaca

SEQ ID NO: 225 - DNA, 33 bp, synthetic construct
  source 1..33: mol_type = other DNA
tcccacatcgtccagcggttaggattcctggtt

SEQ ID NO: 226 - DNA, 34 bp, synthetic construct
  source 1..34: mol_type = other DNA
tccctggtggtctagtgactaggattcggcgctt

SEQ ID NO: 227 - DNA, 17 bp, synthetic construct
  source 1..17: mol_type = other DNA
acaaaccggaggaaggt

SEQ ID NO: 228 - DNA, 34 bp, synthetic construct
  source 1..34: mol_type = other DNA
ctcgacccttcgaacgcacttgcggccccgggtt

SEQ ID NO: 229 - DNA, 19 bp, synthetic construct
  source 1..19: mol_type = other DNA
gtagtaccgccatgtctgt

SEQ ID NO: 230 - DNA, 22 bp, synthetic construct
  source 1..22: mol_type = other DNA
cggtggcaccacgttcccgggg

SEQ ID NO: 231 - DNA, 42 bp, synthetic construct
  source 1..42: mol_type = other DNA
gccacgatcgactgagattcagcctttgttctgtagatttgt

SEQ ID NO: 232 - DNA, 21 bp, synthetic construct
  source 1..21: mol_type = other DNA
tagaggttatcacgtctgctt

SEQ ID NO: 233 - DNA, 26 bp, synthetic construct
  source 1..26: mol_type = other DNA
cagatggtagtgggttatcagaactt

SEQ ID NO: 234 - DNA, 33 bp, synthetic construct
  source 1..33: mol_type = other DNA
gcttgcgtagggtagtggttatcacgttcgcct

SEQ ID NO: 235 - DNA, 34 bp, synthetic construct
  source 1..34: mol_type = other DNA
tagaccgcctgggaataccggttgctgtaggctt

SEQ ID NO: 236 - DNA, 31 bp, synthetic construct
  source 1..31: mol_type = other DNA
gggaggctttgaagtgtggacgccagtctgc

SEQ ID NO: 237 - DNA, 22 bp, synthetic construct
  source 1..22: mol_type = other DNA
gggatgaaccgaccgccgggtt

SEQ ID NO: 238 - DNA, 31 bp, synthetic construct
  source 1..31: mol_type = other DNA
gtcggcagttcaatcctgcccatgggcacca

SEQ ID NO: 239 - DNA, 41 bp, synthetic construct
  source 1..41: mol_type = other DNA
atagtgcgtgttcccgtgtgaaagtaggtcatcgtcaggct

SEQ ID NO: 240 - DNA, 19 bp, synthetic construct
  source 1..19: mol_type = other DNA
ggtcatctcgggggaacct

SEQ ID NO: 241 - DNA, 39 bp, synthetic construct
  source 1..39: mol_type = other DNA
cactccagcctgggcaacatagcgcgaccccgtctctta

SEQ ID NO: 242 - DNA, 20 bp, synthetic construct
  source 1..20: mol_type = other DNA
tacgcctgtctgggcgtcgc

SEQ ID NO: 243 - DNA, 21 bp, synthetic construct
  source 1..21: mol_type = other DNA
tgaccggggtaaataagcttg

SEQ ID NO: 244 - DNA, 34 bp, synthetic construct
  source 1..34: mol_type = other DNA
cagcgatccgaggtcaaatctcggtggaacctcc

SEQ ID NO: 245 - DNA, 35 bp, synthetic construct
  source 1..35: mol_type = other DNA
ggctggtccgatgggagggggttatcagaacttat

SEQ ID NO: 246 - DNA, 24 bp, synthetic construct
  source 1..24: mol_type = other DNA
cagttcggtccctatctgccgtgg

SEQ ID NO: 247 - DNA, 22 bp, synthetic construct
  source 1..22: mol_type = other DNA
tcagtgcactaaagcactttgt

SEQ ID NO: 248 - DNA, 26 bp, synthetic construct
  source 1..26: mol_type = other DNA
gacggattgcgtaacttgttcagact

SEQ ID NO: 249 - DNA, 25 bp, synthetic construct
  source 1..25: mol_type = other DNA
tgggagagtaggtcgccgccggaca

SEQ ID NO: 250 - DNA, 29 bp, synthetic construct
  source 1..29: mol_type = other DNA
gacgaagactgacgctcaggtgcgaaagc

SEQ ID NO: 251 - DNA, 19 bp, synthetic construct
  source 1..19: mol_type = other DNA
ggggtagagcactgtttag

SEQ ID NO: 252 - DNA, 27 bp, synthetic construct
  source 1..27: mol_type = other DNA
gaagtagaaaagagcacatggtggatg

SEQ ID NO: 253 - DNA, 21 bp, synthetic construct
  source 1..21: mol_type = other DNA
tattacactcgtcccggcctc

SEQ ID NO: 254 - DNA, 35 bp, synthetic construct
  source 1..35: mol_type = other DNA
tacctggtggtatagtggttaggattcggcgctct

SEQ ID NO: 255 - DNA, 18 bp, synthetic construct
  source 1..18: mol_type = other DNA
cgtgttcggactggggtc

SEQ ID NO: 256 - DNA, 34 bp, synthetic construct
  source 1..34: mol_type = other DNA
tgtgattagagggctggaactttcacccccaccc

SEQ ID NO: 257 - DNA, 43 bp, synthetic construct
  source 1..43: mol_type = other DNA
tctgttacggaactgtactctctgagggcctcccacctgattc

SEQ ID NO: 258 - DNA, 44 bp, synthetic construct
  source 1..44: mol_type = other DNA
cacctgtgcgtgtgggtgctgctgcgggctgtcagatgctgacc

SEQ ID NO: 259 - DNA, 18 bp, synthetic construct
  source 1..18: mol_type = other DNA
ctcagatcagacgtggcg

SEQ ID NO: 260 - DNA, 31 bp, synthetic construct
  source 1..31: mol_type = other DNA
tttgagaggatgatcagccacactgggactg

SEQ ID NO: 261 - DNA, 34 bp, synthetic construct
  source 1..34: mol_type = other DNA
ctgtttcaaccaacgcttgactgagaactctttc

SEQ ID NO: 262 - DNA, 33 bp, synthetic construct
  source 1..33: mol_type = other DNA
tcagggtcagtctaagtgaagacaaagagaggc

SEQ ID NO: 263 - DNA, 30 bp, synthetic construct
  source 1..30: mol_type = other DNA
agtgcgagtttgagggctgtgaccggcgct

SEQ ID NO: 264 - DNA, 19 bp, synthetic construct
  source 1..19: mol_type = other DNA
catgttgctttatttatca

SEQ ID NO: 265 - DNA, 27 bp, synthetic construct
  source 1..27: mol_type = other DNA
tgtgggagagtaggacgccgccggaca

SEQ ID NO: 266 - DNA, 43 bp, synthetic construct
  source 1..43: mol_type = other DNA
tctgttacggaactgtactctctgagggcctcccacctgactc

SEQ ID NO: 267 - DNA, 23 bp, synthetic construct
  source 1..23: mol_type = other DNA
aggactggtggagcgcttagaag

SEQ ID NO: 268 - DNA, 39 bp, synthetic construct
  source 1..39: mol_type = other DNA
gccccagtggcctaatggataaggcattggcttagggac

SEQ ID NO: 269 - DNA, 43 bp, synthetic construct
  source 1..43: mol_type = other DNA
cagggcacggtatttcttgttacttccctgcacacggactgtg

SEQ ID NO: 270 - DNA, 29 bp, synthetic construct
  source 1..29: mol_type = other DNA
tacaaggaaggtcactaccgttctttcac

SEQ ID NO: 271 - DNA, 30 bp, synthetic construct
  source 1..30: mol_type = other DNA
ctgctttcttctttggatcgtcgttcaact

SEQ ID NO: 272 - DNA, 30 bp, synthetic construct
  source 1..30: mol_type = other DNA
ttagcaacaacaggaagccccttttatcct

SEQ ID NO: 273 - DNA, 23 bp, synthetic construct
  source 1..23: mol_type = other DNA
tctgaatcaacccttattactct

SEQ ID NO: 274 - DNA, 34 bp, synthetic construct
  source 1..34: mol_type = other DNA
tctcatttgggcagaatatgtcagagggaagatc

SEQ ID NO: 275 - DNA, 18 bp, synthetic construct
  source 1..18: mol_type = other DNA
cctcctaagtattacacc

SEQ ID NO: 276 - DNA, 22 bp, synthetic construct
  source 1..22: mol_type = other DNA
cccatcttgctgagatgaggcc

SEQ ID NO: 277 - DNA, 26 bp, synthetic construct
  source 1..26: mol_type = other DNA
ccttgtaataacctctagtcctttcc

SEQ ID NO: 278 - DNA, 29 bp, synthetic construct
  source 1..29: mol_type = other DNA
attcatggtgctttcaagtcaggttttct

SEQ ID NO: 279 - DNA, 17 bp, synthetic construct
  source 1..17: mol_type = other DNA
catcagagacagtggca

SEQ ID NO: 280 - DNA, 20 bp, synthetic construct
  source 1..20: mol_type = other DNA
ccctgaagatgtaactgtca

SEQ ID NO: 281 - DNA, 24 bp, synthetic construct
  source 1..24: mol_type = other DNA
ccctgaagcataccaaaatgtgtc

SEQ ID NO: 282 - DNA, 25 bp, synthetic construct
  source 1..25: mol_type = other DNA
tgaaaaggactttgaaaagagagtc

SEQ ID NO: 283 - DNA, 20 bp, synthetic construct
  source 1..20: mol_type = other DNA
ctgtcgggacccgaaagatg

SEQ ID NO: 284 - DNA, 17 bp, synthetic construct
  source 1..17: mol_type = other DNA
tcatctcatcctggggc

SEQ ID NO: 285 - DNA, 21 bp, synthetic construct
  source 1..21: mol_type = other DNA
ctactctgaacgattgagacc

SEQ ID NO: 286 - DNA, 23 bp, synthetic construct
  source 1..23: mol_type = other DNA
cggcgggctgtcagattctcacc

SEQ ID NO: 287 - DNA, 32 bp, synthetic construct
  source 1..32: mol_type = other DNA
gggtgattagctcagctgggagagcgtctgcc

SEQ ID NO: 288 - DNA, 38 bp, synthetic construct
  source 1..38: mol_type = other DNA
ccctagtcttcatttgttgttatgtcattgcctgcctt

SEQ ID NO: 289 - DNA, 42 bp, synthetic construct
  source 1..42: mol_type = other DNA
cccaggttcaagtgattctcctgcctcagcctccagagtacc

SEQ ID NO: 290 - DNA, 17 bp, synthetic construct
  source 1..17: mol_type = other DNA
cttcacctgagagtgtc

SEQ ID NO: 291 - DNA, 20 bp, synthetic construct
  source 1..20: mol_type = other DNA
ccccagaagcaggtgtcaat

SEQ ID NO: 292 - DNA, 40 bp, synthetic construct
  source 1..40: mol_type = other DNA
cccatatttcataatttcacgcttctgtcttgcatgcttc

SEQ ID NO: 293 - DNA, 44 bp, synthetic construct
  source 1..44: mol_type = other DNA
cacttgtgcttgtgggtgctactgcgggcggtcagatgctcacc

SEQ ID NO: 294 - DNA, 44 bp, synthetic construct
  source 1..44: mol_type = other DNA
tgggcagtggcttatgggaagatgacctctgattaaataattcc

SEQ ID NO: 295 - DNA, 42 bp, synthetic construct
  source 1..42: mol_type = other DNA
cgcgacctcagatccgacgtggcgacccgctgaatttaagcc

SEQ ID NO: 296 - DNA, 23 bp, synthetic construct
  source 1..23: mol_type = other DNA
cgtgagagaactcgggtgaagga

SEQ ID NO: 297 - DNA, 31 bp, synthetic construct
  source 1..31: mol_type = other DNA
aagcactgaaccgggcgactagtactagagt

SEQ ID NO: 298 - DNA, 38 bp, synthetic construct
  source 1..38: mol_type = other DNA
ctgtctggactacttctttctctgattaatgccttgct

SEQ ID NO: 299 - DNA, 19 bp, synthetic construct
  source 1..19: mol_type = other DNA
catttcctccattgtgtcc

SEQ ID NO: 300 - DNA, 41 bp, synthetic construct
  source 1..41: mol_type = other DNA
tatttgcgtagaggtgtttgtagtattctctgatggtagta

SEQ ID NO: 301 - DNA, 32 bp, synthetic construct
  source 1..32: mol_type = other DNA
cctcctggagagatctcttgagttcctgcctc

SEQ ID NO: 302 - DNA, 25 bp, synthetic construct
  source 1..25: mol_type = other DNA
cgggagagtaggtcgcgccaggtcc

SEQ ID NO: 303 - DNA, 27 bp, synthetic construct
  source 1..27: mol_type = other DNA
ctgtaagtgtttggagttggaatttac

SEQ ID NO: 304 - DNA, 41 bp, synthetic construct
  source 1..41: mol_type = other DNA
ccatgcctgtggcacacttctgtccttcacgctgtcttctc

SEQ ID NO: 305 - DNA, 42 bp, synthetic construct
  source 1..42: mol_type = other DNA
ccctctctcagcatttttgctgttcgtgaaatgaggacatag

SEQ ID NO: 306 - DNA, 24 bp, synthetic construct
  source 1..24: mol_type = other DNA
ccgagatggatctggctgggaccc

SEQ ID NO: 307 - DNA, 43 bp, synthetic construct
  source 1..43: mol_type = other DNA
tctgttacggaagtgtactctctgagggcctcccacctgagtc

SEQ ID NO: 308 - DNA, 17 bp, synthetic construct
  source 1..17: mol_type = other DNA
agaagaagaagaggaag

SEQ ID NO: 309 - DNA, 20 bp, synthetic construct
  source 1..20: mol_type = other DNA
cccagagtccatatcaatgg

SEQ ID NO: 310 - DNA, 20 bp, synthetic construct
  source 1..20: mol_type = other DNA
gagaggaccgggttggacga

SEQ ID NO: 311 - DNA, 21 bp, synthetic construct
  source 1..21: mol_type = other DNA
aaagggaaggctgaactgctg

SEQ ID NO: 312 - DNA, 27 bp, synthetic construct
  source 1..27: mol_type = other DNA
atggggtgcaagctcttgatcgaagcc

SEQ ID NO: 313 - DNA, 17 bp, synthetic construct
  source 1..17: mol_type = other DNA
actgtagtaactcctac

SEQ ID NO: 314 - DNA, 22 bp, synthetic construct
  source 1..22: mol_type = other DNA
tctttaggatcaatttccattc

SEQ ID NO: 315 - DNA, 31 bp, synthetic construct
  source 1..31: mol_type = other DNA
aagcgagtctgaacagggcgactgagtttga

SEQ ID NO: 316 - DNA, 31 bp, synthetic construct
  source 1..31: mol_type = other DNA
ccttcctaattcttctttcaatagctattta

SEQ ID NO: 317 - DNA, 32 bp, synthetic construct
  source 1..32: mol_type = other DNA
ggctggtccgatgggagtgggtgatccgaact

SEQ ID NO: 318 - DNA, 32 bp, synthetic construct
  source 1..32: mol_type = other DNA
ggctggtccgatggtagtgggttatagggatt

SEQ ID NO: 319 - DNA, 36 bp, synthetic construct
  source 1..36: mol_type = other DNA
gaaaagacatggagggtgtagaataagtgggagctt

SEQ ID NO: 320 - DNA, 37 bp, synthetic construct
  source 1..37: mol_type = other DNA
cctgcatcagaggacaaacccgctaataacttgatcc

SEQ ID NO: 321 - DNA, 20 bp, synthetic construct
  source 1..20: mol_type = other DNA
cagggagctggagagggttc

SEQ ID NO: 322 - DNA, 23 bp, synthetic construct
  source 1..23: mol_type = other DNA
tgcgagtgtagaggtgaaattcg

SEQ ID NO: 323 - DNA, 24 bp, synthetic construct
  source 1..24: mol_type = other DNA
ctgtgtccccacccaaatctcatc

SEQ ID NO: 324 - DNA, 24 bp, synthetic construct
  source 1..24: mol_type = other DNA
gtgtccatgttgaaaactcgcctg

SEQ ID NO: 325 - DNA, 26 bp, synthetic construct
  source 1..26: mol_type = other DNA
cccttcccatttttaatagttgtagc

SEQ ID NO: 326 - DNA, 26 bp, synthetic construct
  source 1..26: mol_type = other DNA
tgctgcgggctgtcaggatgctcacc

SEQ ID NO: 327 - DNA, 27 bp, synthetic construct
  source 1..27: mol_type = other DNA
gttatttggattctgggtatgctctgg

SEQ ID NO: 328 - DNA, 29 bp, synthetic construct
  source 1..29: mol_type = other DNA
cagcccgggttccctctttctgccatctc

SEQ ID NO: 329 - DNA, 30 bp, synthetic construct
  source 1..30: mol_type = other DNA
taggtggatggtggatgggtggatgatgga

SEQ ID NO: 330 - DNA, 44 bp, synthetic construct
  source 1..44: mol_type = other DNA
cacctgtgcgtgtgggtgatgctgcgggctgtcagatgctgacc

SEQ ID NO: 331 - DNA, 24 bp, synthetic construct
  source 1..24: mol_type = other DNA
cctatctcagaatgcctgaaccac

SEQ ID NO: 332 - DNA, 35 bp, synthetic construct
  source 1..35: mol_type = other DNA
ttctggtagaattcagctgtgaatccgtcttgtcc

SEQ ID NO: 333 - DNA, 40 bp, synthetic construct
  source 1..40: mol_type = other DNA
cccattcattcatttcaatatccttcaaacatttcttttc

SEQ ID NO: 334 - DNA, 17 bp, synthetic construct
  source 1..17: mol_type = other DNA
aggactgtcctcgggaa

SEQ ID NO: 335 - DNA, 20 bp, synthetic construct
  source 1..20: mol_type = other DNA
atttgagaggggctgacctt

SEQ ID NO: 336 - DNA, 21 bp, synthetic construct
  source 1..21: mol_type = other DNA
ccccagaatgatcttgccttc

SEQ ID NO: 337 - DNA, 25 bp, synthetic construct
  source 1..25: mol_type = other DNA
atacatgagttgggcttactgagtg

SEQ ID NO: 338 - DNA, 26 bp, synthetic construct
  source 1..26: mol_type = other DNA
taaatgggtaagaagcccggctcgct

SEQ ID NO: 339 - DNA, 27 bp, synthetic construct
  source 1..27: mol_type = other DNA
cagaactggaacttgaacccacatttc

SEQ ID NO: 340 - DNA, 38 bp, synthetic construct
  source 1..38: mol_type = other DNA
gcattggtggttcagtggtagaattctcgcctggtgga

SEQ ID NO: 341 - DNA, 39 bp, synthetic construct
  source 1..39: mol_type = other DNA
caaaggtcaaacaacacaagtgagtctcaaactctcaac

SEQ ID NO: 342 - DNA, 42 bp, synthetic construct
  source 1..42: mol_type = other DNA
cctcgcgtcgcttcctcttctccttcaggagcgttttatccc

SEQ ID NO: 343 - DNA, 44 bp, synthetic construct
  source 1..44: mol_type = other DNA
caagtgcaaagggaattcattttgaagagttttatgcaactgtg

SEQ ID NO: 344 - DNA, 20 bp, synthetic construct
  source 1..20: mol_type = other DNA
agttctacagtcggccgatc

SEQ ID NO: 345 - DNA, 21 bp, synthetic construct
  source 1..21: mol_type = other DNA
aatggaggagtggtcggagga

SEQ ID NO: 346 - DNA, 21 bp, synthetic construct
  source 1..21: mol_type = other DNA
caaatgactatctcactgctc

SEQ ID NO: 347 - DNA, 24 bp, synthetic construct
  source 1..24: mol_type = other DNA
catattgttctgtgatcttaactg

SEQ ID NO: 348 - DNA, 25 bp, synthetic construct
  source 1..25: mol_type = other DNA
gggacgttagctcagttggtagagc

SEQ ID NO: 349 - DNA, 31 bp, synthetic construct
  source 1..31: mol_type = other DNA
ttgatctctggactgaggctttgtgtgtgcc

SEQ ID NO: 350 - DNA, 33 bp, synthetic construct
  source 1..33: mol_type = other DNA
acacgatctcggctcactgcaacctctgcctcc

SEQ ID NO: 351 - DNA, 34 bp, synthetic construct
  source 1..34: mol_type = other DNA
ccctggctccctgctgggcttggggagcctcttc

SEQ ID NO: 352 - DNA, 36 bp, synthetic construct
  source 1..36: mol_type = other DNA
tgcgagcggtcccgggttcacatcccggacgagccc

SEQ ID NO: 353 - DNA, 37 bp, synthetic construct
  source 1..37: mol_type = other DNA
ccctcaatccctggtcgagggagagggacttcctgtc

SEQ ID NO: 354 - DNA, 39 bp, synthetic construct
  source 1..39: mol_type = other DNA
gattaggatacaaggtcttgctagaactccctatctccc

SEQ ID NO: 355 - DNA, 43 bp, synthetic construct
  source 1..43: mol_type = other DNA
ctgtggaacggggtgagatgggatgggatgggacaggatagga

SEQ ID NO: 356 - DNA, 17 bp, synthetic construct
  source 1..17: mol_type = other DNA
ctggaaggtttgactgt

SEQ ID NO: 357 - DNA, 23 bp, synthetic construct
  source 1..23: mol_type = other DNA
tgccctttgtcatccctatgcct

SEQ ID NO: 358 - DNA, 24 bp, synthetic construct
  source 1..24: mol_type = other DNA
ccccatgaccctattcaagacttc

SEQ ID NO: 359 - DNA, 24 bp, synthetic construct
  source 1..24: mol_type = other DNA
cggtagctcgtcaggctcataacc

SEQ ID NO: 360 - DNA, 24 bp, synthetic construct
  source 1..24: mol_type = other DNA
ttccctttgtcatccttatgcctg

SEQ ID NO: 361 - DNA, 25 bp, synthetic construct
  source 1..25: mol_type = other DNA
cttcaacatcacctgtagccatcac

SEQ ID NO: 362 - DNA, 26 bp, synthetic construct
  source 1..26: mol_type = other DNA
ccttccaccttggcctcccaaagtgc

SEQ ID NO: 363 - DNA, 27 bp, synthetic construct
  source 1..27: mol_type = other DNA
aggggaatggaatggaatggaatgcaa

SEQ ID NO: 364 - DNA, 28 bp, synthetic construct
  source 1..28: mol_type = other DNA
cgcgggtgagtaggtcgctgccaggtct

SEQ ID NO: 365 - DNA, 29 bp, synthetic construct
  source 1..29: mol_type = other DNA
agggaccctctgtggcgggtagtttgact

SEQ ID NO: 366 - DNA, 29 bp, synthetic construct
  source 1..29: mol_type = other DNA
tatatggaagacataaaaagagaagctcc

SEQ ID NO: 367 - DNA, 34 bp, synthetic construct
  source 1..34: mol_type = other DNA
aggaatttcggtccagattgtttcttgagtcact

SEQ ID NO: 368 - DNA, 35 bp, synthetic construct
  source 1..35: mol_type = other DNA
aaaaagtctttaactccaccattagcacccaaagc

SEQ ID NO: 369 - DNA, 36 bp, synthetic construct
  source 1..36: mol_type = other DNA
ctaaggggtcgggagttcgaatctctctgagcgcac

SEQ ID NO: 370 - DNA, 37 bp, synthetic construct
  source 1..37: mol_type = other DNA
cgtagtgtcggtggttcgattccgcccctgggcacca

SEQ ID NO: 371 - DNA, 37 bp, synthetic construct
  source 1..37: mol_type = other DNA
gagctgattggtactaatcggtcgtgaggcttgacct

SEQ ID NO: 372 - DNA, 38 bp, synthetic construct
  source 1..38: mol_type = other DNA
gctctaagttcgagtctctctttcacttcttctcttgg

SEQ ID NO: 373 - DNA, 39 bp, synthetic construct
  source 1..39: mol_type = other DNA
cccaggttgagtttatgggggtagtgctgtaaggtcatt

SEQ ID NO: 374 - DNA, 43 bp, synthetic construct
  source 1..43: mol_type = other DNA
aatcggactgttcaactcacctggcaaccactcccagagcccc

SEQ ID NO: 375 - DNA, 44 bp, synthetic construct
  source 1..44: mol_type = other DNA
tttcaaggactgtgtttaatttccttttggatttgtttattttg

SEQ ID NO: 376 - DNA, 18 bp, synthetic construct
  source 1..18: mol_type = other DNA
cgaataagctttgatcca

SEQ ID NO: 377 - DNA, 20 bp, synthetic construct
  source 1..20: mol_type = other DNA
cactggaattctgagcccct

SEQ ID NO: 378 - DNA, 20 bp, synthetic construct
  source 1..20: mol_type = other DNA
caggagtcgggggtgggacg

SEQ ID NO: 379 - DNA, 21 bp, synthetic construct
  source 1..21: mol_type = other DNA
aaaagaggaccaccaccaaga

SEQ ID NO: 380 - DNA, 21 bp, synthetic construct
  source 1..21: mol_type = other DNA
ggtggtggcggcggtggtggc

SEQ ID NO: 381 - DNA, 21 bp, synthetic construct
  source 1..21: mol_type = other DNA
gtcttactctgttgctcaggc

SEQ ID NO: 382 - DNA, 23 bp, synthetic construct
  source 1..23: mol_type = other DNA
cctcctctggatcacatgggctc

SEQ ID NO: 383 - DNA, 23 bp, synthetic construct
  source 1..23: mol_type = other DNA
ccttcgggcctgtccagaacctc

SEQ ID NO: 384 - DNA, 23 bp, synthetic construct
  source 1..23: mol_type = other DNA
ttcgaatctcaccgcttccgcca

SEQ ID NO: 385 - DNA, 29 bp, synthetic construct
  source 1..29: mol_type = other DNA
ccatcacataggggattagatttcaatgc

SEQ ID NO: 386 - DNA, 29 bp, synthetic construct
  source 1..29: mol_type = other DNA
tgtaagggctgggtcggtcgggctggggc

SEQ ID NO: 387 - DNA, 31 bp, synthetic construct
  source 1..31: mol_type = other DNA
cagcgcctttgcacacgctattctctctgcc

SEQ ID NO: 388 - DNA, 31 bp, synthetic construct
  source 1..31: mol_type = other DNA
cgcggagcccagggttcgattccctgtaccg

SEQ ID NO: 389 - DNA, 31 bp, synthetic construct
  source 1..31: mol_type = other DNA
ctgatgggctgggcagggctccctggatggg

SEQ ID NO: 390 - DNA, 33 bp, synthetic construct
  source 1..33: mol_type = other DNA
ccccacttccgtactgagtttctcacctgtttg

SEQ ID NO: 391 - DNA, 34 bp, synthetic construct
  source 1..34: mol_type = other DNA
agtactgttatttagcgtgctaaatatattgtcc

SEQ ID NO: 392 - DNA, 34 bp, synthetic construct
  source 1..34: mol_type = other DNA
agtgcatcgcgcgaaagtaggtcgtcgccggctt

SEQ ID NO: 393 - DNA, 36 bp, synthetic construct
  source 1..36: mol_type = other DNA
cctgattttttttgcaatttctttgtattgttttta

SEQ ID NO: 394 - DNA, 37 bp, synthetic construct
  source 1..37: mol_type = other DNA
tgatggagtggcctggactcacattaaaataagtact

SEQ ID NO: 395 - DNA, 38 bp, synthetic construct
  source 1..38: mol_type = other DNA
ccccttacccatcaaattttccttaaaaactccaatcc

SEQ ID NO: 396 - DNA, 39 bp, synthetic construct
  source 1..39: mol_type = other DNA
ctctttggggcggggtgggggagggggagcctcgcgtcc

SEQ ID NO: 397 - DNA, 40 bp, synthetic construct
  source 1..40: mol_type = other DNA
cctgagctcttgttcgatgtccaaggataatgaggtggca

SEQ ID NO: 398 - DNA, 40 bp, synthetic construct
  source 1..40: mol_type = other DNA
taaggaggaggaacattgtgagcaggagaaggatctgggg

SEQ ID NO: 399 - DNA, 40 bp, synthetic construct
  source 1..40: mol_type = other DNA
tcctgtccggttgaggcctttctcttggggtcttgctgtc

SEQ ID NO: 400 - DNA, 41 bp, synthetic construct
  source 1..41: mol_type = other DNA
cctttcatatcttctcaaatactgatttaattttatactgg

SEQ ID NO: 401 - DNA, 42 bp, synthetic construct
  source 1..42: mol_type = other DNA
cctaggttcaagtgatcctcctgcttcagcttcctgagtagc

SEQ ID NO: 402 - DNA, 42 bp, synthetic construct
  source 1..42: mol_type = other DNA
cctggcctcaagcaatcctcccaccttggcctccacaagtac

SEQ ID NO: 403 - DNA, 44 bp, synthetic construct
  source 1..44: mol_type = other DNA
catctcagctccaaacccacaggttgggttcagttcttgcatcc

SEQ ID NO: 404 - DNA, 23 bp, synthetic construct
  source 1..23: mol_type = other DNA
tggaattctcgggtgccaaggaa

SEQ ID NO: 405 - DNA, 21 bp, synthetic construct
  source 1..21: mol_type = other DNA
gctgattgtcacgttctgatt

SEQ ID NO: 406 - DNA, 20 bp, synthetic construct
  source 1..20: mol_type = other DNA
gcccctgggcctatcctaga

SEQ ID NO: 407 - DNA, 19 bp, synthetic construct
  source 1..19: mol_type = other DNA
agttcttcagtggcaagct

SEQ ID NO: 408 - DNA, 22 bp, synthetic construct
  source 1..22: mol_type = other DNA
accctgtagaaccgaatttgta

SEQ ID NO: 409 - DNA, 23 bp, synthetic construct
  source 1..23: mol_type = other DNA
taggtagtttcctgttgttggat

SEQ ID NO: 410 - DNA, 21 bp, synthetic construct
  source 1..21: mol_type = other DNA
accctgtagatctgaatttgt

SEQ ID NO: 411 - DNA, 19 bp, synthetic construct
  source 1..19: mol_type = other DNA
tgagatgaagctgtagctc

SEQ ID NO: 412 - DNA, 22 bp, synthetic construct
  source 1..22: mol_type = other DNA
taccctgtagaaccgaattggt

SEQ ID NO: 413 - DNA, 21 bp, synthetic construct
  source 1..21: mol_type = other DNA
accctgtagaaccgaatttgg

SEQ ID NO: 414 - DNA, 22 bp, synthetic construct
  source 1..22: mol_type = other DNA
taacagtctacagccatggtcg

SEQ ID NO: 415 - DNA, 20 bp, synthetic construct
  source 1..20: mol_type = other DNA
agttcttcagtggcaagctt

SEQ ID NO: 416 - DNA, 22 bp, synthetic construct
  source 1..22: mol_type = other DNA
taccctgtagaaccgaatttgg

SEQ ID NO: 417 - DNA, 22 bp, synthetic construct
  source 1..22: mol_type = other DNA
cagtgcaatgatgaaagggcat

SEQ ID NO: 418 - DNA, 21 bp, synthetic construct
  source 1..21: mol_type = other DNA
taccctgtagaaccgaattta

SEQ ID NO: 419 - DNA, 22 bp, synthetic construct
  source 1..22: mol_type = other DNA
tacagttgttcaaccagttact

SEQ ID NO: 420 - DNA, 22 bp, synthetic construct
  source 1..22: mol_type = other DNA
accctgtagaaccgaatttggg

SEQ ID NO: 421 - DNA, 22 bp, synthetic construct
  source 1..22: mol_type = other DNA
taccctgtaggaccgaatttgt

SEQ ID NO: 422 - DNA, 20 bp, synthetic construct
  source 1..20: mol_type = other DNA
tggcagtgtcttagctggtt

SEQ ID NO: 423 - DNA, 20 bp, synthetic construct
  source 1..20: mol_type = other DNA
accctgtagaaccgaattta

SEQ ID NO: 424 - DNA, 22 bp, synthetic construct
  source 1..22: mol_type = other DNA
accctgtagaaccgaatttgtt

SEQ ID NO: 425 - DNA, 22 bp, synthetic construct
  source 1..22: mol_type = other DNA
taccctgtagatccgattttgt

SEQ ID NO: 426 - DNA, 22 bp, synthetic construct
  source 1..22: mol_type = other DNA
taccctgtagaaccgagtttgt

SEQ ID NO: 427 - DNA, 23 bp, synthetic construct
  source 1..23: mol_type = other DNA
ttcaagtaatccaggataggcct

SEQ ID NO: 428 - DNA, 22 bp, synthetic construct
  source 1..22: mol_type = other DNA
taccctgtagaaccgaatttat

SEQ ID NO: 429 - DNA, 21 bp, synthetic construct
  source 1..21: mol_type = other DNA
taccctgtagaaccggatttg

SEQ ID NO: 430 - DNA, 25 bp, synthetic construct
  source 1..25: mol_type = other DNA
tattgcacttgtcccggcctgtagc

SEQ ID NO: 431 - DNA, 23 bp, synthetic construct
  source 1..23: mol_type = other DNA
accctgtagatctgaatttgtga

SEQ ID NO: 432 - DNA, 19 bp, synthetic construct
  source 1..19: mol_type = other DNA
cactagattgtgagctcct

SEQ ID NO: 433 - DNA, 22 bp, synthetic construct
  source 1..22: mol_type = other DNA
taccctgtagtaccgaatttgt

SEQ ID NO: 434 - DNA, 22 bp, synthetic construct
  source 1..22: mol_type = other DNA
cagtgcaatgttaaaagggcaa

SEQ ID NO: 435 - DNA, 21 bp, synthetic construct
  source 1..21: mol_type = other DNA
ctgacctatgatttgacagcc

SEQ ID NO: 436 - DNA, 23 bp, synthetic construct
  source 1..23: mol_type = other DNA
ctgacctatgaattgacagccct

SEQ ID NO: 437 - DNA, 22 bp, synthetic construct
  source 1..22: mol_type = other DNA
ccactgccccaggtgctgctgg

SEQ ID NO: 438 - DNA, 22 bp, synthetic construct
  source 1..22: mol_type = other DNA
tgaggtagtaggttgtgtgggt

SEQ ID NO: 439 - DNA, 21 bp, synthetic construct
  source 1..21: mol_type = other DNA
actgtgcgtgtgacagcggct

SEQ ID NO: 440 - DNA, 20 bp, synthetic construct
  source 1..20: mol_type = other DNA
ctgcgcaagctactgccttg

SEQ ID NO: 441 - DNA, 23 bp, synthetic construct
  source 1..23: mol_type = other DNA
cacccgtagaaccgaccttgcga

SEQ ID NO: 442 - DNA, 21 bp, synthetic construct
  source 1..21: mol_type = other DNA
ctgacctatgtattgacagcc

SEQ ID NO: 443 - DNA, 22 bp, synthetic construct
  source 1..22: mol_type = other DNA
taccctgtagaaccgaatttgc

SEQ ID NO: 444 - DNA, 25 bp, synthetic construct
  source 1..25: mol_type = other DNA
tgagaactgaattccataggctgaa

SEQ ID NO: 445 - DNA, 24 bp, synthetic construct
  source 1..24: mol_type = other DNA
tgacctatgaattgacagccaatt

SEQ ID NO: 446 - DNA, 23 bp, synthetic construct
  source 1..23: mol_type = other DNA
taccctgtagaaccgaatttgta

SEQ ID NO: 447 - DNA, 21 bp, synthetic construct
  source 1..21: mol_type = other DNA
tgagatgaagcactgtagatc

SEQ ID NO: 448 - DNA, 22 bp, synthetic construct
  source 1..22: mol_type = other DNA
taccctgtagaaccgaacttgt

SEQ ID NO: 449 - DNA, 21 bp, synthetic construct
  source 1..21: mol_type = other DNA
ctgacctatgaactgacagcc

SEQ ID NO: 450 - DNA, 18 bp, synthetic construct
  source 1..18: mol_type = other DNA
gattgtcacgttctgatt

SEQ ID NO: 451 - DNA, 22 bp, synthetic construct
  source 1..22: mol_type = other DNA
ttacagtctacagccatggtcg

SEQ ID NO: 452 - DNA, 24 bp, synthetic construct
  source 1..24: mol_type = other DNA
cattgcacttgtctcggtctgaat

SEQ ID NO: 453 - DNA, 22 bp, synthetic construct
  source 1..22: mol_type = other DNA
taccctgttgaaccgaatttgt

SEQ ID NO: 454 - DNA, 22 bp, synthetic construct
  source 1..22: mol_type = other DNA
caaagtgctgttcgtgcaggta

SEQ ID NO: 455 - DNA, 26 bp, synthetic construct
  source 1..26: mol_type = other DNA
ctcgcttctggcgccaagcgcccggc

SEQ ID NO: 456 - DNA, 20 bp, synthetic construct
  source 1..20: mol_type = other DNA
aactggccctcaaagtcccg

SEQ ID NO: 457 - DNA, 23 bp, synthetic construct
  source 1..23: mol_type = other DNA
tgagaactgaattccataggcaa

SEQ ID NO: 458 - DNA, 24 bp, synthetic construct
  source 1..24: mol_type = other DNA
tgaggtagtagattgtatagtttt

SEQ ID NO: 459 - DNA, 17 bp, synthetic construct
  source 1..17: mol_type = other DNA
accctgtagatccgaat

SEQ ID NO: 460 - DNA, 25 bp, synthetic construct
  source 1..25: mol_type = other DNA
aggctgtgatgctctcctgagccct

SEQ ID NO: 461 - DNA, 17 bp, synthetic construct
  source 1..17: mol_type = other DNA
taacactgtctggtaac

SEQ ID NO: 462 - DNA, 22 bp, synthetic construct
  source 1..22: mol_type = other DNA
taccctgtagatccgaattcgt

SEQ ID NO: 463 - DNA, 23 bp, synthetic construct
  source 1..23: mol_type = other DNA
ccgccccaccccgcgcgcgccgc

SEQ ID NO: 464 - DNA, 27 bp, synthetic construct
  source 1..27: mol_type = other DNA
cgcttctggcgccaagcgcccggccgc

SEQ ID NO: 465 - DNA, 30 bp, synthetic construct
  source 1..30: mol_type = other DNA
agattgagggttcgtcccttcgtggtcgcc

SEQ ID NO: 466 - DNA, 30 bp, synthetic construct
  source 1..30: mol_type = other DNA
ggcttggtctaggggtatgattctcgcttt

SEQ ID NO: 467 - DNA, 29 bp, synthetic construct
  source 1..29: mol_type = other DNA
ggctttgtctaggggtatgattctcgctt

SEQ ID NO: 468 - DNA, 25 bp, synthetic construct
  source 1..25: mol_type = other DNA
cccgccccaccccgcgcgcgccgct

SEQ ID NO: 469 - DNA, 30 bp, synthetic construct
  source 1..30: mol_type = other DNA
cgtacggaagacccgctccccggcgccgct

SEQ ID NO: 470 - DNA, 27 bp, synthetic construct
  source 1..27: mol_type = other DNA
gtacggaagacccgctccccggcgccg

SEQ ID NO: 471 - DNA, 28 bp, synthetic construct
  source 1..28: mol_type = other DNA
tggtctagcggttaggattcctggtttt

SEQ ID NO: 472 - DNA, 22 bp, synthetic construct
  source 1..22: mol_type = other DNA
cgccccaccccgcgcgcgccgc

SEQ ID NO: 473 - DNA, 22 bp, synthetic construct
  source 1..22: mol_type = other DNA
cccgcgaggggggcccgggcac

SEQ ID NO: 474 - DNA, 28 bp, synthetic construct
  source 1..28: mol_type = other DNA
gcgccgccgccccccccacgcccggggc

SEQ ID NO: 475 - DNA, 23 bp, synthetic construct
  source 1..23: mol_type = other DNA
gctccccgtcctcccccctcccc

SEQ ID NO: 476 - DNA, 25 bp, synthetic construct
  source 1..25: mol_type = other DNA
gcgcaatgaaggtgaaggccggcgc

SEQ ID NO: 477 - DNA, 22 bp, synthetic construct
  source 1..22: mol_type = other DNA
acgctgccagttgaagaactgt

SEQ ID NO: 478 - DNA, 23 bp, synthetic construct
  source 1..23: mol_type = other DNA
gcccctgggcctatcctagaaaa

SEQ ID NO: 479 - DNA, 23 bp, synthetic construct
  source 1..23: mol_type = other DNA
gcgggtccggccgtgtcggcggc

SEQ ID NO: 480 - DNA, 28 bp, synthetic construct
  source 1..28: mol_type = other DNA
ggcttggtctaggggtatgattctcgct

SEQ ID NO: 481 - DNA, 21 bp, synthetic construct
  source 1..21: mol_type = other DNA
ccacctcccctgcaaacgtcc

SEQ ID NO: 482 - DNA, 19 bp, synthetic construct
  source 1..19: mol_type = other DNA
ggttaggattcctggtttt

SEQ ID NO: 483 - DNA, 30 bp, synthetic construct
  source 1..30: mol_type = other DNA
tctggcatgctaactagttacgcgaccccc

SEQ ID NO: 484 - DNA, 30 bp, synthetic construct
  source 1..30: mol_type = other DNA
cgcgtcccccgaagagggggacggcggagc

SEQ ID NO: 485 - DNA, 30 bp, synthetic construct
  source 1..30: mol_type = other DNA
gcggagcgagcgcacggggtcggcggcgac

SEQ ID NO: 486 - DNA, 30 bp, synthetic construct
  source 1..30: mol_type = other DNA
cccccgccccaccccgcgcgcgccgctcgc

SEQ ID NO: 487 - DNA, 29 bp, synthetic construct
  source 1..29: mol_type = other DNA
ccgtaggtgaacctgcggaaggatcatta

SEQ ID NO: 488 - DNA, 26 bp, synthetic construct
  source 1..26: mol_type = other DNA
gggctacgcctgtctgagcgtcgctt

SEQ ID NO: 489 - DNA, 24 bp, synthetic construct
  source 1..24: mol_type = other DNA
gctacgcctgtctgagcgtcgctt

SEQ ID NO: 490 - DNA, 26 bp, synthetic construct
  source 1..26: mol_type = other DNA
cccccacaaccgcgcttgactagctt

SEQ ID NO: 491 - DNA, 21 bp, synthetic construct
  source 1..21: mol_type = other DNA
ccctacccccccggccccgtc

SEQ ID NO: 492 - DNA, 28 bp, synthetic construct
  source 1..28: mol_type = other DNA
cccgccccaccccgcgcgcgccgctcgc

SEQ ID NO: 493 - DNA, 30 bp, synthetic construct
  source 1..30: mol_type = other DNA
gggggtatagctcagtggtagagcgtgctt

SEQ ID NO: 494 - DNA, 30 bp, synthetic construct
  source 1..30: mol_type = other DNA
gtcggtcgggctggggcgcgaagcggggct

SEQ ID NO: 495 - DNA, 20 bp, synthetic construct
  source 1..20: mol_type = other DNA
tcagtggagagcatttgact

SEQ ID NO: 496 - DNA, 22 bp, synthetic construct
  source 1..22: mol_type = other DNA
cacccctagaaccgaccttgcg

SEQ ID NO: 497 - DNA, 23 bp, synthetic construct
  source 1..23: mol_type = other DNA
cctcaccatcccttctgcctgca

SEQ ID NO: 498 - DNA, 21 bp, synthetic construct
  source 1..21: mol_type = other DNA
gtcaggatggccgagcggtct

SEQ ID NO: 499 - DNA, 30 bp, synthetic construct
  source 1..30: mol_type = other DNA
tccctggtctagtggttaggattcggcgcg

SEQ ID NO: 500 - DNA, 21 bp, synthetic construct
  source 1..21: mol_type = other DNA
tgagatgaagcactgtagatc

SEQ ID NO: 501 - DNA, 23 bp, synthetic construct
  source 1..23: mol_type = other DNA
ggatcggccccgccggggtcggc

SEQ ID NO: 502 - DNA, 22 bp, synthetic construct
  source 1..22: mol_type = other DNA
ggaacctgcggaaggatcatta

SEQ ID NO: 503 - DNA, 23 bp, synthetic construct
  source 1..23: mol_type = other DNA
tgaggtagtaggttgtatggttg

SEQ ID NO: 504 - DNA, 24 bp, synthetic construct
  source 1..24: mol_type = other DNA
gtctagtggttaggattcggcgct

SEQ ID NO: 505 - DNA, 30 bp, synthetic construct
  source 1..30: mol_type = other DNA
tccctggtctagtggctaggattcggcgct

SEQ ID NO: 506 - DNA, 23 bp, synthetic construct
  source 1..23: mol_type = other DNA
gccgccccccccacgcccggggc

SEQ ID NO: 507 - DNA, 24 bp, synthetic construct
  source 1..24: mol_type = other DNA
tgtcagtttgtcaaataccccaag

SEQ ID NO: 508 - DNA, 23 bp, synthetic construct
  source 1..23: mol_type = other DNA
cagcagcaattcatgttttgaat

SEQ ID NO: 509 - DNA, 26 bp, synthetic construct
  source 1..26: mol_type = other DNA
gtggttgtagtccgtgcgagaatacc

SEQ ID NO: 510 - DNA, 23 bp, synthetic construct
  source 1..23: mol_type = other DNA
ggatatcatcatatactgtaagt

SEQ ID NO: 511 - DNA, 20 bp, synthetic construct
  source 1..20: mol_type = other DNA
taacagtctccagtcacggc

SEQ ID NO: 512 - DNA, 23 bp, synthetic construct
  source 1..23: mol_type = other DNA
tcagtgcactacagaactttttt

SEQ ID NO: 513 - DNA, 23 bp, synthetic construct
  source 1..23: mol_type = other DNA
ccagtggggctgctgttatctgt

SEQ ID NO: 514 - DNA, 21 bp, synthetic construct
  source 1..21: mol_type = other DNA
gataaagtagaaagcactact

SEQ ID NO: 515 - DNA, 23 bp, synthetic construct
  source 1..23: mol_type = other DNA
taggtagtttcctgttgttggat

SEQ ID NO: 516 - DNA, 22 bp, synthetic construct
  source 1..22: mol_type = other DNA
atgcttatcagactgatgttga

SEQ ID NO: 517 - DNA, 22 bp, synthetic construct
  source 1..22: mol_type = other DNA
tagtgcaatattgcttataggg

SEQ ID NO: 518 - DNA, 23 bp, synthetic construct
  source 1..23: mol_type = other DNA
cccataaagtagaaagcactact

SEQ ID NO: 519 - DNA, 21 bp, synthetic construct
  source 1..21: mol_type = other DNA
tacccattgcatatcggagtt

SEQ ID NO: 520 - DNA, 23 bp, synthetic construct
  source 1..23: mol_type = other DNA
actggacttggagtcagaaggaa

SEQ ID NO: 521 - DNA, 21 bp, synthetic construct
  source 1..21: mol_type = other DNA
aagcagcaattcatgttttga

SEQ ID NO: 522 - DNA, 22 bp, synthetic construct
  source 1..22: mol_type = other DNA
ctgcagcacgtaaatattggcg

SEQ ID NO: 523 - DNA, 20 bp, synthetic construct
  source 1..20: mol_type = other DNA
tggcagtgtcttagctggtt

SEQ ID NO: 524 - DNA, 23 bp, synthetic construct
  source 1..23: mol_type = other DNA
actggacttggagtcagaaggtt

SEQ ID NO: 525 - DNA, 26 bp, synthetic construct
  source 1..26: mol_type = other DNA
tgagaactgaattccataggctgtaa

SEQ ID NO: 526 - DNA, 23 bp, synthetic construct
  source 1..23: mol_type = other DNA
actggacttggagtcagaaggat

SEQ ID NO: 527 - DNA, 27 bp, synthetic construct
  source 1..27: mol_type = other DNA
tgagaactgaattccataggctgtaat

SEQ ID NO: 528 - DNA, 22 bp, synthetic construct
  source 1..22: mol_type = other DNA
gttgagactctgaaatctgatt

SEQ ID NO: 529 - DNA, 19 bp, synthetic construct
  source 1..19: mol_type = other DNA
ttaatgctaatcgtgatag

SEQ ID NO: 530 - DNA, 22 bp, synthetic construct
  source 1..22: mol_type = other DNA
tgagaactgaattccataggaa

SEQ ID NO: 531 - DNA, 22 bp, synthetic construct
  source 1..22: mol_type = other DNA
ctatacgacctgctgcctttca

SEQ ID NO: 532 - DNA, 23 bp, synthetic construct
  source 1..23: mol_type = other DNA
taccctgtagaaccgaatttgcg

SEQ ID NO: 533 - DNA, 19 bp, synthetic construct
  source 1..19: mol_type = other DNA
tggcagtgtcttagctggt

SEQ ID NO: 534 - DNA, 21 bp, synthetic construct
  source 1..21: mol_type = other DNA
ccagtggggctgctgttatct

SEQ ID NO: 535 - DNA, 23 bp, synthetic construct
  source 1..23: mol_type = other DNA
ttgagaactgaattccatgggtt

SEQ ID NO: 536 - DNA, 22 bp, synthetic construct
  source 1..22: mol_type = other DNA
ttacagtctacagccatggtcg

SEQ ID NO: 537 - DNA, 23 bp, synthetic construct
  source 1..23: mol_type = other DNA
actggacttggagtcagaaggct

SEQ ID NO: 538 - DNA, 26 bp, synthetic construct
  source 1..26: mol_type = other DNA
tgagaactgaattccataggctgtag

SEQ ID NO: 539 - DNA, 23 bp, synthetic construct
  source 1..23: mol_type = other DNA
cccataaagtagaaagcactaca

SEQ ID NO: 540 - DNA, 19 bp, synthetic construct
  source 1..19: mol_type = other DNA
tgaggtagtagtttgtgct

SEQ ID NO: 541 - DNA, 20 bp, synthetic construct
  source 1..20: mol_type = other DNA
cggcgcaagctactgccttg

SEQ ID NO: 542 - DNA, 19 bp, synthetic construct
  source 1..19: mol_type = other DNA
agttcttcagtggcaagct

SEQ ID NO: 543 - DNA, 22 bp, synthetic construct
  source 1..22: mol_type = other DNA
tcccctgtagaaccgaatttgt

SEQ ID NO: 544 - DNA, 25 bp, synthetic construct
  source 1..25: mol_type = other DNA
actggacttggagtcagaaggcatt

SEQ ID NO: 545 - DNA, 22 bp, synthetic construct
  source 1..22: mol_type = other DNA
aagctcggtctgaggcccctca

SEQ ID NO: 546 - DNA, 23 bp, synthetic construct
  source 1..23: mol_type = other DNA
ccagtggggctgctgttatctga

SEQ ID NO: 547 - DNA, 23 bp, synthetic construct
  source 1..23: mol_type = other DNA
tgagggagtagtttgtgctgtta

SEQ ID NO: 548 - DNA, 21 bp, synthetic construct
  source 1..21: mol_type = other DNA
aagaaagtagaaagcactact

SEQ ID NO: 549 - DNA, 21 bp, synthetic construct
  source 1..21: mol_type = other DNA
cgctgccagttgaagaactgt

SEQ ID NO: 550 - DNA, 18 bp, synthetic construct
  source 1..18: mol_type = other DNA
ggctggtccgatggtagt

SEQ ID NO: 551 - DNA, 22 bp, synthetic construct
  source 1..22: mol_type = other DNA
ctgggagaaggctgtttactct

SEQ ID NO: 552 - DNA, 19 bp, synthetic construct
  source 1..19: mol_type = other DNA
aagcaattctcaaaggagc

SEQ ID NO: 553 - DNA, 22 bp, synthetic construct
  source 1..22: mol_type = other DNA
ctcggcgccccctcgatgctct

SEQ ID NO: 554 - DNA, 20 bp, synthetic construct
  source 1..20: mol_type = other DNA
tgtcttgcaggccgtcatgc

SEQ ID NO: 555 - DNA, 19 bp, synthetic construct
  source 1..19: mol_type = other DNA
cgaatcattatttgctgct

SEQ ID NO: 556 - DNA, 23 bp, synthetic construct
  source 1..23: mol_type = other DNA
cagcagcaattcatgttttgaaa

SEQ ID NO: 557 - DNA, 20 bp, synthetic construct
  source 1..20: mol_type = other DNA
accaatattactgtgctgct

SEQ ID NO: 558 - DNA, 24 bp, synthetic construct
  source 1..24: mol_type = other DNA
ttcaagtaatccaggataggcttt

SEQ ID NO: 559 - DNA, 22 bp, synthetic construct
  source 1..22: mol_type = other DNA
ttgagaactgaattccatgggt

SEQ ID NO: 560 - DNA, 20 bp, synthetic construct
  source 1..20: mol_type = other DNA
tattgcacattactaagttg

SEQ ID NO: 561 - DNA, 22 bp, synthetic construct
  source 1..22: mol_type = other DNA
tgacctatgaattgacagccta

SEQ ID NO: 562 - DNA, 20 bp, synthetic construct
  source 1..20: mol_type = other DNA
actgtaaacgctttctgatg

SEQ ID NO: 563 - DNA, 24 bp, synthetic construct
  source 1..24: mol_type = other DNA
cattgcacttgtctcggtctgaat

SEQ ID NO: 564 - DNA, 20 bp, synthetic construct
  source 1..20: mol_type = other DNA
ataaagtagaaagcactact

SEQ ID NO: 565 - DNA, 21 bp, synthetic construct
  source 1..21: mol_type = other DNA
aagtgcaatgatgaaagggca

SEQ ID NO: 566 - DNA, 21 bp, synthetic construct
  source 1..21: mol_type = other DNA
accataaagtagaaagcacta

SEQ ID NO: 567 - DNA, 26 bp, synthetic construct
  source 1..26: mol_type = other DNA
ccccactgctaaatttgactggcttt

SEQ ID NO: 568 - DNA, 25 bp, synthetic construct
  source 1..25: mol_type = other DNA
tgtcagtttgtcaaataccccaaga

SEQ ID NO: 569 - DNA, 22 bp, synthetic construct
  source 1..22: mol_type = other DNA
tacccagtagaaccgaatttgt

SEQ ID NO: 570 - DNA, 22 bp, synthetic construct
  source 1..22: mol_type = other DNA
tttgttcgttcggctcgcgtaa

SEQ ID NO: 571 - DNA, 23 bp, synthetic construct
  source 1..23: mol_type = other DNA
atgctgccagttgaagaactgta

SEQ ID NO: 572 - DNA, 20 bp, synthetic construct
  source 1..20: mol_type = other DNA
tgagaaccacgtctgctctg

SEQ ID NO: 573 - DNA, 23 bp, synthetic construct
  source 1..23: mol_type = other DNA
ctgccaattccataggtcacagt

SEQ ID NO: 574 - DNA, 24 bp, synthetic construct
  source 1..24: mol_type = other DNA
tagcttatcagactgatgttgaga

SEQ ID NO: 575 - DNA, 25 bp, synthetic construct
  source 1..25: mol_type = other DNA
gtagcttatcagactgatgttgact

SEQ ID NO: 576 - DNA, 23 bp, synthetic construct
  source 1..23: mol_type = other DNA
tttggtccccttcaaccagctga

SEQ ID NO: 577 - DNA, 23 bp, synthetic construct
  source 1..23: mol_type = other DNA
tgtaatagcaactccatgtggaa

SEQ ID NO: 578 - DNA, 21 bp, synthetic construct
  source 1..21: mol_type = other DNA
gggacctatgaattgacagac

SEQ ID NO: 579 - DNA, 22 bp, synthetic construct
  source 1..22: mol_type = other DNA
taaggtgcatctagtgcagata

SEQ ID NO: 580 - DNA, 27 bp, synthetic construct
  source 1..27: mol_type = other DNA
gtactggaaagtgcacttggacgaaca

SEQ ID NO: 581 - DNA, 29 bp, synthetic construct
  source 1..29: mol_type = other DNA
cccggggctacgcctgtctgagcgtcgct

SEQ ID NO: 582 - DNA, 23 bp, synthetic construct
  source 1..23: mol_type = other DNA
aaagctgggttgagagggcgaaa

SEQ ID NO: 583 - DNA, 19 bp, synthetic construct
  source 1..19: mol_type = other DNA
cataaagtagaaagcacta

SEQ ID NO: 584 - DNA, 18 bp, synthetic construct
  source 1..18: mol_type = other DNA
tgtcagtttgtcaaatac

SEQ ID NO: 585 - DNA, 21 bp, synthetic construct
  source 1..21: mol_type = other DNA
tccggtgagctctcgctggcc

SEQ ID NO: 586 - DNA, 20 bp, synthetic construct
  source 1..20: mol_type = other DNA
tataaagtagaaagcactac

SEQ ID NO: 587 - DNA, 21 bp, synthetic construct
  source 1..21: mol_type = other DNA
tgctgccagttgaagaactgt

SEQ ID NO: 588 - DNA, 25 bp, synthetic construct
  source 1..25: mol_type = other DNA
agctcggtctgaggcccctcagttt

SEQ ID NO: 589 - DNA, 24 bp, synthetic construct
  source 1..24: mol_type = other DNA
tgtcagtttgtcaaataccccatg

SEQ ID NO: 590 - DNA, 19 bp, synthetic construct
  source 1..19: mol_type = other DNA
atcacagtggctaagttcc

SEQ ID NO: 591 - DNA, 23 bp, synthetic construct
  source 1..23: mol_type = other DNA
tgagaactgaattccataggcaa

SEQ ID NO: 592 - DNA, 22 bp, synthetic construct
  source 1..22: mol_type = other DNA
tgggtctttgcgggcgagatga

SEQ ID NO: 593 - DNA, 21 bp, synthetic construct
  source 1..21: mol_type = other DNA
taccctgtagaaccggatttg

SEQ ID NO: 594 - DNA, 21 bp, synthetic construct
  source 1..21: mol_type = other DNA
tgagggagtagattgtatagt

SEQ ID NO: 595 - DNA, 22 bp, synthetic construct
  source 1..22: mol_type = other DNA
taccctgttgaaccgaatttgt

SEQ ID NO: 596 - DNA, 21 bp, synthetic construct
  source 1..21: mol_type = other DNA
taaggtgcatctagtgcagat

SEQ ID NO: 597 - DNA, 23 bp, synthetic construct
  source 1..23: mol_type = other DNA
gagaactgaattccataggctgt

SEQ ID NO: 598 - DNA, 22 bp, synthetic construct
  source 1..22: mol_type = other DNA
tagcagcacgcaaatattggcg

SEQ ID NO: 599 - DNA, 18 bp, synthetic construct
  source 1..18: mol_type = other DNA
ggctcgttggtctagggg

SEQ ID NO: 600 - DNA, 20 bp, synthetic construct
  source 1..20: mol_type = other DNA
cagcagcaattcatgttttg

SEQ ID NO: 601 - DNA, 20 bp, synthetic construct
  source 1..20: mol_type = other DNA
aacattcaacgctgtcggtg

SEQ ID NO: 602 - DNA, 22 bp, synthetic construct
  source 1..22: mol_type = other DNA
atgcagcacgtaaatattggcg

SEQ ID NO: 603 - DNA, 23 bp, synthetic construct
  source 1..23: mol_type = other DNA
tgccgacgggcgctgaccccctt

SEQ ID NO: 604 - DNA, 29 bp, synthetic construct
  source 1..29: mol_type = other DNA
attggtcgtggttgtagtccgtgcgagaa

SEQ ID NO: 605 - DNA, 21 bp, synthetic construct
  source 1..21: mol_type = other DNA
tggcagtgtcttagctggttg

SEQ ID NO: 606 - DNA, 17 bp, synthetic construct
  source 1..17: mol_type = other DNA
tgtcagtttgtcaaata

SEQ ID NO: 607 - DNA, 22 bp, synthetic construct
  source 1..22: mol_type = other DNA
accctgagaccctaacttgtga

SEQ ID NO: 608 - DNA, 19 bp, synthetic construct
  source 1..19: mol_type = other DNA
tggcagtttgtcaaatacc

SEQ ID NO: 609 - DNA, 26 bp, synthetic construct
  source 1..26: mol_type = other DNA
actggacttggagtcagaaggcatat

SEQ ID NO: 610 - DNA, 24 bp, synthetic construct
  source 1..24: mol_type = other DNA
tcgaccggacctcgaccggctaga

SEQ ID NO: 611 - DNA, 22 bp, synthetic construct
  source 1..22: mol_type = other DNA
tcagcaccaggatattgttgga

SEQ ID NO: 612 - DNA, 22 bp, synthetic construct
  source 1..22: mol_type = other DNA
tgtaaccgcaactccatgtgga

SEQ ID NO: 613 - DNA, 26 bp, synthetic construct
  source 1..26: mol_type = other DNA
actggacttggagtcagaaggcatta

SEQ ID NO: 614 - DNA, 22 bp, synthetic construct
  source 1..22: mol_type = other DNA
aacactgtctggtaaagatggc

SEQ ID NO: 615 - DNA, 26 bp, synthetic construct
  source 1..26: mol_type = other DNA
tgtaaacatcctacactctcagctta

SEQ ID NO: 616 - DNA, 23 bp, synthetic construct
  source 1..23: mol_type = other DNA
actggactttgagtcagaaggca

SEQ ID NO: 617 - DNA, 24 bp, synthetic construct
  source 1..24: mol_type = other DNA
actggacttggagccagaaggcaa

SEQ ID NO: 618 - DNA, 23 bp, synthetic construct
  source 1..23: mol_type = other DNA
gtaacagcaactccatgtggaaa

SEQ ID NO: 619 - DNA, 26 bp, synthetic construct
  source 1..26: mol_type = other DNA
actggacttggagtcagaaggcaata

SEQ ID NO: 620 - DNA, 24 bp, synthetic construct
  source 1..24: mol_type = other DNA
ctggacttggagtcagaaggcaga

SEQ ID NO: 621 - DNA, 24 bp, synthetic construct
  source 1..24: mol_type = other DNA
tgatatgtttgatatattaggtta

SEQ ID NO: 622 - DNA, 24 bp, synthetic construct
  source 1..24: mol_type = other DNA
tgaaatgtttaggaccactagaat

SEQ ID NO: 623 - DNA, 22 bp, synthetic construct
  source 1..22: mol_type = other DNA
tggacttggagtcagaaggcat

SEQ ID NO: 624 - DNA, 25 bp, synthetic construct
  source 1..25: mol_type = other DNA
tgtaacagcaactccatgtggacta

SEQ ID NO: 625 - DNA, 22 bp, synthetic construct
  source 1..22: mol_type = other DNA
tcgaccggacctcgaccggcta

SEQ ID NO: 626 - DNA, 24 bp, synthetic construct
  source 1..24: mol_type = other DNA
tgagatgaagcactgtagctcata

SEQ ID NO: 627 - DNA, 23 bp, synthetic construct
  source 1..23: mol_type = other DNA
tttcagtcggatgtttgcagcaa

SEQ ID NO: 628 - DNA, 21 bp, synthetic construct
  source 1..21: mol_type = other DNA
gacctatgaattgacagccat

SEQ ID NO: 629 - DNA, 23 bp, synthetic construct
  source 1..23: mol_type = other DNA
ccactgccccaggtgctgctgga

SEQ ID NO: 630 - DNA, 25 bp, synthetic construct
  source 1..25: mol_type = other DNA
ctgacctatgaattgacagccatga

SEQ ID NO: 631 - DNA, 24 bp, synthetic construct
  source 1..24: mol_type = other DNA
accacagggtagaaccacggacga

SEQ ID NO: 632 - DNA, 23 bp, synthetic construct
  source 1..23: mol_type = other DNA
tcgaccggacctcgaccggctga

SEQ ID NO: 633 - DNA, 24 bp, synthetic construct
  source 1..24: mol_type = other DNA
tggctcagttcagcaggaacagga

SEQ ID NO: 634 - DNA, 23 bp, synthetic construct
  source 1..23: mol_type = other DNA
agcttatcagactgatgttgaaa

SEQ ID NO: 635 - DNA, 21 bp, synthetic construct
  source 1..21: mol_type = other DNA
atcacattgccagggataaaa

SEQ ID NO: 636 - DNA, 23 bp, synthetic construct
  source 1..23: mol_type = other DNA
tcaacaaaatcactgatgctgga

SEQ ID NO: 637 - DNA, 19 bp, synthetic construct
  source 1..19: mol_type = other DNA
acattgccagggatttcca

SEQ ID NO: 638 - DNA, 20 bp, synthetic construct
  source 1..20: mol_type = other DNA
aacactgtctggtaaagatg